(19) United States
(12) Patent Application Publication
(10) Pub. No.: US 2010/0190687 A1
(43) Pub. Date: Jul. 29, 2010

(54) PYRIMIDINONE DERIVATIVES AND METHODS OF USE THEREOF

(76) Inventors: Craig D. Boyle, Branchburg, NJ (US); Santhosh Francis Neelamkavil, Scotch Plains, NJ (US); Samuel Chackalamannil, Califon, NJ (US); Bernard R. Neustadt, West Orange, NJ (US); Jinsong Hao, Belle Mead, NJ (US); Unmesh G. Shah, Green Brook, NJ (US); Joel M. Harris, Summit, NJ (US); Hong Liu, Hillsborough, NJ (US); Andrew W. Stamford, Chatham Township, NJ (US)

Correspondence Address: MERCK;PATENT DEPARTMENT (K-6-1, 1990), 2000 GALLOPING HILL ROAD, KENILWORTH, NJ, 07033-0530, US

(21) Appl. No.: 12/596,341

(22) PCT Filed: Apr. 17, 2008
(86) PCT No.: PCT/US08/04933

§ 371 Date: Mar. 26, 2010

Related U.S. Application Data
(60) Provisional application No. 60/925,450, filed on Apr. 20, 2007.
(60) Provisional application No. 60/953,342, filed on Aug. 1, 2007.

(51) Int. Cl. A61K 38/28; C07D 487/04; A61K 31/519; A61K 31/55; A61K 38/22; A61P 3/10; A61P 3/04

(52) U.S. Cl. 514/4; 544/279; 514/264.1; 514/264.11; 540/578; 514/215; 544/280; 514/265.1; 514/12

(57) ABSTRACT

The present invention relates to Pyrimidinone Derivatives, compositions comprising a Pyrimidinone Derivative, and methods of using the Pyrimidinone Derivatives for treating or preventing obesity, diabetes, a metabolic disorder, a cardiovascular disease or a disorder related to the activity of G protein-coupled receptor 119 (“GPR119”) in a patient.

### FIELD OF THE INVENTION

The present invention relates to Pyrimidinone Derivatives, compositions comprising a Pyrimidinone Derivative, and methods of using the Pyrimidinone Derivatives for treating or preventing obesity, diabetes, a metabolic disorder, a cardiovascular disease or a disorder related to the activity of G protein-coupled receptor 119 (“GPR119”) in a patient.

### BACKGROUND OF THE INVENTION

Although a number of receptor classes exist in humans, by far the most abundant and therapeutically relevant is represented by the G protein-coupled receptor (GPCR or GPCRs) class. It is estimated that there are some 100,000 genes within the human genome, and of these, approximately 2% or 2,000 genes, are estimated to code for GPCRs. Receptors, including GPCRs, for which the endogenous ligand has been identified are referred to as “known” receptors, while receptors for which the endogenous ligand has not been identified are referred to as “orphan” receptors. GPCRs represent an important area for the development of pharmaceutical products, as evidenced by the fact that pharmaceutical products have been developed from approximately 20 of the 100 known GPCRs. This distinction is not merely semantic, particularly in the case of GPCRs. Thus, the orphan GPCRs are to the pharmaceutical industry what gold was to California in the late 19th century—an opportunity to drive growth, expansion, enhancement and development.

GPCRs share a common structural motif. All these receptors have seven sequences of between 22 to 24 hydrophobic amino acids that form seven alpha helices, each of which spans the membrane (each span is identified by number, i.e., transmembrane-1 (TM-1), transmembrane-2 (TM-2), etc.). The transmembrane helices are joined by strands of amino acids between transmembrane-2 and transmembrane-3, transmembrane-4 and transmembrane-5, and transmembrane-6 and transmembrane-7 on the exterior, or “extracellular” side, of the cell membrane (these are referred to as “extracellular” regions 1, 2 and 3 (EC-1, EC-2 and EC-3), respectively). The transmembrane helices are also joined by strands of amino acids between transmembrane-1 and transmembrane-2, transmembrane-3 and transmembrane-4, and transmembrane-5 and transmembrane-6 on the interior, or “intracellular” side, of the cell membrane (these are referred to as “intracellular” regions 1, 2 and 3 (IC-1, IC-2 and IC-3), respectively). The “carboxy” (“C”) terminus of the receptor lies in the intracellular space within the cell, and the “amino” (“N”) terminus of the receptor lies in the extracellular space outside of the cell.

Generally, when an endogenous ligand binds with the receptor (often referred to as “activation” of the receptor), there is a change in the conformation of the intracellular region that allows for coupling between the intracellular region and an intracellular “G-protein.” It has been reported that GPCRs are “promiscuous” with respect to G proteins, i.e., that a GPCR can interact with more than one G protein. See, Kenakin, T., Life Sciences 43:1095 (1988). Although other G proteins exist, currently, Gq, Gs, Gi, and Go are G proteins that have been identified. Endogenous ligand-activated GPCR coupling with the G-protein begins a signaling cascade process (referred to as “signal transduction”). Under normal conditions, signal transduction ultimately results in cellular activation or cellular inhibition. It is thought that the IC-3 loop as well as the carboxy terminus of the receptor interact with the G protein.

Under physiological conditions, GPCRs exist in the cell membrane in equilibrium between two different conformations: an “inactive” state and an “active” state. A receptor in an inactive state is unable to link to the intracellular signaling transduction pathway to produce a biological response. Changing the receptor conformation to the active state allows linkage to the transduction pathway (via the G-protein) and produces a biological response. A receptor can be stabilized in an active state by an endogenous ligand or a compound such as a drug.

Modulation of G-protein coupled receptors has been well-studied for controlling various metabolic disorders. Small molecule modulators of the receptor GPR119, a G-protein coupled-receptor described in, for example, GenBank (see, e.g., accession numbers XM.sub.-066873 and AY288416), have been shown to be useful for treating or preventing certain metabolic disorders. GPR119 is a G protein-coupled receptor that is selectively expressed on pancreatic beta cells. GPR119 activation leads to elevation of a level of intracellular cAMP, consistent with GPR119 being coupled to Gs. Agonists to GPR119 stimulate glucose-dependent insulin secretion in vitro and lower an elevated blood glucose level in vivo. See, e.g., International Publication Nos. WO 04/065380 and WO 04/076413, and European Patent Application No. EP 1338651, the disclosure of each of which is herein incorporated by reference in its entirety.

U.S. Pat. No. 7,132,426 discloses pyrazolo[3,4-d]pyrimidine ethers and related compounds as modulators of the GPR119 receptor that are useful for the treatment of various metabolic-related disorders such as type I diabetes, type II diabetes, inadequate glucose tolerance, insulin resistance, hyperglycemia, hyperlipidemia, hypertriglyceridemia, hypercholesterolemia, dyslipidemia or syndrome X. The compounds are also reported as being useful for controlling weight gain, controlling food intake, and inducing satiety in mammals. The promising nature of these GPR119 modulators indicates a need in the art for additional small molecule GPR119 modulators with improved efficacy and safety profiles. This invention addresses that need.

### SUMMARY OF THE INVENTION

In one aspect, the present invention provides compounds of Formula (I):

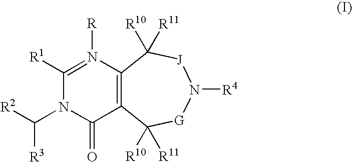

and pharmaceutically acceptable salts, solvates, esters and prodrugs thereof,
wherein

J is a single bond, —C(R¹⁰)(R¹¹)— or —C(R¹⁰)(R¹¹)—C(R¹⁰)(R¹¹)—;

G is a single bond, —C(R¹⁰)(R¹¹)— or —C(R¹⁰)(R¹¹)—C(R¹⁰)(R¹¹)—, such that: (i) if J is —C(R¹⁰)(R¹¹)—, then G is —C(R¹⁰)(R¹¹)— or —C(R¹⁰)(R¹¹)—C(R¹⁰)(R¹¹)—; and (ii) if J is —C(R¹⁰)(R¹¹)—C(R¹⁰)(R¹¹)—, then G is a single bond;

R is absent or R is oxygen, such that when R is oxygen, this is understood to represent the N-oxide form of the nitrogen atom to which R is attached;

R¹ is —H, alkyl, haloalkyl, —N(R⁹)₂, —SR⁹, —S(O)_{q}N(R⁶)₂, —S(O)ₚR⁷, —OR⁹, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heteroaryl, -(alkylene)ₙ-heterocycloalkenyl, —C(O)-aryl, —C(O)-alkyl, -alkylene-O-aryl, -alkylene-O-alkyl or —C(O)NH₂, wherein an aryl, cycloalkyl, cycloalkenyl, heterocycloalkyl, heterocycloalkenyl or heteroaryl group can be optionally substituted with up to 3 substituents, which can be the same or different, and are selected from alkyl, haloalkyl, hydroxyalkyl, aryl, halo, —OH, —O-haloalkyl, —O-alkyl, -alkylene-O-alkyl, —S(O)ₚR⁷, —CN, —N(R⁶)₂, —C(O)R⁵, —C(O)OR⁵, —C(O)N(R⁶)₂, —NHC(O)R⁵, —NHS(O)_{q}R⁷ and —S(O)_{q}N(R⁶)₂;

R² is alkyl, -alkenyl, -alkynyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heteroaryl, -(alkylene)ₙ-heterocycloalkenyl, -(alkylene)ₙ-OC(O)N(R⁶)₂, hydroxyalkyl, haloalkyl, -alkylene-alkenyl, —C(O)-aryl, —C(O)-alkyl, —C(O)-heterocycloalkyl, —C(O)-heteroaryl, -alkylene-O-aryl, -alkylene-O-alkyl, -alkylene-O-haloalkyl, —C(O)OR⁵, or —C(O)N(R⁶)₂, wherein an aryl, cycloalkyl, cycloalkenyl, heterocycloalkyl, heterocycloalkenyl or heteroaryl group can be optionally substituted with up to 3 substituents, which can be the same or different, and are selected from alkyl, haloalkyl, hydroxyalkyl, aryl, halo, —OH, —O-haloalkyl, —O-alkyl, -alkylene-O-alkyl, —Si(alkyl)₃, —S(O)ₚR⁷, —CN, —N(R⁶)₂, —C(O)R⁵, —C(O)OR⁵, —C(O)N(R⁶)₂, —NHC(O)R⁵, —NHS(O)_{q}R⁷ and —S(O)_{q}N(R⁶)₂, and wherein a cycloalkyl group may form a spirocycle with a heterocycloalkyl group or with another cycloalkyl group, or R² and R³ and the carbon atom to which they are both attached, combine to form an aryl, cycloalkyl, cycloalkenyl, heterocycloalkyl, heterocycloalkenyl or heteroaryl group, wherein any of these groups is unsubstituted or substituted with up to 3 substituents, which can be the same or different, and which are selected from alkyl, haloalkyl, hydroxyalkyl, halo, —OH, —O-haloalkyl, —O-alkyl, -D-aryl, -alkylene-O-alkyl, —CN, —N(R⁶)₂, —C(O)R⁵, —C(O)OR⁵, —C(O)N(R⁶)₂, —NHC(O)R⁵, —NHS(O)_{q}R⁷, —S(O)ₚR⁷ and —S(O)_{q}N(R⁶)₂;

R³ is alkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heteroaryl, -(alkylene)ₙ-heterocycloalkenyl, —C(O)-aryl, —C(O)-alkyl, -alkylene-O-aryl, -alkylene-O-alkyl, —C(O)OR⁵, or —C(O)N(R⁶)₂, wherein an aryl, cycloalkyl, cycloalkenyl, heterocycloalkyl, heterocycloalkenyl or heteroaryl group can be optionally substituted with up to 3 substituents, which can be the same or different, and are selected from alkyl, haloalkyl, hydroxyalkyl, aryl, halo, —OH, —O-haloalkyl, —O-alkyl, -alkylene-O-alkyl, —S(O)ₚR⁷, —CN, —N(R⁶)₂, —C(O)R⁵, —C(O)OR⁵, —C(O)N(R⁶)₂, —NHC(O)R⁵, —NHS(O)_{q}R⁷ and —S(O)_{q}N(R⁶)₂, or R² and R³ and the carbon atom to which they are both attached, combine to form an aryl, cycloalkyl, cycloalkenyl, heterocycloalkyl, heterocycloalkenyl or heteroaryl group, wherein any of these groups is unsubstituted or substituted with up to 3 substituents, which can be the same or different, and which are selected from alkyl, haloalkyl, hydroxyalkyl, halo, —OH, —O-haloalkyl, —O-alkyl, —O-aryl, -alkylene-O-alkyl, —CN, —N(R⁶)₂, —C(O)R⁵, —C(O)OR⁵, —C(O)N(R⁶)₂, —NHC(O)R⁵, —NHS(O)_{q}R⁷, —S(O)ₚR⁷ and —S(O)_{q}N(R⁶)₂;

R⁴ is H, alkyl, alkenyl, —C(O)R⁵, —S(O)_{q}R⁷, -alkylene-O-alkyl, -alkylene-O-aryl, -alkylene-S-alkyl, -alkylene-S-aryl, -alkylene-NH-alkyl, -alkylene-NH-aryl, -alkylene-NC(O)O-alkyl, —C(O)OR⁵, —C(O)N(R⁶)₂, —C(O)NH—OR⁸, -alkylene-O-haloalkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl, -(alkylene)ₙ-heteroaryl, -(alkenylene)ₙ-aryl, -(alkenylene)ₙ-cycloalkyl, -(alkenylene)ₙ-cycloalkenyl, -(alkenylene)ₙ-heterocycloalkyl, -(alkenylene)ₙ-heterocycloalkenyl or -(alkenylene)ₙ-heteroaryl, wherein any alkylene or alkenylene group can be optionally substituted with one or more substituents independently selected from alkyl, haloalkyl, hydroxyalkyl, —O-alkyl, aryl, cycloalkyl, cycloalkenyl, heterocycloalkyl, heterocycloalkenyl or heteroaryl, and wherein any aryl, cycloalkyl, cycloalkenyl, heterocycloalkyl, heterocycloalkenyl or heteroaryl group can be optionally substituted with up to 3 substituents, which can be the same or different, and are selected from: alkyl, aryl, heterocycloalkyl, heteroaryl, -alkylene-O-alkylene-Si(alkyl)₃, —NH₂, —NH-alkyl, —N(alkyl)₂, —OH, -hydroxyalkyl, —S(O)ₚR⁷, —O-alkyl, —O-aryl, —C(O)O-alkyl, —C(O)O-haloalkyl, halo, —NO₂, —CN, heteroaryl, haloalkyl, —O-haloalkyl, and -(alkynylene)ₙ-aryl;

R⁵ is alkyl, alkenyl, alkynyl, haloalkyl, haloalkenyl, -alkylene-O-aryl, -alkylene-S-aryl, -alkylene-N(R⁸)C(O)O-alkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl or -(alkylene)ₙ-heteroaryl, wherein a cycloalkyl group may form a spirocycle with a heterocycloalkyl group or with another cycloalkyl group, and wherein an aryl, alkenyl, alkynyl, cycloalkyl, cycloalkenyl, heterocycloalkyl, heterocycloalkenyl or heteroaryl group can be unsubstituted or substituted with up to 4 substituents, which can be the same or different, and are selected from alkyl, haloalkyl, hydroxyalkyl, halo, —OH, —O-haloalkyl, —O-alkyl, —O-aryl, —S-haloalkyl, -alkylene-O-alkyl, —CN, —N(R⁹)₂, —C(O)H, —C(O)R⁹, —C(O)OR⁹, —C(O)N(R⁹)₂, —NHC(O)R⁹, —NHS(O)_{q}R⁹, —S(O)ₚR⁹ and —S(O)_{q}N(R⁹)₂;

each occurrence of R⁶ is independently H, alkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl or -(alkylene)ₙ-heteroaryl, wherein any of the above groups, excluding H, can be unsubstituted or substituted with from 1 to 3 substituents, which can be the same or different, and which are selected from alkyl, haloalkyl, hydroxyalkyl, halo, —OH, —O-haloalkyl, —O-alkyl, —O-aryl, -alkylene-O-alkyl, —CN, —N(R⁹)₂, —C(O)H, —C(O)R⁹, —C(O)OR⁹, —C(O)N(R⁹)₂, —NHC(O)R⁹, —NHS(O)_{q}R⁹, —S(O)ₚR⁹ and —S(O)_{q}N(R⁹)₂;

each occurrence of R⁷ is independently alkyl, aryl, heterocycloalkyl, heteroaryl or cycloalkyl, wherein any of the above groups, can be unsubstituted or substituted with from 1 to 3 substituents, which can be the same or different, and which are selected from alkyl, haloalkyl, hydroxyalkyl, halo, —OH, —O-haloalkyl, —O-alkyl, —O-aryl, -alkylene-O-alkyl, —CN, —N(R⁹)₂, —C(O)H, —C(O)R⁹, —C(O)OR⁹, —C(O)N(R⁹)₂, —NHC(O)R⁹, —NHS(O)_{q}R⁹, —S(O)ₚR⁹ and —S(O)_{q}N(R⁹)₂;

each occurrence of R⁸ is independently H or alkyl;

each occurrence of R⁹ is independently H, alkyl, -(alkylene)ₙ-aryl, heterocycloalkyl, heteroaryl or cycloalkyl;

each occurrence of R¹⁰ is independently H, alkyl, -(alkylene)ₙ-aryl, heterocycloalkyl, heteroaryl or cycloalkyl;

each occurrence of R¹¹ is independently H, alkyl, -(alkylene)ₙ-aryl, heterocycloalkyl, heteroaryl or cycloalkyl;

each occurrence of n is independently 0 or 1;

each occurrence of p is independently 0, 1 or 2; and

each occurrence of q is independently 1 or 2.

The compounds of formula (I) or pharmaceutically acceptable salts, solvates, esters or prodrugs thereof (referred to herein as the “Pyrimidinone Derivatives”) can be useful for treating or preventing obesity, diabetes, metabolic syndrome, a cardiovascular disease or a disorder related to the activity of GPR119 (each being a “Condition”) in a patient.

Also provided by the invention are methods for treating or preventing a Condition in a patient, comprising administering to the patient an effective amount of one or more Pyrimidinone Derivatives.

The present invention further provides pharmaceutical compositions comprising an effective amount of one or more Pyrimidinone Derivatives or a pharmaceutically acceptable salt, solvate, ester or prodrug thereof, and a pharmaceutically acceptable carrier. The compositions can be useful for treating or preventing a Condition in a patient.

The details of the invention are set forth in the accompanying detailed description below.

Although any methods and materials similar to those described herein can be used in the practice or testing of the present invention, illustrative methods and materials are now described. Other features, objects, and advantages of the invention will be apparent from the description and the claims. All patents and publications cited in this specification are incorporated herein by reference.

### DETAILED DESCRIPTION OF THE INVENTION

In an embodiment, the present invention provides Pyrimidinone Derivatives of Formula (I), pharmaceutical compositions comprising one or more Pyrimidinone Derivatives, and methods of using the Pyrimidinone Derivatives for treating or preventing a Condition in a patient.

### Definitions and Abbreviations

As used above, and throughout this disclosure, the following terms, unless otherwise indicated, shall be understood to have the following meanings:

A “patient” is a human or non-human mammal. In one embodiment, a patient is a human. In another embodiment, a patient is a non-human mammal, including, but not limited to, a monkey, dog, baboon, rhesus, mouse, rat, horse, cat or rabbit. In another embodiment, a patient is a companion animal, including but not limited to a dog, cat, rabbit, horse or ferret. In one embodiment, a patient is a dog. In another embodiment, a patient is a cat.

The term “obesity” as used herein, refers to a patient being overweight and having a body mass index (BMI) of 25 or greater. In one embodiment, an obese patient has a BMI of about 25 or greater. In another embodiment, an obese patient has a BMI of between about 25 and about 30. In another embodiment, an obese patient has a BMI of between about 35 and about 40. In still another embodiment, an obese patient has a BMI greater than 40.

The term “obesity-related disorder” as used herein refers to: (i) disorders which result from a patient having a BMI of about 25 or greater; and (ii) eating disorders and other disorders associated with excessive food intake. Non-limiting examples of an obesity-related disorder include edema, shortness of breath, sleep apnea, skin disorders and high blood pressure.

The term “metabolic syndrome” as used herein, refers to a set of risk factors that make a patient more susceptible to cardiovascular disease and/or type 2 diabetes. As defined herein, a patient is considered to have metabolic syndrome if the patient has one or more of the following five risk factors:
1) central/abdominal obesity as measured by a waist circumference of greater than 40 inches in a male and greater than 35 inches in a female;
2) a fasting triglyceride level of greater than or equal to 150 mg/dL;
3) an HDL cholesterol level in a male of less than 40 mg/dL or in a female of less than 50 mg/dL;
4) blood pressure greater than or equal to 130/85 mm Hg; and
5) a fasting glucose level of greater than or equal to 110 mg/dL.

The term “effective amount” as used herein, refers to an amount of compound of formula (I) and/or an additional therapeutic agent, or a composition thereof that is effective in producing the desired therapeutic, ameliorative, inhibitory or preventative effect when administered to a patient suffering from a Condition. In the combination therapies of the present invention, an effective amount can refer to each individual agent or to the combination as a whole, wherein the amounts of all agents administered are together effective, but wherein the component agent of the combination may not be present individually in an effective amount.

The term “alkyl,” as used herein, refers to an aliphatic hydrocarbon group which may be straight or branched and which contains from about 1 to about 20 carbon atoms. In one embodiment, an alkyl group contains from about 1 to about 12 carbon atoms. In another embodiment, an alkyl group contains from about 1 to about 6 carbon atoms. Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is unsubstituted. In another embodiment, an alkyl group is linear. In another embodiment, an alkyl group is branched.

The term “alkenyl,” as used herein, refers to an aliphatic hydrocarbon group containing at least one carbon-carbon double bond and which may be straight or branched and contains from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkyl, aryl, cycloalkyl, cyano, alkoxy and —S(alkyl). In one embodiment, an alkenyl group is unsubstituted.

The term “alkynyl,” as used herein, refers to an aliphatic hydrocarbon group containing at least one carbon-carbon triple bond and which may be straight or branched and contains from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of alkyl, aryl and cycloalkyl. In one embodiment, an alkynyl group is unsubstituted.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂— and —CH₂CH(CH₃)CH₂—. An alkylene group may be unsubstituted or substituted by one or more substituents which may be the same or different, each substituent being independently selected from the group consisting of halo, alkyl, aryl, cycloalkyl, cyano, alkoxy and —S(alkyl). In one embodiment, an alkylene group is unsubstituted. In another embodiment, an alkylene group has from 1 to about 6 carbon atoms. In another embodiment, an alkylene group is branched. In still another embodiment, an alkylene group is linear.

The term “alkenylene,” as used herein, refers to an alkenyl group, as defined above, wherein one of the alkenyl group's hydrogen atoms has been replaced with a bond. Non-limiting examples of alkenylene groups include —CH═CH—, —CH₂CH═CH—, —CH₂CH═CHCH₂—, —CH═CHCH₂CH₂—, —CH₂CHCH═CH—, —CH(CH₃)CH═CH— and —CH═C(CH₃)CH₂—. In one embodiment, an alkenylene group has from 2 to about 6 carbon atoms. In another embodiment, an alkenylene group is branched. In another embodiment, an alkenylene group is linear.

The term “alkynylene,” as used herein, refers to an alkynyl group, as defined above, wherein one of the alkynyl group's hydrogen atoms has been replaced with a bond. Non-limiting examples of alkynylene groups include —C≡C—, —CH₂C≡C—, —CH₂C≡CCH₂—, —C≡CCH₂CH₂—, —CH₂CHC≡C—, —CH(CH₃)C≡C— and —C≡CCH₂—. In one embodiment, an alkynylene group has from 2 to about 6 carbon atoms. In another embodiment, an alkynylene group is branched. In another embodiment, an alkynylene group is linear.

“Aryl” means an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. Non-limiting examples of aryl groups include phenyl and naphthyl. In one embodiment, an aryl group is unsubstituted. In another embodiment, an aryl group is phenyl.

The term “cycloalkyl,” as used herein, refers to a non-aromatic mono- or multicyclic ring system comprising from about 3 to about 10 ring carbon atoms. In one embodiment, a cycloalkyl contains from about 3 to about 7 ring carbon atoms. In another embodiment, a cycloalkyl contains from about 5 to about 7 ring atoms. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. A cycloalkyl group can also have one or more of its ring carbon atoms replaced with a carbonyl group to form, for example, a cyclopentanoyl or cyclohexanoyl group. In one embodiment, a cycloalkyl group is unsubstituted.

The term “cycloalkenyl,” as used herein, refers to a non-aromatic mono- or multicyclic ring system comprising from about 3 to about 10 ring carbon atoms and containing at least one endocyclic double bond. In one embodiment, a cycloalkenyl contains from about 5 to about 10 ring carbon atoms. In another embodiment, a cycloalkenyl contains 5 or 6 ring atoms. Non-limiting examples of monocyclic cycloalkenyls include cyclopentenyl, cyclohexenyl, cyclohepta-1,3-dienyl, and the like. A cycloalkenyl group can be optionally substituted with one or more “ring system substituents” which may be the same or different, and are as defined herein below. In one embodiment, a cycloalkenyl group is unsubstituted. In another embodiment, a cycloalkenyl group is a 5-membered cycloalkenyl.

The term “5-membered cycloalkenyl,” as used herein, refers to a cycloalkenyl group, as defined above, which has 5 ring carbon atoms.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. A heteroaryl group can be optionally substituted by one or more “ring system substituents” which may be the same or different, and are as defined herein below. A heteroaryl group is joined via a ring carbon atom, and any nitrogen atom of a heteroaryl can be optionally oxidized to the corresponding N-oxide. The term “heteroaryl” also encompasses a heteroaryl group, as defined above, which has been fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like. In one embodiment, a heteroaryl group is unsubstituted. In another embodiment, a heteroaryl group is a 5-membered heteroaryl.

The term “5-membered heteroaryl,” as used herein, refers to a heteroaryl group, as defined above, which has 5 ring atoms.

The term “heterocycloalkyl,” as used herein, refers to a non-aromatic saturated monocyclic or multicyclic ring system comprising 3 to about 10 ring atoms, wherein from 1 to 4 of the ring atoms are independently O, S or N and the remainder of the ring atoms are carbon atoms. In one embodiment, a heterocycloalkyl group has from about 5 to about 10 ring atoms. In another embodiment, a heterocycloalkyl group has 5 or 6 ring atoms. There are no adjacent oxygen and/or sulfur atoms present in the ring system. Any —NH group in a heterocycloalkyl ring may exist protected such as, for example, as an —N(BOC), —N(Cbz), —N(Tos) group and the like; such protected heterocycloalkyl groups are considered part of this invention. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. Non-limiting examples of monocyclic heterocycloalkyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. A ring carbon atom of a heterocycloalkyl group may be functionalized as a carbonyl group. An illustrative example of such a heterocycloalkyl group is pyrrolidonyl:

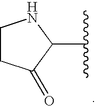

In one embodiment, a heterocycloalkyl group is unsubstituted. In another embodiment, a heterocycloalkyl group is a 5-membered heterocycloalkyl.

The term “5-membered heterocycloalkyl,” as used herein, refers to a heterocycloalkyl group, as defined above, which has 5 ring atoms.

The term “heterocycloalkenyl,” as used herein, refers to a heterocycloalkyl group, as defined above, wherein the heterocycloalkyl group contains from 3 to 10 ring atoms, and at least one endocyclic carbon-carbon or carbon-nitrogen double bond. In one embodiment, a heterocycloalkenyl group has from 5 to 10 ring atoms. In another embodiment, a heterocycloalkenyl group is monocyclic and has 5 or 6 ring atoms. A heterocycloalkenyl group can be optionally substituted by one or more ring system substituents, wherein “ring system substituent” is as defined above. The nitrogen or sulfur atom of the heterocycloalkenyl can be optionally oxidized to the corresponding N-oxide, S-oxide or S,S-dioxide. Non-limiting examples of heterocycloalkenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluoro-substituted dihydrofuranyl, 7-oxabicyclo[2.2.1]heptenyl, dihydrothiophenyl, dihydrothiopyranyl, and the like. A ring carbon atom of a heterocycloalkenyl group may be functionalized as a carbonyl group. An illustrative example of such a heterocycloalkenyl group is:

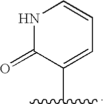

In one embodiment, a heterocycloalkenyl group is unsubstituted. In another embodiment, a heterocycloalkenyl group is a 5-membered heterocycloalkenyl.

The term “5-membered heterocycloalkenyl,” as used herein, refers to a heterocycloalkenyl group, as defined above, which has 5 ring atoms.

It should also be noted that tautomeric forms such as, for example, the moieties:

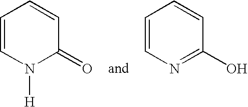

are considered equivalent in certain embodiments of this invention.

The term “ring system substituent,” as used herein, refers to a substituent group attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, -alkylene-aryl, -alkylene-heteroaryl, -alkenylene-heteroaryl, -alkynylene-heteroaryl, hydroxy, hydroxyalkyl, haloalkyl, —O-alkyl, -alkylene-O-alkyl, —O-aryl, aralkoxy, acyl, aroyl, halo, nitro, cyano, carboxy, —C(O)O-alkyl, —C(O)O-aryl, —C(O)O-alkelene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), Y₁Y₂N—, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, cycloalkyl, and alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

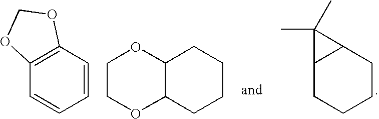

“Halo” means —Cl, —Br or —I. In one embodiment, halo refers to —Cl or —Br.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 6 F atoms. In another embodiment, a haloalkyl group is substituted with from 1 to 3 F atoms. Non-limiting examples of haloalkyl groups include CH₂F, —CF₃, —CH₂Cl and —CCl₃.

The term “haloalkenyl,” as used herein, refers to an alkenyl group as defined above, wherein one or more of the alkenyl group's hydrogen atoms has been replaced with a halogen. In one embodiment, a haloalkenyl group has from 1 to 6 carbon atoms. In another embodiment, a haloalkenyl group is substituted with from 1 to 6 F atoms. In another embodiment, a haloalkenyl group is substituted with from 1 to 3 F atoms. Non-limiting examples of haloalkenyl groups include —CH═CF₂ and —CH═CHCF₃.

The term “hydroxyalkyl,” as used herein, refers to an alkyl group as defined above, wherein one or more of the alkyl group's hydrogen atoms has been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃.

The term “alkoxy” as used herein, refers to an —O-alkyl group, wherein an alkyl group is as defined above. Non-limiting examples of alkoxy groups include methoxy, ethoxy, n-propoxy, isopropoxy, n-butoxy and t-butoxy. An alkoxy group is bonded via its oxygen atom.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “purified”, “in purified form” or “in isolated and purified form” for a compound refers to the physical state of the compound after being isolated from a synthetic process (e.g. from a reaction mixture), or natural source or combination thereof. Thus, the term “purified”, “in purified form” or “in isolated and purified form” for a compound refers to the physical state of the compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any variable (e.g., aryl, heterocycle, R², etc.) occurs more than one time in any constituent or in Formula (I), its definition on each occurrence is independent of its definition at every other occurrence, unless otherwise noted.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a Pyrimidinone Derivative or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a Pyrimidinone Derivative or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a Pyrimidinone Derivative contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a Pyrimidinone Derivative incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. “Hydrate” is a solvate wherein the solvent molecule is H₂O.

One or more compounds of the invention may optionally be converted to a solvate. Preparation of Solvates is Generally Known. Thus, for Example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The Pyrimidinone Derivatives can form salts which are also within the scope of this invention. Reference to a Pyrimidinone Derivative herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Pyrimidinone Derivative contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula (I) may be formed, for example, by reacting a Pyrimidinone Derivative with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (for example, methyl, ethyl, n-propyl, isopropyl, t-butyl, sec-butyl or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the Pyrimidinone Derivatives may be atropisomers (e.g., substituted biaryls) and are considered as part of this invention. Enantiomers can also be separated by use of chiral HPLC column.

It is also possible that the Pyrimidinone Derivatives may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a Pyrimidinone Derivative incorporates a double bond or a fused ring, both the cis- and trans-forms, as well as mixtures, are embraced within the scope of the invention. Also, for example, all keto-enol and imine-enamine forms of the compounds are included in the invention).

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled Pyrimidinone Derivatives (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled Pyrimidinone Derivatives can generally be prepared using synthetic chemical procedures analogous to those disclosed herein for making the Compounds of Formula (I), by substituting an appropriate isotopically labelled starting material or reagent for a non-isotopically labelled starting material or reagent.

Polymorphic forms of the Pyrimidinone Derivatives, and of the salts, solvates, hydrates, esters and prodrugs of the Pyrimidinone Derivatives, are intended to be included in the present invention.

The following abbreviations are used below and have the following meanings: AcOH is acetic acid, Boc or BOC is —C(O)O-(t-butyl), n-BuLi is n-butyllithium, t-butyl is tertiary butyl, DAST is diethylaminosulfur trifluoride, dba is dibenzylidene acetone, DCE is dichloroethane, DCM is dichloromethane, DIAD is diisopropylazodicarboxylate, DIEA is diisopropylethylamine, DMEM is Dulbecco's modified eagle medium, DMF is N,N-dimethylformamide, DMSO is dimethylsulfoxide, dppf is 1,1′-bis(diphenylphosphino)ferrocene, EtOAc is ethyl acetate, EtOH is ethanol, Et₃N is triethylamine, EtNH₂ is ethylamine, HOBt is 1-hydroxy-benzotriazole, LCMS is liquid chromatography mass spectrometry, LDA is lithiumdiisopropylamide, mCPBA is meta-chloroperoxybenzoic acid, MeOH is methanol, NaOEt is sodium ethoxide, NaOtBu is sodium t-butoxide, NMM is N-methylmorpholine, NMR is nuclear magnetic resonance, Ph is phenyl, PhMe is toluene, PLC is preparative layer chromatography, PS-EDC is polystyrene functionalized with EDC-1-(dimethylaminopropyl)-3-ethylcarbodiimide—available from Polymer Laboratories, PS-DIEA is polystyrene functionalized with disopropylethylamine, TBAF is tetra-n-butyl-ammonium fluoride, THF is tetrahydrofuran, and TLC is thin-layer chromatography.

### The Pyrimidinone Derivatives of Formula (I)

The present invention provides Pyrimidinone Derivatives of Formula (I):

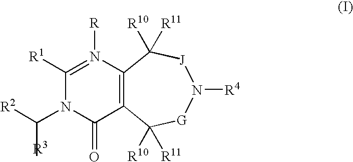

and pharmaceutically acceptable salts, solvates, esters and prodrugs thereof, wherein J, G, R, R¹, R², R³, R⁴, R¹⁰ and R¹¹ are defined above for the compounds of formula (I).

In one embodiment, J is a single bond.

In another embodiment, J is —C(R¹⁰)(R¹¹)— and G is other than a single bond.

In another embodiment, J is —C(R¹⁰)(R¹¹)—C(R¹⁰)(R¹¹)— and G is —C(R¹⁰)(R¹¹)— or —C(R¹⁰)(R¹¹)—C(R¹⁰)(R¹¹)—.

In still another embodiment, J is —CH₂—.

In another embodiment, G is —C(R¹⁰)(R¹¹)—.

In another embodiment, G is —C(R¹⁰)(R¹¹)—C(R¹⁰)(R¹¹)—.

In still another embodiment, G is —CH₂—.

In one embodiment, J and G are each —C(R¹⁰)(R¹¹)—.

In one embodiment, J and G are each —C(R¹⁰)(R¹¹)— and each occurrence of R¹⁰ and R¹¹ is H.

In another embodiment, J and G are each a single bond.

In another embodiment, J and G are each a single bond and each occurrence of R¹⁰ and R¹¹ is H.

In another embodiment, J is a single bond and G is —C(R¹⁰)(R¹¹)—.

In another embodiment, J is a single bond, G is —C(R¹⁰)(R¹¹)— and each occurrence of R¹⁰ and R¹¹ is H.

In still another embodiment, J is a single bond and G is —CH₂—.

In still another embodiment, J is a single bond, G is —CH₂— and each occurrence of R¹⁰ and R¹¹ is H.

In one embodiment, R is absent.

In another embodiment, R is oxygen.

In one embodiment, R¹ is —H.

In one embodiment, R¹ is other than —H.

In another embodiment, R¹ is alkyl.

In another embodiment, R¹ is —N(R⁹)₂.

In still another embodiment, R¹ is —OR⁹.

In yet another embodiment, R¹ is —SR⁹.

In one embodiment, R¹ is —NH₂.

In another embodiment, R¹ is —NH-alkyl.

In another embodiment, R¹ is —N(alkyl)₂.

In still another embodiment, R¹ is —O-alkyl.

In a further embodiment, R¹ is —S-alkyl.

In another embodiment, R¹ is aryl.

In still another embodiment, R¹ is cycloalkyl.

In yet another embodiment, R¹ is cycloalkenyl.

In a further embodiment, R¹ is heterocycloalkyl.

In another embodiment, R¹ is heterocycloalkenyl.

In another embodiment, R¹ is heteroaryl.

In another embodiment, R¹ is -(alkylene)-aryl.

In still another embodiment, R¹ is -(alkylene)-cycloalkyl.

In yet another embodiment, R¹ is -(alkylene)-cycloalkenyl.

In a further embodiment, R¹ is -(alkylene)-heterocycloalkyl.

In another embodiment, R¹ is -(alkylene)-heterocycloalkenyl.

In another embodiment, R¹ is -(alkylene)-heteroaryl.

In still another embodiment, R¹ is haloalkyl.

In another embodiment, R¹ is fluoromethyl.

In another embodiment, R¹ is difluoromethyl.

In a further embodiment, R¹ is cyclopropyl.

In another embodiment, R¹ is alkenyl.

In another embodiment, R¹ is alkynyl.

In yet another embodiment, R¹ is propynyl.

In one embodiment, R¹ is methyl.

In another embodiment, R¹ is ethyl.

In another embodiment, R¹ is n-propyl.

In still another embodiment, R¹ isopropyl.

In a further embodiment, R¹ is benzyl.

In another embodiment, R¹ is phenyl.

In one embodiment, R² is aryl.

In another embodiment, R² is heteroaryl.

In still another embodiment, R² is alkyl.

In another embodiment, R² is benzyl.

In yet another embodiment, R² is cycloalkyl.

In another embodiment, R² is cyclopropyl, cyclobutyl, cyclopentyl or cyclohexyl.

In another embodiment, R² is heterocycloalkyl.

In a further embodiment, R² is —C(O)-aryl.

In another embodiment, R² is alkylene-aryl.

In another embodiment, R² is alkylene-O-aryl.

In another embodiment, R² is alkylene-O-alkyl.

In still another embodiment, R² is methyl.

In another embodiment, R² is phenyl.

In yet another embodiment, R² is 4-trifluoromethyl-phenyl.

In one embodiment, R² is 4-fluorophenyl.

In another embodiment, R² is 2-(4-fluorophenyl)ethyl.

In another embodiment, R² is pyridyl.

In still another embodiment, R² is 2-pyridyl.

In another embodiment, R² is —C(O)NH₂.

In another embodiment, R² is —C(O)OR⁵.

In another embodiment, R² is —C(O)N(R⁶)₂.

In one embodiment, R² is C(O)O-alkyl.

In another embodiment, R² is C(O)β-cycloalkyl.

In another embodiment, R² is C(O)O-alkylene-cycloalkyl.

In still another embodiment, R² is C(O)O—CH₂-phenyl.

In one embodiment, R² is C(O)NH-alkyl.

In another embodiment, R² is C(O)NH-cycloalkyl.

In another embodiment, R² is C(O)NH-alkylene-cycloalkyl.

In still another embodiment, R² is C(O)NH—CH₂-phenyl.

In another embodiment, R² is trifluoromethyl.

In yet another embodiment, R² is cyclopropyl.

In still another embodiment, R² is cyclobutyl.

In another embodiment, R² is cyclopentyl.

In one embodiment, R² is cyclohexyl.

In another embodiment, R² is alkylene-N(R⁹)₂

In another embodiment, R² is —CH₂—O-phenyl.

In one embodiment, R³ is aryl.

In another embodiment, R³ is heteroaryl.

In still another embodiment, R³ is alkyl.

In another embodiment, R³ is benzyl.

In yet another embodiment, R³ is cycloalkyl.

In one embodiment, R³ is phenyl, pyridyl, 4-fluorophenyl, 3-fluorophenyl, cyclopropylmethyl, ethoxymethyl, trifluoroethoxymethyl, n-butyl, cyclopropyl, cyclobutyl, cyclopentyl or cyclohexyl.

In another embodiment, R³ is cyclopropyl, cyclobutyl, cyclopentyl or cyclohexyl.

In another embodiment, R³ is heterocycloalkyl.

In a further embodiment, R³ is —C(O)-aryl.

In another embodiment, R³ is alkylene-aryl.

In another embodiment, R³ is alkylene-O-aryl.

In another embodiment, R³ is alkylene-O-alkyl.

In still another embodiment, R³ is methyl.

In another embodiment, R³ is phenyl.

In yet another embodiment, R³ is 4-trifluoromethyl-phenyl.

In one embodiment, R³ is 4-fluorophenyl.

In another embodiment, R³ is 2-(4-fluorophenyl)ethyl.

In another embodiment, R³ is pyridyl.

In still another embodiment, R³ is 2-pyridyl.

In another embodiment, R³ is —C(O)NH₂.

In another embodiment, R³ is —C(O)OR⁵.

In another embodiment, R³ is —C(O)N(R⁶)₂.

In still another embodiment, R³ is trifluoromethyl.

In yet another embodiment, R³ is cyclopropyl.

In still another embodiment, R³ is cyclobutyl.

In another embodiment, R³ is cyclopentyl.

In one embodiment, R³ is cyclohexyl.

In another embodiment, R³ is alkylene-N(R⁹)₂

In another embodiment, R³ is —CH₂—O-phenyl.

In one embodiment, R⁴ is H.

In another embodiment, R⁴ is alkyl.

In another embodiment, R⁴ is —S(O)_{q}R⁷.

In another embodiment, R⁴ is —C(O)R⁵.

In still another embodiment, R⁴ is -alkylene-O-alkyl.

In yet another embodiment, R⁴ is -alkylene-O-aryl.

In another embodiment, R⁴ is -alkylene-S-alkyl.

In another embodiment, R⁴ is -alkylene-S-aryl.

In another embodiment, R⁴ is -alkylene-NH-alkyl.

In yet another embodiment, R⁴ is -alkylene-NH-aryl.

In a further embodiment, R⁴ is C(O)OR⁵.

In one embodiment, R⁴ is C(O)O-(t-butyl).

In another embodiment, R⁴ is —C(O)N(R⁶)₂.

In another embodiment, R⁴ is -(alkylene)-aryl.

In another embodiment, R⁴ is -(alkylene)-cycloalkyl.

In still another embodiment, R⁴ is -(alkylene)-cycloalkenyl.

In yet another embodiment, R⁴ is -(alkylene)-heterocycloalkyl.

In a further embodiment, R⁴ is -(alkylene)-heterocycloalkenyl.

In another embodiment, R⁴ is -(alkylene)-heteroaryl.

In another embodiment, R⁴ is aryl.

In another embodiment, R⁴ is benzyl.

In another embodiment, R⁴ is cycloalkyl.

In still another embodiment, R⁴ is cycloalkenyl.

In yet another embodiment, R⁴ is heterocycloalkyl.

In a further embodiment, R⁴ is heterocycloalkenyl.

In another embodiment, R⁴ is heteroaryl.

In another embodiment, R⁴ is —CH₂-heteroaryl.

In still another embodiment, R⁴ is phenyl.

In yet another embodiment, R⁴ is pyrimidinyl.

In a further embodiment, R⁴ is 4-trifluoromethyl-phenyl.

In another embodiment, R⁴ is —C(O)O-2,2,3,3-tetrafluorocyclobutyl.

In another embodiment, R⁴ is —C(O)O-trans-4-(trifluoromethyl)cyclohexyl.

In one embodiment, R⁴ is —C(O)OR⁵, wherein R⁵ is alkyl, aryl, haloalkyl, -alkylene-aryl, -cycloalkyl, -alkylene-O-alkylene-aryl, -alkylene-O-alkyl, or alkynyl.

In another embodiment, R⁴ is —C(O)OR⁵, wherein R⁵ is methyl, ethyl, n-propyl, isopropyl, n-butyl, tert-butyl, -neopentyl, —CH₂CH(—CH₂CH₃)—(CH₂)₃CH₃, —CH₂CHCH₃)₂, n-hexyl or —CH₂—C≡CCH₃.

In another embodiment, R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

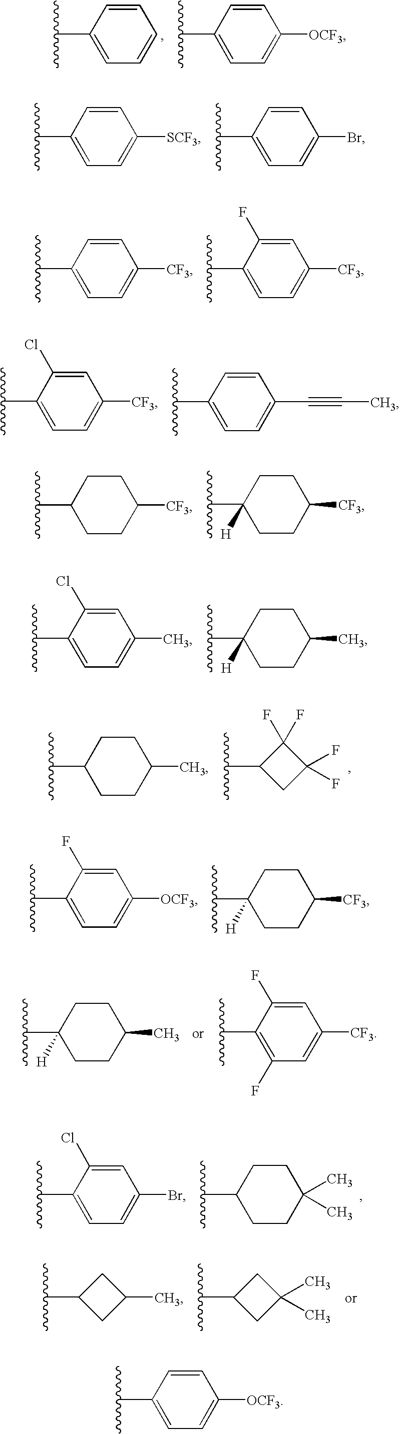

In still another embodiment, R⁴ is —C(O)OR⁵, wherein R⁵ is cyclopropyl, cyclobutyl, cyclopentyl or cyclohexyl.

In yet another embodiment, R⁴ is —C(O)OR⁵, wherein R⁵ is benzyl or 2-chlorobenzyl.

In another embodiment, R⁴ is —C(O)OR⁵, wherein R⁵ is —(CH₂)₂—O-benzyl or —(CH₂)₂—O—CH₃.

In another embodiment, R⁴ is —C(O)NHR⁵.

In still another embodiment, R⁴ is —C(O)NH-alkyl.

In another embodiment, R⁴ is —S(O)₂R⁷.

In another embodiment, R⁴ is —S(O)₂-alkyl.

In still another embodiment, R⁴ is —S(O)₂-aryl.

In still another embodiment, R⁴ is —S(O)₂-phenyl.

In one embodiment, each occurrence of R¹⁰ is H.

In another embodiment, each occurrence of R¹¹ is H.

In another embodiment, each occurrence of R¹⁰ and R¹¹ is H.

In another embodiment, one occurrence of R¹⁰ or R¹¹ is other than hydrogen.

In yet another embodiment, at least one occurrence of R¹⁰ or R¹¹ is alkyl.

In still another embodiment, at least one occurrence of R¹⁰ or R¹¹ is methyl.

In another embodiment, R⁴ is benzyl, wherein the phenyl ring of the benzyl group can be unsubstituted or substituted with up to 3 substituents, which may be the same or different, and are selected from: F, Br, Cl, —NO₂, —CH₃, —CF₃, —SCF₃, —C(O)O-alkyl, pyrrolyl, thiazolyl, —C≡C-phenyl, —OCHF₂, piperidinyl, pyridyl, pyrrolidinyl, pyrazolyl, methoxy, piperazinyl, morpholinyl, —OCF₂CHF₂, 1,3,4-triazolyl, —CH(OH)CH₃, —OH, —SO₂CH₃, —C(O)OH or -phenyl.

In one embodiment, R⁴ is —CH₂-heteroaryl, wherein the heteroaryl is thienyl, benzthienyl, thiazolyl, benzthiazolyl, furanyl, benzofuranyl, pyridyl, isoxazolyl or benzimidazolyl.

In one embodiment, one or more occurrences of n is 1.

In another embodiment, one or more occurrences of n is 0.

In another embodiment, one or more occurrences of p is 0.

In still another embodiment, one or more occurrences of p is 1.

In yet another embodiment, one or more occurrences of p is 2.

In one embodiment, one or more occurrences of q is 1.

In another embodiment, one or more occurrences of q is 2.

In one embodiment, R² and R³ are each independently aryl, heteroaryl or cycloalkyl.

In another embodiment, R² and R³ are each aryl.

In yet another embodiment, R² and R³ are each heteroaryl.

In another embodiment, R² and R³ are each phenyl.

In another embodiment, R² is aryl and R³ is heteroaryl.

In still another embodiment, R² is phenyl and R³ is heteroaryl.

In yet another embodiment, R² is phenyl and R³ is pyridyl.

In a further embodiment, R² is phenyl and R³ is 2-pyridyl.

In another embodiment, R² and R³ are each 4-trifluoromethylphenyl.

In another embodiment, R² and R³ are each 4-chlorophenyl.

In one embodiment, R² and R³ are each 4-fluorophenyl.

In another embodiment, R² is aryl and R³ is cycloalkyl.

In still another embodiment, R² is phenyl and R³ is cycloalkyl.

In a further embodiment, R² is phenyl and R³ is cyclopentyl.

In another embodiment, R² is phenyl and R³ is cyclobutyl.

In still another embodiment, R² is phenyl and R³ is 4-fluorophenyl.

In yet another embodiment, R² is phenyl and R³ is pyrimidinyl.

In still another embodiment, R² is phenyl and R³ is thienyl.

In another embodiment, R² is —C(O)OR⁵ and R³ is phenyl.

In another embodiment, R² is —C(O)N(R⁶)₂ and R³ is phenyl.

In another embodiment, R¹ is alkyl, R² is aryl and R³ is heteroaryl.

In still another embodiment, R¹ is alkyl, R² is phenyl and R³ is heteroaryl.

In yet another embodiment, R¹ is alkyl, R² is phenyl and R³ is pyridyl.

In another embodiment, R¹ is alkyl, R² is phenyl and R³ is 2-pyridyl.

In a further embodiment, R¹ is alkyl, and R² and R³ are each aryl.

In another embodiment, R¹ is alkyl, and R² and R³ are each heteroaryl.

In yet another embodiment, R¹ is alkyl, and R² and R³ are each phenyl.

In another embodiment, R¹ is alkyl, and R² and R³ are each 4-trifluoromethylphenyl.

In a further embodiment, R¹ is alkyl, and R² and R³ are each 4-chlorophenyl.

In one embodiment, R¹ is alkyl, and R² and R³ are each 4-fluorophenyl.

In still another embodiment, R¹ is alkyl, R² is phenyl and R³ is 4-fluorophenyl.

In another embodiment, R¹ is benzyl, R² is aryl and R³ is heteroaryl.

In still another embodiment, R¹ is benzyl, R² is phenyl and R³ is heteroaryl.

In yet another embodiment, R¹ is benzyl, R² is phenyl and R³ is pyridyl.

In another embodiment, R¹ is benzyl, R² is phenyl and R³ is 2-pyridyl.

In another embodiment, R¹ is benzyl, R² is phenyl and R³ is 4-fluorophenyl.

In a further embodiment, R¹ is benzyl, and R² and R³ are each aryl.

In another embodiment, R¹ is benzyl, and R² and R³ are each heteroaryl.

In yet another embodiment, R¹ is benzyl, and R² and R³ are each phenyl.

In another embodiment, R¹ is benzyl, and R² and R³ are each 4-trifluoromethylphenyl.

In a further embodiment, R¹ is benzyl, and R² and R³ are each 4-chlorophenyl.

In one embodiment, R¹ is benzyl, and R² and R³ are each 4-fluorophenyl.

In one embodiment, R¹ is —N(R⁹)₂, R² is aryl and R³ is heteroaryl.

In another embodiment, R¹ is —N(R⁹)₂, R² is phenyl and R³ is heteroaryl.

In yet another embodiment, R¹ is —N(R⁹)₂, R² is phenyl and R³ is pyridyl.

In another embodiment, R¹ is —N(R⁹)₂, R² is phenyl and R³ is 2-pyridyl.

In yet another embodiment, R¹ is —N(R⁹)₂, R² is phenyl and R³ is 4-fluorophenyl.

In a further embodiment, R¹ is —N(R⁹)₂, and R² and R³ are each aryl.

In another embodiment, R¹ is —N(R⁹)₂, and R² and R³ are each heteroaryl.

In yet another embodiment, R¹ is —N(R⁹)₂, and R² and R³ are each phenyl.

In another embodiment, R¹ is —N(R⁹)₂, and R² and R³ are each 4-trifluoromethylphenyl.

In another embodiment, R¹ is —N(R⁹)₂, and R² and R³ are each 4-chlorophenyl.

In still another embodiment, R¹ is —N(R⁹)₂, and R² and R³ are each 4-fluorophenyl.

In one embodiment, R¹ is —NH₂, R² is aryl and R³ is heteroaryl.

In another embodiment, R¹ is —NH₂, R² is phenyl and R³ is heteroaryl.

In yet another embodiment, R¹ is —NH₂, R² is phenyl and R³ is pyridyl.

In another embodiment, R¹ is —NH₂, R² is phenyl and R³ is 2-pyridyl.

In another embodiment, R¹ is —NH₂, R² is phenyl and R³ is 4-fluorophenyl.

In a further embodiment, R¹ is —NH₂, and R² and R³ are each aryl.

In another embodiment, R¹ is —NH₂, and R² and R³ are each heteroaryl.

In yet another embodiment, R¹ is —NH₂, and R² and R³ are each phenyl.

In another embodiment, R¹ is —NH₂, and R² and R³ are each 4-trifluoromethylphenyl.

In a further embodiment, R¹ is —NH₂, and R² and R³ are each 4-chlorophenyl.

In another embodiment, R¹ is —NH₂, and R² and R³ are each 4-fluorophenyl.

In one embodiment, R¹ is methyl, R² is aryl and R³ is heteroaryl.

In still another embodiment, R¹ is methyl, R² is phenyl and R³ is heteroaryl.

In yet another embodiment, R¹ is methyl, R² is phenyl and R³ is pyridyl.

In another embodiment, R¹ is methyl, R² is phenyl and R³ is 2-pyridyl.

In another embodiment, R¹ is methyl, R² is phenyl and R³ is 4-fluorophenyl.

In a further embodiment, R¹ is methyl and R² and R³ are each aryl.

In another embodiment, R¹ is methyl and R² and R³ are each heteroaryl.

In yet another embodiment, R¹ is alkyl and R² and R³ are each phenyl.

In another embodiment, R¹ is methyl and R² and R³ are each phenyl.

In another embodiment, R¹ is methyl and R² and R³ are each 4-trifluoromethylphenyl.

In a further embodiment, R¹ is methyl and R² and R³ are each 4-chlorophenyl.

In another embodiment, R¹ is methyl and R² and R³ are each 4-fluorophenyl.

In one embodiment, R¹ is methyl, R² and R³ are each unsubstituted or substituted phenyl, and R⁴ is —C(O)OR⁵.

In another embodiment, R¹ is alkyl; R² and R³ are each unsubstituted or substituted phenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is alkyl; R² and R³ are each unsubstituted or substituted phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

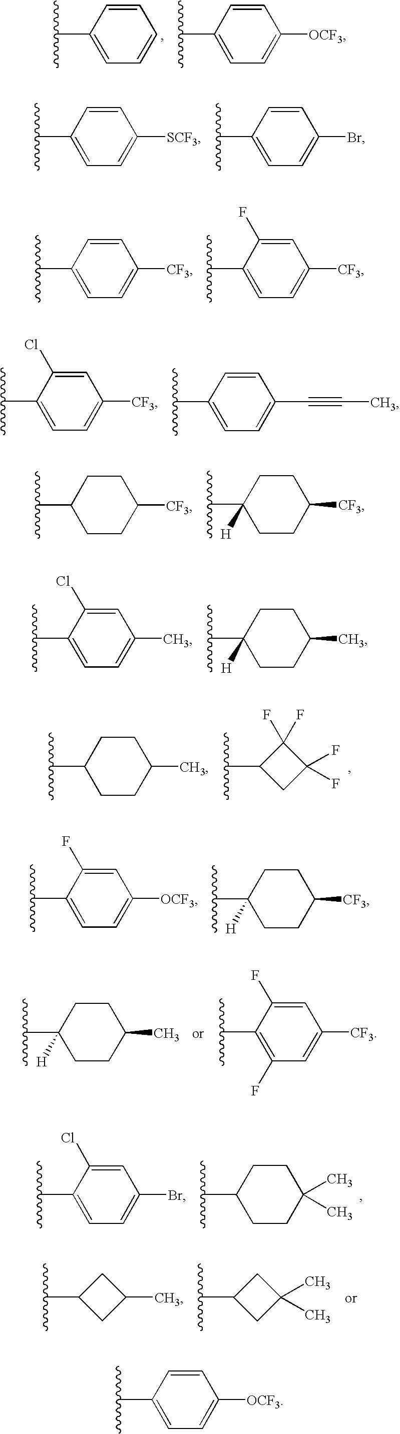

In one embodiment, R¹ is alkyl; R² is phenyl; R³ is 4-fluorophenyl; and R⁴ is —C(O)OR⁵.

In another embodiment, R¹ is alkyl; R² is phenyl; R³ is 4-fluorophenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is alkyl; R² is phenyl; R³ is 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

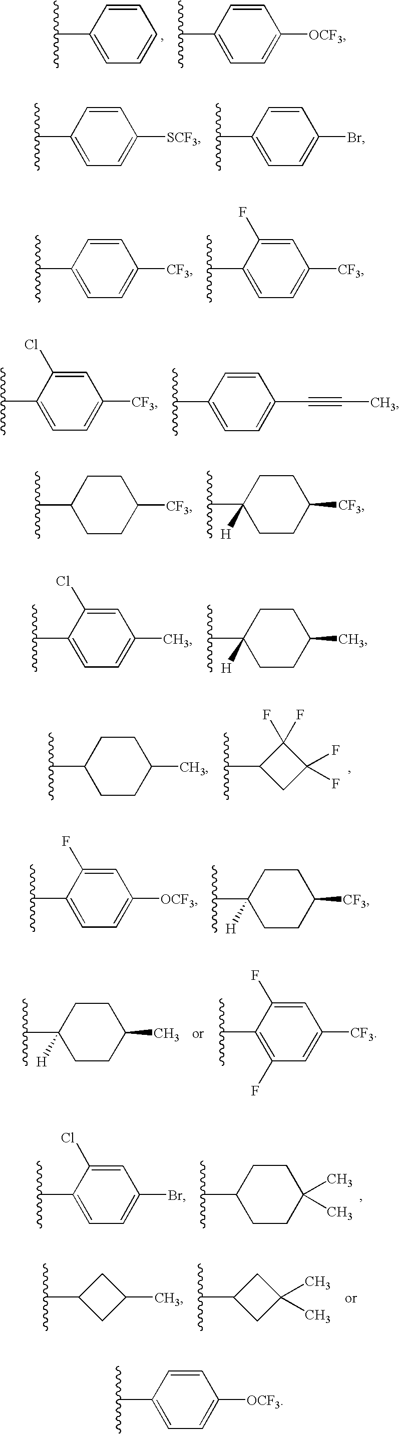

In one embodiment, R¹ is alkyl; R² and R³ are each independently cyclopentyl, cyclobutyl, 3-fluorophenyl or 4-fluorophenyl; and R⁴ is —C(O)OR⁵.

In another embodiment, R¹ is alkyl; R² and R³ are each independently cyclopentyl, cyclobutyl, 3-fluorophenyl or 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is alkyl; R² and R³ are each independently cyclopentyl, cyclobutyl, 3-fluorophenyl or 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

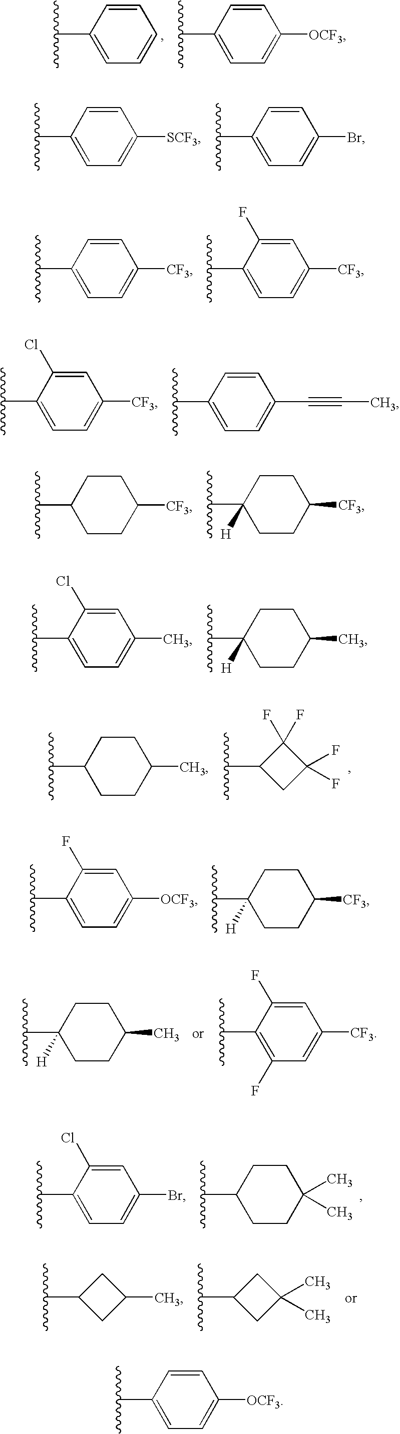

In one embodiment, R¹ is alkyl; R² and R³ are each 4-fluorophenyl; and R⁴ is —C(O)OR⁵.

In another embodiment, R¹ is alkyl; R² and R³ are each 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is alkyl; R² and R³ are each 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

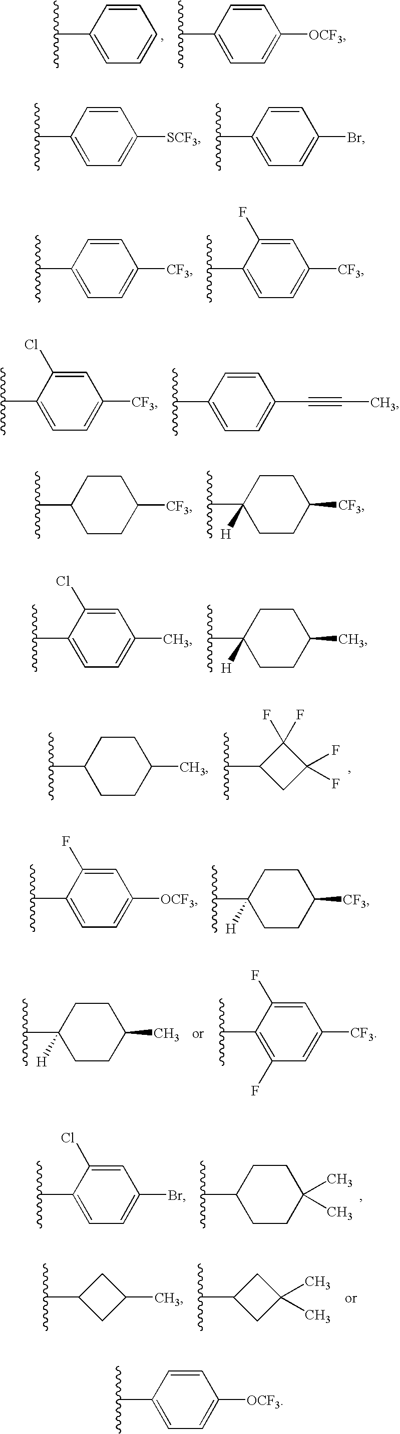

In one embodiment, R¹ is methyl; R² and R³ are each unsubstituted or substituted phenyl; and R⁴ is —C(O)OR⁵.

In another embodiment, R¹ is methyl; R² and R³ are each unsubstituted or substituted phenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is methyl; R² and R³ are each unsubstituted or substituted phenyl; and R⁴ is —C(O)OR⁵, wherein R⁵ is -tert-butyl, —CH₂CO₃, —C(CH₃)₂CCl₃, —CH₂CF₂CF₃, —CH(CF₃)₂, —CH₂CH(CF₃)₂,

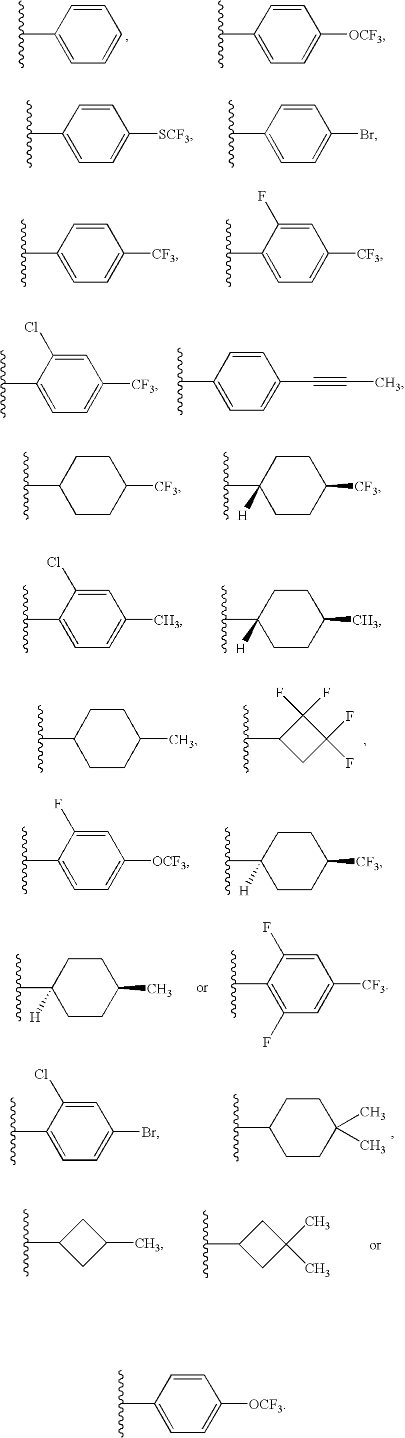

In one embodiment, R¹ is methyl; R² and R³ are each phenyl; and R⁴ is —C(O)OR⁵.

In another embodiment, R¹ is methyl; R² and R³ are each phenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is methyl; R² and R³ are each phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

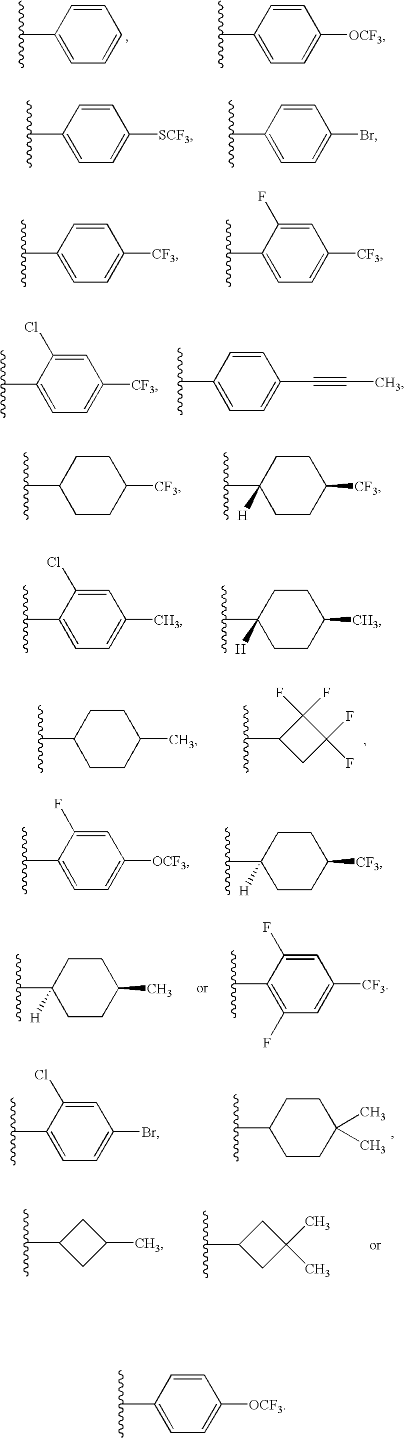

In one embodiment, R¹ is methyl; R² is phenyl; R³ is 4-fluorophenyl; and R⁴ is —C(O)OR⁵.

In another embodiment, R¹ is methyl; R² is phenyl; R³ is 4-fluorophenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is methyl; R² is phenyl; R³ is 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

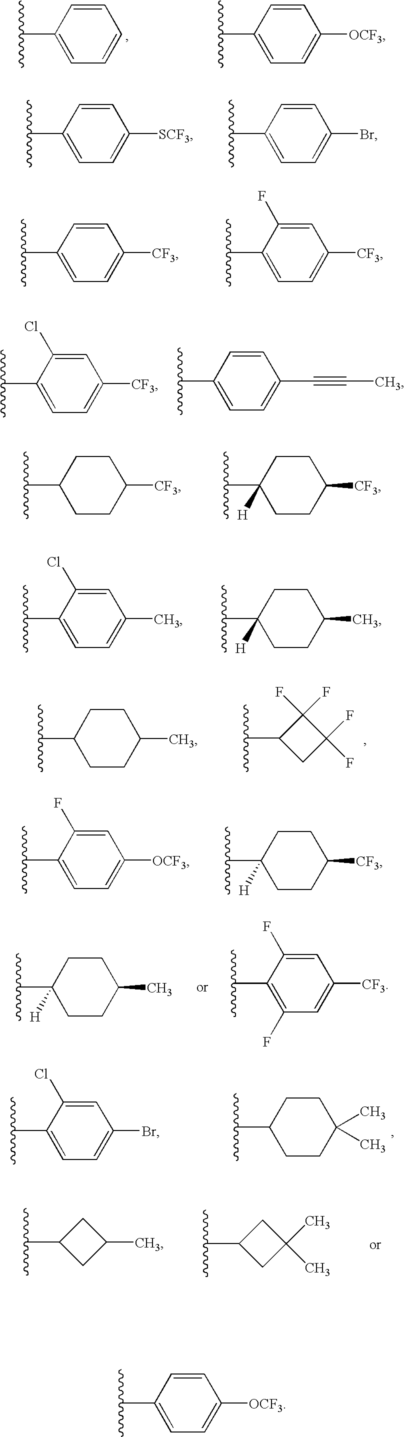

In one embodiment, R¹ is methyl; R² and R³ are each independently cyclopentyl, cyclobutyl, 3-fluorophenyl or 4-fluorophenyl; and R⁴ is —C(O)OR⁵.

In another embodiment, R¹ is methyl; R² and R³ are each independently cyclopentyl, cyclobutyl, 3-fluorophenyl or 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is methyl; R² and R³ are each independently cyclopentyl, cyclobutyl, 3-fluorophenyl or 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

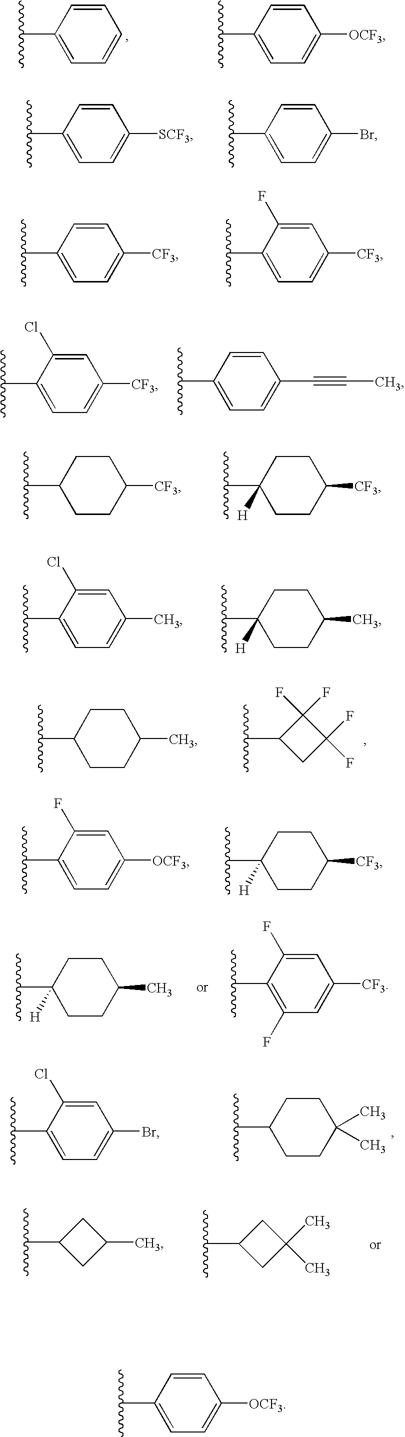

In one embodiment, R¹ is methyl; R² and R³ are each 4-fluorophenyl; and R⁴ is —C(O)OR⁵.

In another embodiment, R¹ is methyl; R² and R³ are each 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is methyl; R² and R³ are each 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

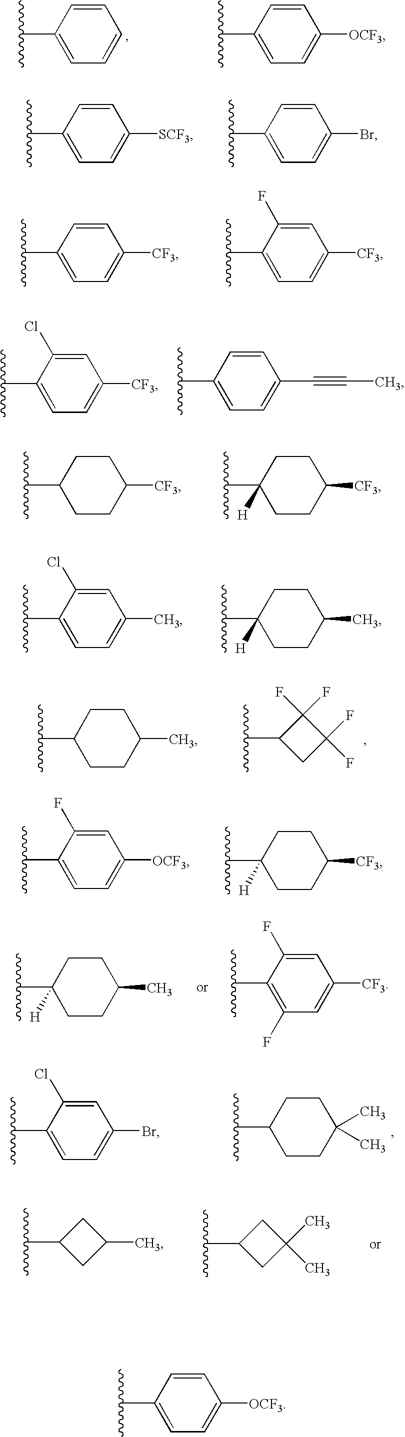

In one embodiment, R¹ is —N(R⁹)₂; R² and R³ are each unsubstituted or substituted phenyl; and R⁴ is —C(O)OR⁵.

In another embodiment, R¹ is —N(R⁹)₂; R² and R³ are each unsubstituted or substituted phenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —N(R⁹)₂; R² and R³ are each unsubstituted or substituted phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

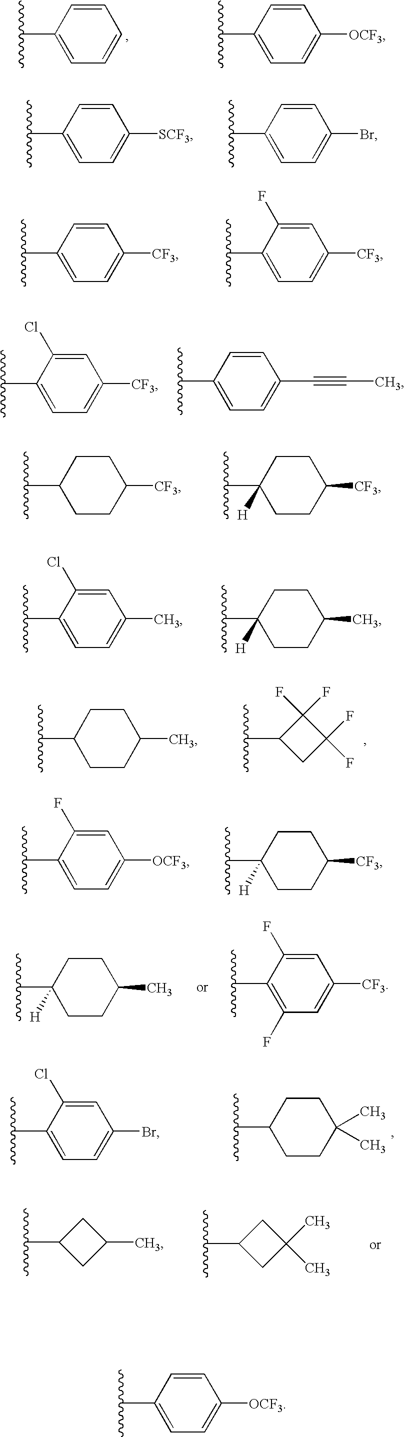

In one embodiment, R¹ is —N(R⁹)₂; R² and R³ are each phenyl; and R⁴ is —C(O)OR⁵.

In another embodiment, R¹ is —N(R⁹)₂; R² and R³ are each phenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —N(R⁹)₂; R² and R³ are each phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

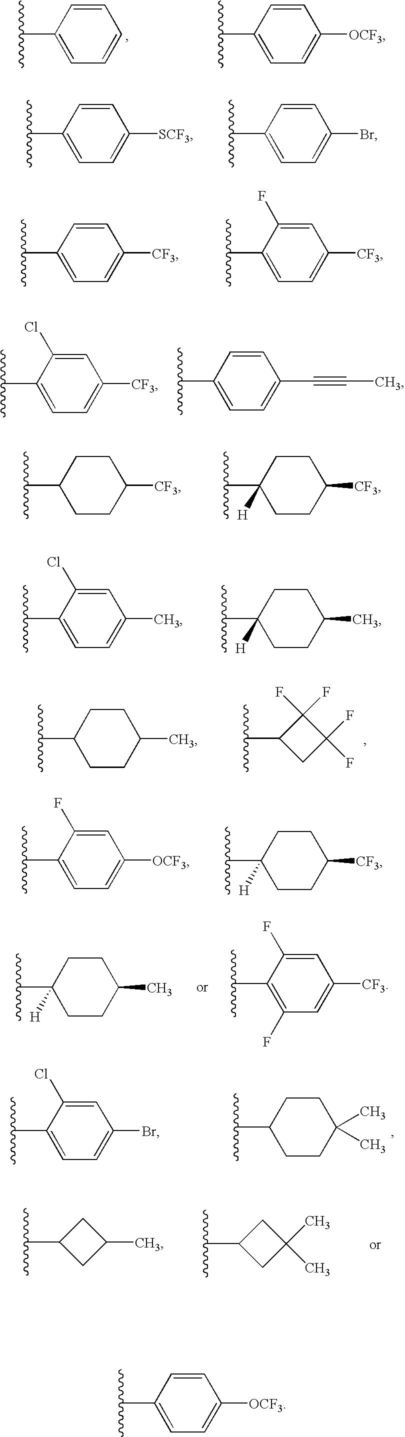

In one embodiment, R¹ is —N(R⁹)₂; R² is phenyl; R³ is 4-fluorophenyl; and R⁴ is —C(O)OR⁵.

In another embodiment, R¹ is —N(R⁹)₂; R² is phenyl; R³ is 4-fluorophenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —N(R⁹)₂; R² is phenyl; R³ is 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

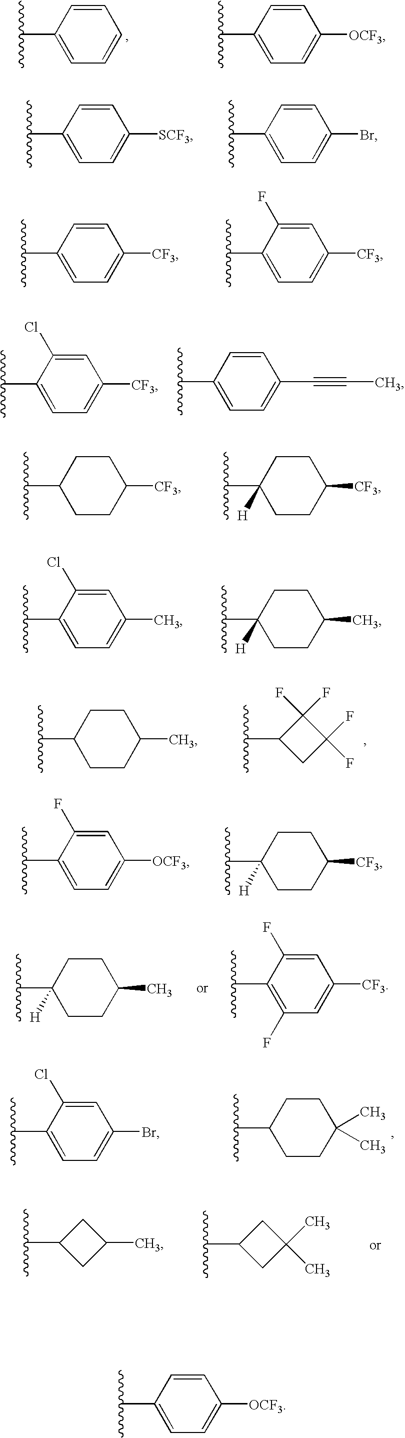

In one embodiment, R¹ is —N(R⁹)₂; R² and R³ are each independently cyclopentyl, cyclobutyl, 3-fluorophenyl or 4-fluorophenyl; and R⁴ is —C(O)OR⁵.

In another embodiment, R¹ is —N(R⁹)₂; R² and R³ are each independently cyclopentyl, cyclobutyl, 3-fluorophenyl or 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —N(R⁹)₂; R² and R³ are each independently cyclopentyl, cyclobutyl, 3-fluorophenyl or 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

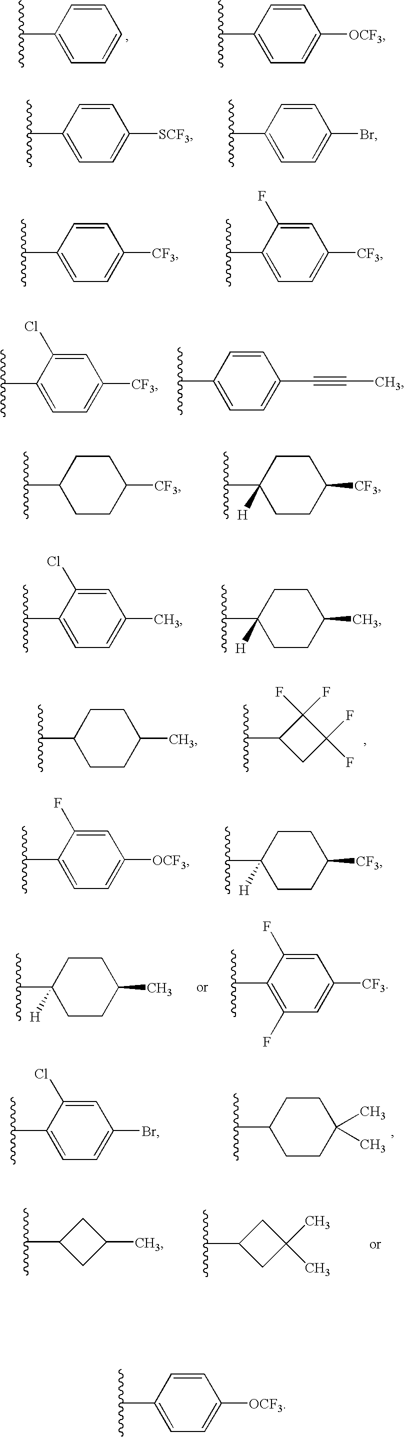

In one embodiment, R¹ is —N(R⁹)₂; R² and R³ are each 4-fluorophenyl; and R⁴ is —C(O)OR⁵.

In another embodiment, R¹ is —N(R⁹)₂; R² and R³ are each 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —N(R⁹)₂; R² and R³ are each 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

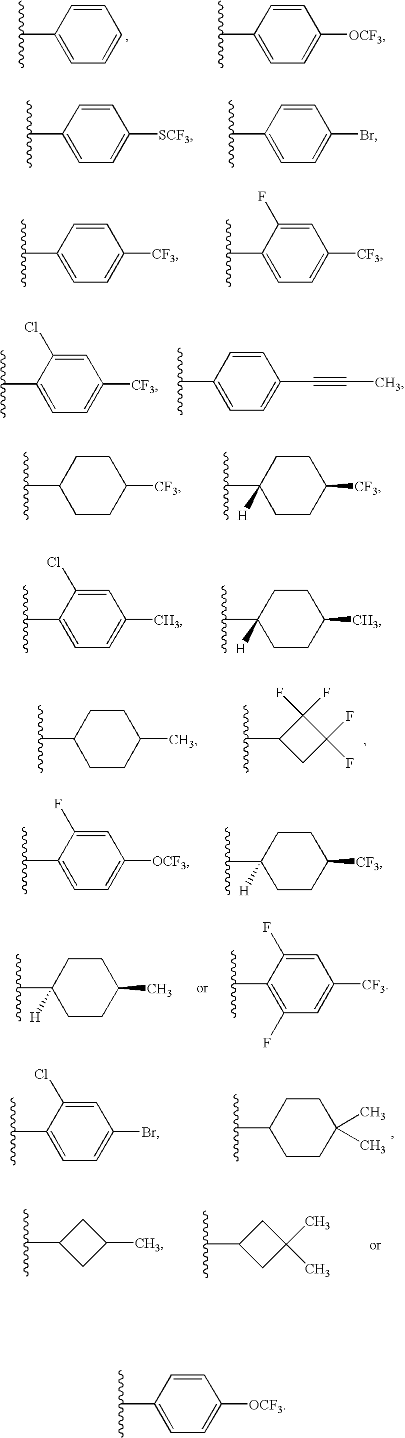

In one embodiment, R¹ is —NH₂; R² and R³ are each unsubstituted or substituted phenyl; and R⁴ is —C(O)OR⁵.

In another embodiment, R¹ is —NH₂; R² and R³ are each unsubstituted or substituted phenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —NH₂; R² and R³ are each unsubstituted or substituted phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

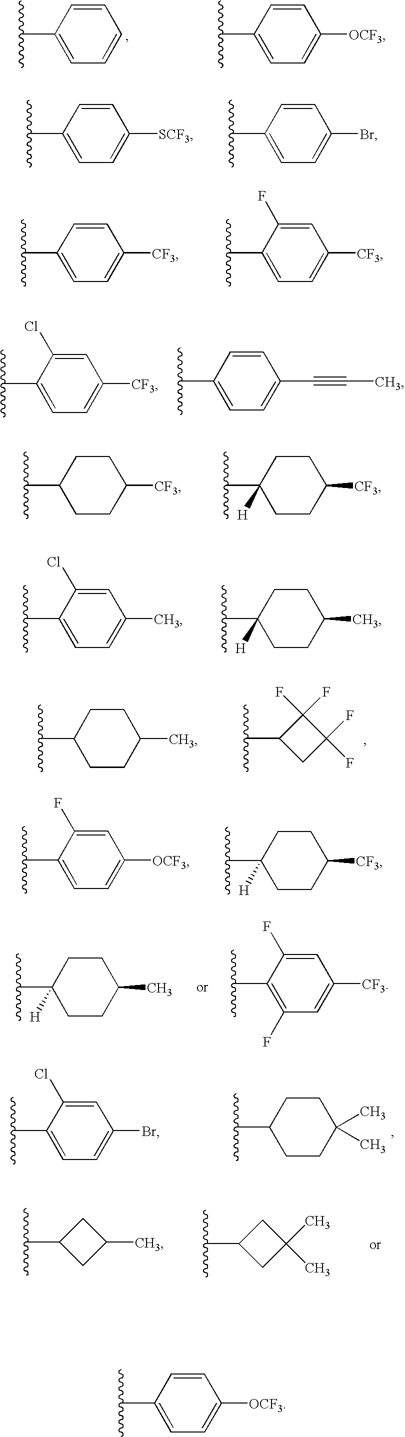

In one embodiment, R¹ is —NH₂; R² and R³ are each phenyl; and R⁴ is —C(O)OR⁵.

In another embodiment, R¹ is —NH₂; R² and R³ are each phenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —NH₂; R² and R³ are each phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

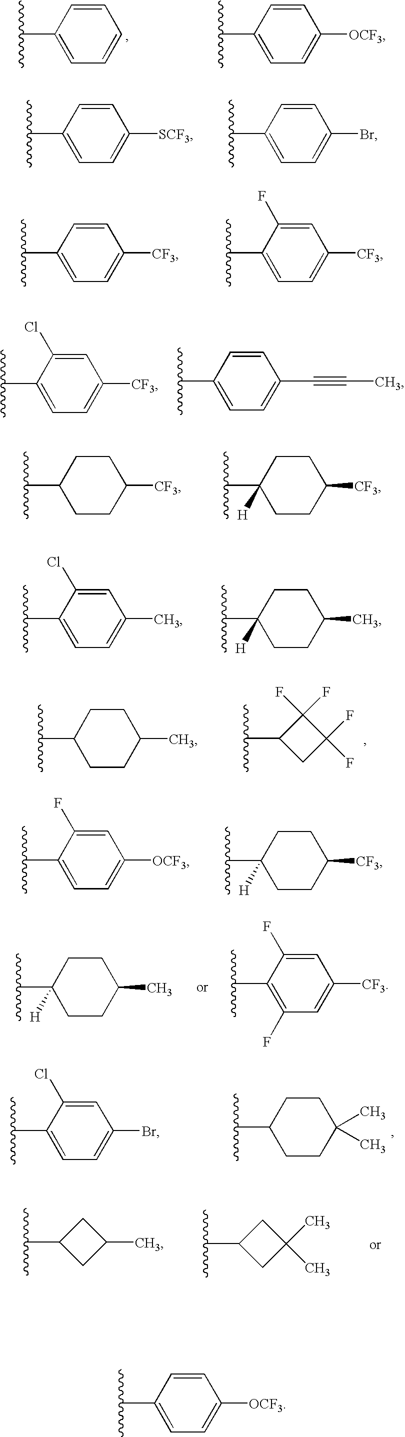

In one embodiment, R¹ is —NH₂; R² is phenyl; R³ is 4-fluorophenyl; and R⁴ is —C(O)OR⁵.

In another embodiment, R¹ is —NH₂; R² is phenyl; R³ is 4-fluorophenyl; and R⁴ is — C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —NH₂; R² is phenyl; R³ is 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, CH₂CH(CF₃)₂,

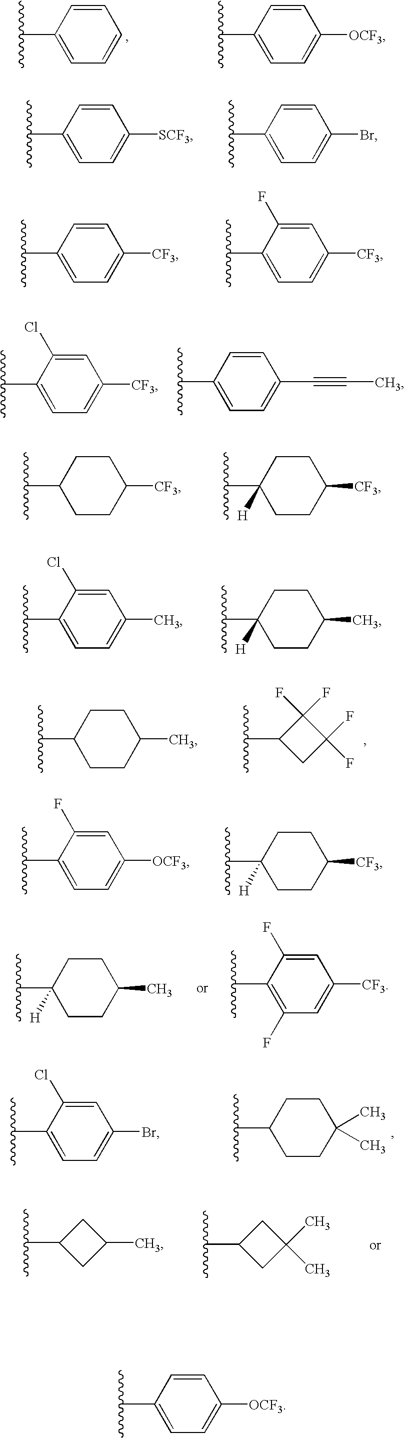

In one embodiment, R¹ is —NH₂; R² and R³ are each independently cyclopentyl, cyclobutyl, 3-fluorophenyl or 4-fluorophenyl; and R⁴ is —C(O)OR⁵.

In another embodiment, R¹ is —NH₂; R² and R³ are each independently cyclopentyl, cyclobutyl, 3-fluorophenyl or 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —NH₂; R² and R³ are each independently cyclopentyl, cyclobutyl, 3-fluorophenyl or 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

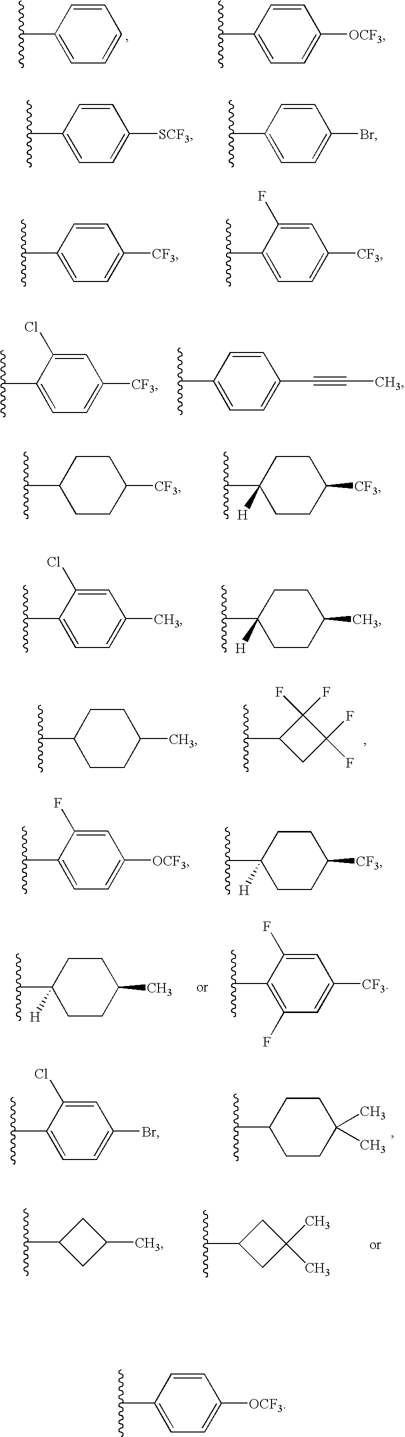

In one embodiment, R¹ is —NH₂; R² and R³ are each 4-fluorophenyl; and R⁴ is —C(O)OR⁵.

In another embodiment, R¹ is —NH₂; R² and R³ are each 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —NH₂; R² and R³ are each 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

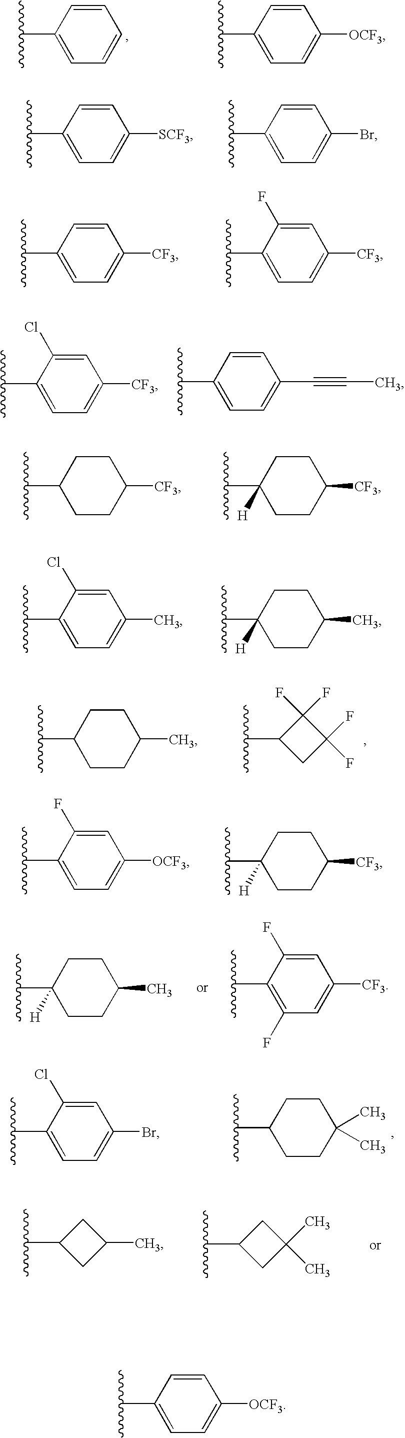

In one embodiment, J is a single bond; G is —C(R¹⁰)(R¹¹)—; R¹ is methyl; R² and R³ are each unsubstituted or substituted phenyl; and R⁴ is —C(O)OR⁵.

In another embodiment, J is a single bond; G is —C(R¹⁰)(R¹¹)—; R¹ is methyl; R² and R³ are each unsubstituted or substituted phenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —C(R¹⁰)(R¹¹)—; R¹ is methyl; R² and R³ are each unsubstituted or substituted phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

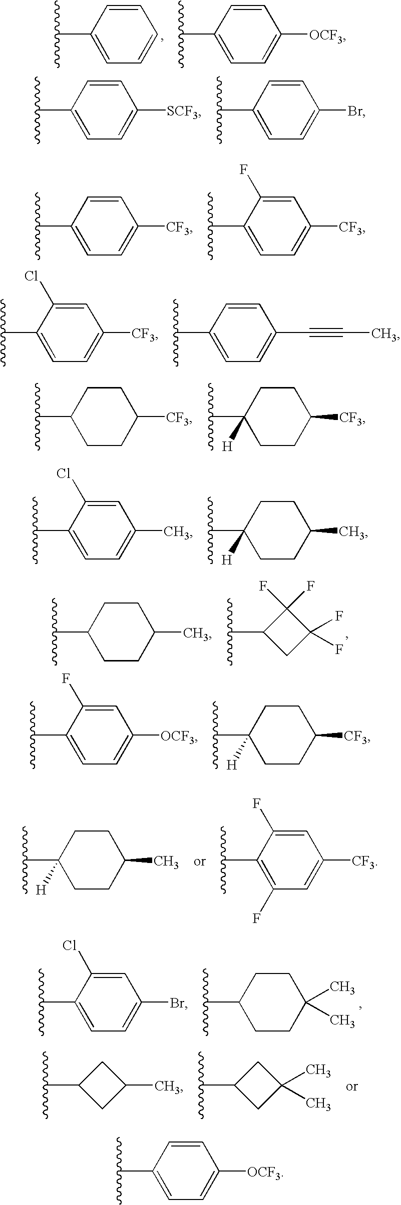

In one embodiment, J is a single bond; G is —C(R¹⁰)(R¹¹)—; R¹ is methyl; R² and R³ are each phenyl; and R⁴ is —C(O)OR⁵.

In another embodiment, J is a single bond; G is —C(R¹⁰)(R¹¹)—; R¹ is methyl; R² and R³ are each phenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —C(R¹⁰)(R¹¹)—; R¹ is methyl; R² and R³ are each phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

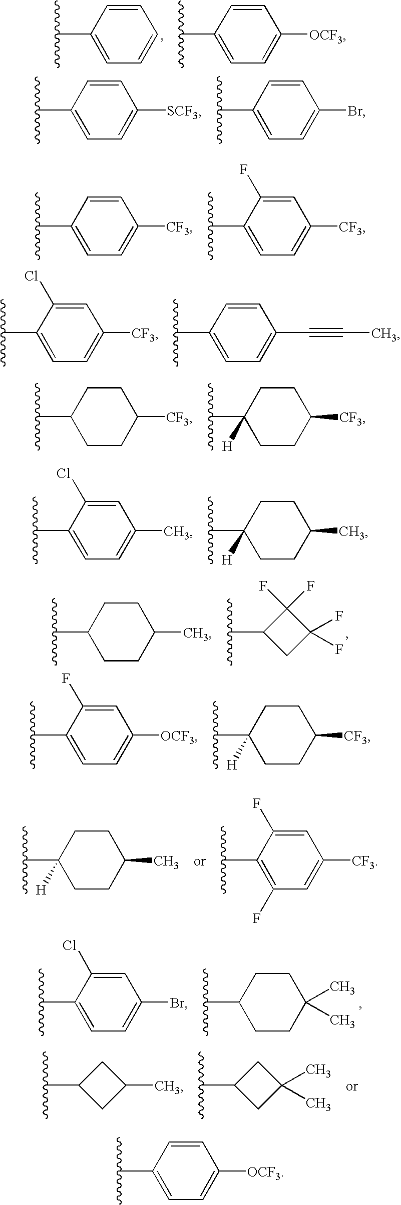

In one embodiment, J is a single bond; G is —C(R¹⁰)(R¹¹)—; R¹ is methyl; R² is phenyl; R³ is 4-fluorophenyl; and R⁴ is —C(O)OR⁵.

In another embodiment, J is a single bond; G is —C(R¹⁰)(R¹¹)—; R¹ is methyl; R² is phenyl; R³ is 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —C(R¹⁰)(R¹¹)—; R¹ is methyl; R² is phenyl; R³ is 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

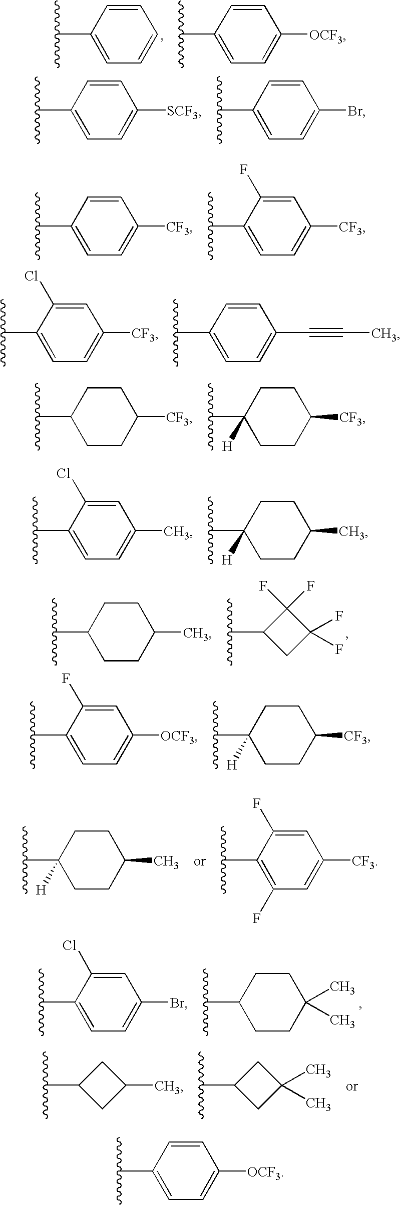

In one embodiment, J is a single bond; G is —C(R¹⁰)(R¹¹)—; R¹ is methyl; R² and R³ are each independently cyclopentyl, cyclobutyl, 3-fluorophenyl or 4-fluorophenyl; and R⁴ is —C(O)OR⁵.

In another embodiment, J is a single bond; G is —C(R¹⁰)(R¹¹)—; R¹ is methyl; R² and R³ are each independently cyclopentyl, cyclobutyl, 3-fluorophenyl or 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —C(R¹⁰)(R¹¹)—; R¹ is methyl; R² and R³ are each independently cyclopentyl, cyclobutyl, 3-fluorophenyl or 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

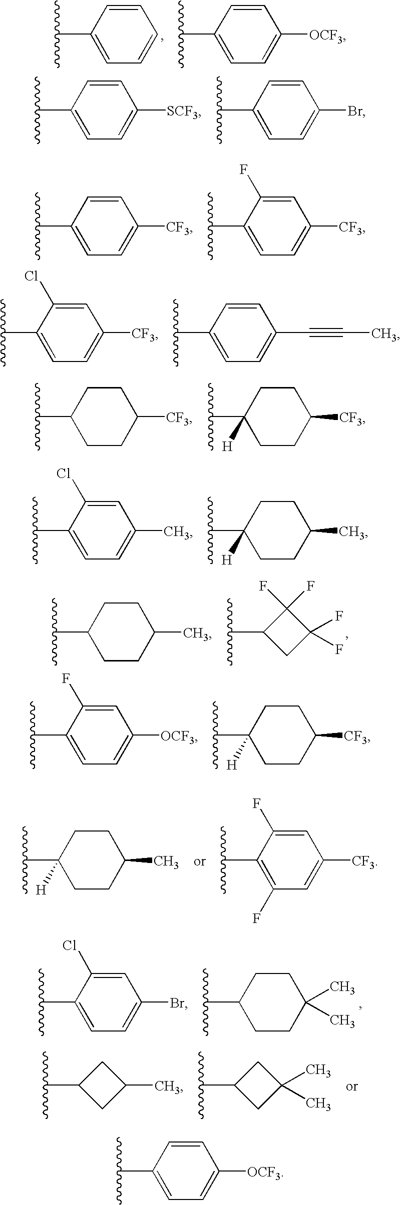

In one embodiment, J is a single bond; G is —C(R¹⁰)(R¹¹)—; R¹ is methyl; R² and R³ are each 4-fluorophenyl; and R⁴ is —C(O)OR⁵.

In another embodiment, J is a single bond; G is —C(R¹⁰)(R¹¹)—; R¹ is methyl; R² and R³ are each 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —C(R¹⁰)(R¹¹)—; R¹ is methyl; R² and R³ are each 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

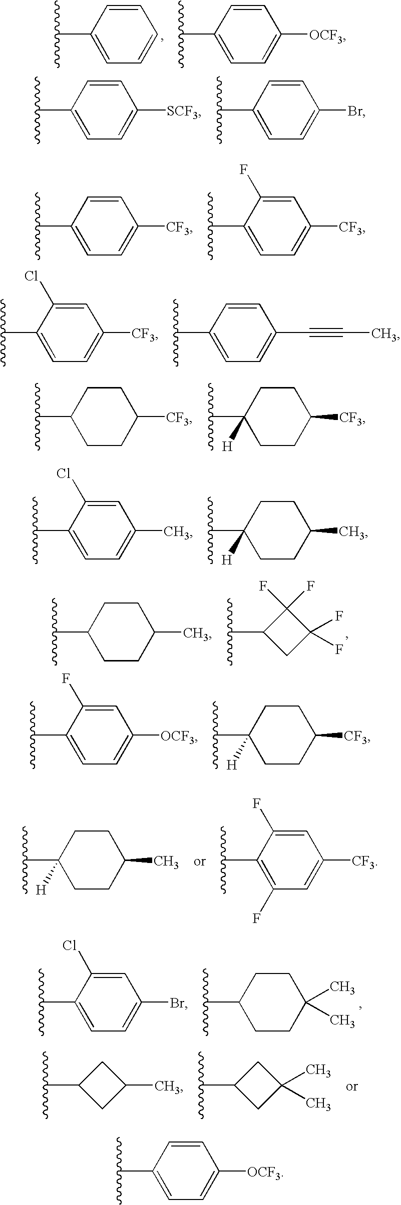

In one embodiment, J is a single bond; G is —CH₂—; R¹ is methyl; R² and R³ are each unsubstituted or substituted phenyl; and R⁴ is —C(O)OR⁵.

In another embodiment, J is a single bond; G is —CH₂—; R¹ is methyl; R² and R³ are each unsubstituted or substituted phenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —CH₂—; R¹ is methyl; R² and R³ are each unsubstituted or substituted phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

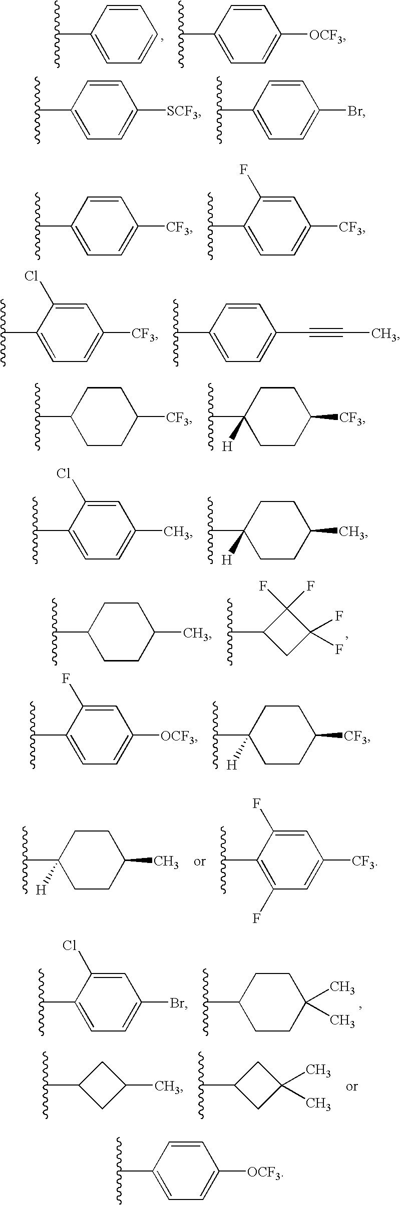

In one embodiment, J is a single bond; G is —CH₂—; R¹ is methyl; R² and R³ are each phenyl; and R⁴ is —C(O)OR⁵.

In another embodiment, J is a single bond; G is —CH₂—; R¹ is methyl; R² and R³ are each phenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —CH₂—; R¹ is methyl; R² and R³ are each phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

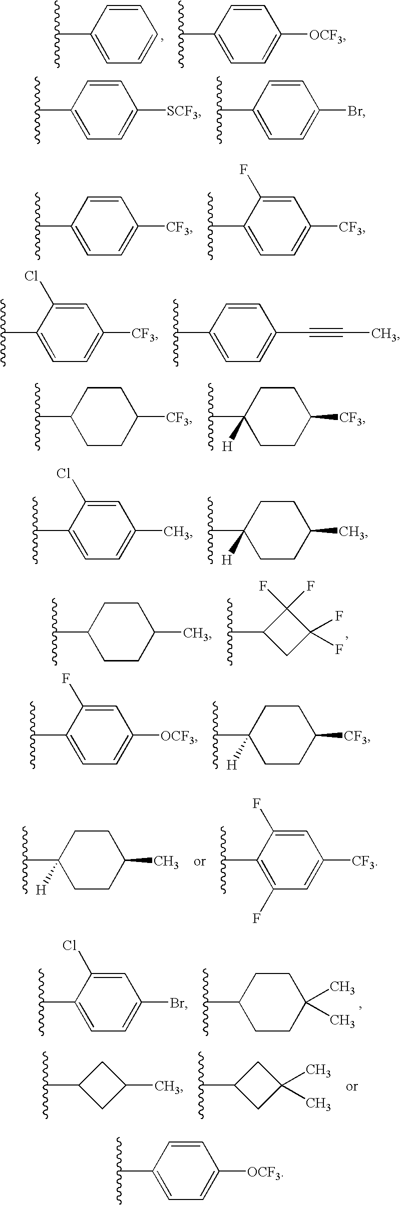

In one embodiment, J is a single bond; G is —CH₂—; R¹ is methyl; R² is phenyl; R³ is 4-fluorophenyl; and R⁴ is —C(O)OR⁵.

In another embodiment, J is a single bond; G is —CH₂—; R¹ is methyl; R² is phenyl; R³ is 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —CH₂—; R¹ is methyl; R² is phenyl; R³ is 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

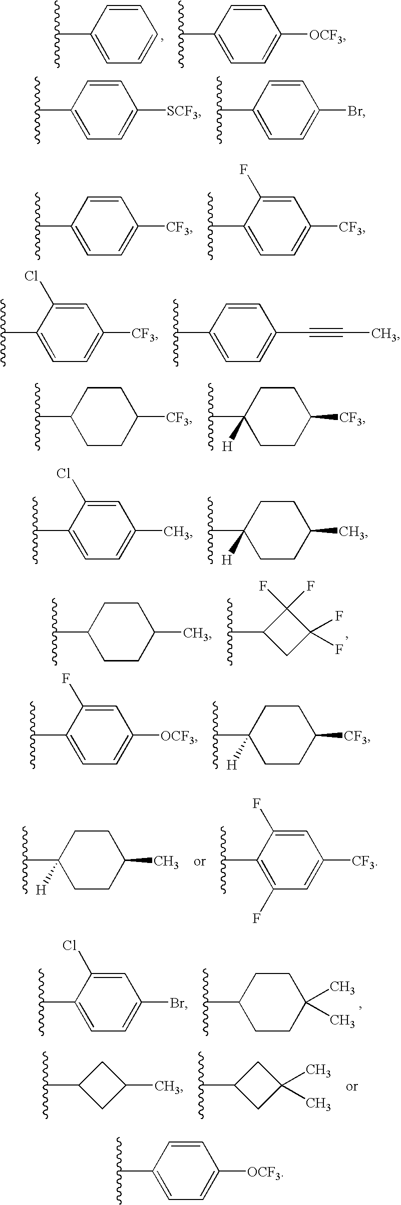

In one embodiment, J is a single bond; G is —CH₂—; R¹ is methyl; R² and R³ are each independently cyclopentyl, cyclobutyl, 3-fluorophenyl or 4-fluorophenyl; and R⁴ is —C(O)OR⁵.

In another embodiment, J is a single bond; G is —CH₂—; R¹ is methyl; R² and R³ are each independently cyclopentyl, cyclobutyl, 3-fluorophenyl or 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —CH₂—; R¹ is methyl; R² and R³ are each independently cyclopentyl, cyclobutyl, 3-fluorophenyl or 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

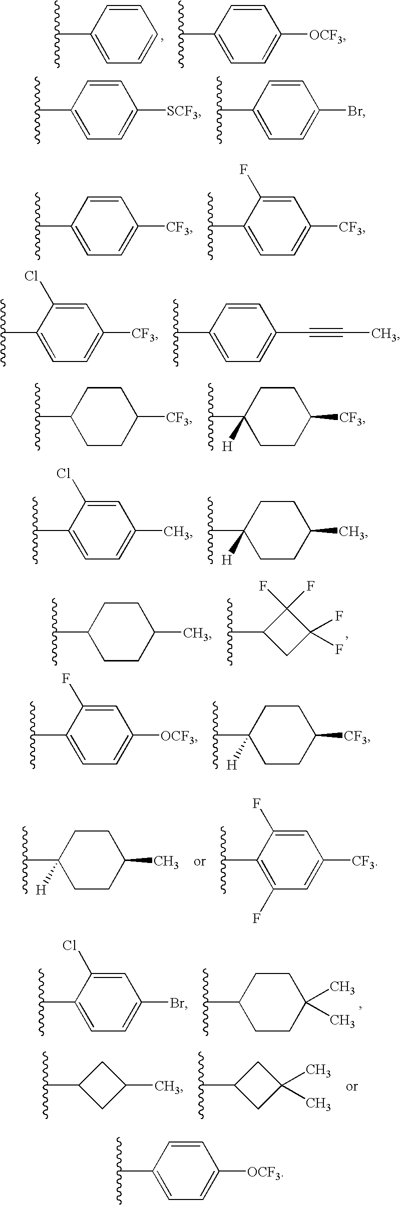

In one embodiment, J is a single bond; G is —CH₂—; R¹ is methyl; R² and R³ are each 4-fluorophenyl; and R⁴ is —C(O)OR⁵.

In another embodiment, J is a single bond; G is —CH₂—; R¹ is methyl; R² and R³ are each 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —CH₂—; R¹ is methyl; R² and R³ are each 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

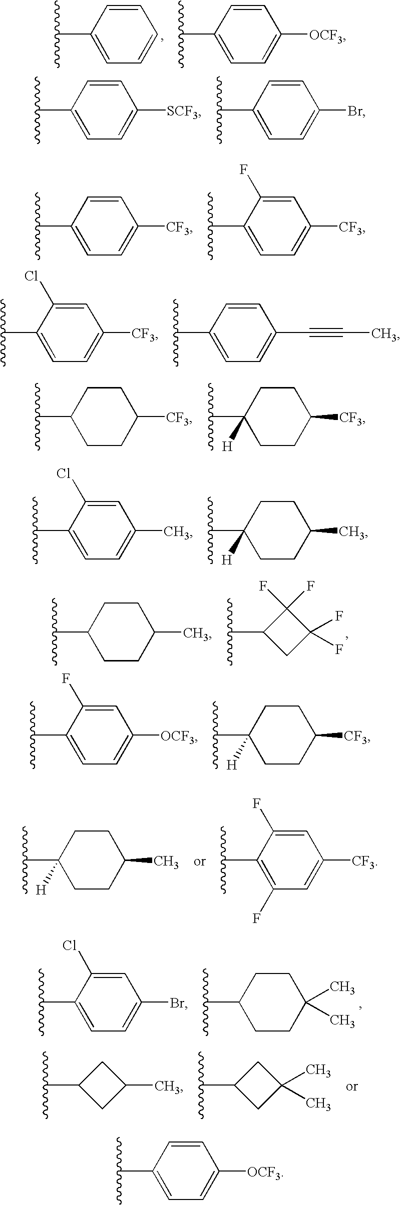

In one embodiment, J is a single bond; G is —C(R¹⁰)(R¹¹)—; R¹ is —NH₂; R² and R³ are each unsubstituted or substituted phenyl; and R⁴ is —C(O)OR⁵.

In another embodiment, J is a single bond; G is —C(R¹⁰)(R¹¹)—; R¹ is —NH₂; R² and R³ are each unsubstituted or substituted phenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —C(R¹⁰)(R¹¹)—; R¹ is —NH₂; R² and R³ are each unsubstituted or substituted phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

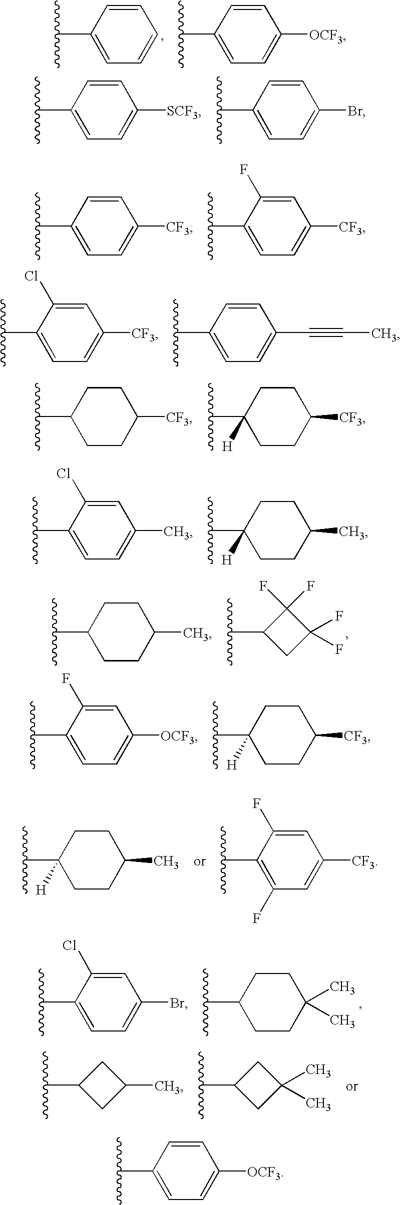

In one embodiment, J is a single bond; G is —C(R¹⁰)(R¹¹)—; R¹ is —NH₂; R² and R³ are each phenyl; and R⁴ is —C(O)OR⁵.

In another embodiment, J is a single bond; G is —C(R¹⁰)(R¹¹)—; R¹ is —NH₂; R² and R³ are each phenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —C(R¹⁰)(R¹¹)—; R¹ is —NH₂; R² and R³ are each phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

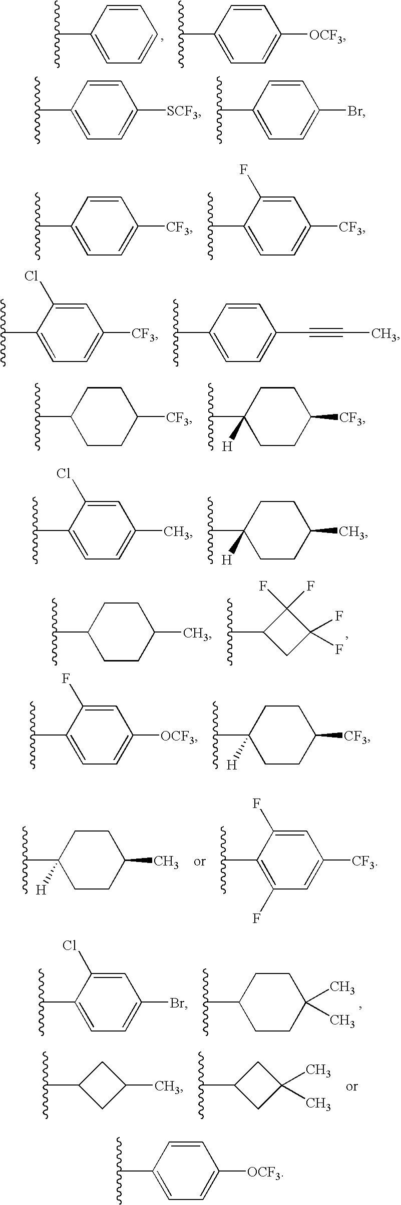

In one embodiment, J is a single bond; G is —C(R¹⁰)(R¹¹)—; R¹ is —NH₂; R² is phenyl; R³ is 4-fluorophenyl; and R⁴ is —C(O)OR⁵.

In another embodiment, J is a single bond; G is —C(R¹⁰)(R¹¹)—; R¹ is —NH₂; R² is phenyl; R³ is 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —C(R¹⁰)(R¹¹)—; R¹ is —NH₂; R² is phenyl; R³ is 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

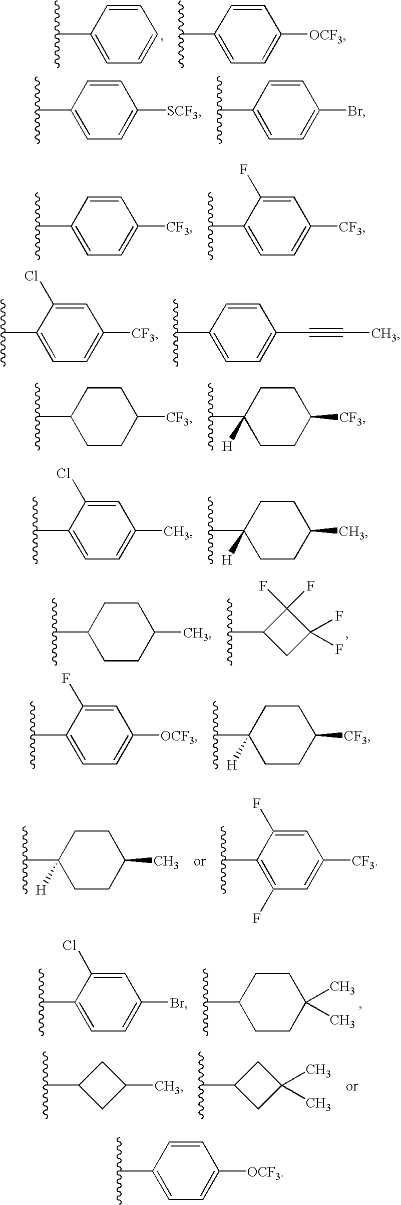

In one embodiment, J is a single bond; G is —C(R¹⁰)(R¹¹)—; R¹ is —NH₂, R² and R³ are each independently cyclopentyl, cyclobutyl, 3-fluorophenyl or 4-fluorophenyl; and R⁴ is —C(O)OR⁵.

In another embodiment, J is a single bond; G is —C(R¹⁰)(R¹¹)—; R¹ is —NH₂; R² and R³ are each independently cyclopentyl, cyclobutyl, 3-fluorophenyl or 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —C(R¹⁰)(R¹¹)—; R¹ is —NH₂; R² and R³ are each independently cyclopentyl, cyclobutyl, 3-fluorophenyl or 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

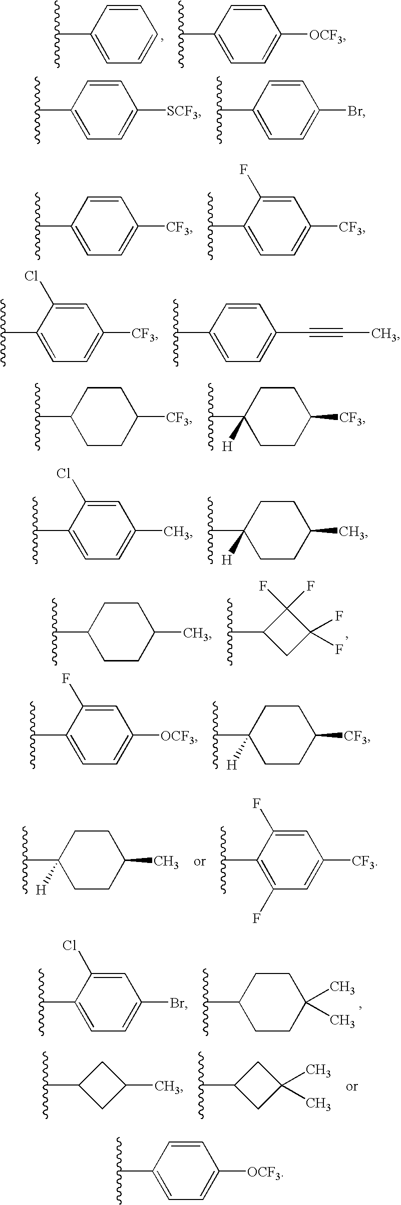

In one embodiment, J is a single bond; G is —C(R¹⁰)(R¹¹)—; R¹ is —NH₂; R² and R³ are each 4-fluorophenyl; and R⁴ is —C(O)OR⁵.

In another embodiment, J is a single bond; G is —C(R¹⁰)(R¹¹)—; R¹ is —NH₂; R² and R³ are each 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —C(R¹⁰)(R¹¹)—; R¹ is —NH₂; R² and R³ are each 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

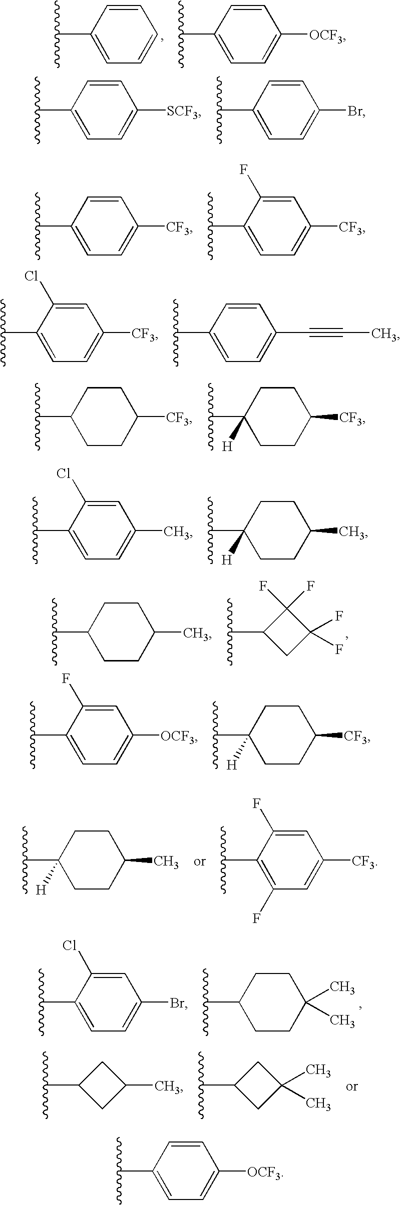

In one embodiment, J is a single bond; G is —CH₂—; R¹ is —NH₂; R² and R³ are each unsubstituted or substituted phenyl; and R⁴ is —C(O)OR⁵.

In another embodiment, J is a single bond; G is —CH₂—; R¹ is —NH₂; R² and R³ are each unsubstituted or substituted phenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —CH₂—; R¹ is —NH₂; R² and R³ are each unsubstituted or substituted phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

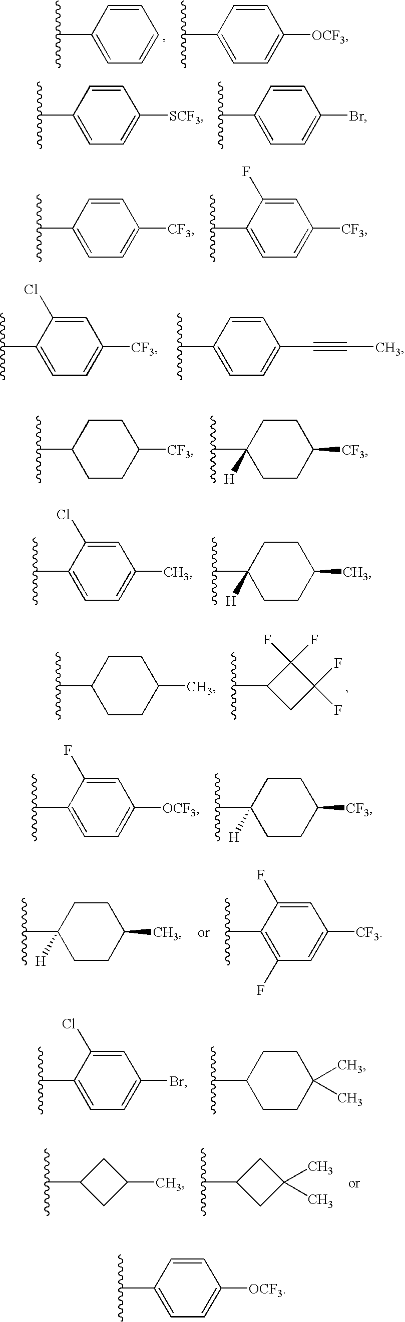

In one embodiment, J is a single bond; G is —CH₂—; R¹ is —NH₂; R² and R³ are each phenyl; and R⁴ is —C(O)OR⁵.

In another embodiment, J is a single bond; G is —CH₂—; R¹ is —NH₂; R² and R³ are each phenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —CH₂—; R¹ is —NH₂; R² and R³ are each phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

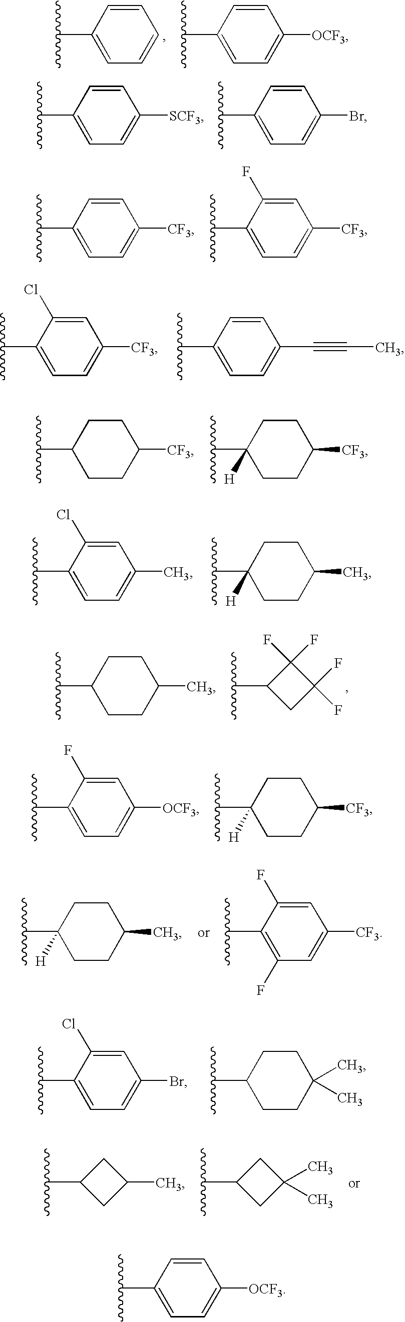

In one embodiment, J is a single bond; G is —CH₂—; R¹ is —NH₂; R² is phenyl; R³ is 4-fluorophenyl; and R⁴ is —C(O)OR⁵.

In another embodiment, J is a single bond; G is —CH₂—; R¹ is —NH₂; R² is phenyl; R³ is 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —CH₂—; R¹ is —NH₂; R² is phenyl; R³ is 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

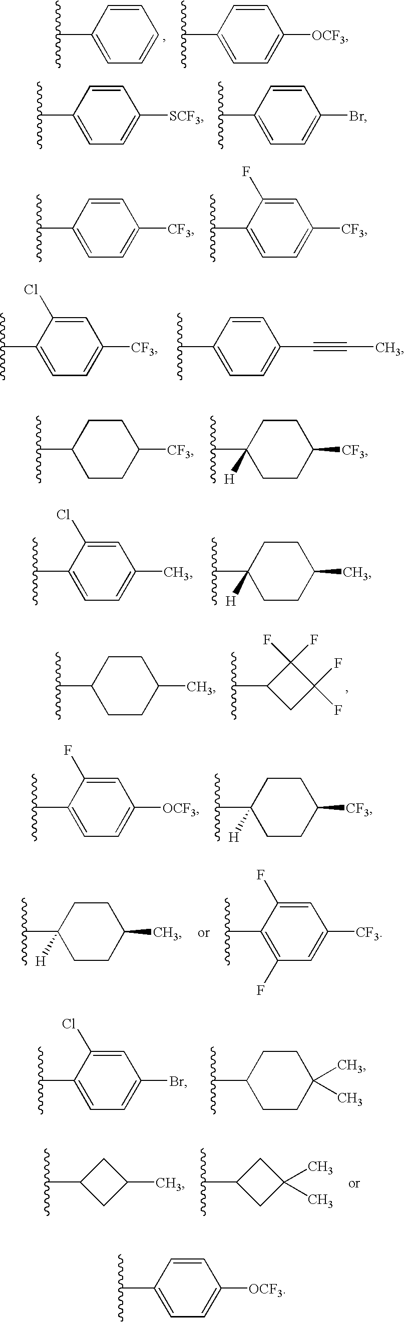

In one embodiment, J is a single bond; G is —CH₂—; R¹ is —NH₂; R² and R³ are each independently cyclobutyl, 3-fluorophenyl, cyclopentyl or 4-fluorophenyl; and R⁴ is —C(O)OR⁵.

In another embodiment, J is a single bond; G is —CH₂—; R¹ is —NH₂; R² and R³ are each independently cyclobutyl, 3-fluorophenyl, cyclopentyl or 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —CH₂—; R¹ is —NH₂; R² and R³ are each independently cyclobutyl, 3-fluorophenyl, cyclopentyl or 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

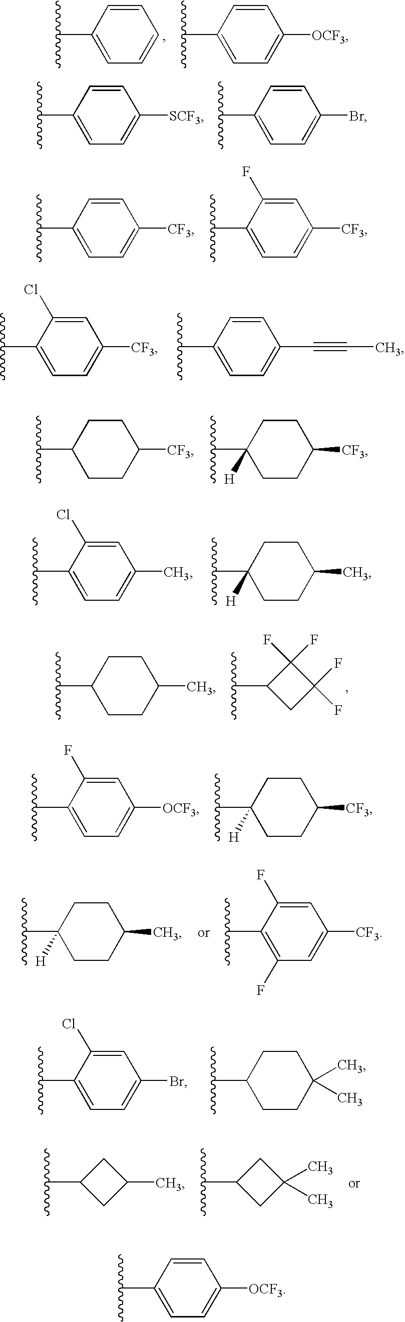

In one embodiment, J is a single bond; G is —CH₂—; R¹ is —NH₂; R² and R³ are each 4-fluorophenyl; and R⁴ is —C(O)OR⁵.

In another embodiment, J is a single bond; G is —CH₂—; R¹ is —NH₂; R² and R³ are each 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, J is a single bond; G is —CH₂—; R¹ is —NH₂; R² and R³ are each 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

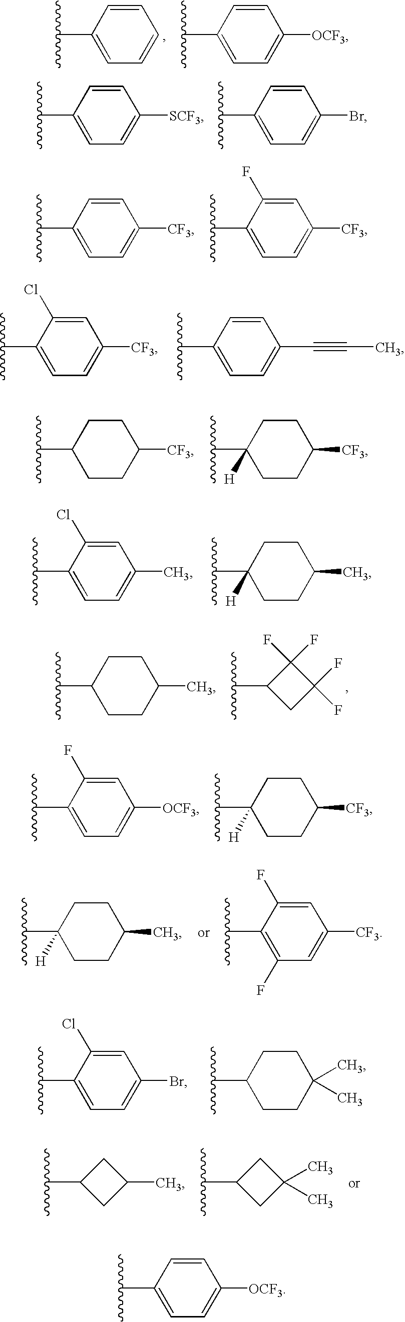

In one embodiment, for the compounds of formula (I), variables J, G, R, R¹, R², R³, R⁴, R¹⁰ and R¹¹ are selected independently of each other.

In another embodiment, the compounds of formula (I) are in purified form.

In one embodiment, the compounds of formula (I) have the formula (Ia):

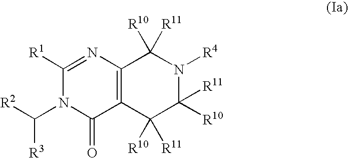

and pharmaceutically acceptable salts, solvates, esters and prodrugs thereof, wherein R¹, R², R³, R⁴, R¹⁰ and R¹¹ are defined above for the compounds of formula (I).

In one embodiment, R¹ is —H.

In one embodiment, R¹ is other than —H.

In another embodiment, R¹ is alkyl.

In another embodiment, R¹ is —N(R⁹)₂.

In still another embodiment, R¹ is —OR⁹.

In yet another embodiment, R¹ is —SR⁹.

In one embodiment, R¹ is —NH₂.

In another embodiment, R¹ is —NH-alkyl.

In another embodiment, R¹ is —N(alkyl)₂.

In still another embodiment, R¹ is —O-alkyl.

In a further embodiment, R¹ is —S-alkyl.

In another embodiment, R¹ is aryl.

In still another embodiment, R¹ is cycloalkyl.

In yet another embodiment, R¹ is cycloalkenyl.

In a further embodiment, R¹ is heterocycloalkyl.

In another embodiment, R¹ is heterocycloalkenyl.

In another embodiment, R¹ is heteroaryl.

In another embodiment, R¹ is -(alkylene)-aryl.

In still another embodiment, R¹ is -(alkylene)-cycloalkyl.

In yet another embodiment, R¹ is -(alkylene)-cycloalkenyl.

In a further embodiment, R¹ is -(alkylene)-heterocycloalkyl.

In another embodiment, R¹ is -(alkylene)-heterocycloalkenyl.

In another embodiment, R¹ is -(alkylene)-heteroaryl.

In still another embodiment, R¹ is haloalkyl.

In another embodiment, R¹ is fluoromethyl.

In another embodiment, R¹ is difluoromethyl.

In a further embodiment, R¹ is cyclopropyl.

In another embodiment, R¹ is alkenyl.

In another embodiment, R¹ is alkynyl.

In yet another embodiment, R¹ is propynyl.

In one embodiment, R¹ is methyl.

In another embodiment, R¹ is ethyl.

In another embodiment, R¹ is n-propyl.

In still another embodiment, R¹ isopropyl.

In a further embodiment, R¹ is benzyl.

In another embodiment, R¹ is phenyl.

In one embodiment, R² is aryl.

In another embodiment, R² is heteroaryl.

In still another embodiment, R² is alkyl.

In another embodiment, R² is benzyl.

In yet another embodiment, R² is cycloalkyl.

In another embodiment, R² is cyclopentyl or cyclohexyl.

In another embodiment, R² is heterocycloalkyl.

In a further embodiment, R² is —C(O)-aryl.

In another embodiment, R² is alkylene-aryl.

In another embodiment, R² is alkylene-O-aryl.

In another embodiment, R² is alkylene-O-alkyl.

In still another embodiment, R² is methyl.

In one embodiment, R² is phenyl, pyridyl or 4-fluorophenyl.

In another embodiment, R² is phenyl.

In yet another embodiment, R² is 4-trifluoromethyl-phenyl.

In one embodiment, R² is 4-fluorophenyl.

In another embodiment, R² is 2-(4-fluorophenyl)ethyl.

In another embodiment, R² is pyridyl.

In still another embodiment, R² is 2-pyridyl.

In one embodiment, R² is phenyl, pyridyl, 4-fluorophenyl, 3-fluorophenyl, cyclobutyl, benzyl or 3,4-difluorophenyl.

In another embodiment, R² is —C(O)NH₂.

In another embodiment, R² is —C(O)OR⁵.

In another embodiment, R² is —C(O)N(R⁶)₂.

In still another embodiment, R² is trifluoromethyl.

In yet another embodiment, R² is cyclopropyl.

In still another embodiment, R² is cyclobutyl.

In another embodiment, R² is cyclopentyl.

In one embodiment, R² is cyclohexyl.

In another embodiment, R² is alkylene-N(R⁹)₂

In another embodiment, R² is —CH₂—O-phenyl.

In one embodiment, R³ is aryl.

In another embodiment, R³ is heteroaryl.

In still another embodiment, R³ is alkyl.

In another embodiment, R³ is benzyl.

In still another embodiment, R³ is alkyl.

In yet another embodiment, R³ is cycloalkyl.

In another embodiment, R³ is cyclopentyl or cyclohexyl.

In another embodiment, R³ is heterocycloalkyl.

In a further embodiment, R³ is —C(O)-aryl.

In another embodiment, R³ is alkylene-aryl.

In another embodiment, R³ is alkylene-O-aryl.

In another embodiment, R³ is alkylene-O-alkyl.

In still another embodiment, R³ is methyl.

In another embodiment, R³ is phenyl.

In yet another embodiment, R³ is 4-trifluoromethyl-phenyl.

In one embodiment, R³ is 4-fluorophenyl.

In another embodiment, R³ is 2-(4-fluorophenyl)ethyl.

In another embodiment, R³ is pyridyl.

In still another embodiment, R³ is 2-pyridyl.

In another embodiment, R³ is —C(O)NH₂.

In another embodiment, R³ is —C(O)OR⁵.

In another embodiment, R³ is —C(O)N(R⁶)₂.

In still another embodiment, R³ is trifluoromethyl.

In yet another embodiment, R³ is cyclopropyl.

In still another embodiment, R³ is cyclobutyl.

In another embodiment, R³ is cyclopentyl.

In one embodiment, R³ is cyclohexyl.

In another embodiment, R³ is alkylene-N(R⁹)₂

In another embodiment, R³ is —CH₂—O-phenyl.

In one embodiment, R⁴ is H.

In another embodiment, R⁴ is alkyl.

In another embodiment, R⁴ is —S(O)_{q}R⁷.

In another embodiment, R⁴ is —C(O)R⁵.

In still another embodiment, R⁴ is -alkylene-O-alkyl.

In yet another embodiment, R⁴ is -alkylene-O-aryl.

In another embodiment, R⁴ is -alkylene-S-alkyl.

In another embodiment, R⁴ is -alkylene-S-aryl.

In another embodiment, R⁴ is -alkylene-NH-alkyl.

In yet another embodiment, R⁴ is -alkylene-NH-aryl.

In a further embodiment, R⁴ is C(O)OR⁵.

In another embodiment, R⁴ is —C(O)N(R⁶)₂.

In another embodiment, R⁴ is -(alkylene)-aryl.

In another embodiment, R⁴ is -(alkylene)-cycloalkyl.

In still another embodiment, R⁴ is -(alkylene)-cycloalkenyl.

In yet another embodiment, R⁴ is -(alkylene)-heterocycloalkyl.

In a further embodiment, R⁴ is -(alkylene)-heterocycloalkenyl.

In another embodiment, R⁴ is -(alkylene)-heteroaryl.

In another embodiment, R⁴ is aryl.

In another embodiment, R⁴ is benzyl.

In another embodiment, R⁴ is cycloalkyl.

In still another embodiment, R⁴ is cycloalkenyl.

In yet another embodiment, R⁴ is heterocycloalkyl.

In a further embodiment, R⁴ is heterocycloalkenyl.

In another embodiment, R⁴ is heteroaryl.

In another embodiment, R⁴ is —CH₂-heteroaryl.

In still another embodiment, R⁴ is phenyl.

In yet another embodiment, R⁴ is pyrimidinyl.

In another embodiment, R⁴ is 1,2,4-oxadiazolyl.

In a further embodiment, R⁴ is 4-trifluoromethyl-phenyl.

In another embodiment, R⁴ is —C(O)O-2,2,3,3-tetrafluorocyclobutyl.

In another embodiment, R⁴ is —C(O)O-trans-4-(trifluoromethyl)cyclohexyl.

In one embodiment, R⁴ is —C(O)OR⁵, wherein R⁵ is alkyl, aryl, haloalkyl, -alkylene-aryl, -cycloalkyl, -alkylene-O-alkylene-aryl, -alkylene-O-alkyl, or alkynyl.

In another embodiment, R⁴ is —C(O)OR⁵, wherein R⁵ is methyl, ethyl, n-propyl, isopropyl, n-butyl, tert-butyl, -neopentyl, —CH₂CH(—CH₂CH₃)—(CH₂)₃CH₃, —CH₂CH(CH₃)₂, n-hexyl or —CH₂—C≡CCH₃.

In another embodiment, R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

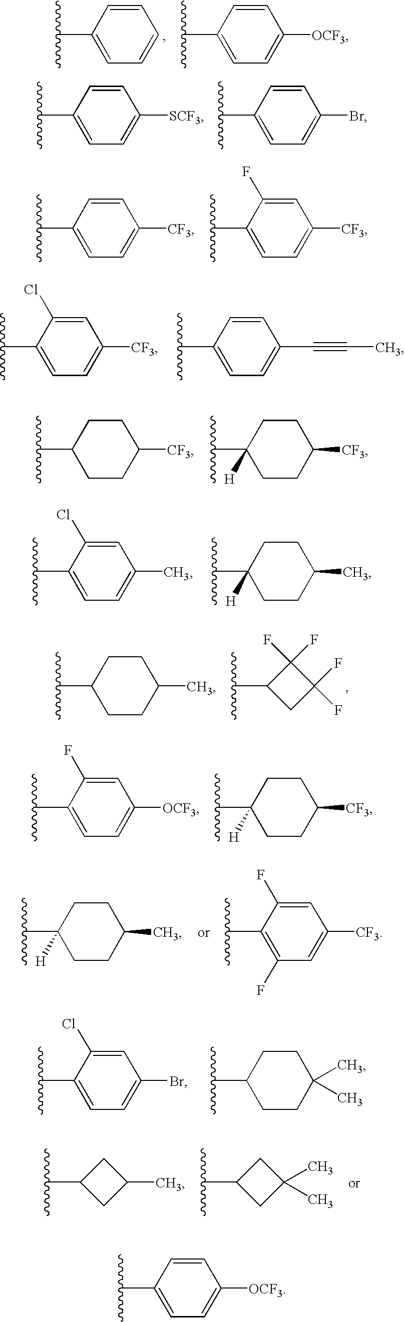

In still another embodiment, R⁴ is —C(O)OR⁵, wherein R⁵ is cyclopropyl, cyclobutyl, cyclopentyl or cyclohexyl.

In yet another embodiment, R⁴ is —C(O)OR⁵, wherein R⁵ is benzyl or 2-chlorobenzyl.

In another embodiment, R⁴ is —C(O)OR⁵, wherein R⁵ is —(CH₂)₂—O-benzyl or —(CH₂)₂—O—CH₃.

In another embodiment, R⁴ is —C(O)NHR⁵.

In still another embodiment, R⁴ is —C(O)NH-alkyl.

In another embodiment, R⁴ is —S(O)₂R⁷.

In another embodiment, R⁴ is —S(O)₂-alkyl.

In still another embodiment, R⁴ is —S(O)₂-aryl.

In still another embodiment, R⁴ is —S(O)₂-phenyl.

In one embodiment, each occurrence of R¹⁰ is H.

In another embodiment, each occurrence of R¹¹ is H.

In another embodiment, each occurrence of R¹⁰ and R¹¹ is H.

In another embodiment, one occurrence of R¹⁰ or R¹¹ is other than hydrogen.

In yet another embodiment, at least one occurrence of R¹⁰ or R¹¹ is alkyl.

In still another embodiment, at least one occurrence of R¹⁰ or R¹¹ is methyl.

In another embodiment, R⁴ is benzyl, wherein the phenyl ring of the benzyl group can be unsubstituted or substituted with up to 3 substituents, which may be the same or different, and are selected from: F, Br, Cl, —NO₂, —CH₃, —CF₃, —SCF₃, —C(O)O-alkyl, pyrrolyl, thiazolyl, —C≡C-phenyl, —OCHF₂, piperidinyl, pyridyl, pyrrolidinyl, pyrazolyl, methoxy, piperazinyl, morpholinyl, —OCF₂CHF₂, 1,3,4-triazolyl, —CH(OH)CH₃, —OH, —SO₂CH₃, —C(O)OH or -phenyl.

In one embodiment, R⁴ is —CH₂-heteroaryl, wherein the heteroaryl is thienyl, benzthienyl, thiazolyl, benzthiazolyl, furanyl, benzofuranyl, pyridyl, isoxazolyl or benzimidazolyl.

In one embodiment, one or more occurrences of n is 1.

In another embodiment, one or more occurrences of n is 0.

In another embodiment, one or more occurrences of p is 0.

In still another embodiment, one or more occurrences of p is 1.

In yet another embodiment, one or more occurrences of p is 2.

In one embodiment, one or more occurrences of q is 1.

In another embodiment, one or more occurrences of q is 2.

In another embodiment, R² and R³ are each aryl.

In yet another embodiment, R² and R³ are each heteroaryl.

In another embodiment, R² and R³ are each phenyl.

In another embodiment, R² is aryl and R³ is heteroaryl.

In still another embodiment, R² is phenyl and R³ is heteroaryl.

In yet another embodiment, R² is phenyl and R³ is pyridyl.

In a further embodiment, R² is phenyl and R³ is 2-pyridyl.

In another embodiment, R² and R³ are each 4-trifluoromethylphenyl.

In another embodiment, R² and R³ are each 4-chlorophenyl.

In one embodiment, R² and R³ are each 4-fluorophenyl.

In another embodiment, R² is aryl and R³ is cycloalkyl.

In still another embodiment, R² is phenyl and R³ is cycloalkyl.

In a further embodiment, R² is phenyl and R³ is cyclopentyl.

In another embodiment, R² is phenyl and R³ is cyclobutyl.

In still another embodiment, R² is phenyl and R³ is 4-fluorophenyl.

In yet another embodiment, R² is phenyl and R³ is pyrimidinyl.

In still another embodiment, R² is phenyl and R³ is thienyl.

In another embodiment, R¹ is alkyl, R² is aryl and R³ is heteroaryl.

In still another embodiment, R¹ is alkyl, R² is phenyl and R³ is heteroaryl.

In yet another embodiment, R¹ is alkyl, R² is phenyl and R³ is pyridyl.

In another embodiment, R¹ is alkyl, R² is phenyl and R³ is 4-fluorophenyl.

In another embodiment, R¹ is alkyl, R² is phenyl and R³ is 2-pyridyl.

In a further embodiment, R¹ is alkyl, and R² and R³ are each aryl.

In another embodiment, R¹ is alkyl, and R² and R³ are each heteroaryl.

In yet another embodiment, R¹ is alkyl, and R² and R³ are each phenyl.

In another embodiment, R¹ is alkyl, and R² and R³ are each 4-trifluoromethylphenyl.

In a further embodiment, R¹ is alkyl, and R² and R³ are each 4-chlorophenyl.

In one embodiment, R¹ is alkyl, and R² and R³ are each 4-fluorophenyl.

In still another embodiment, R¹ is alkyl, R² is phenyl and R³ is 4-fluorophenyl.

In another embodiment, R¹ is benzyl, R² is aryl and R³ is heteroaryl.

In still another embodiment, R¹ is benzyl, R² is phenyl and R³ is heteroaryl.

In yet another embodiment, R¹ is benzyl, R² is phenyl and R³ is pyridyl.

In another embodiment, R¹ is benzyl, R² is phenyl and R³ is 2-pyridyl.

In another embodiment, R¹ is benzyl, R² is phenyl and R³ is 4-fluorophenyl.

In a further embodiment, R¹ is benzyl, and R² and R³ are each aryl.

In another embodiment, R¹ is benzyl, and R² and R³ are each heteroaryl.

In yet another embodiment, R¹ is benzyl, and R² and R³ are each phenyl.

In another embodiment, R¹ is benzyl, and R² and R³ are each 4-trifluoromethylphenyl.

In a further embodiment, R¹ is benzyl, and R² and R³ are each 4-chlorophenyl.

In one embodiment, R¹ is benzyl, and R² and R³ are each 4-fluorophenyl.

In one embodiment, R¹ is —N(R⁹)₂, R² is aryl and R³ is heteroaryl.

In another embodiment, R¹ is —N(R⁹)₂, R² is phenyl and R³ is heteroaryl.

In yet another embodiment, R¹ is —N(R⁹)₂, R² is phenyl and R³ is pyridyl.

In another embodiment, R¹ is —N(R⁹)₂, R² is phenyl and R³ is 2-pyridyl.

In yet another embodiment, R¹ is —N(R⁹)₂, R² is phenyl and R³ is 4-fluorophenyl.

In a further embodiment, R¹ is —N(R⁹)₂, and R² and R³ are each aryl.

In another embodiment, R¹ is —N(R⁹)₂, and R² and R³ are each heteroaryl.

In yet another embodiment, R¹ is —N(R⁹)₂, and R² and R³ are each phenyl.

In another embodiment, R¹ is —N(R⁹)₂, and R² and R³ are each 4-trifluoromethylphenyl.

In another embodiment, R¹ is —N(R⁹)₂, and R² and R³ are each 4-chlorophenyl.

In still another embodiment, R¹ is —N(R⁹)₂, and R² and R³ are each 4-fluorophenyl.

In one embodiment, R¹ is —NH₂, R² is aryl and R³ is heteroaryl.

In another embodiment, R¹ is —NH₂, R² is phenyl and R³ is heteroaryl.

In yet another embodiment, R¹ is —NH₂, R² is phenyl and R³ is pyridyl.

In another embodiment, R¹ is —NH₂, R² is phenyl and R³ is 2-pyridyl.

In another embodiment, R¹ is —NH₂, R² is phenyl and R³ is 4-fluorophenyl.

In a further embodiment, R¹ is —NH₂, and R² and R³ are each aryl.

In another embodiment, R¹ is —NH₂, and R² and R³ are each heteroaryl.

In yet another embodiment, R¹ is —NH₂, and R² and R³ are each phenyl.

In another embodiment, R¹ is —NH₂, and R² and R³ are each 4-trifluoromethylphenyl.

In a further embodiment, R¹ is —NH₂, and R² and R³ are each 4-chlorophenyl.

In another embodiment, R¹ is —NH₂, and R² and R³ are each 4-fluorophenyl.

In one embodiment, R¹ is methyl, R² is aryl and R³ is heteroaryl.

In still another embodiment, R¹ is methyl, R² is phenyl and R³ is heteroaryl.

In yet another embodiment, R¹ is methyl, R² is phenyl and R³ is pyridyl.

In another embodiment, R¹ is methyl, R² is phenyl and R³ is 2-pyridyl.

In another embodiment, R¹ is methyl, R² is phenyl and R³ is 4-fluorophenyl.

In a further embodiment, R¹ is methyl and R² and R³ are each aryl.

In another embodiment, R¹ is methyl and R² and R³ are each heteroaryl.

In another embodiment, R¹ is methyl and R² and R³ are each phenyl.

In another embodiment, R¹ is methyl and R² and R³ are each 4-trifluoromethylphenyl.

In a further embodiment, R¹ is methyl and R² and R³ are each 4-chlorophenyl.

In another embodiment, R¹ is methyl and R² and R³ are each 4-fluorophenyl.

In one embodiment, R¹ is methyl, R² and R³ are each unsubstituted or substituted phenyl, and R⁴ is —C(O)OR⁵.

In another embodiment, R¹ is methyl, R² and R³ are each phenyl, and R⁴ is —C(O)OR⁵.

In another embodiment, R¹ is alkyl; R² and R³ are each unsubstituted or substituted phenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is alkyl; R² and R³ are each unsubstituted or substituted phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

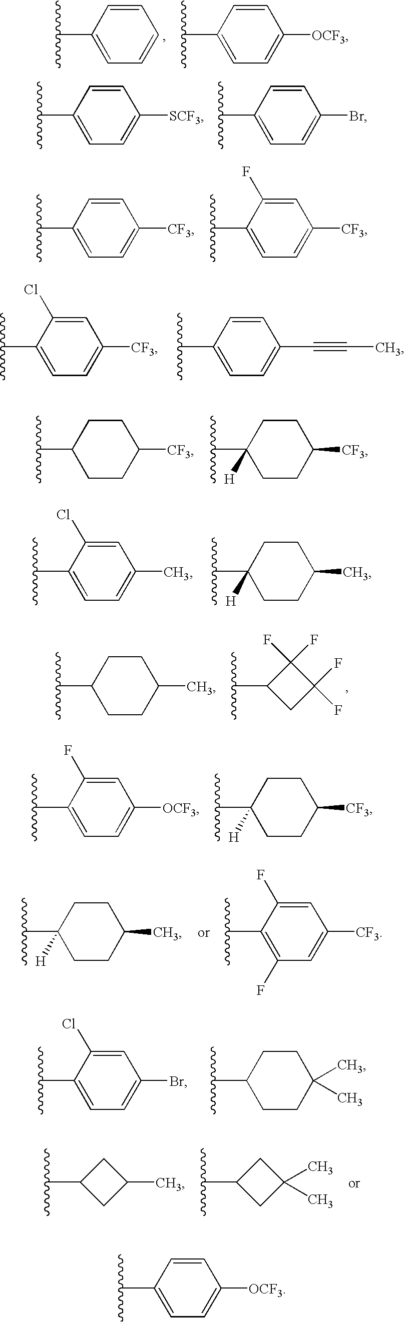

In another embodiment, R¹ is alkyl; R² and R³ are each phenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is alkyl; R² and R³ are each phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

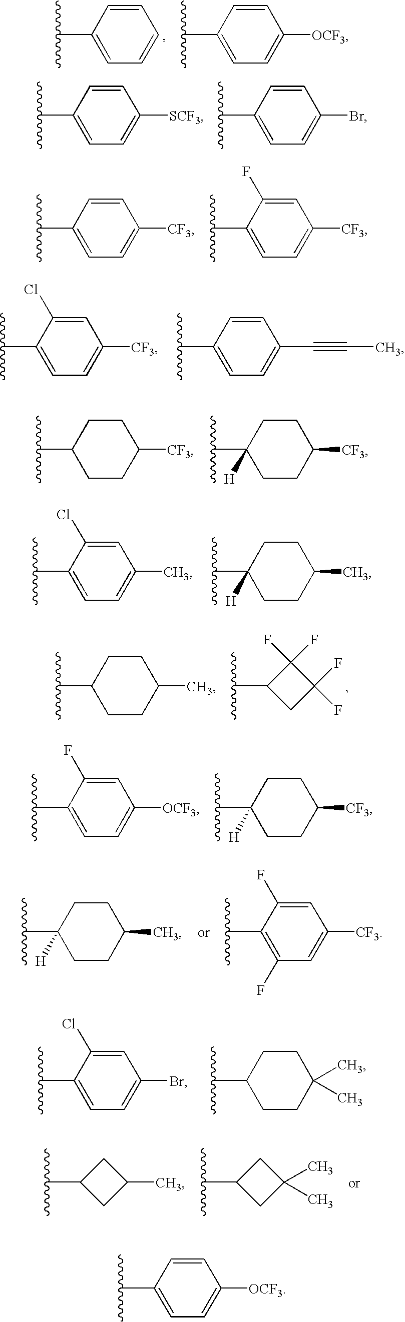

In one embodiment, R¹ is methyl; R² is phenyl; R³ is 4-fluorophenyl; and R⁴ is —C(O)OR⁵.

In another embodiment, R¹ is alkyl; R² is phenyl; R³ is 4-fluorophenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is alkyl; R² is phenyl; R³ is 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

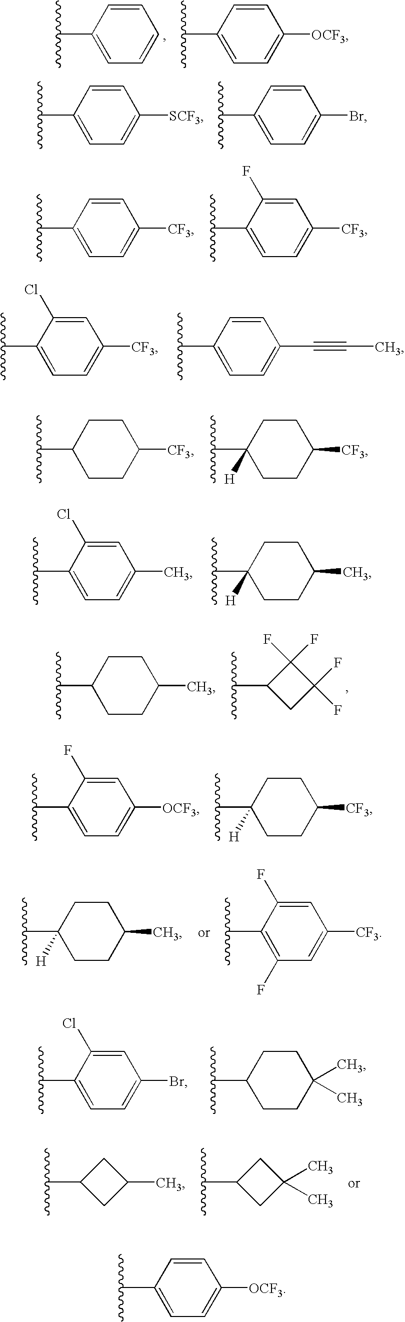

In one embodiment, R¹ is alkyl; R² and R³ are each independently cyclopentyl, cyclobutyl, 3-fluorophenyl or 4-fluorophenyl; and R⁴ is —C(O)OR⁵.

In another embodiment, R¹ is alkyl; R² and R³ are each independently cyclopentyl, cyclobutyl, 3-fluorophenyl or 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is alkyl; R² and R³ are each independently cyclopentyl, cyclobutyl, 3-fluorophenyl or 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

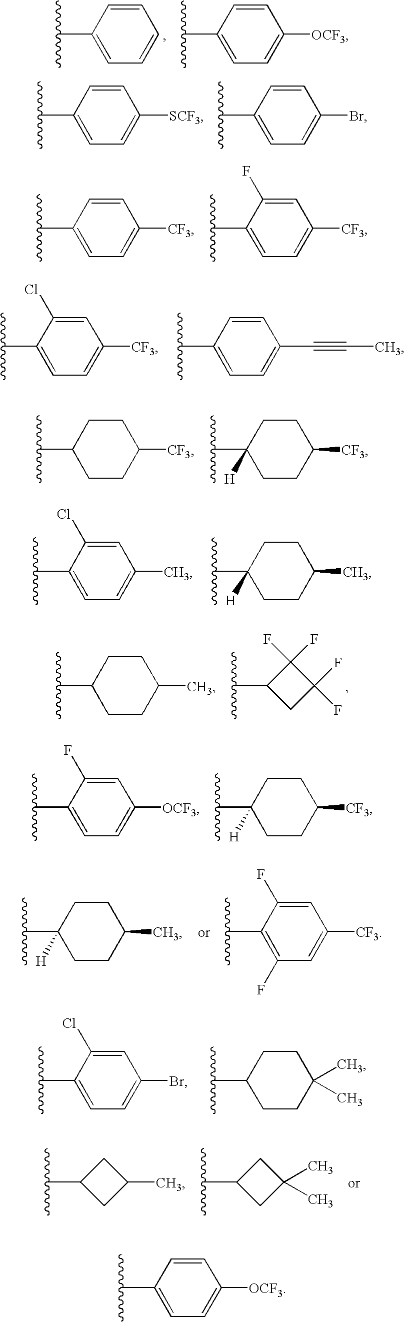

In one embodiment, R¹ is alkyl; R² and R³ are each 4-fluorophenyl; and R⁴ is —C(O)OR⁵.

In another embodiment, R¹ is alkyl; R² and R³ are each 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is alkyl; R² and R³ are each 4-fluorophenyl; and R⁴ is —C(O)OR⁵, wherein R⁵ is -tert-butyl, —CH₂CO₃, —C(CH₃)₂CCl₃, —CH₂CF₂CF₃, —CH(CF₃)₂, —CH₂CH(CF₃)₂,

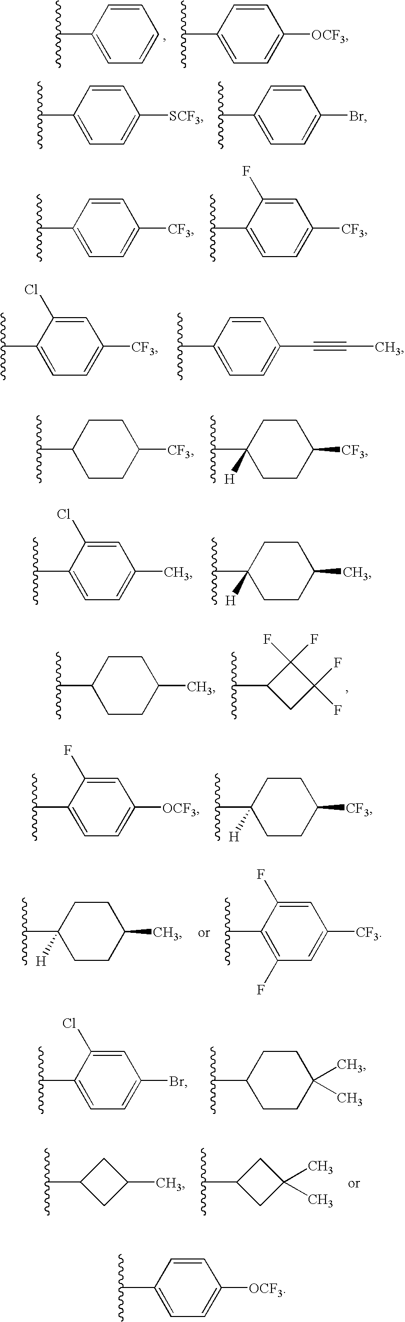

In one embodiment, R¹ is methyl; R² and R³ are each phenyl; and R⁴ is —C(O)OR⁵.

In another embodiment, R¹ is methyl; R² and R³ are each phenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is methyl; R² and R³ are each phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

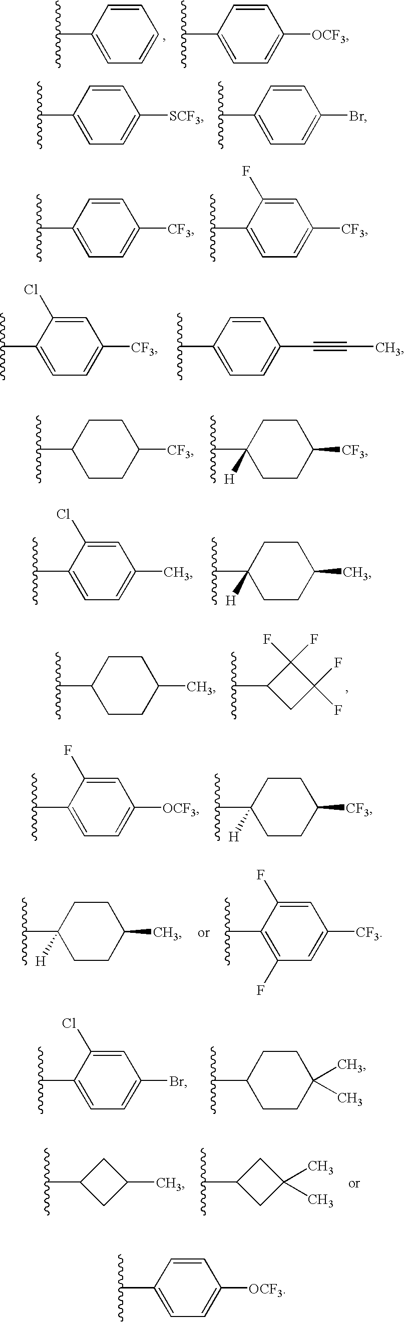

In one embodiment, R¹ is methyl; R² is phenyl; R³ is 4-fluorophenyl; and R⁴ is —C(O)OR⁵.

In another embodiment, R¹ is methyl; R² is phenyl; R³ is 4-fluorophenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is methyl; R² is phenyl; R³ is 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

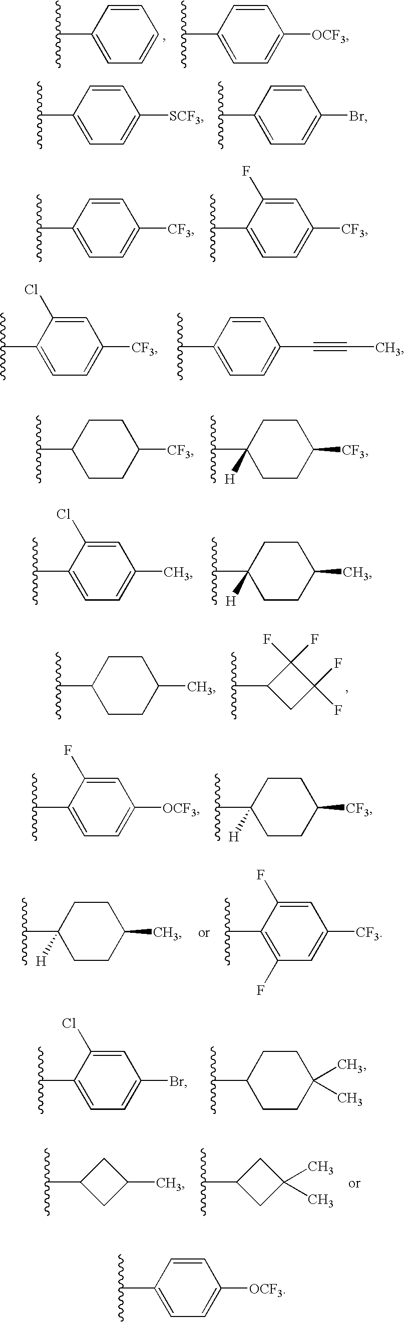

In one embodiment, R¹ is methyl; R² and R³ are each independently cyclopentyl, cyclobutyl, 3-fluorophenyl or 4-fluorophenyl; and R⁴ is —C(O)OR⁵.

In another embodiment, R¹ is methyl; R² and R³ are each independently cyclopentyl, cyclobutyl, 3-fluorophenyl or 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is methyl; R² and R³ are each independently cyclopentyl, cyclobutyl, 3-fluorophenyl or 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

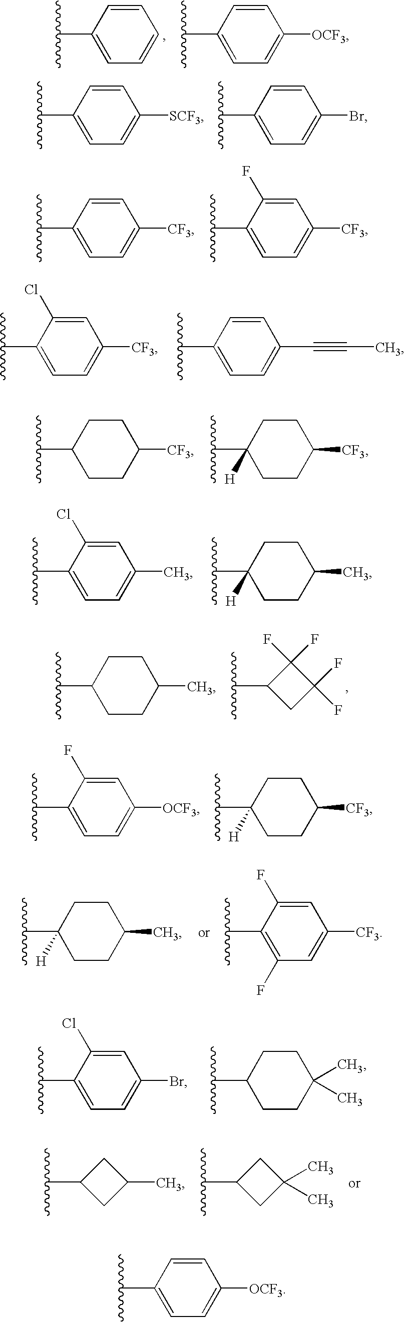

In one embodiment, R¹ is methyl; R² and R³ are each 4-fluorophenyl; and R⁴ is —C(O)OR⁵.

In another embodiment, R¹ is methyl; R² and R³ are each 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is methyl; R² and R³ are each 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

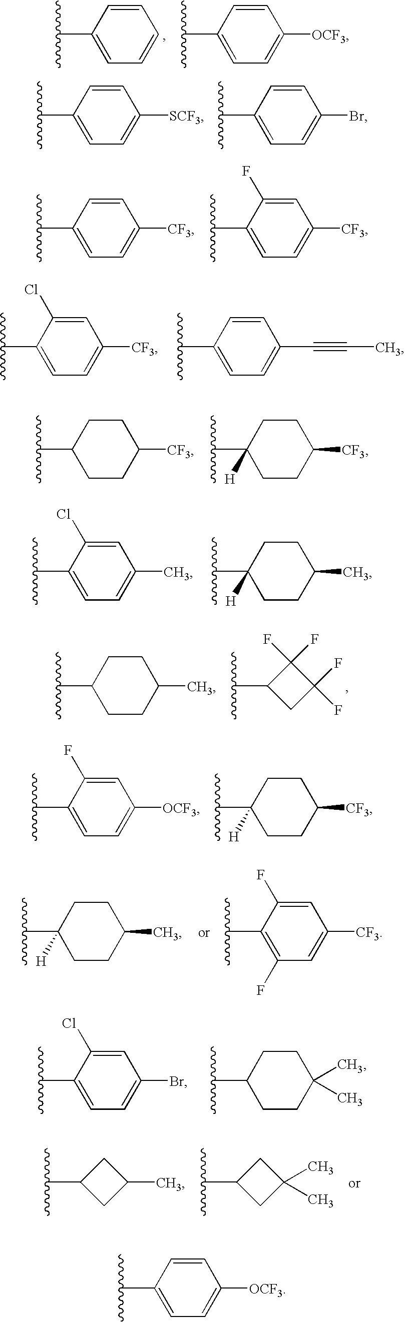

In one embodiment, R¹ is —N(R⁹)₂; R² and R³ are each unsubstituted or substituted phenyl; and R⁴ is —C(O)OR⁵.

In another embodiment, R¹ is —N(R⁹)₂; R² and R³ are each unsubstituted or substituted phenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —N(R⁹)₂; R² and R³ are each unsubstituted or substituted phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

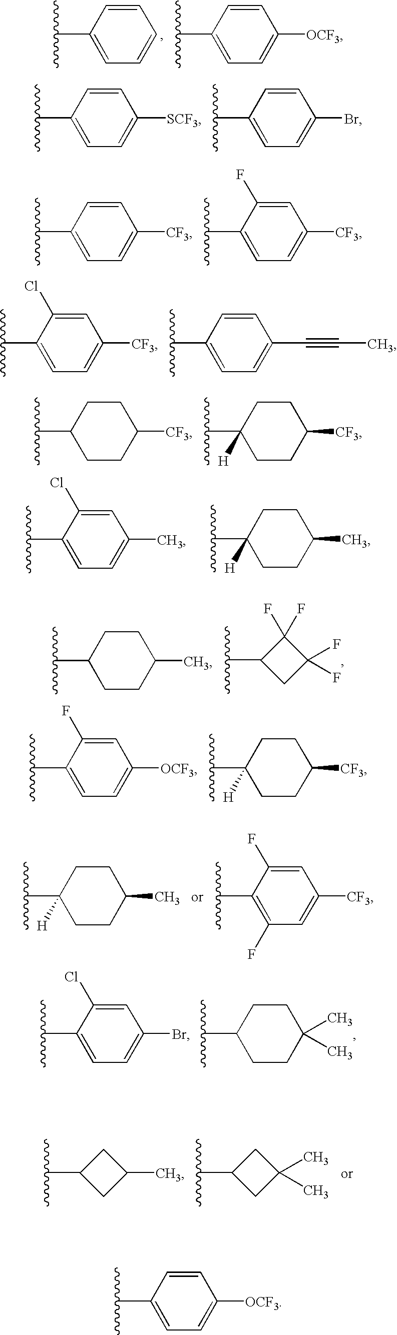

In one embodiment, R¹ is —N(R⁹)₂; R² and R³ are each phenyl; and R⁴ is —C(O)OR⁵.

In another embodiment, R¹ is —N(R⁹)₂; R² and R³ are each phenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —N(R⁹)₂; R² and R³ are each phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

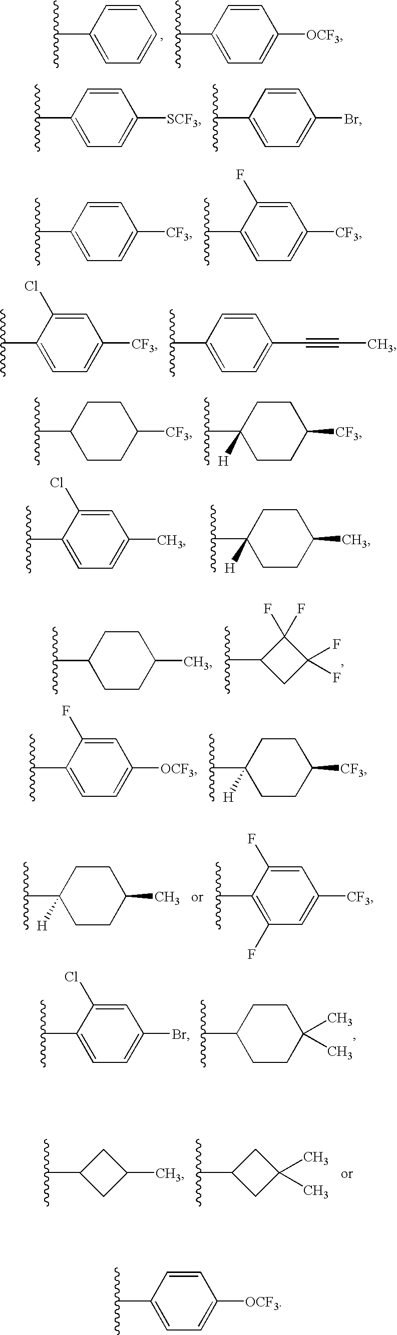

In one embodiment, R¹ is —N(R⁹)₂; R² is phenyl; R³ is 4-fluorophenyl; and R⁴ is —C(O)OR⁵.

In another embodiment, R¹ is —N(R⁹)₂; R² is phenyl; R³ is 4-fluorophenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —N(R⁹)₂; R² is phenyl; R³ is 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, CH₂CH(CF₃)₂,

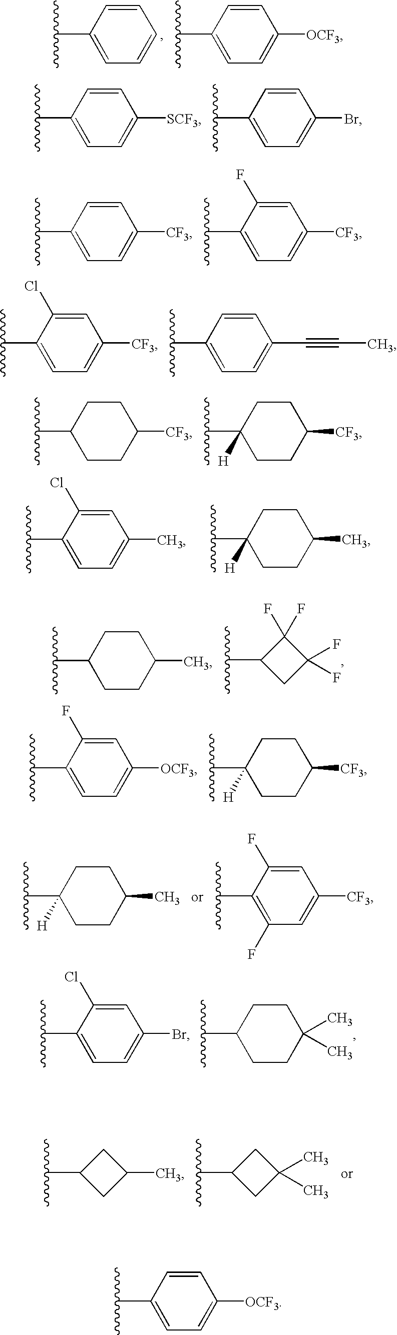

In one embodiment, R¹ is —N(R⁹)₂; R² and R³ are each independently cyclopentyl, cyclobutyl, 3-fluorophenyl or 4-fluorophenyl; and R⁴ is —C(O)OR⁵.

In another embodiment, R¹ is —N(R⁹)₂; R² and R³ are each independently cyclopentyl, cyclobutyl, 3-fluorophenyl or 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —N(R⁹)₂; R² and R³ are each independently cyclopentyl, cyclobutyl, 3-fluorophenyl or 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

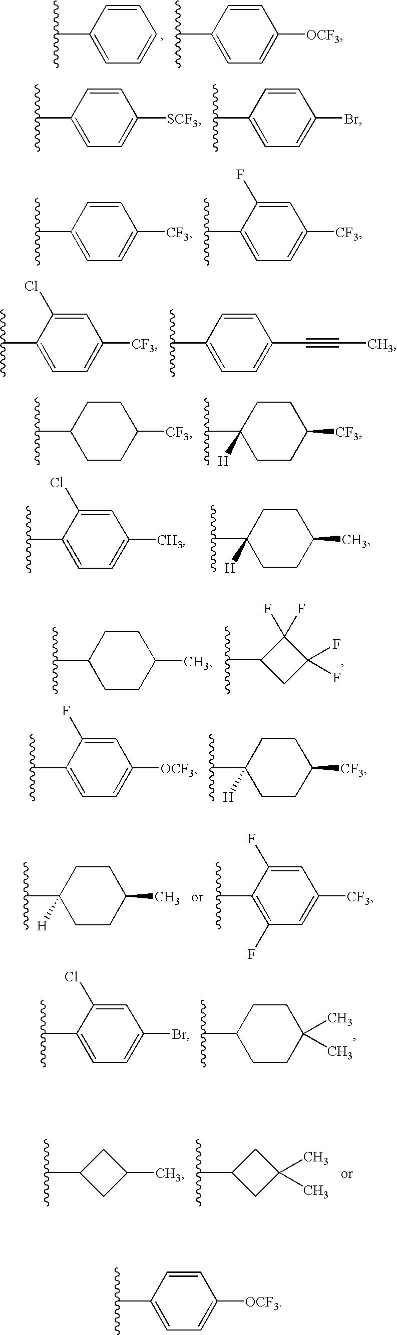

In one embodiment, R¹ is —N(R⁹)₂; R² and R³ are each 4-fluorophenyl; and R⁴ is —C(O)OR⁵.

In another embodiment, R¹ is —N(R⁹)₂; R² and R³ are each 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —N(R⁹)₂; R² and R³ are each 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

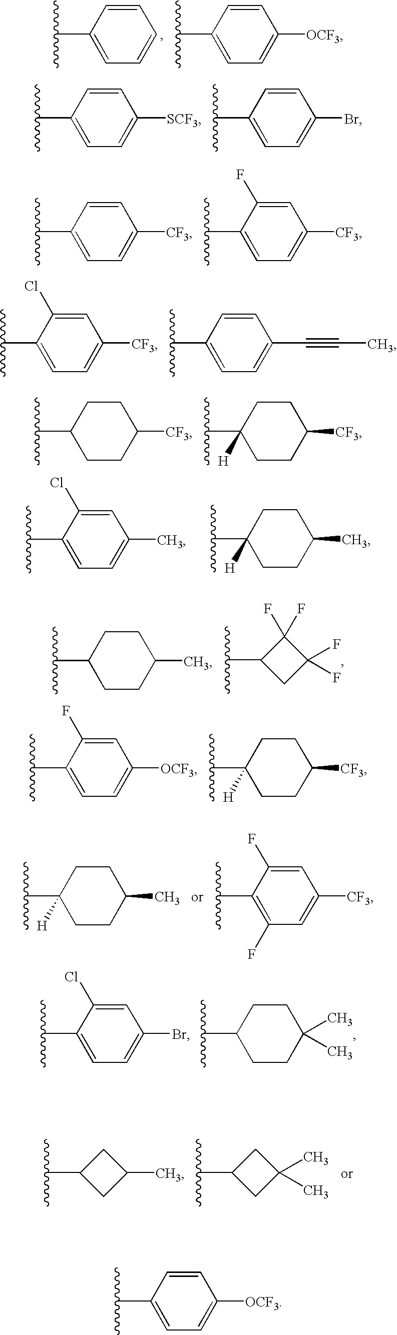

In one embodiment, R¹ is —NH₂; R² and R³ are each unsubstituted or substituted phenyl; and R⁴ is —C(O)OR⁵.

In another embodiment, R¹ is —NH₂; R² and R³ are each unsubstituted or substituted phenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —NH₂; R² and R³ are each unsubstituted or substituted phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

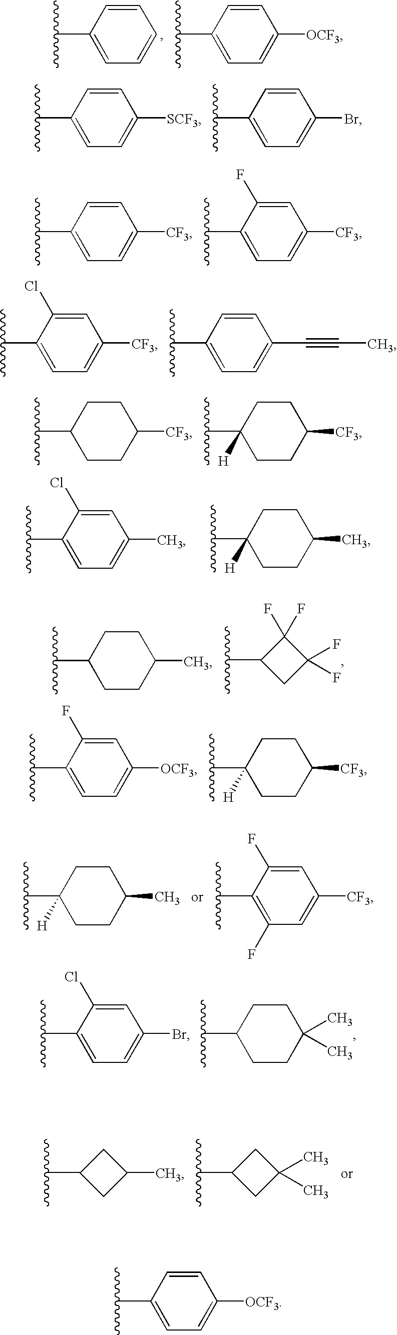

In one embodiment, R¹ is —NH₂; R² and R³ are each phenyl; and R⁴ is —C(O)OR⁵.

In another embodiment, R¹ is —NH₂; R² and R³ are each phenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —NH₂; R² and R³ are each phenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

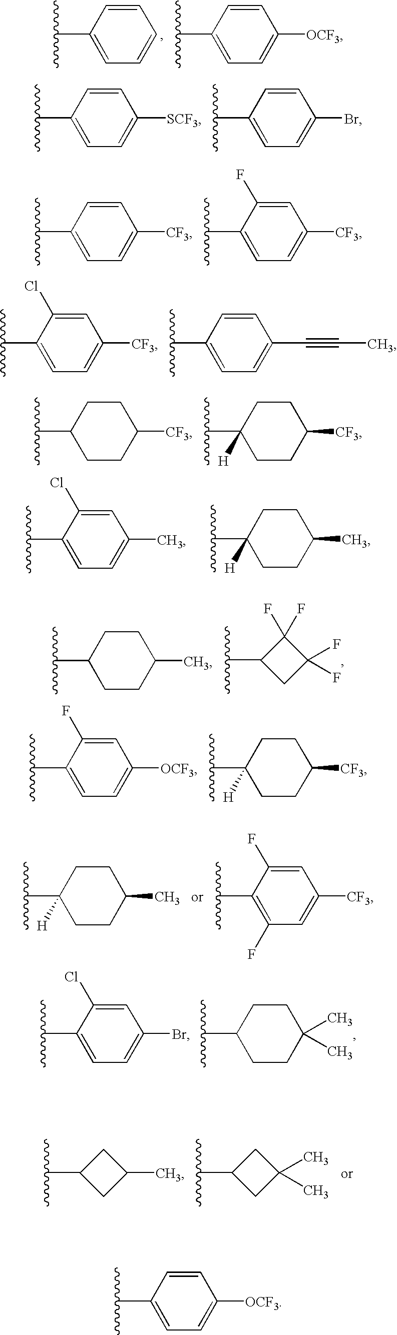

In one embodiment, R¹ is —NH₂; R² is phenyl; R³ is 4-fluorophenyl; and R⁴ is —C(O)OR⁵.

In another embodiment, R¹ is —NH₂; R² is phenyl; R³ is 4-fluorophenyl; and R⁴ is —C(O)O-aryl, wherein the phenyl moiety of the —C(O)O-aryl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —NH₂; R² is phenyl; R³ is 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

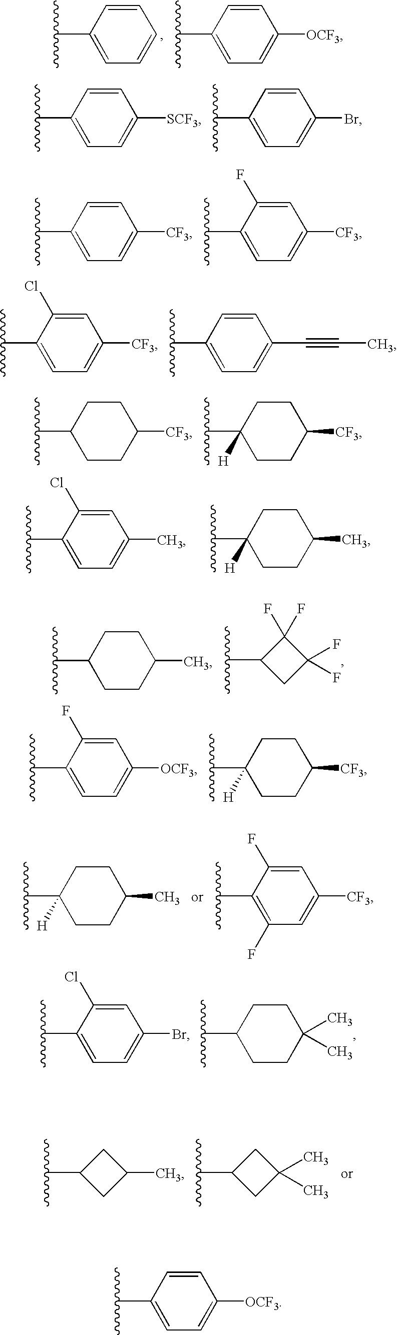

In one embodiment, R¹ is —NH₂; R² and R³ are each independently cyclopentyl, cyclobutyl, 3-fluorophenyl or 4-fluorophenyl; and R⁴ is —C(O)OR⁵.

In another embodiment, R¹ is —NH₂; R² and R³ are each independently cyclopentyl, cyclobutyl, 3-fluorophenyl or 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —NH₂; R² and R³ are each independently cyclopentyl, cyclobutyl, 3-fluorophenyl or 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

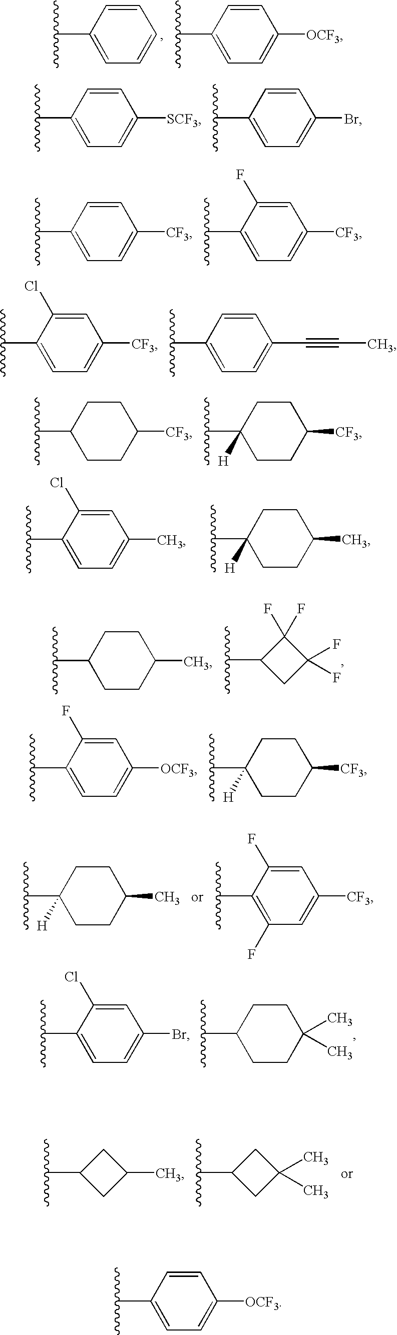

In one embodiment, R¹ is —NH₂; R² and R³ are each 4-fluorophenyl; and R⁴ is —C(O)OR⁵.

In another embodiment, R¹ is —NH₂; R² and R³ are each 4-fluorophenyl; and R⁴ is —C(O)O-phenyl, wherein the phenyl moiety of the —C(O)O-phenyl group is unsubstituted or substituted with up to 2 substituents independently selected from: alkyl, —C(O)O-alkyl, halo, haloalkyl, —O-haloalkyl, —S-alkyl or —O-alkyl.

In another embodiment, R¹ is —NH₂; R² and R³ are each 4-fluorophenyl; and R⁴ is —C(O)OR⁵, wherein R⁵ is -tert-butyl, —CH₂CCl₃, —C(CH₃)₂CCl₃, —CH₂CF₂CF₃, —CH(CF₃)₂, —CH₂CH(CF₃)₂,

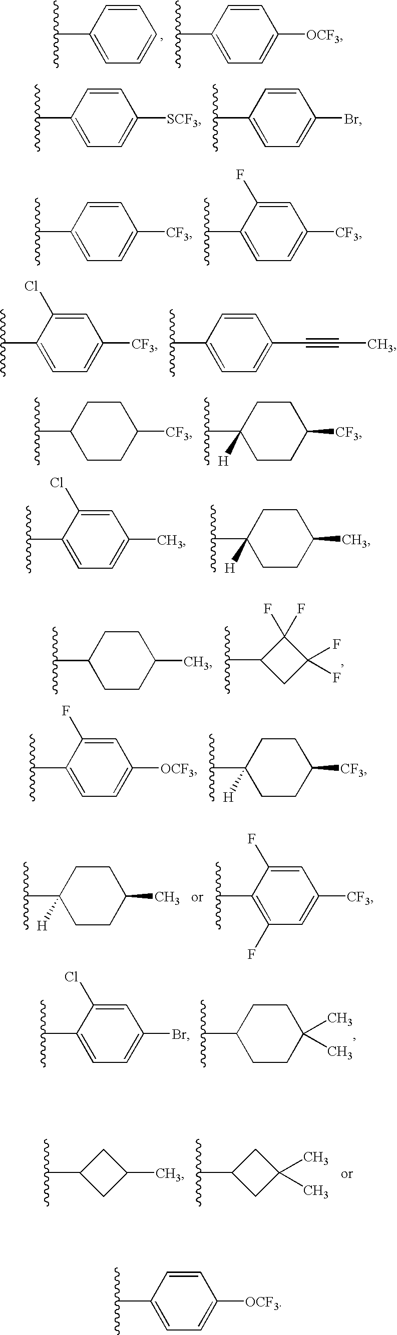

In one embodiment, for the compounds of formula (Ia), variables R¹, R², R³, R⁴, R¹⁰ and R¹¹ are selected independently of each other.

In another embodiment, the compounds of formula (Ia) are in purified form.

Non-limiting examples of the Pyrimidinone Derivatives of formula (I) include the following compounds:

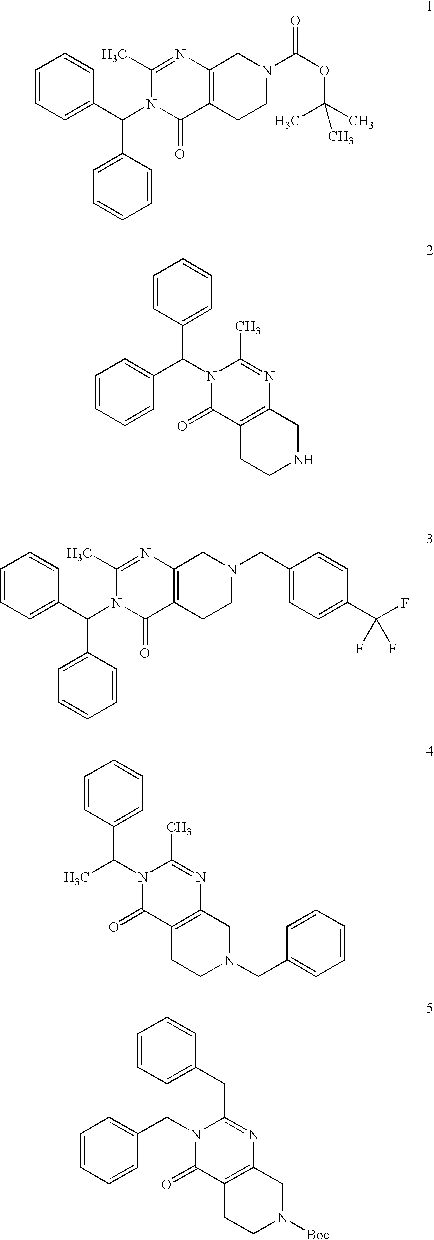

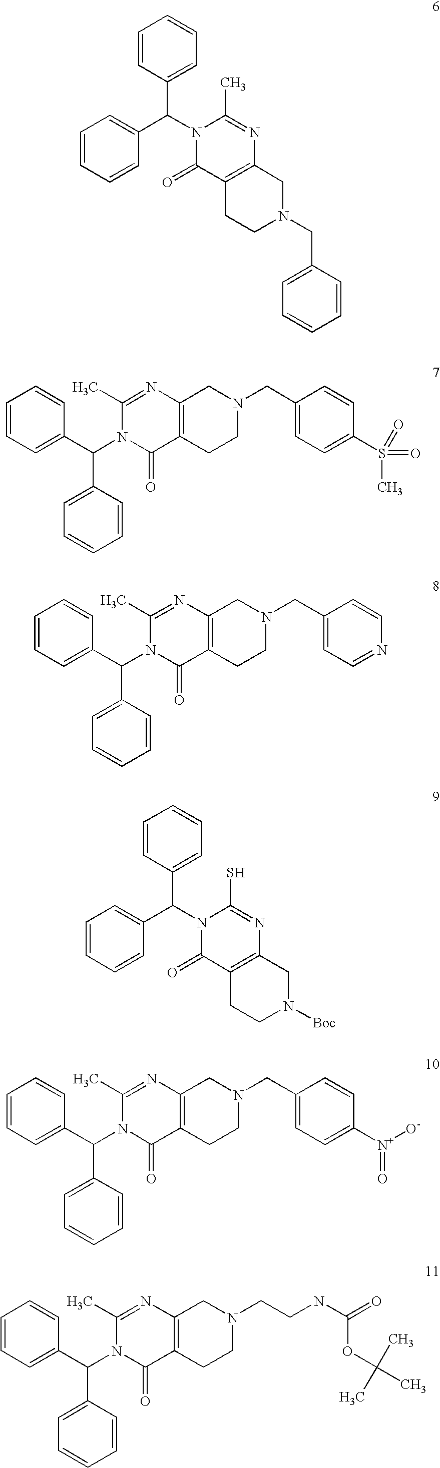

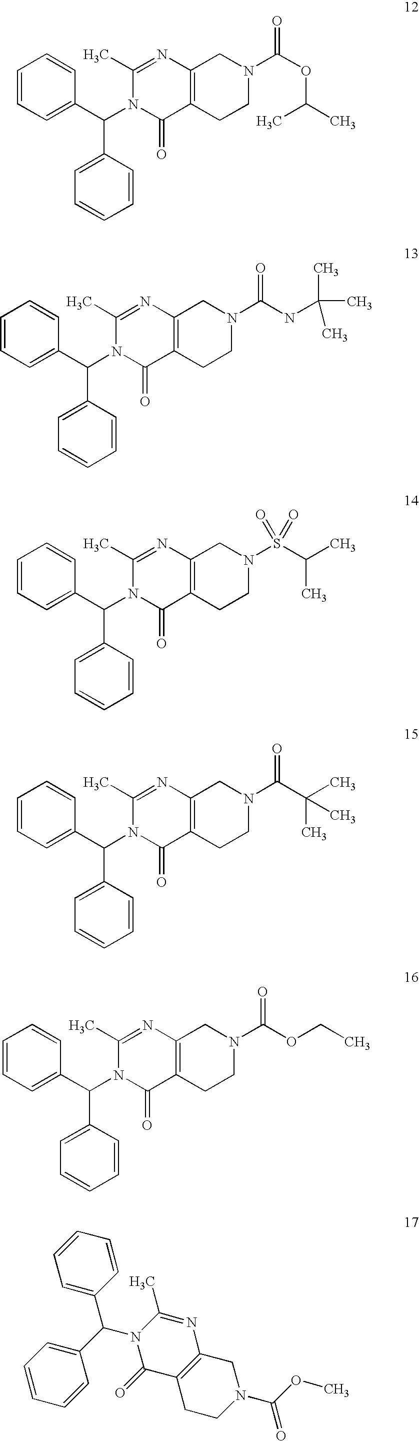

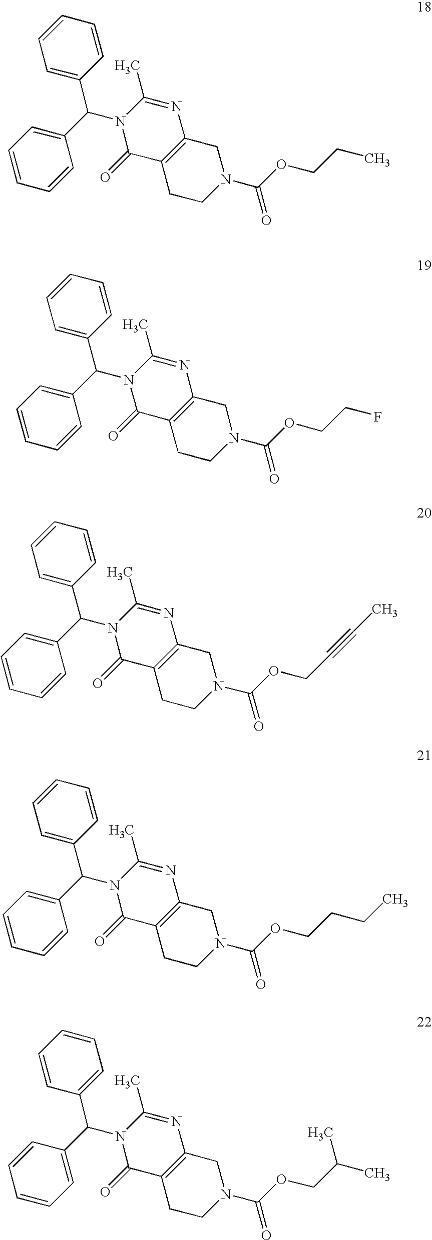

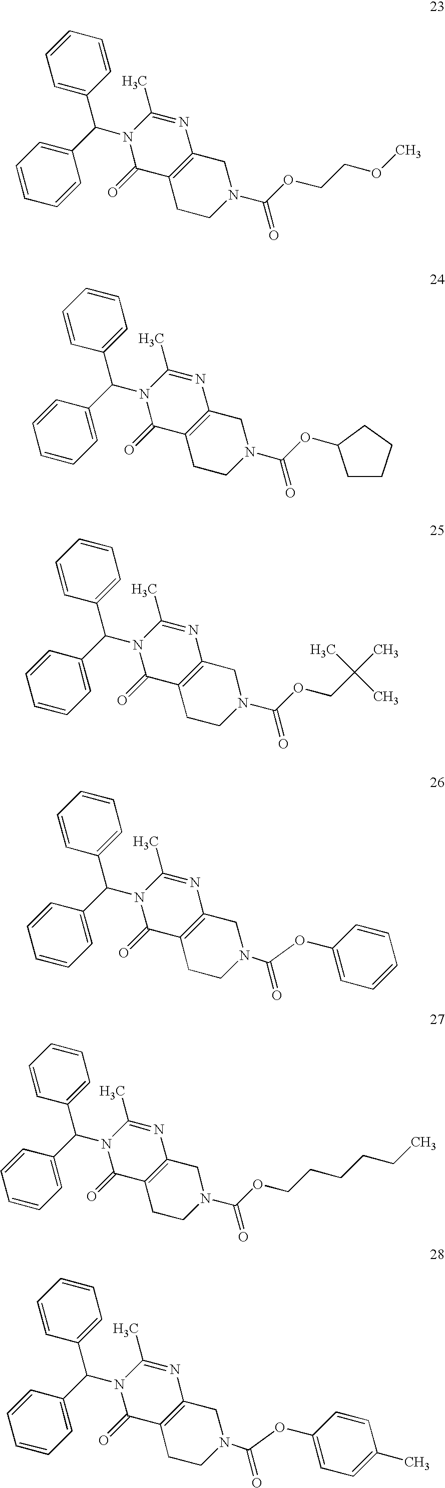

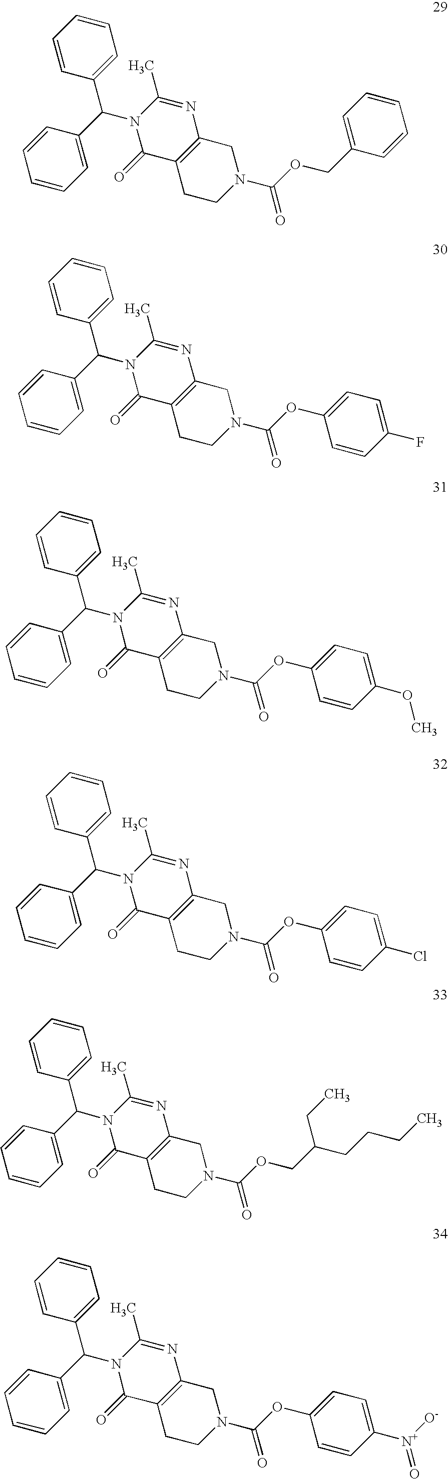

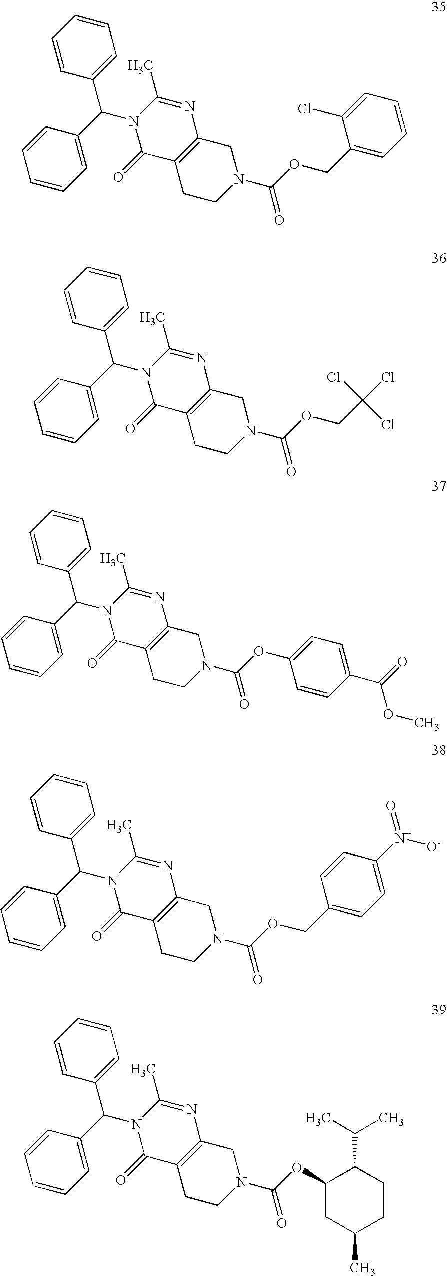

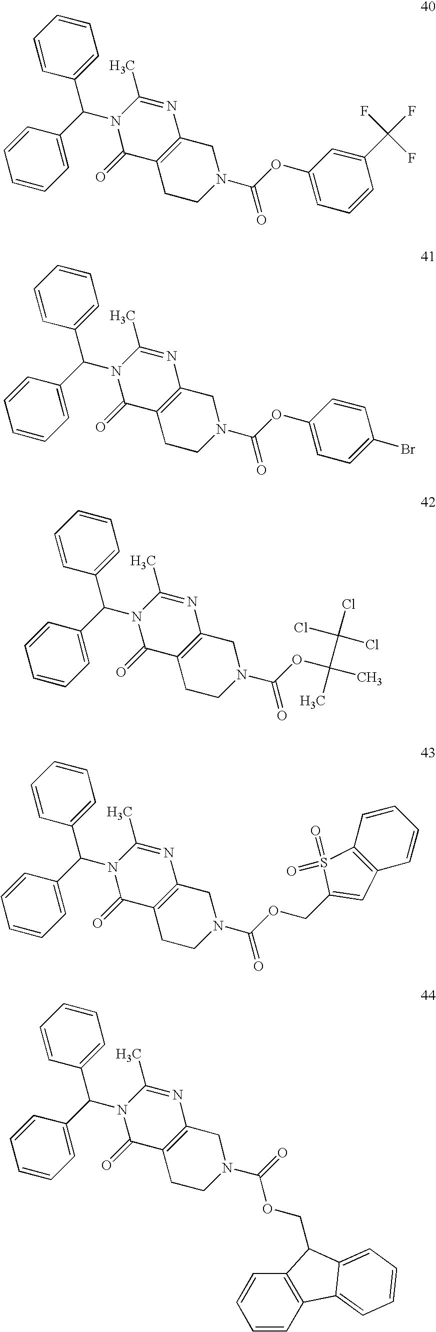

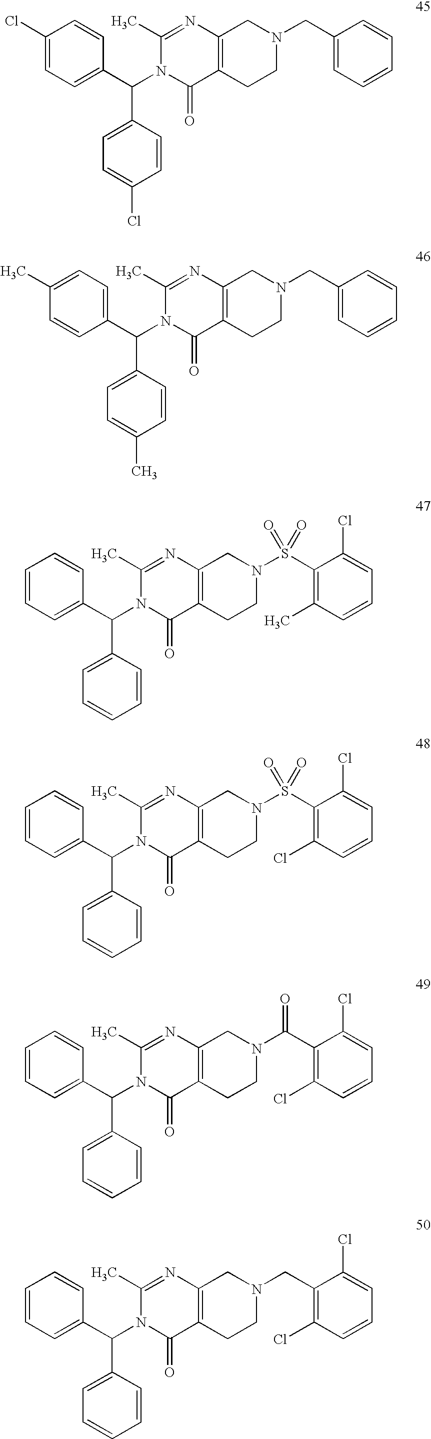

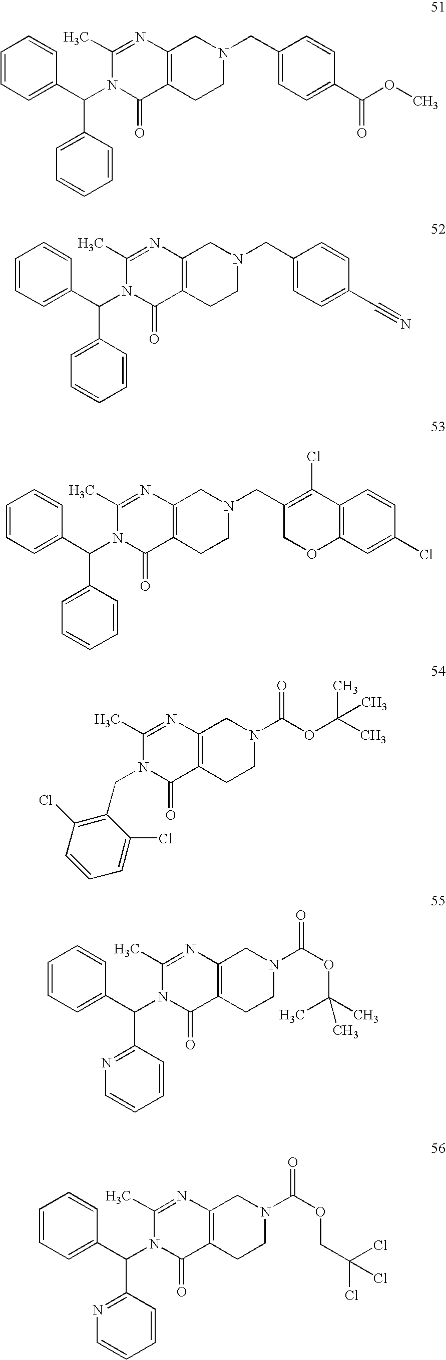

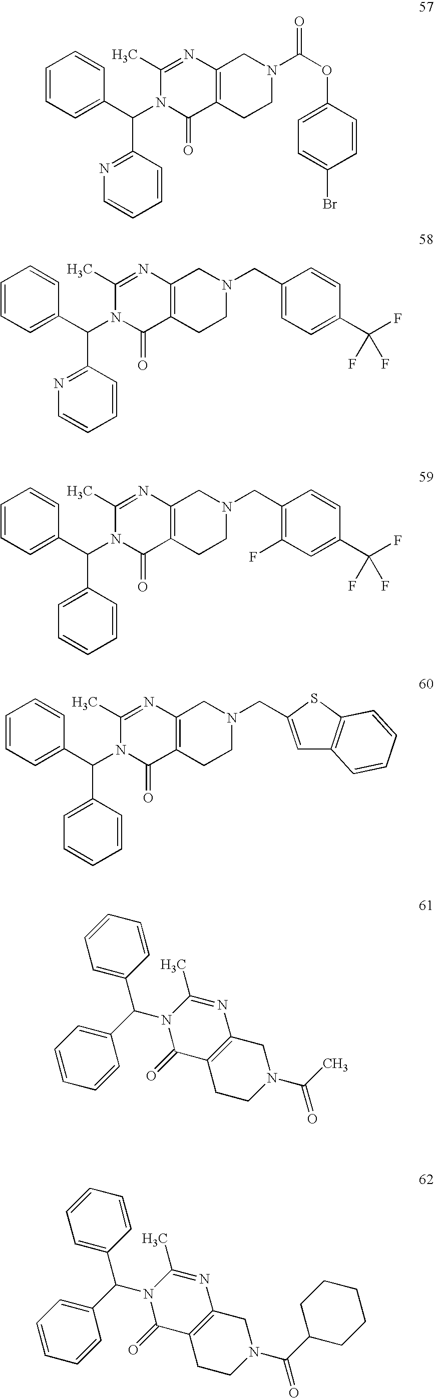

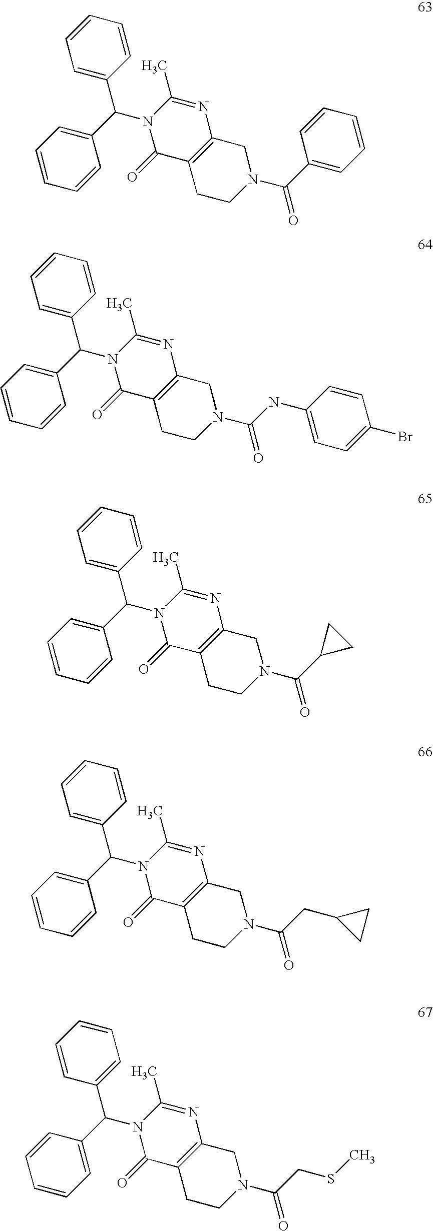

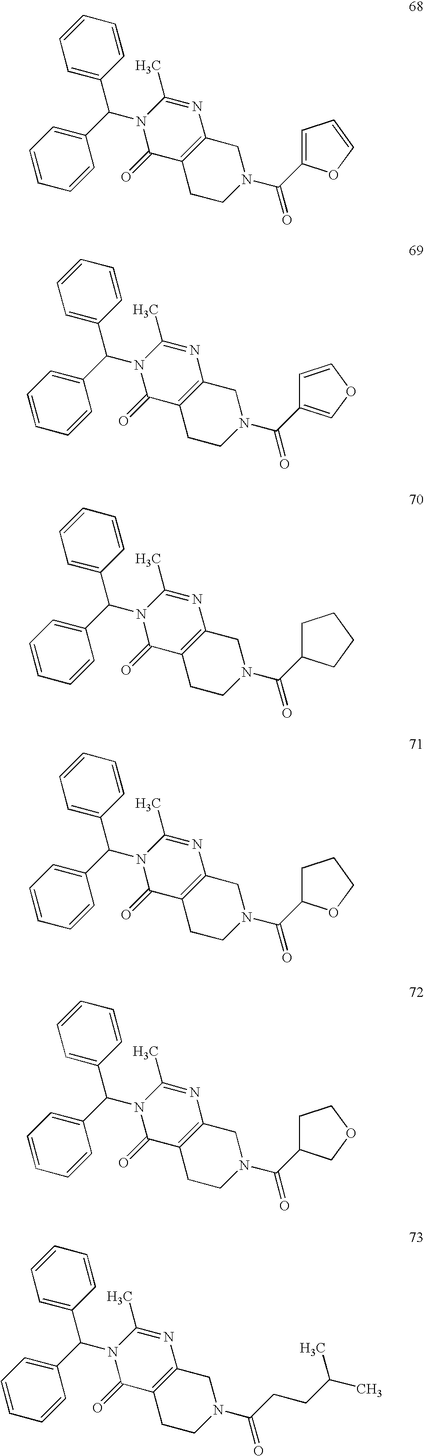

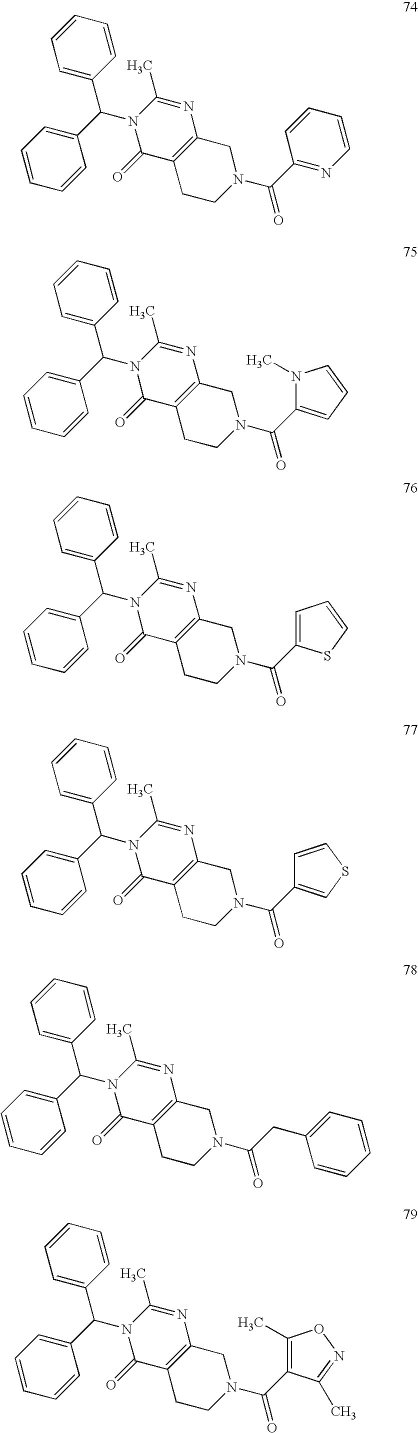

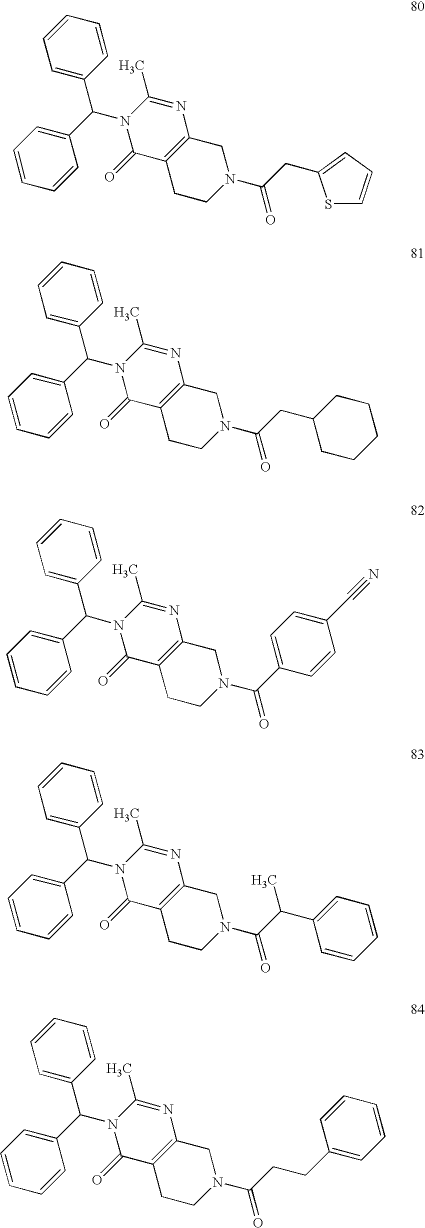

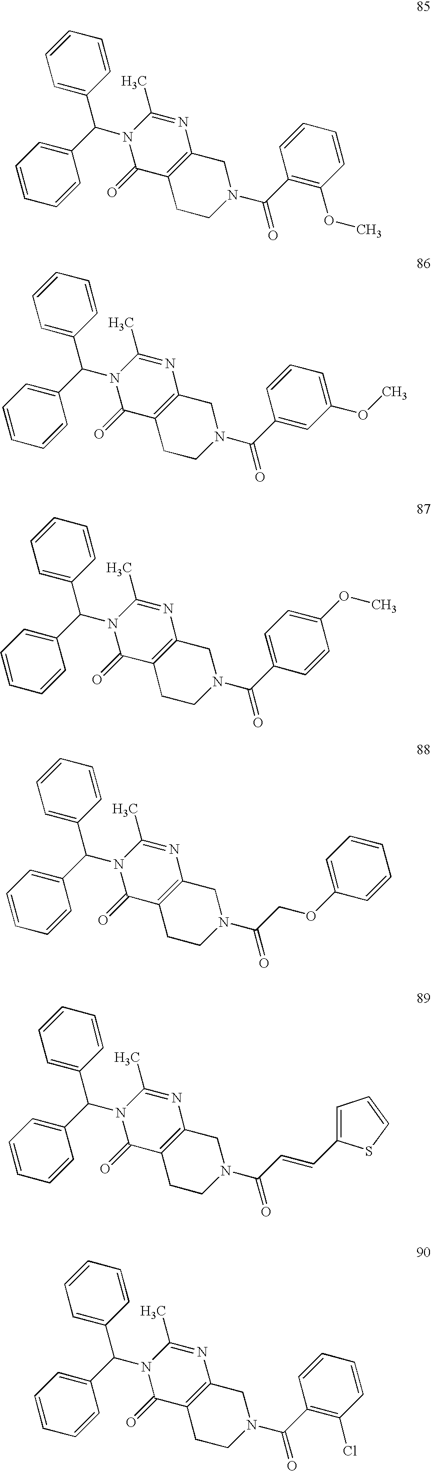

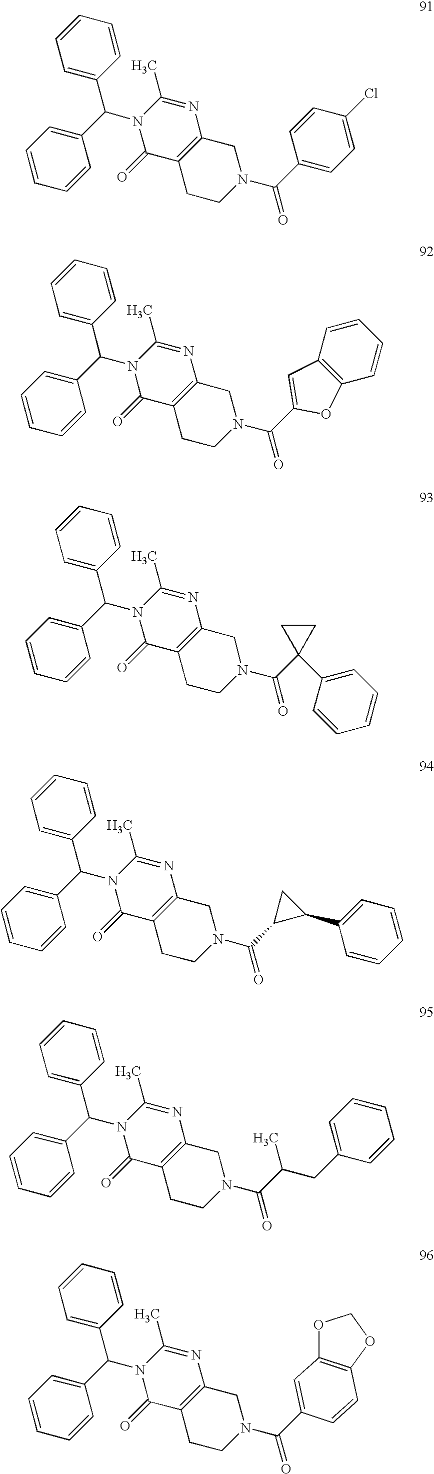

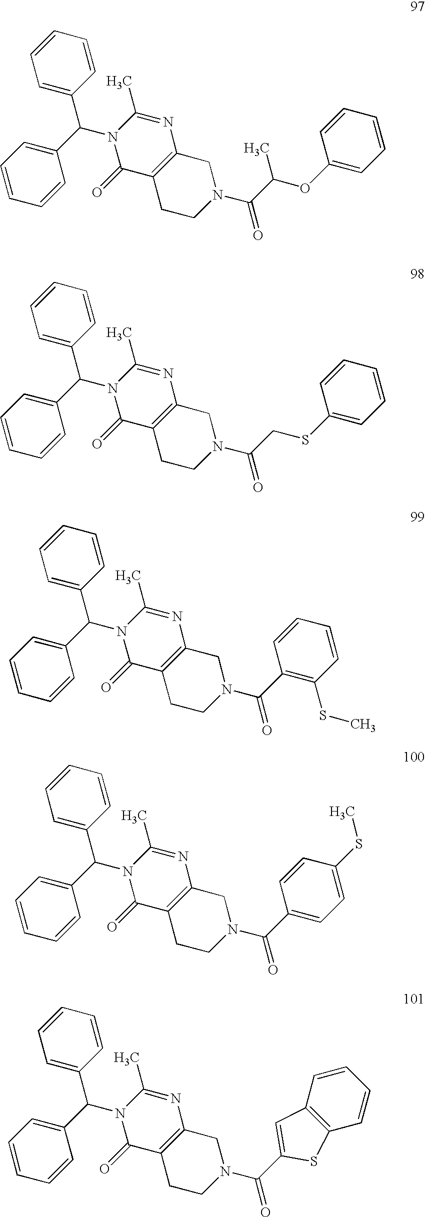

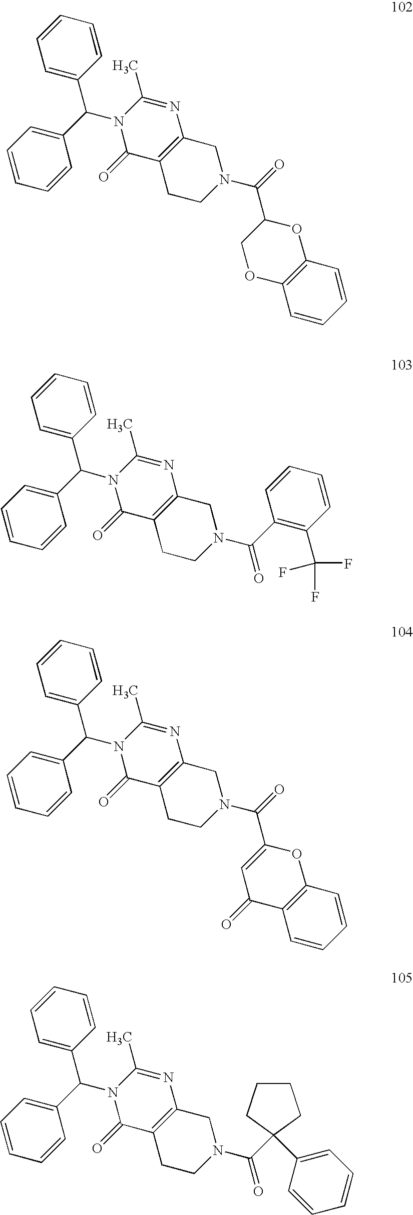

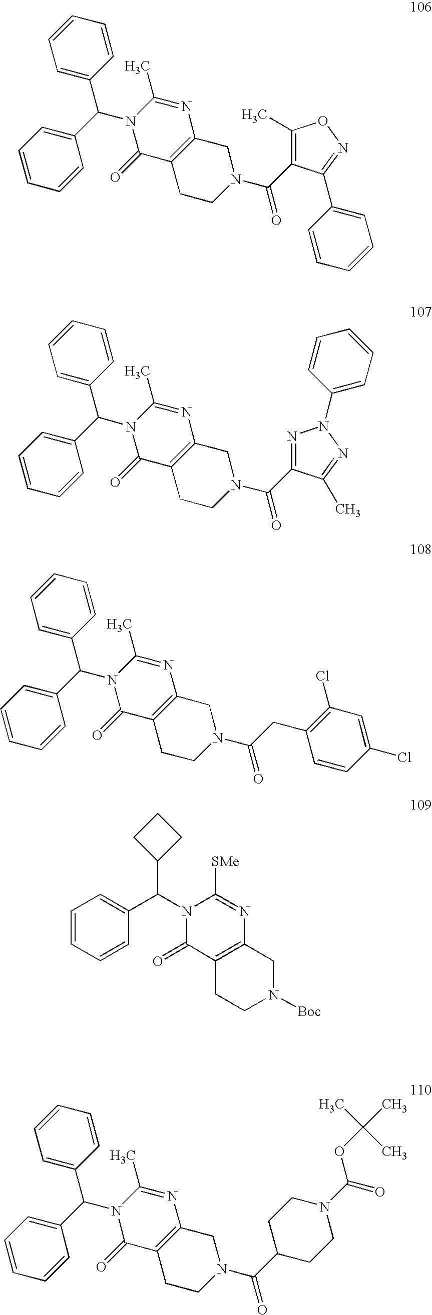

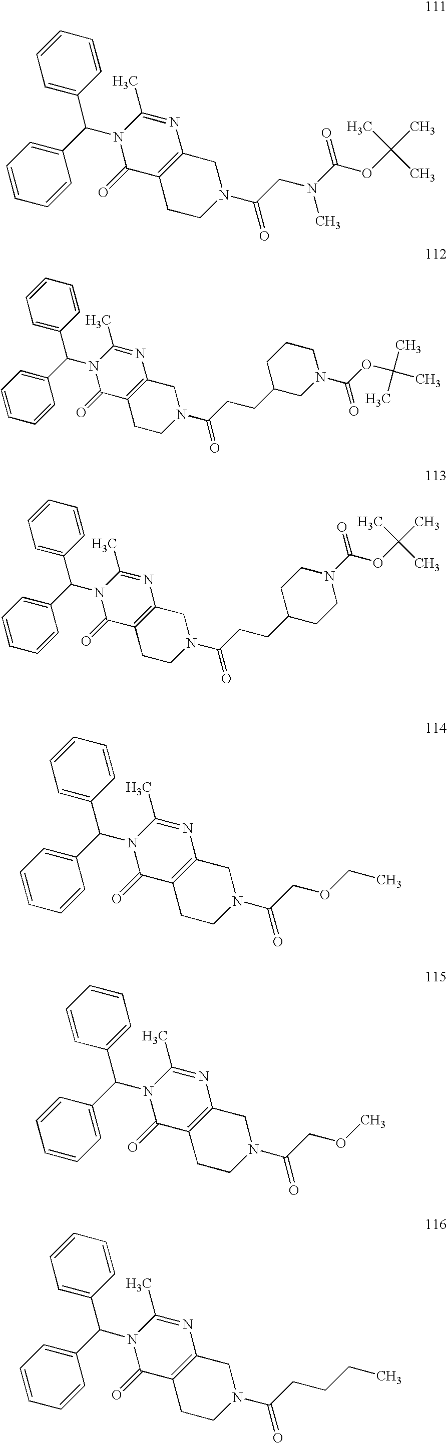

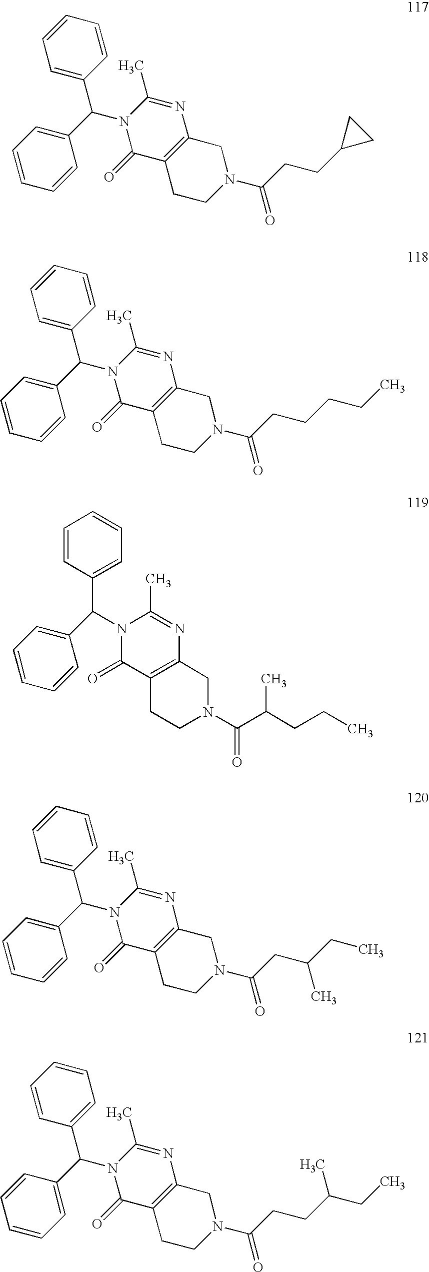

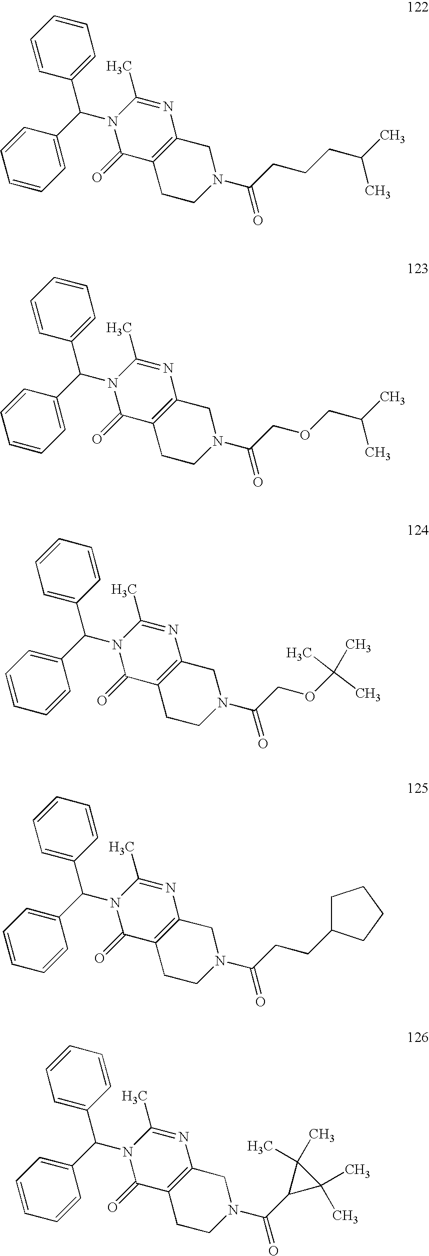

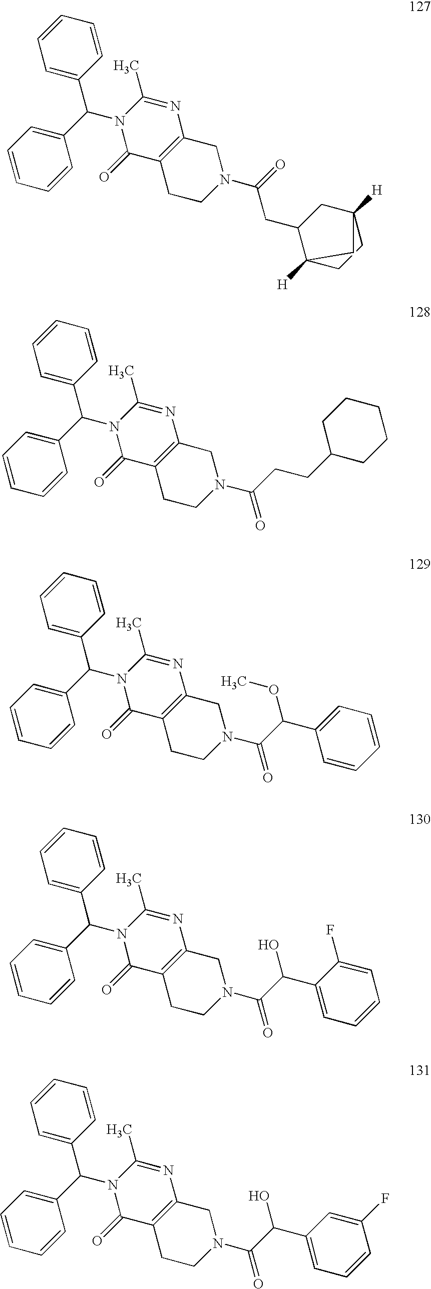

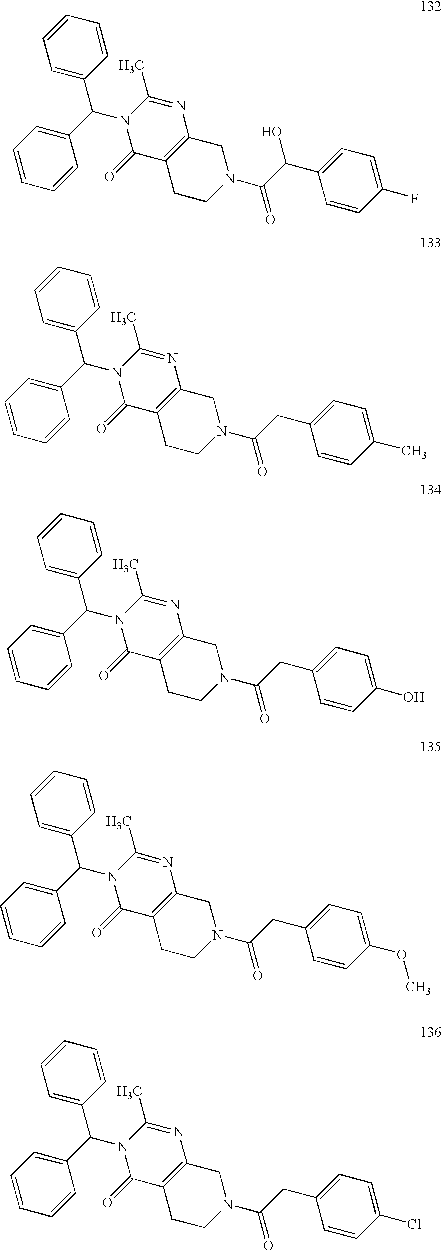

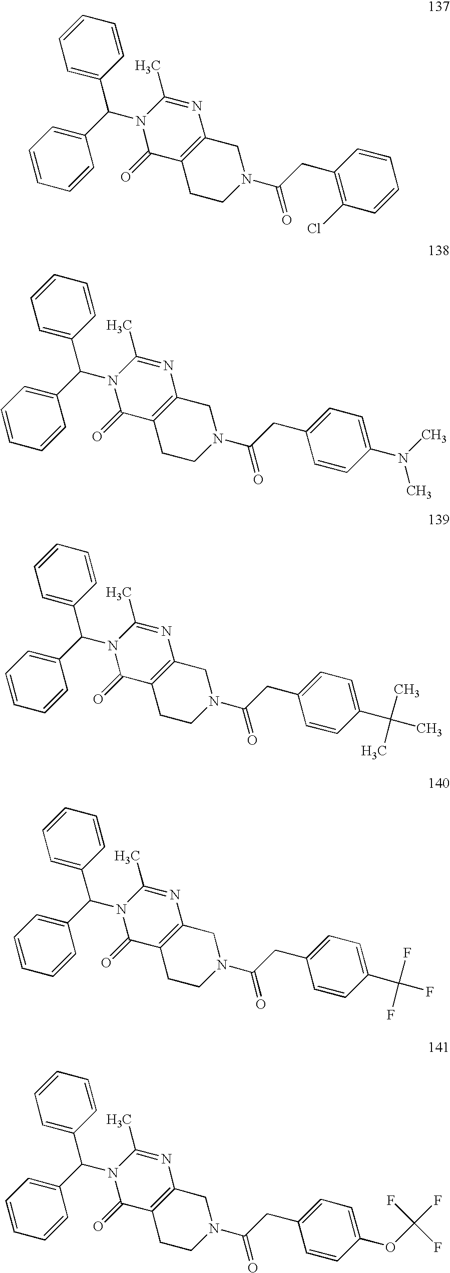

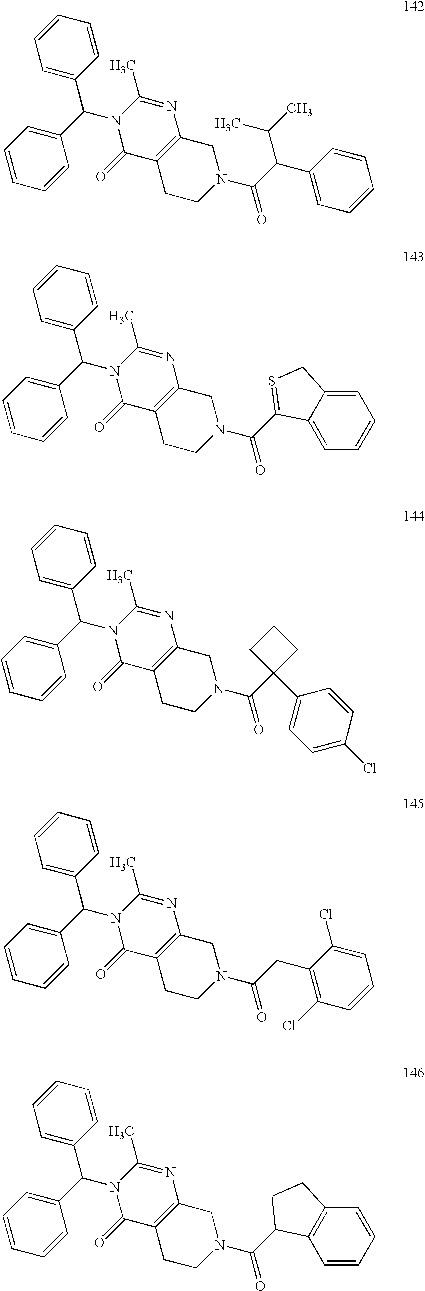

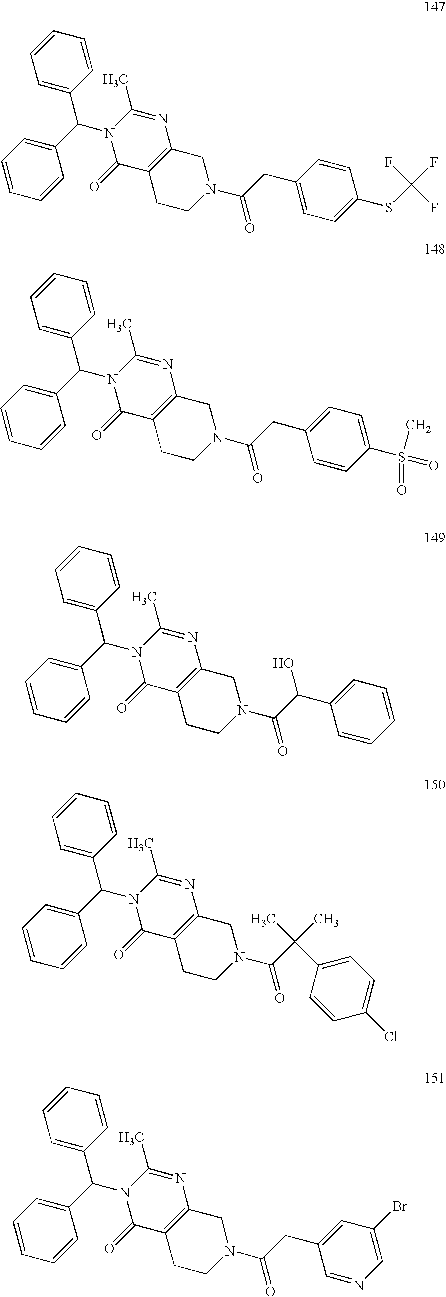

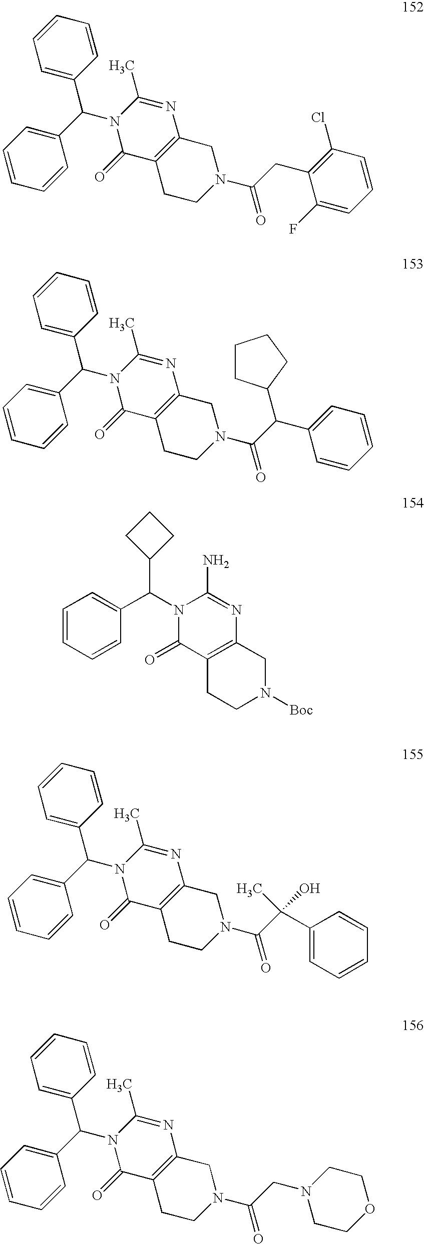

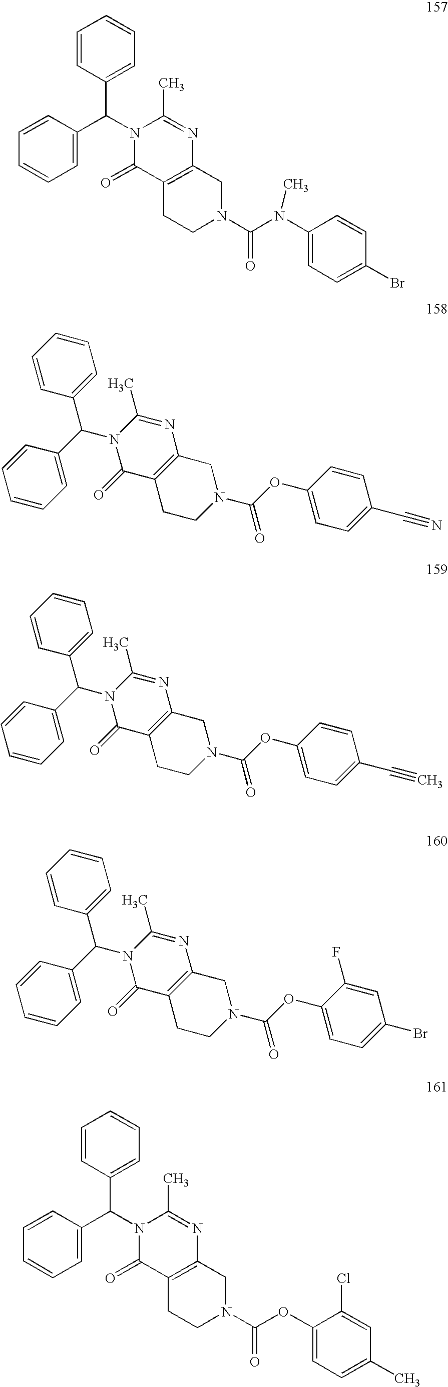

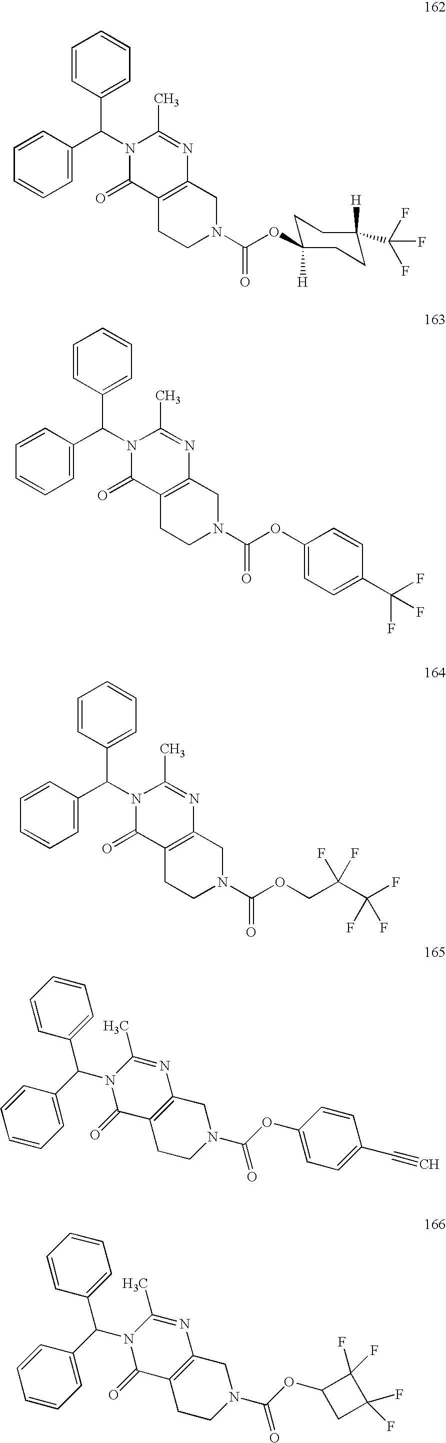

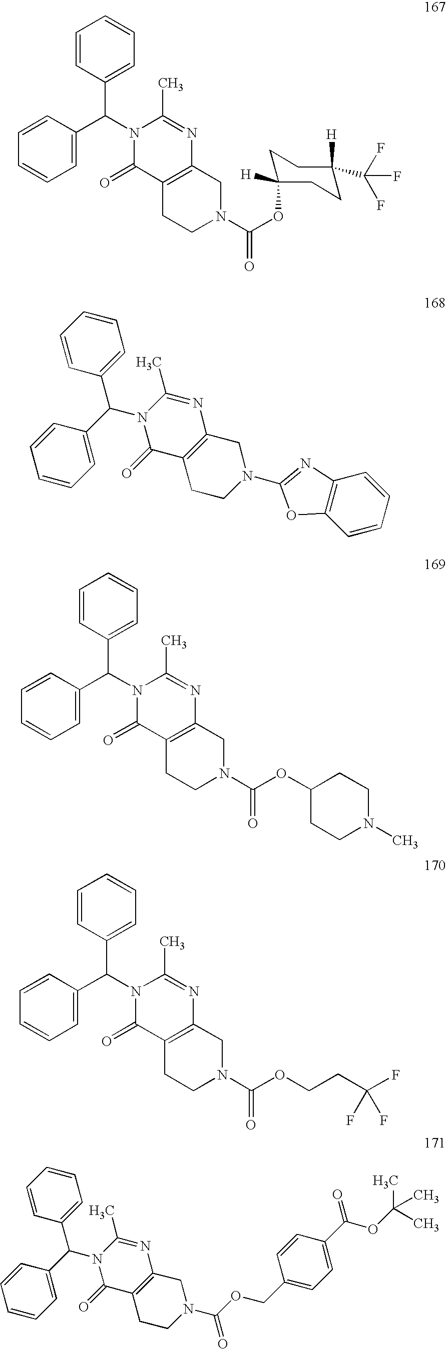

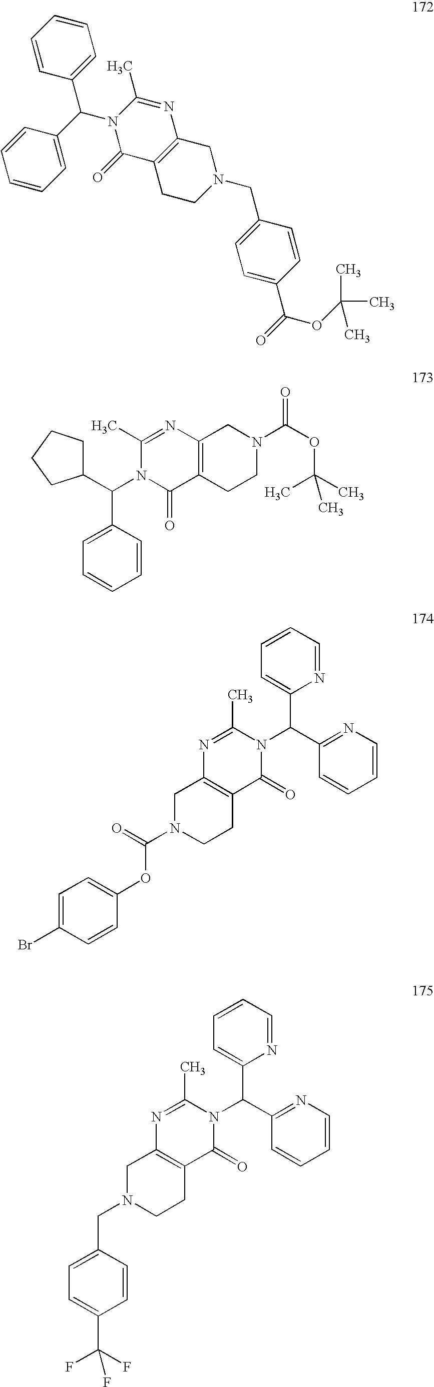

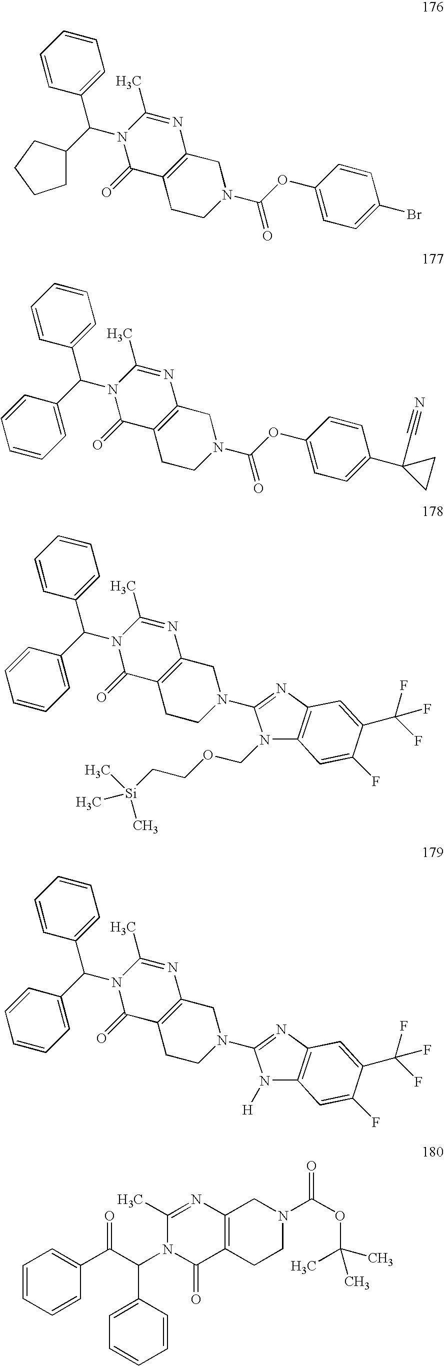

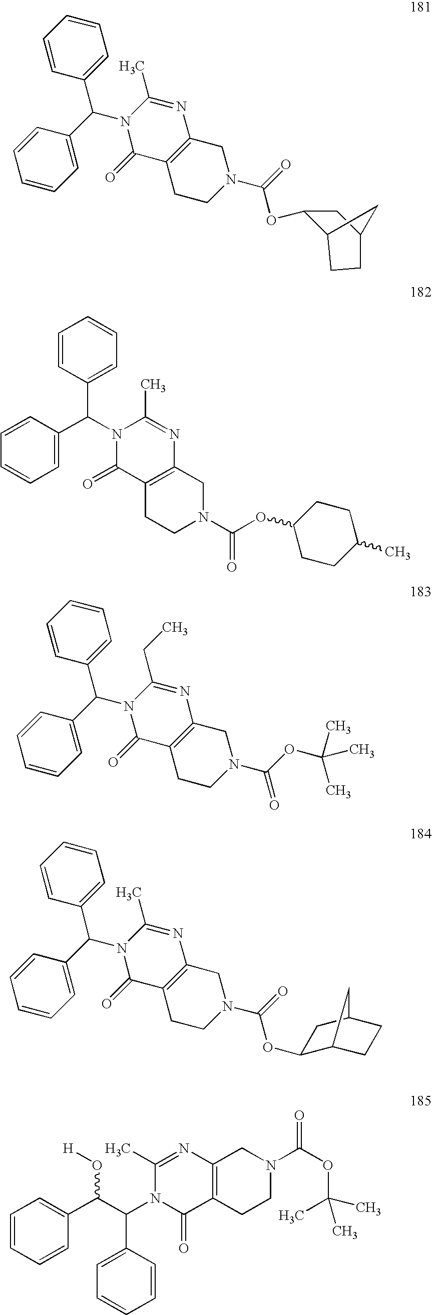

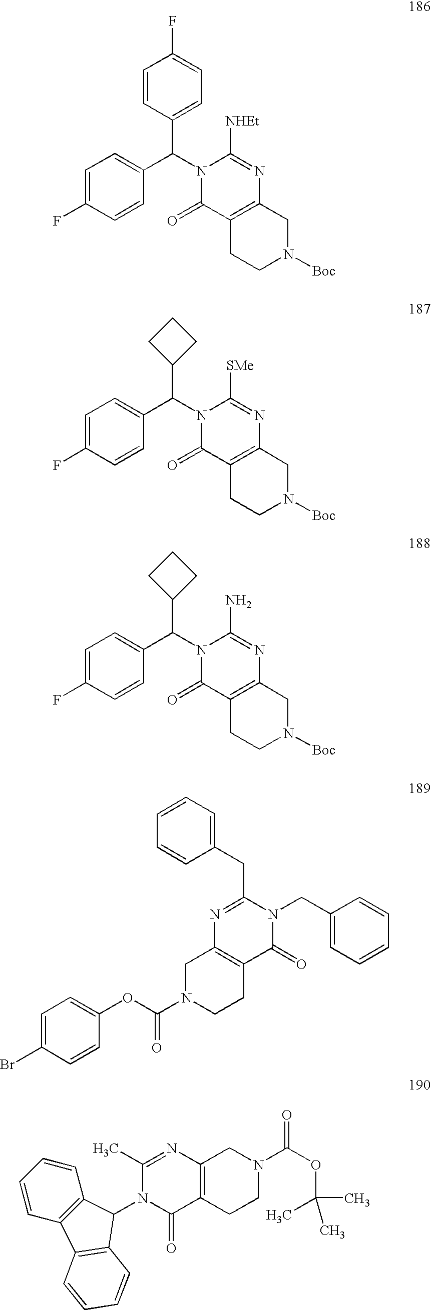

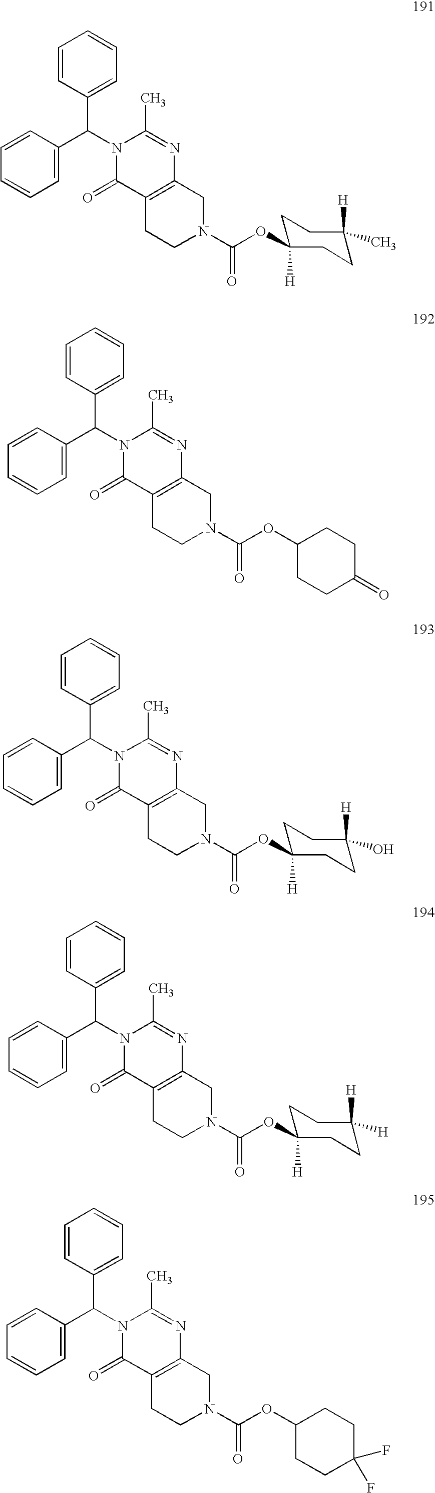

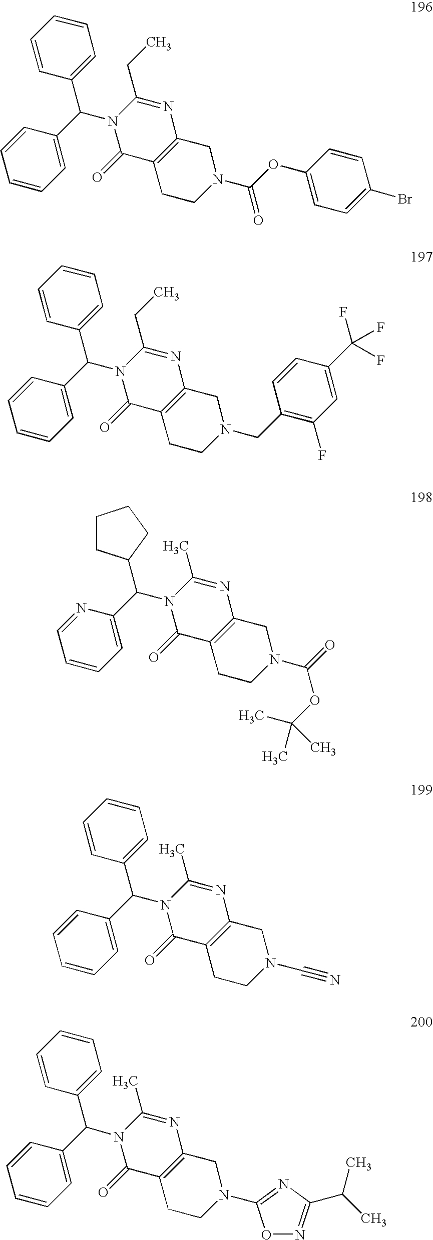

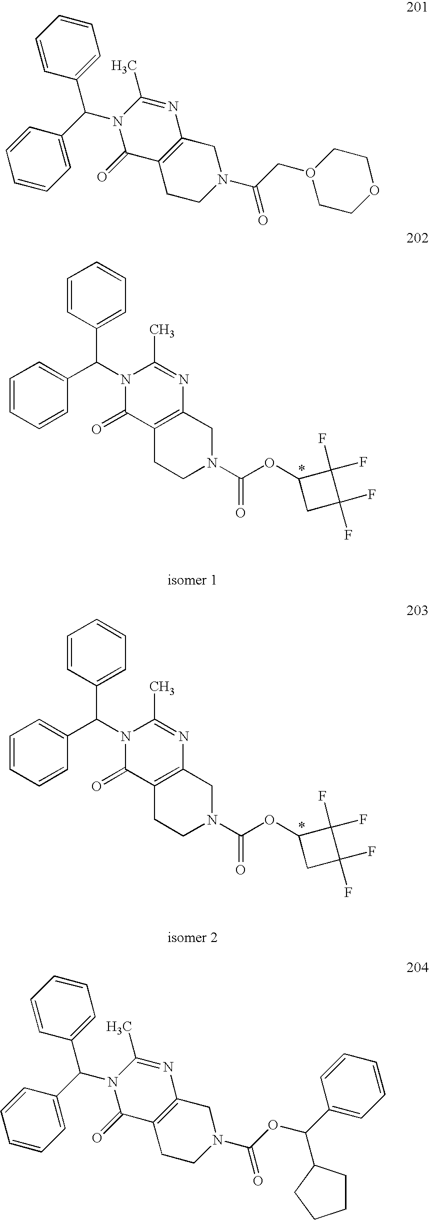

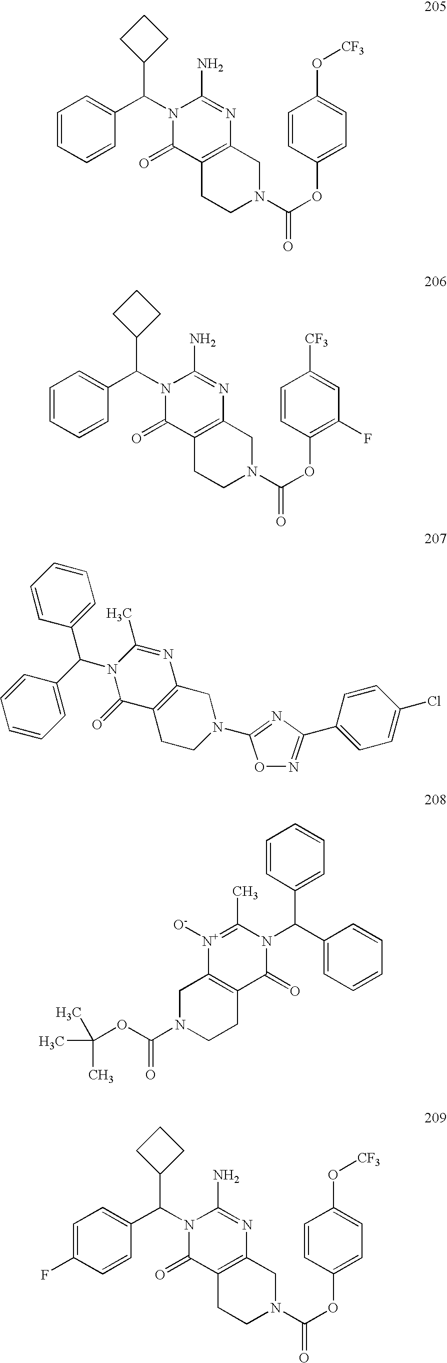

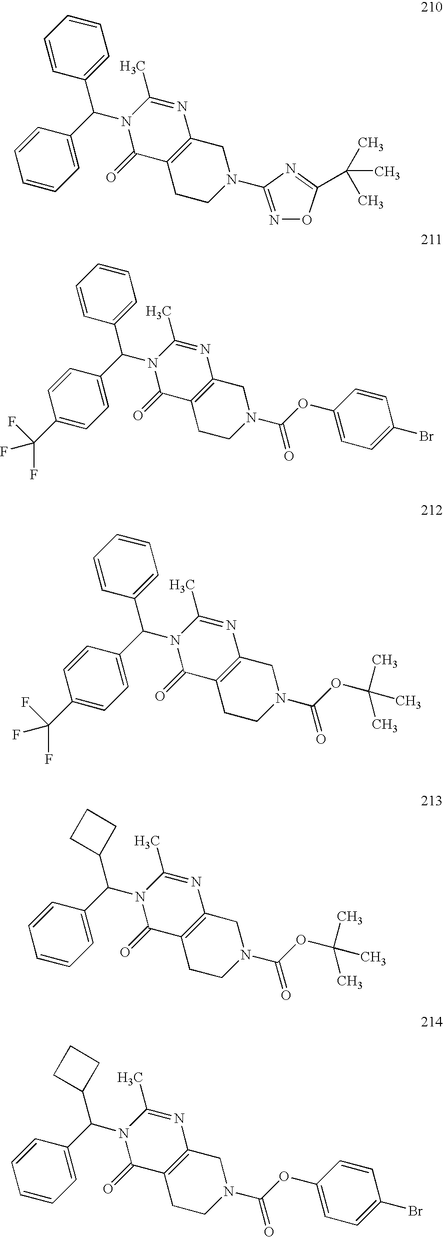

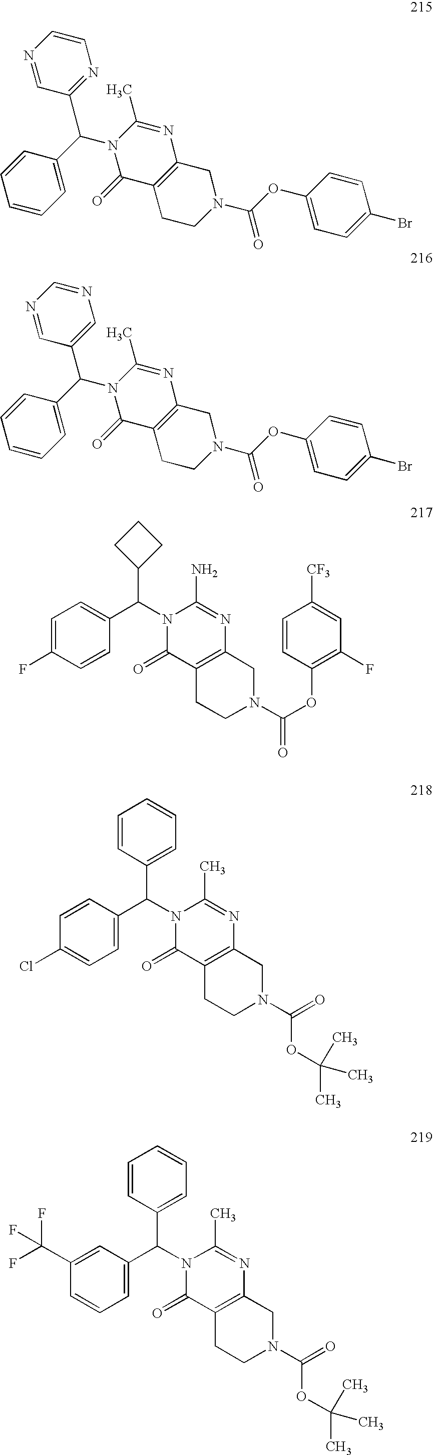

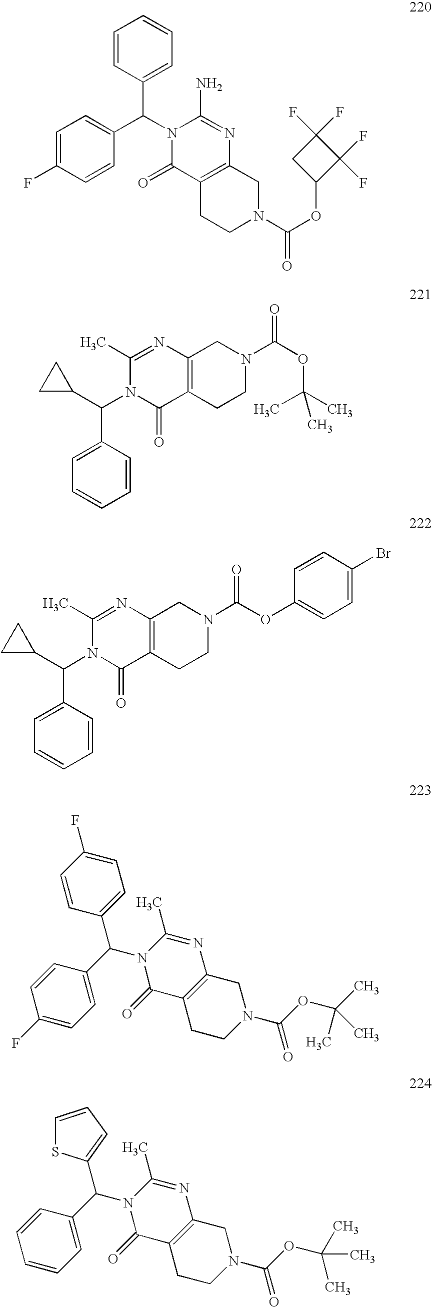

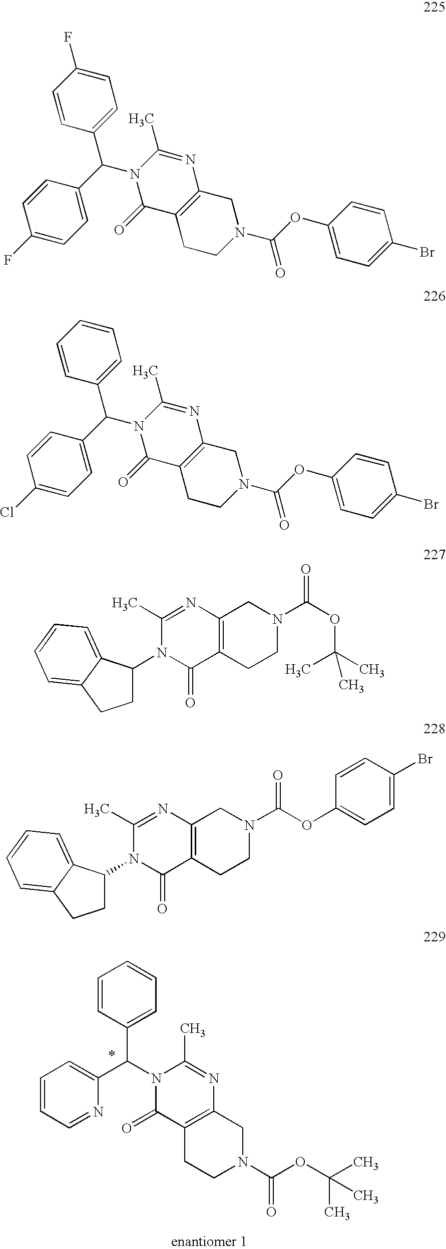

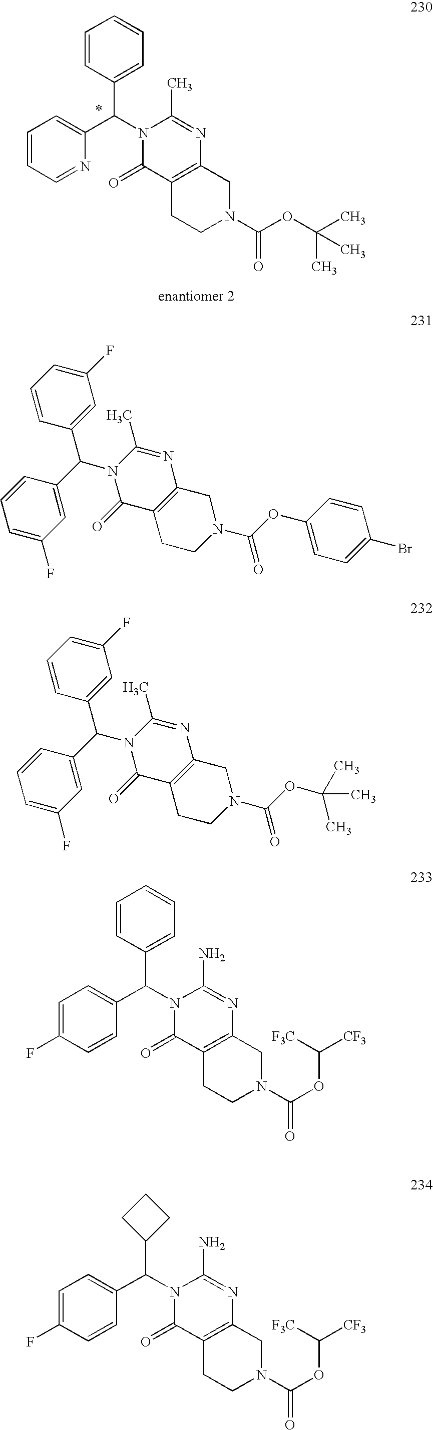

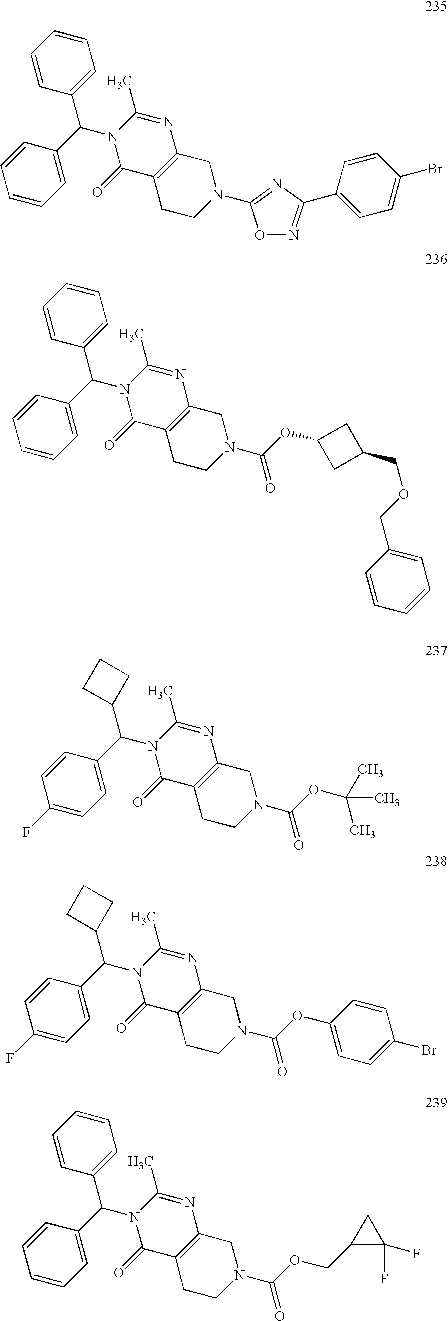

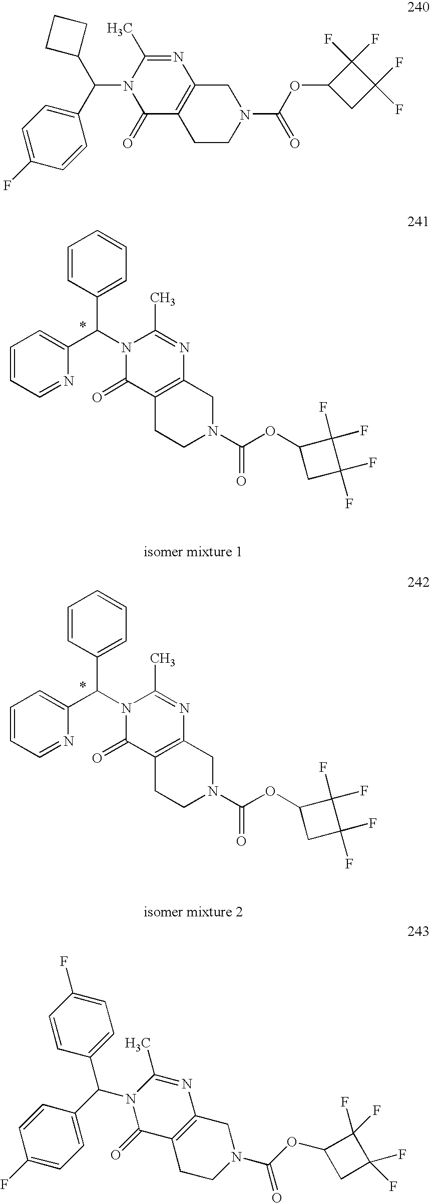

and pharmaceutically acceptable salts, solvates, esters and prodrugs thereof.

Additional non-limiting examples of the Pyrimidinone Derivatives of formula (I) include the following compounds:

| No. | Structure |
|---|---|
| 244 | 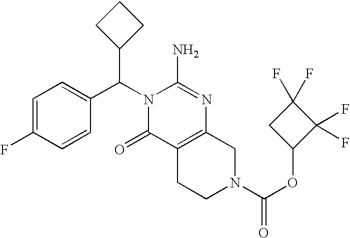 |
| 245 | 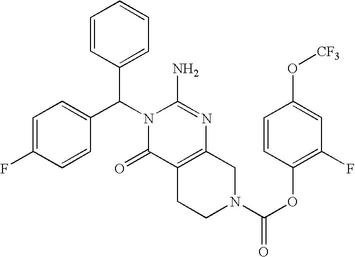 |
| 246 | 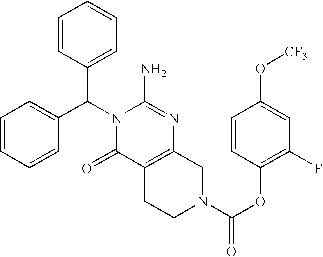 |
| 247 | 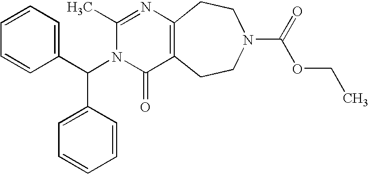 |
| 248 | 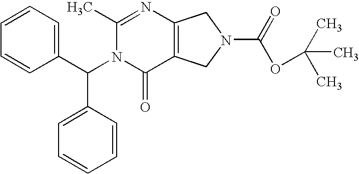 |
| 249 | 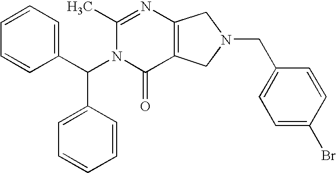 |
| 250 | 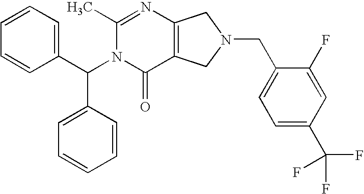 |
| 251 | 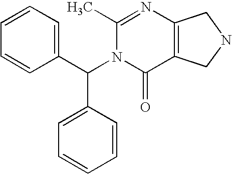 |
| 252 | 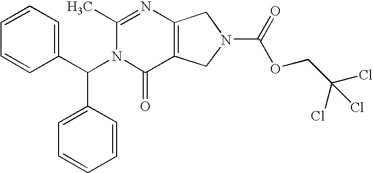 |
| 253 | 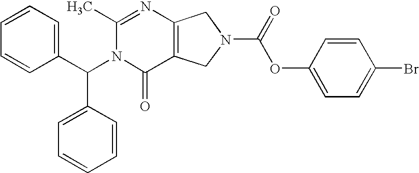 |
| 254 | 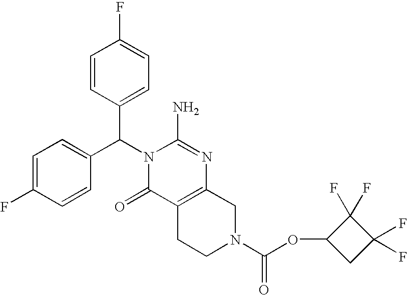 |
| 255 | 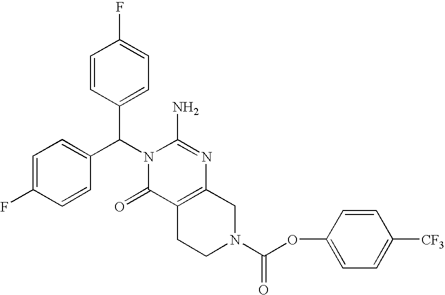 |
| 256 | 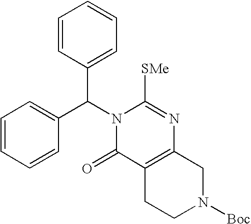 |
| 257 | 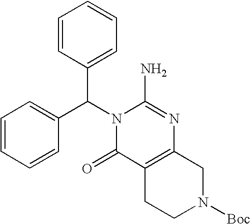 |
| 258 | 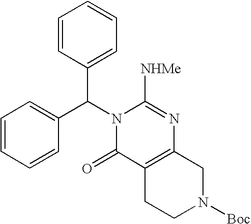 |
| 259 | 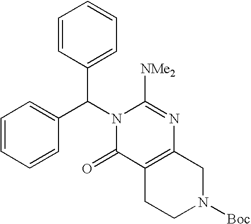 |
| 260 | 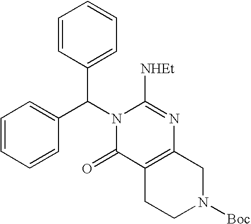 |
| 261 | 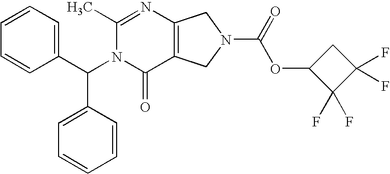 |
| 262 | 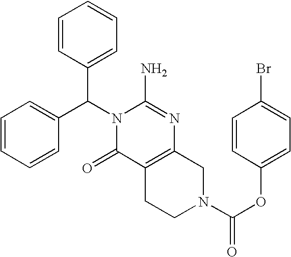 |
| 263 | 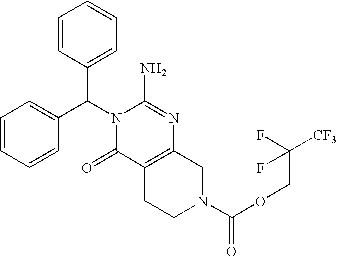 |
| 264 | 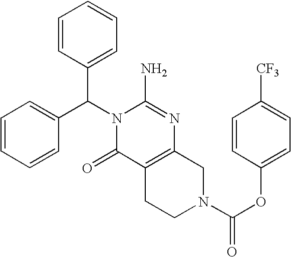 |
| 265 | 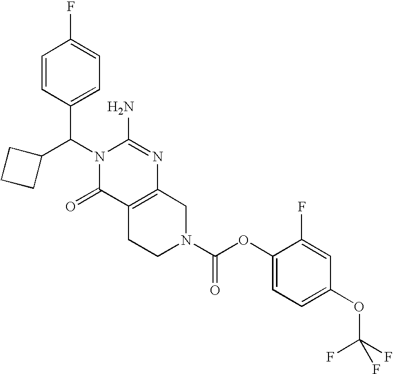 |
| 266 | 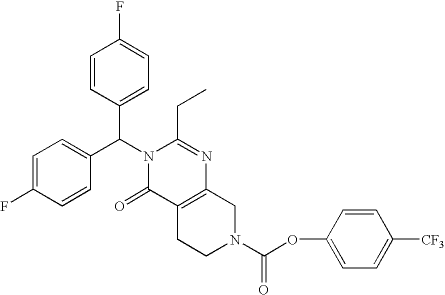 |
| 267 | 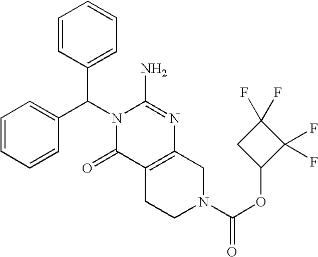 |
| 268 | 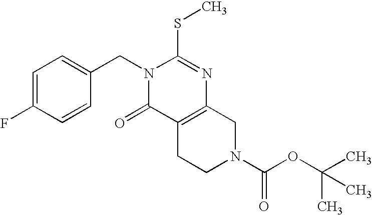 |
| 269 | 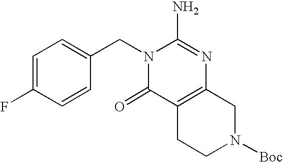 |
| 270 | 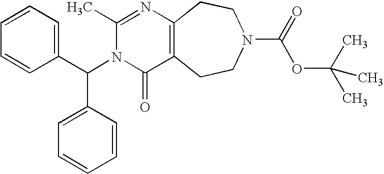 |
| 271 | 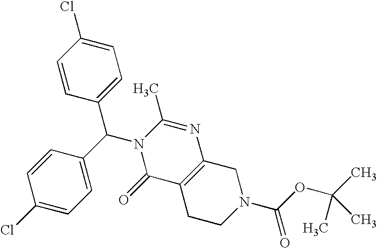 |
| 272 | 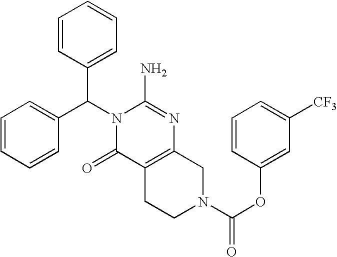 |
| 273 | 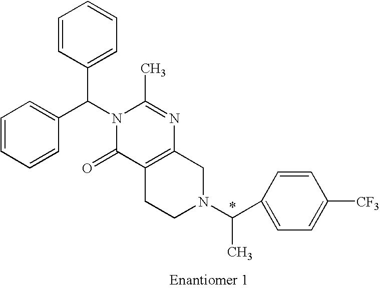 |
| 274 | 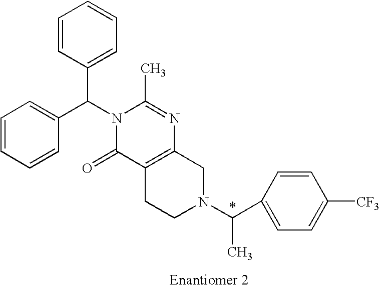 |
| 275 | 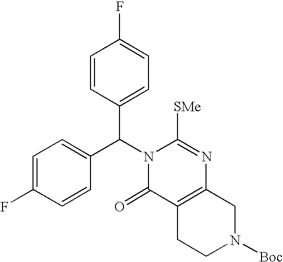 |
| 276 | 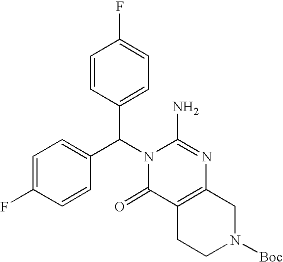 |
| 277 | 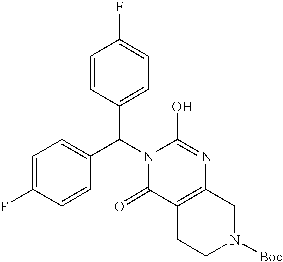 |
| 278 | 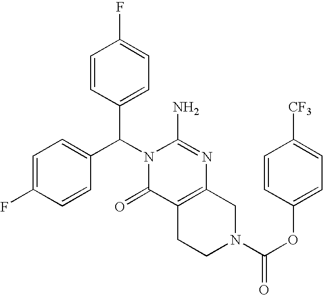 |
| 279 | 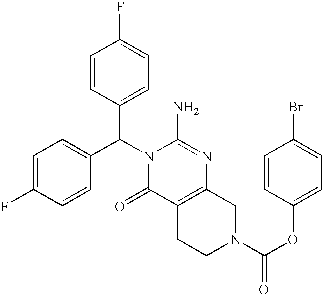 |
| 280 | 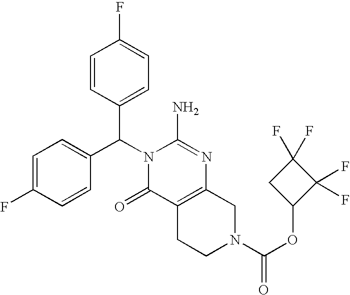 |
| 281 | 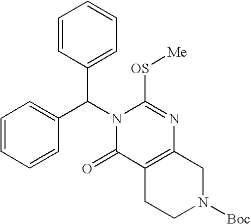 |
| 282 | 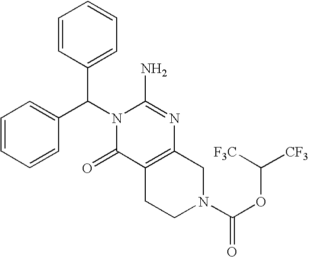 |
| 283 | 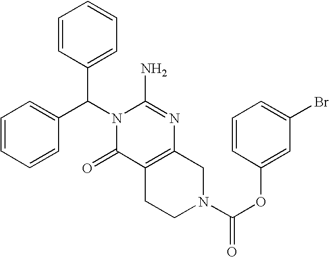 |
| 284 | 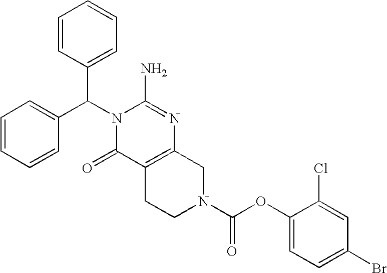 |
| 285 | 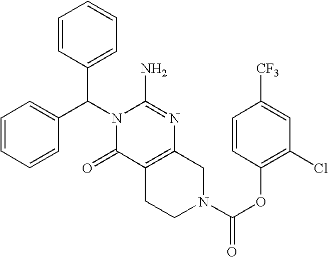 |
| 286 | 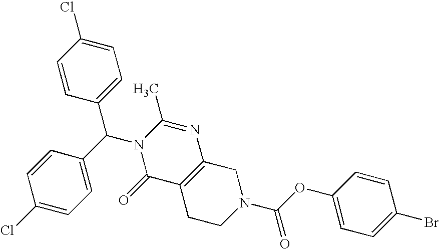 |
| 287 | 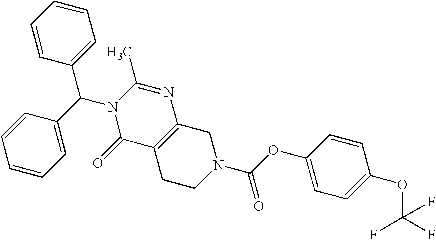 |
| 288 | 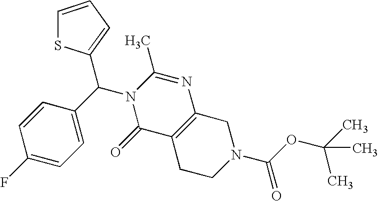 |
| 289 | 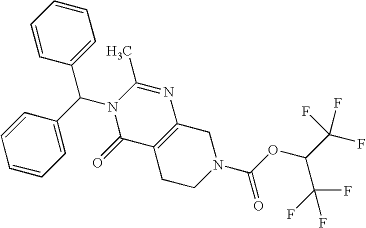 |
| 290 | 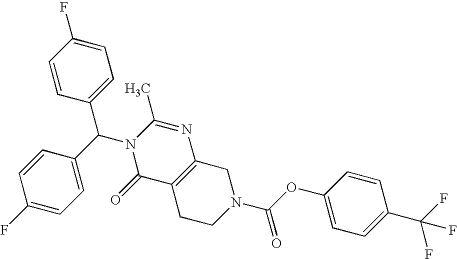 |
| 291 | 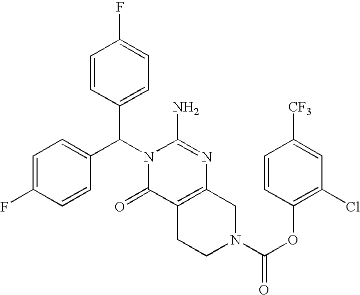 |
| 292 | 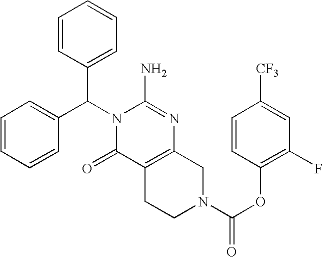 |
| 293 | 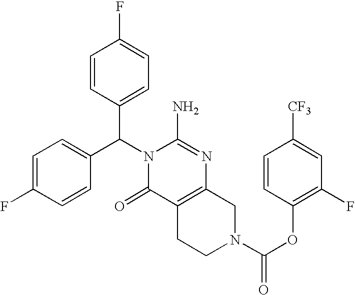 |
| 294 | 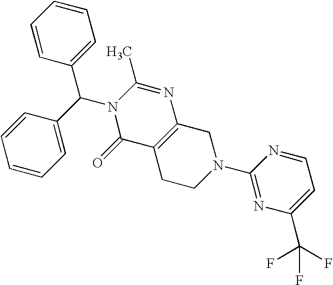 |
| 295 | 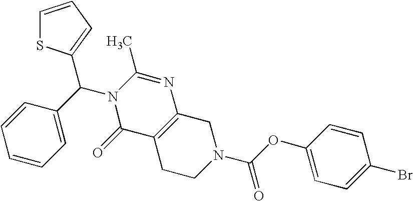 |
| 296 | 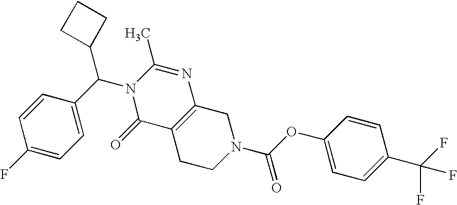 |
| 297 | 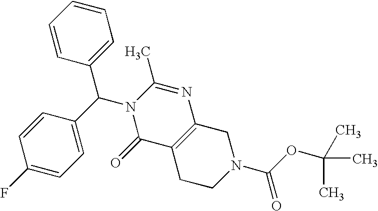 |
| 298 | 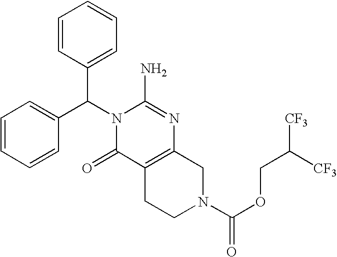 |
| 299 | 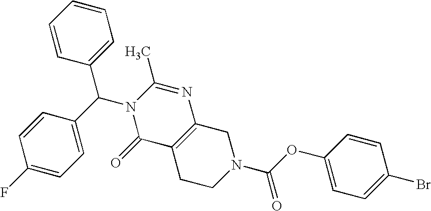 |
| 300 | 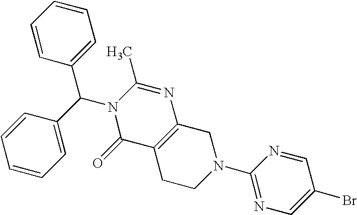 |
| 301 | 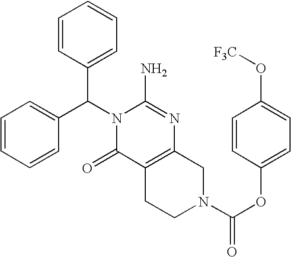 |
| 302 | 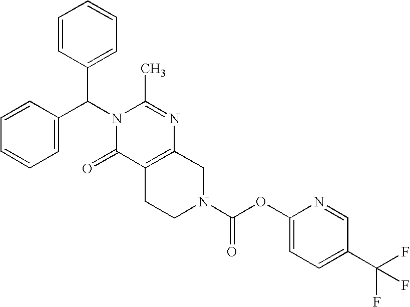 |
| 303 | 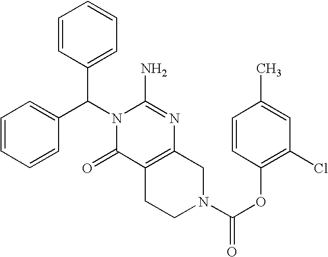 |
| 304 | 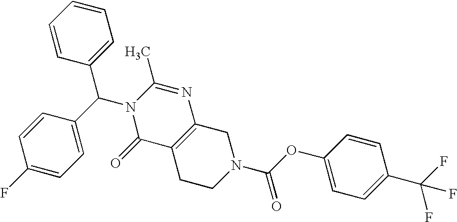 |
| 305 | 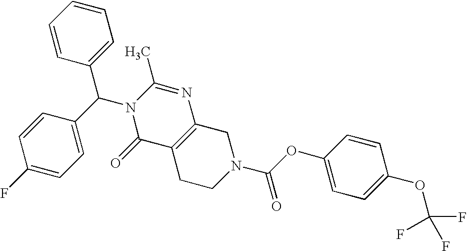 |
| 306 | 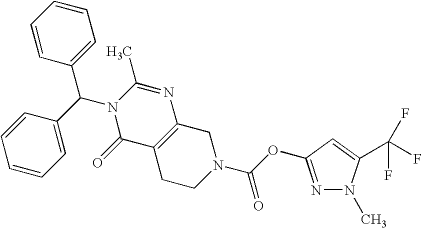 |
| 307 | 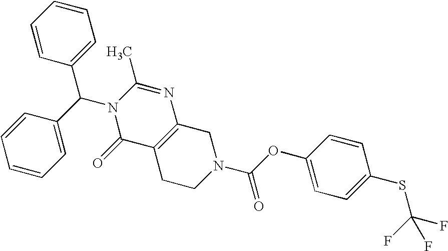 |
| 308 | 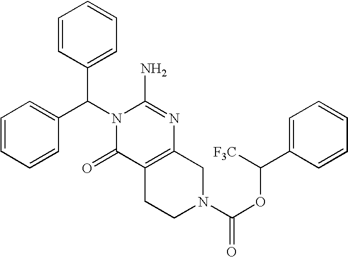 |
| 309 | 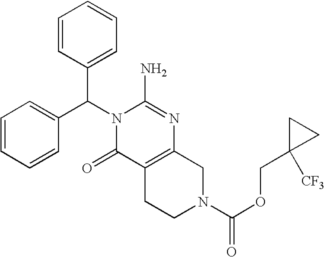 |
| 310 | 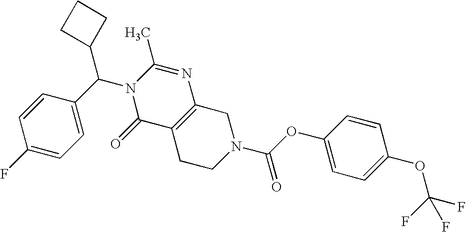 |
| 311 | 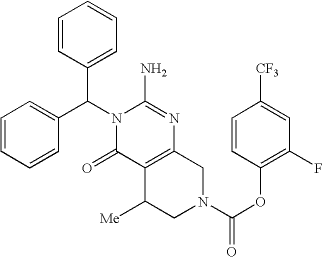 |
| 312 | 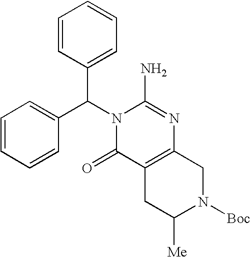 |
| 313 | 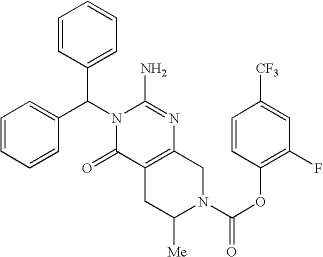 |
| 314 | 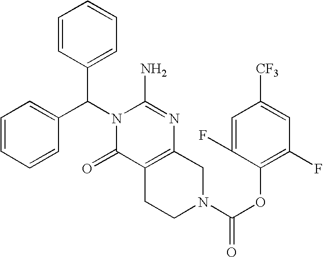 |
| 315 | 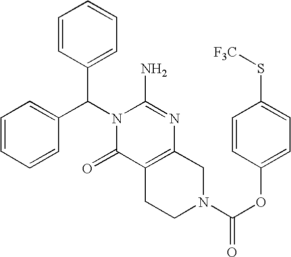 |
| 316 | 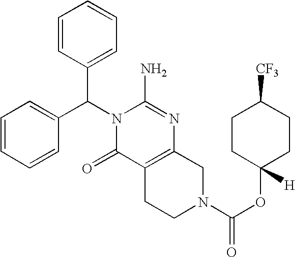 |
| 317 | 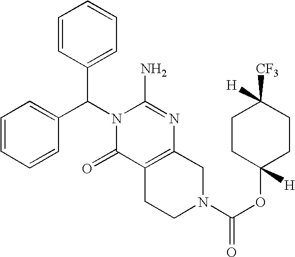 |
| 318 | 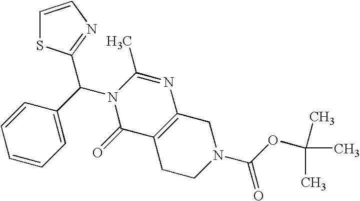 |
| 319 | 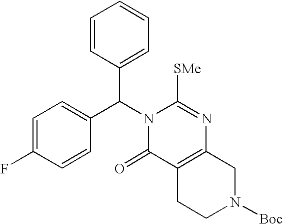 |
| 320 | 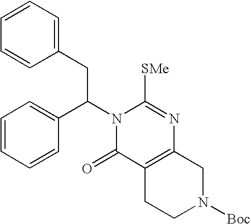 |
| 321 | 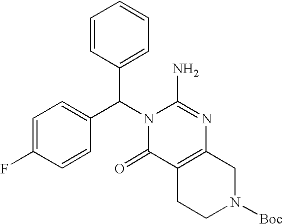 |
| 322 | 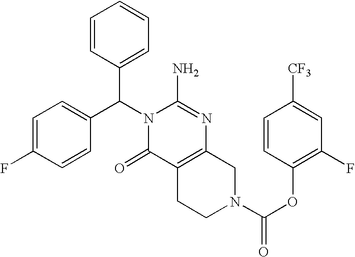 |
| 323 | 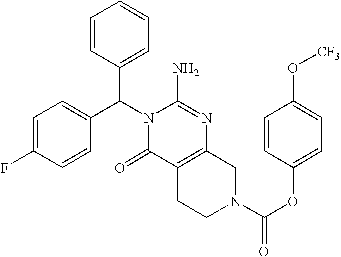 |
| 324 | 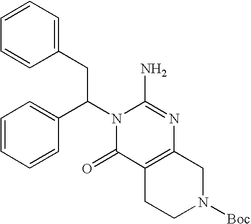 |
| 325 | 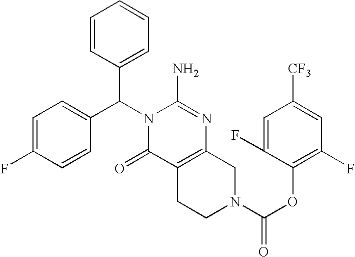 |
| 326 | 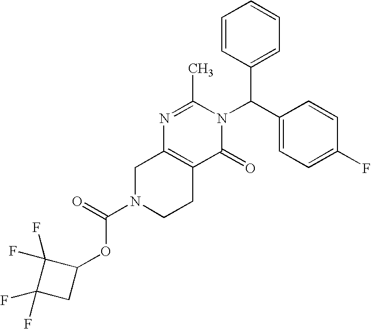 |
| 327 | 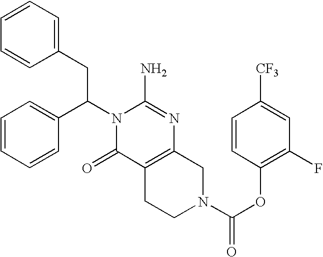 |
| 328 | 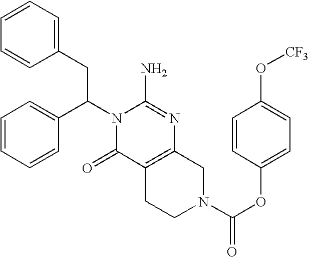 |
| 329 | 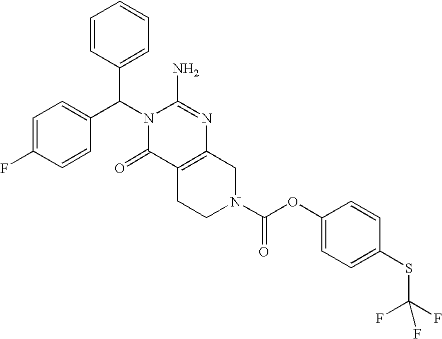 |
| Compound | |
| No. | Structure |
| 330 | 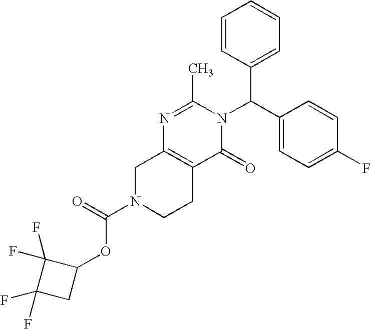 |
| 331 | 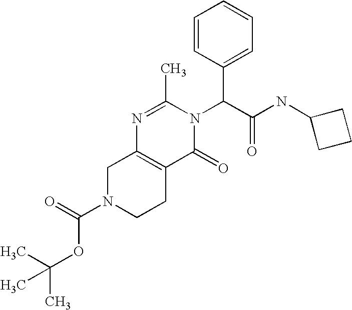 |
| 332 | 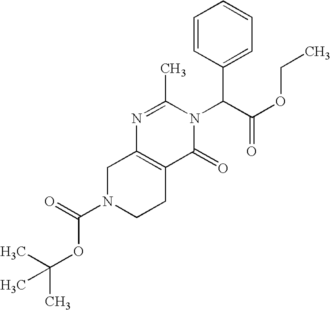 |
| 333 | 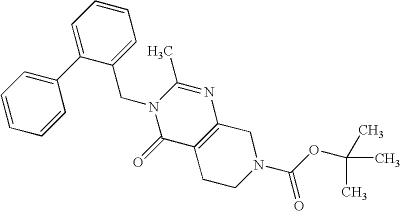 |
| 334 | 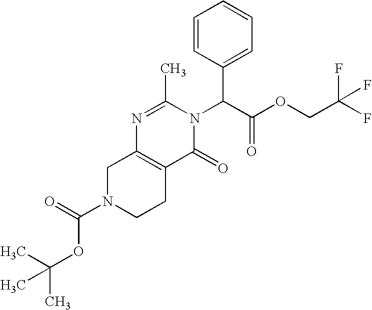 |
| 335 | 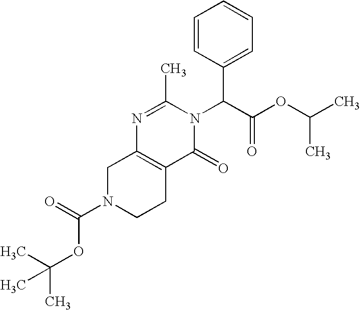 |
| 336 | 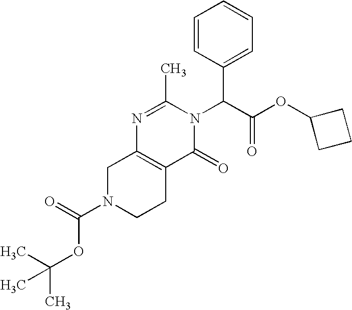 |
| 337 | 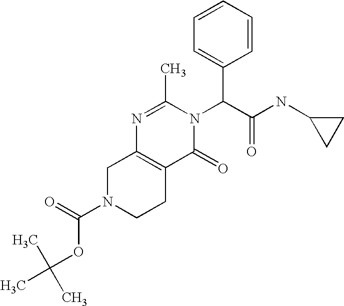 |
| 338 | 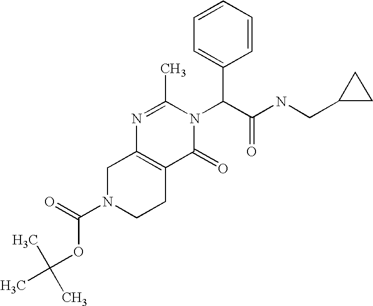 |
| 339 | 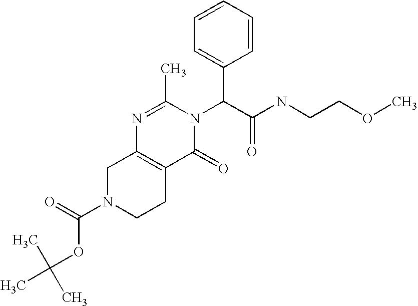 |
| 340 | 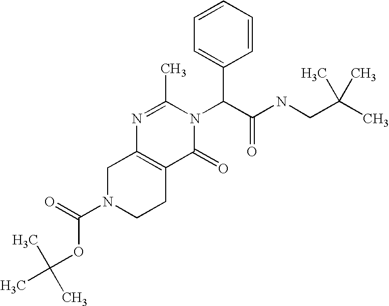 |
| 341 | 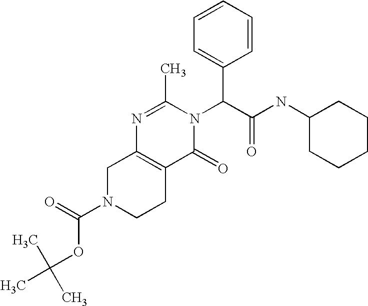 |
| 342 | 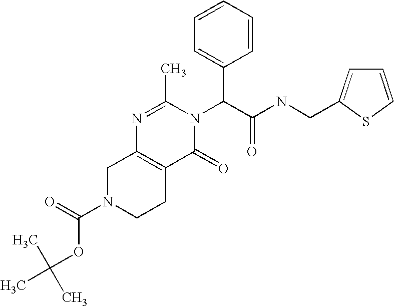 |
| 343 | 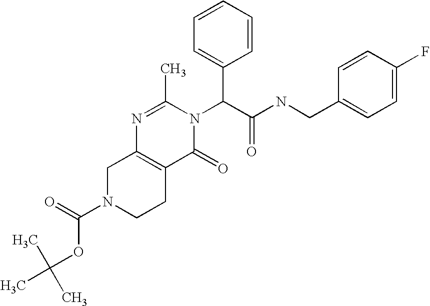 |
| 344 | 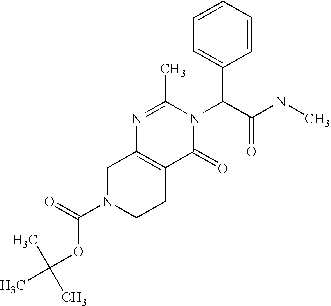 |
| 345 | 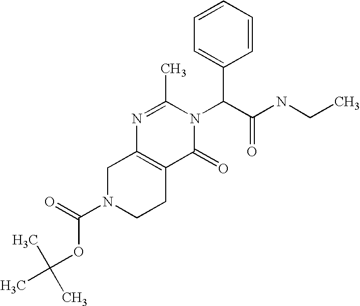 |
| 346 | 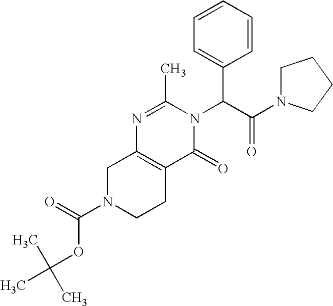 |
| 347 | 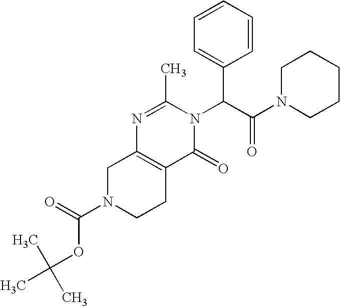 |
| 348 | 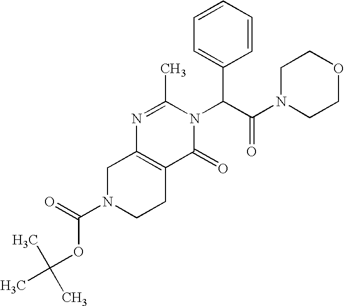 |
| 349 | 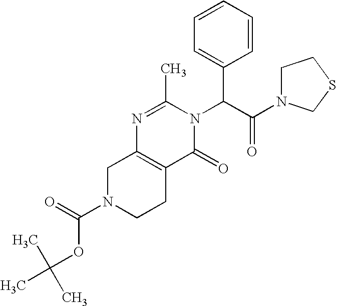 |
| 350 | 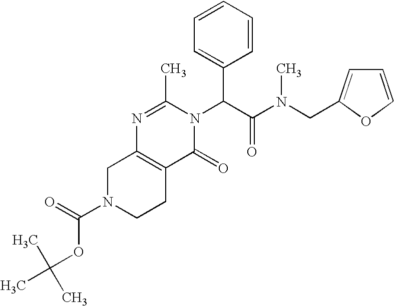 |
| 351 | 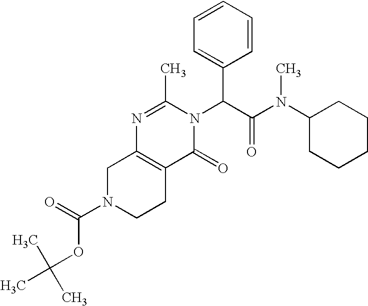 |
| 352 | 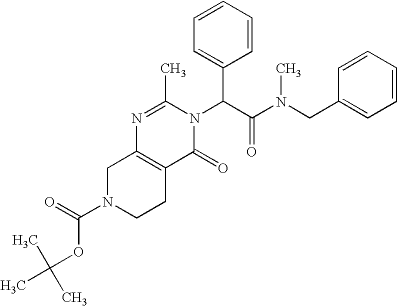 |
| 353 | 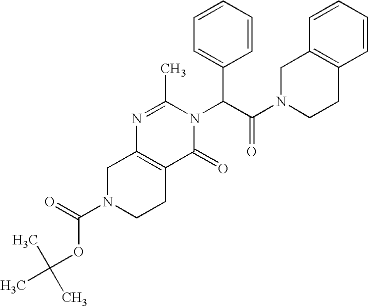 |
| 354 | 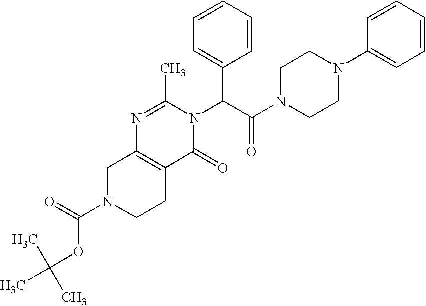 |
| 355 | 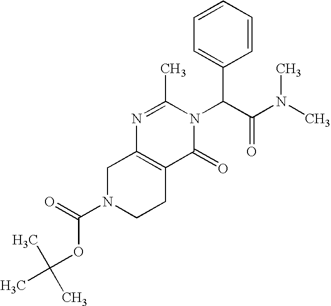 |
| 356 | 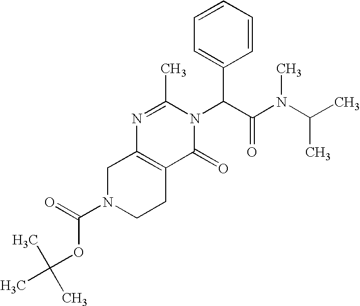 |
| 357 | 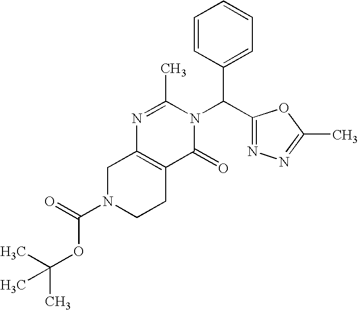 |
| 358 | 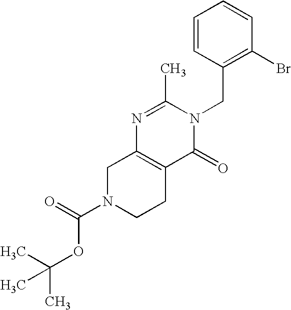 |
| 359 | 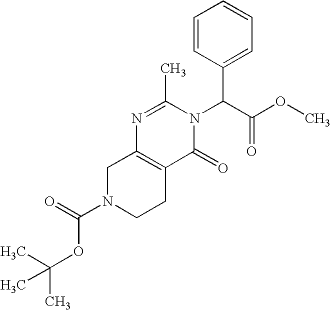 |
| 360 | 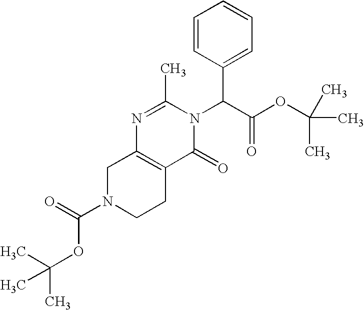 |
| 361 | 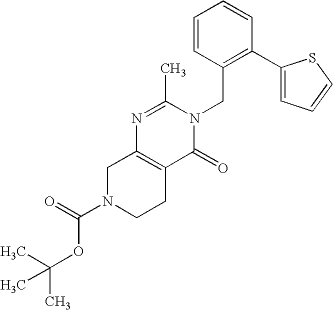 |
| 362 | 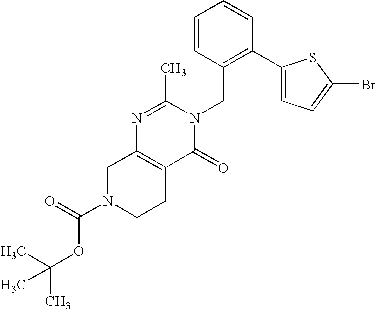 |
| 363 | 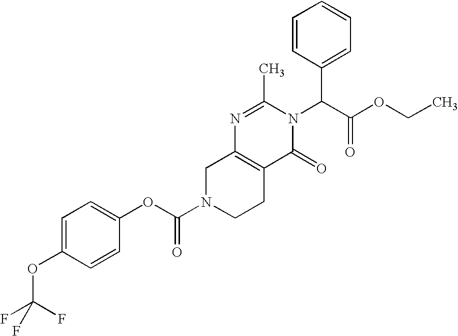 |
| 364 | 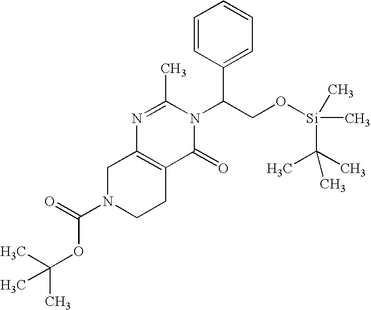 |
| 365 | 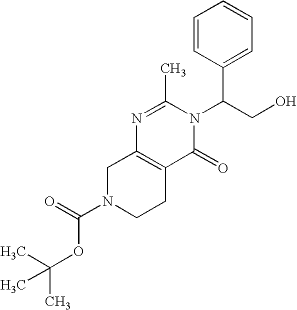 |
| 366 | 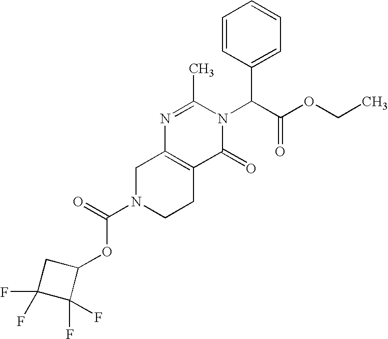 |
| 367 | 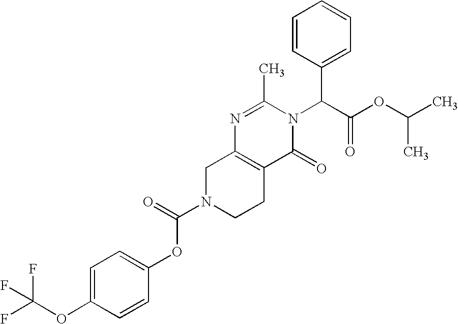 |
| 368 | 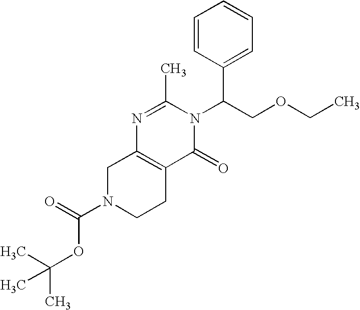 |
| 369 | 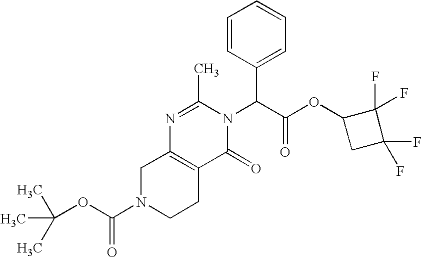 |
| 370 | 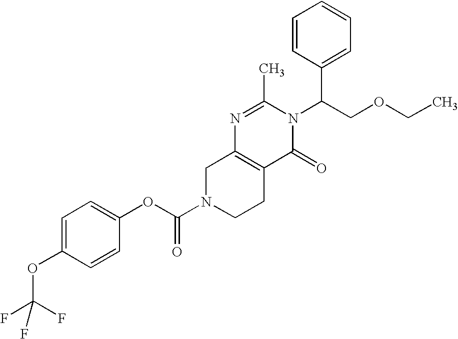 |
| 371 | 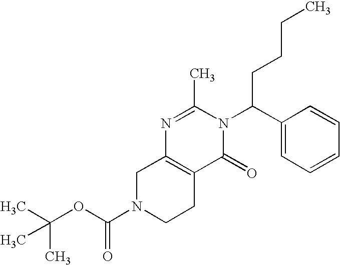 |
| 372 | 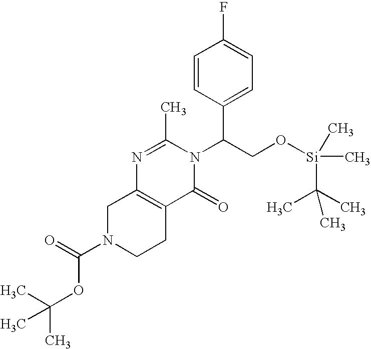 |
| 373 | 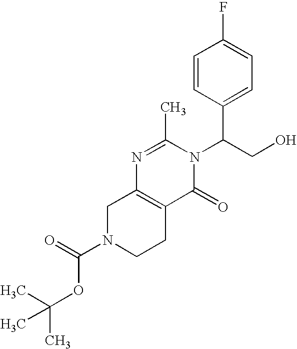 |
| 374 | 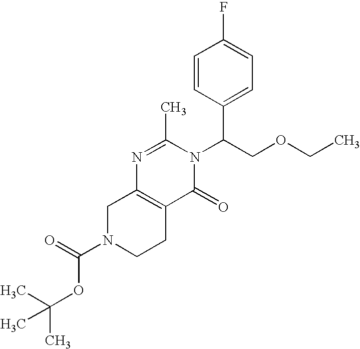 |
| 375 | 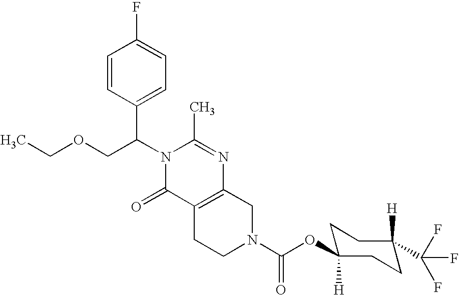 |
| 376 | 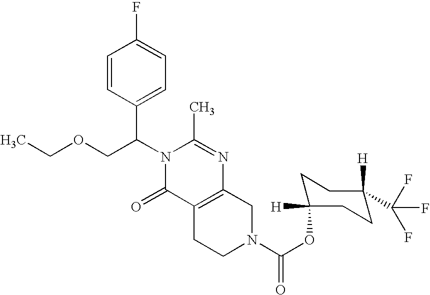 |
| 377 | 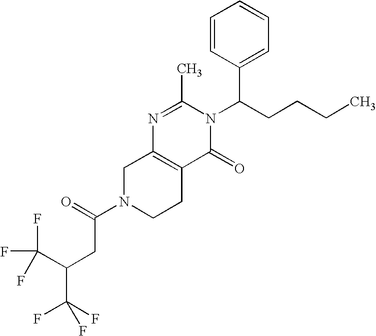 |
| 378 | 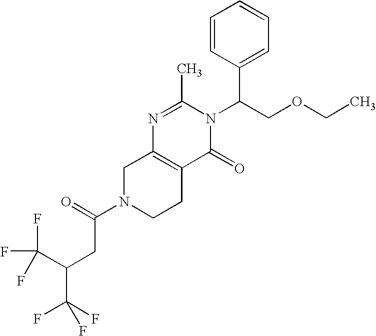 |
| 379 | 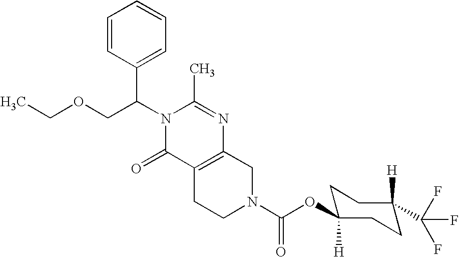 |
| 380 | 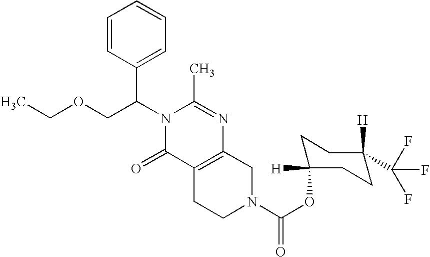 |
| 381 | 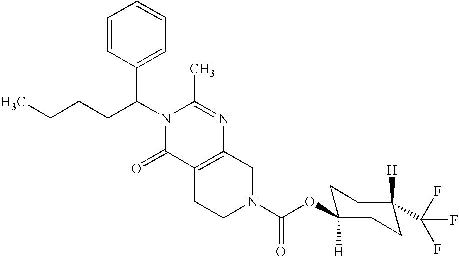 |
| 382 | 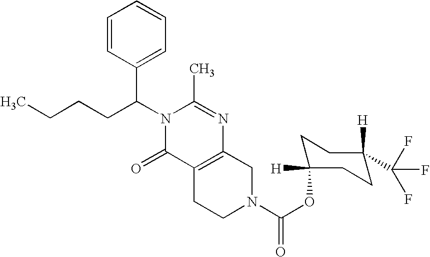 |
| 383 | 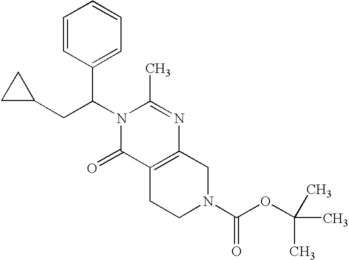 |
| 384 | 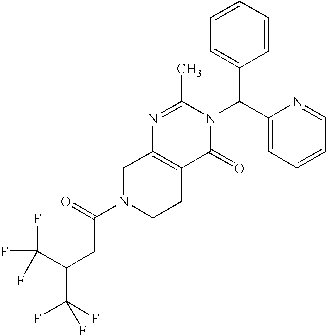 |
| 385 | 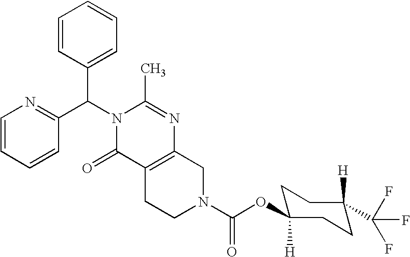 |
| 386 | 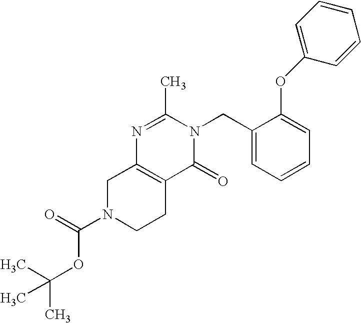 |
| 387 | 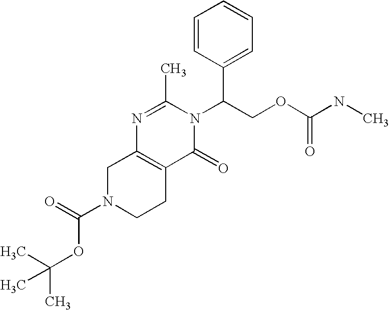 |
| 388 | 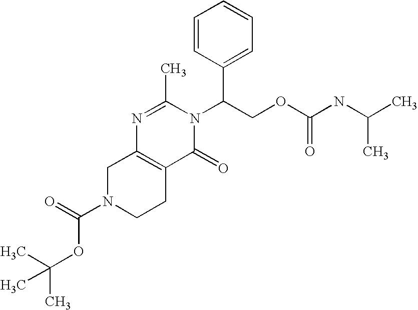 |
| 389 | 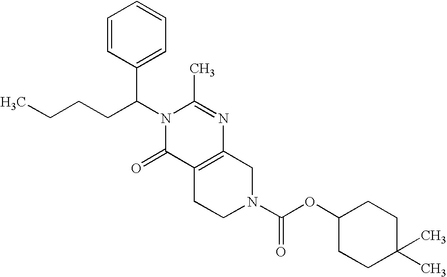 |
| 390 | 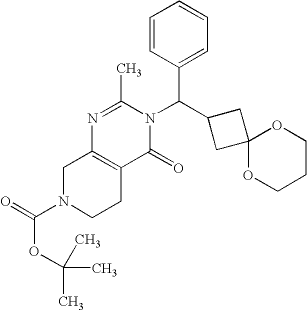 |
| 391 | 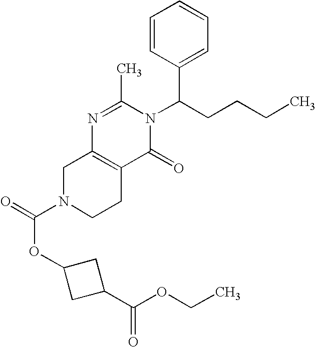 |
| 392 | 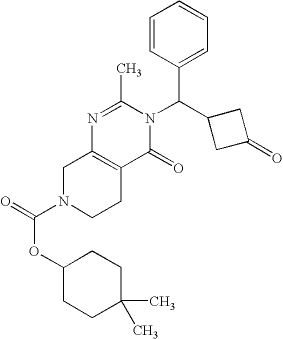 |
| 393 | 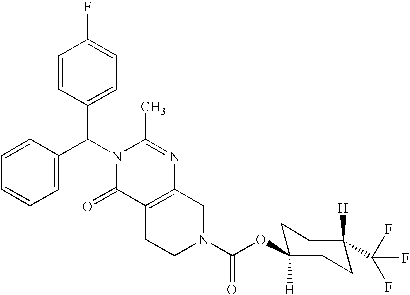 |
| 394 | 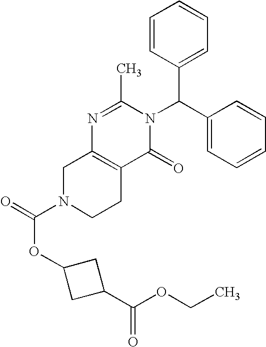 |
| 395 | 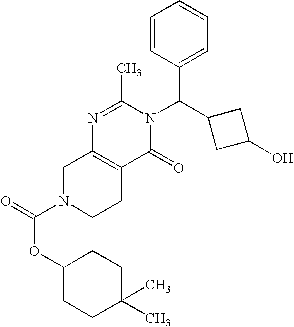 |
| 396 | 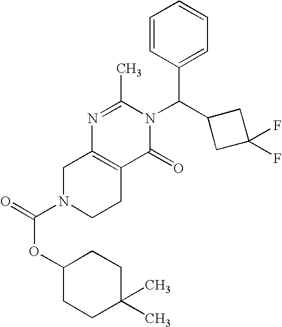 |
| 397 | 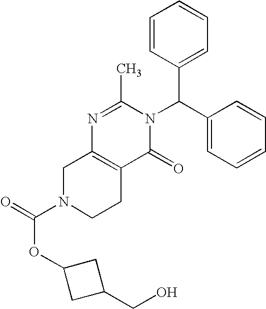 |
| 398 | 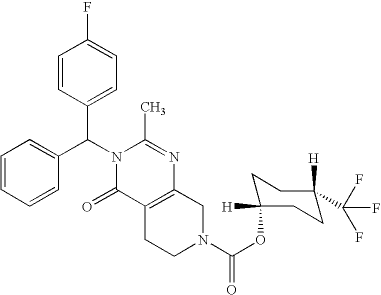 |
| 399 | 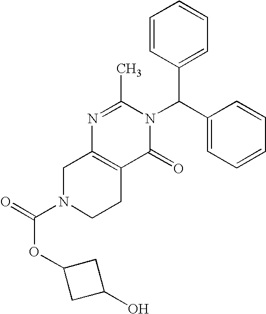 |
| 400 | 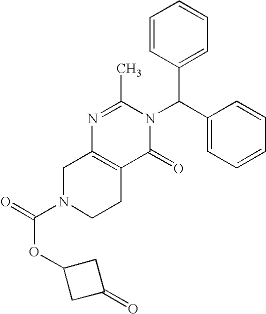 |
| 401 | 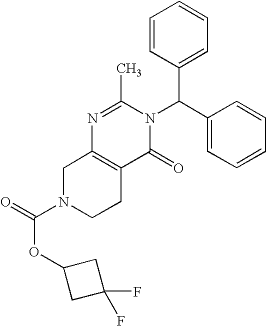 |
| 402 | 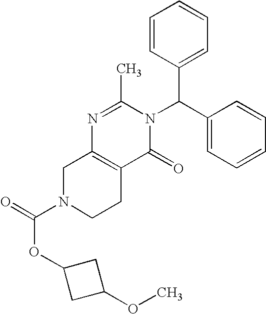 |
| 403 | 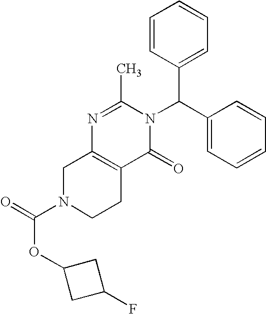 |
| 404 | 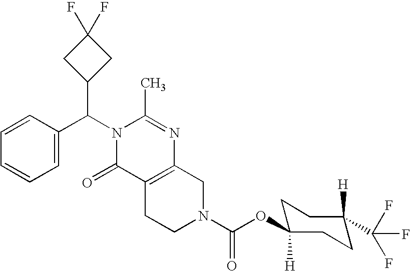 |
| 405 | 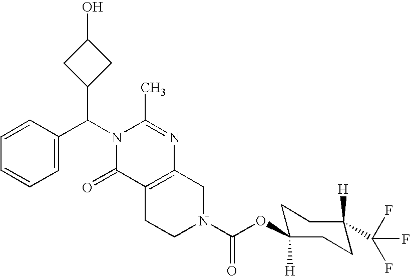 |
| 406 | 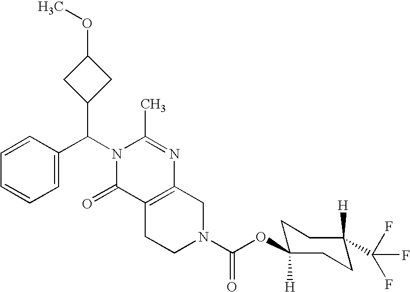 |
| 407 | 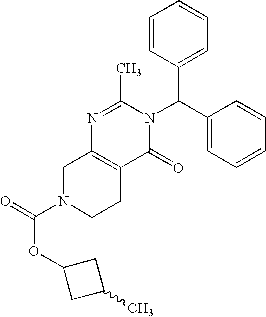 |
| 408 | 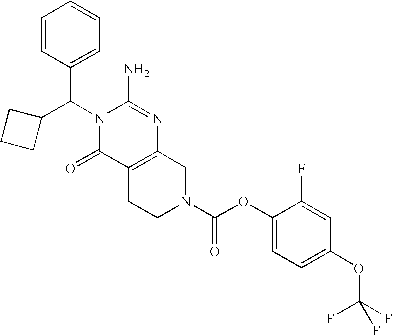 |
| 409 | 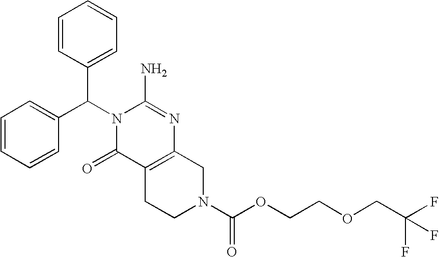 |
| 410 | 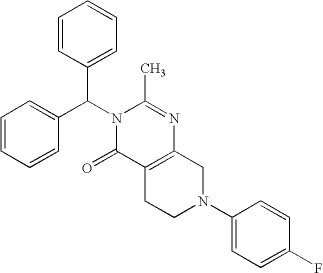 |
| 411 | 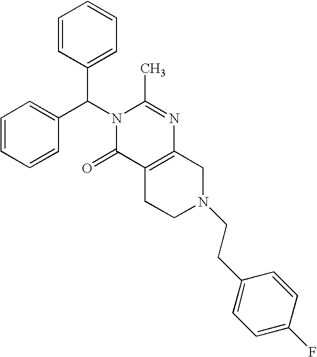 |
| 412 | 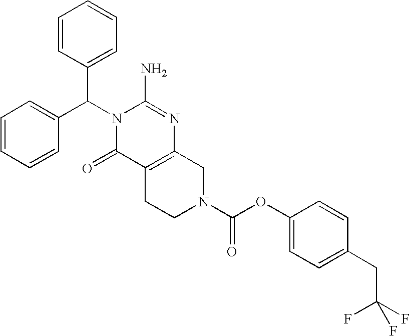 |
| 413 | 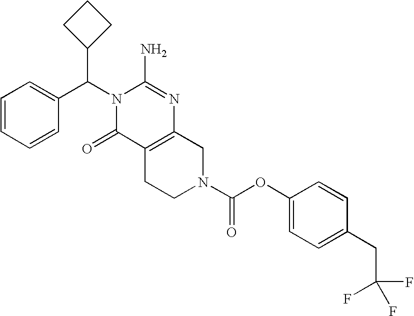 |
| 414 | 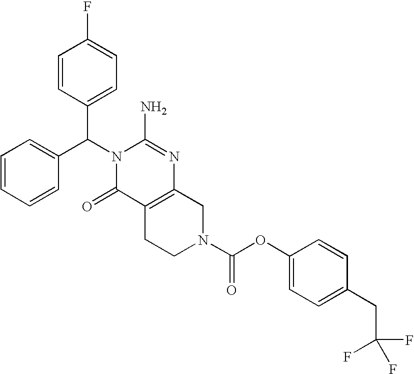 |
| 415 | 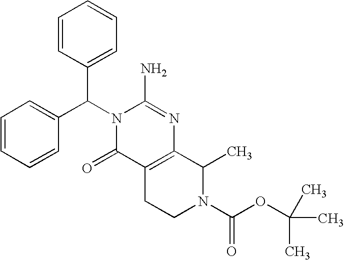 |
| 416 | 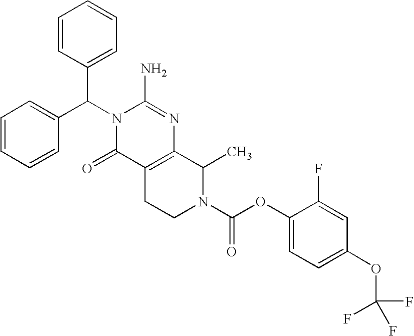 |
| 417 | 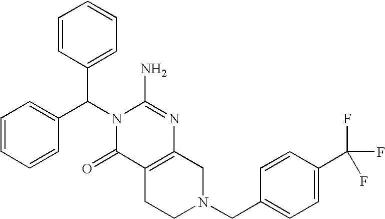 |
| 418 | 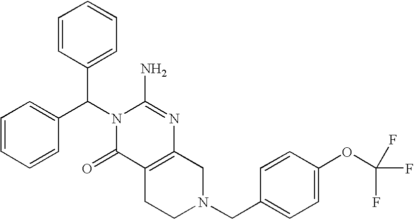 |
| 419 | 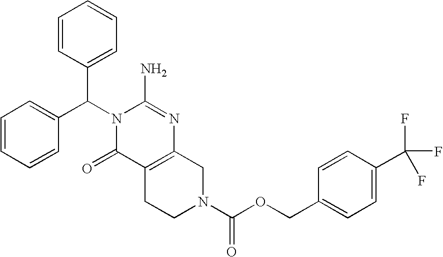 |
| 420 | 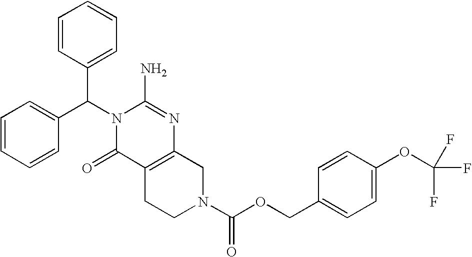 |
| 421 | 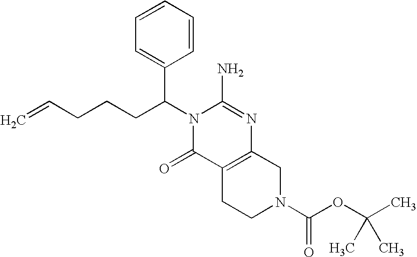 |
| 422 | 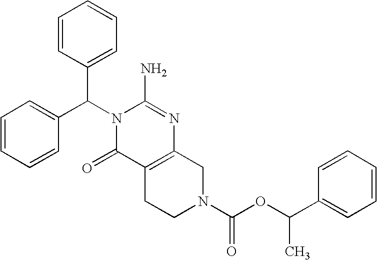 |
| 423 | 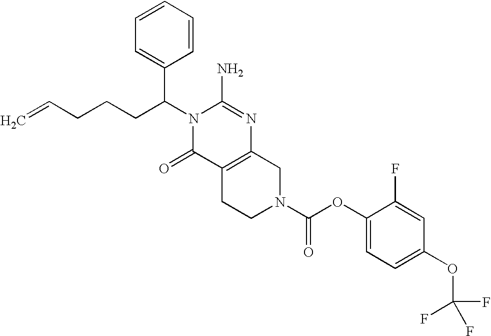 |
| 424 | 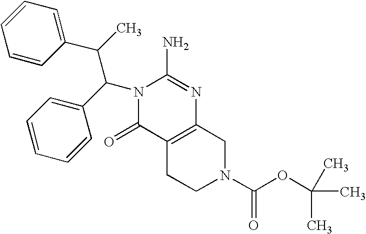 |
| 425 | 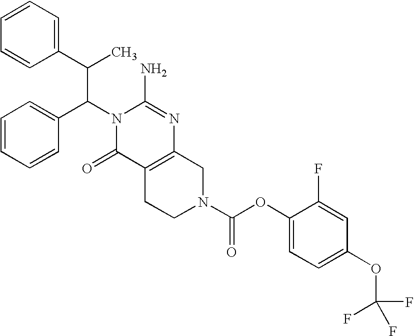 |
| 426 | 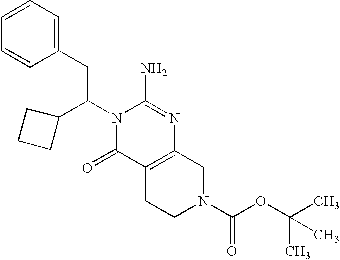 |
| 427 | 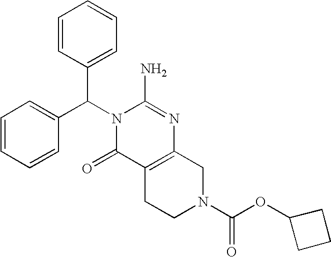 |
| 428 | 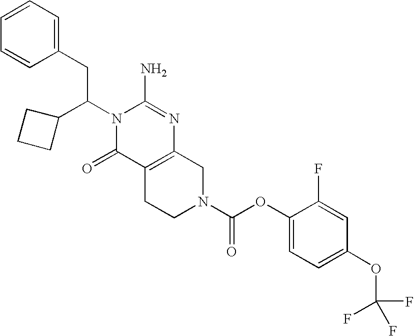 |
| 429 | 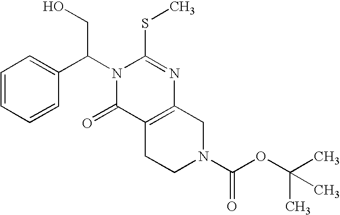 |
| 430 | 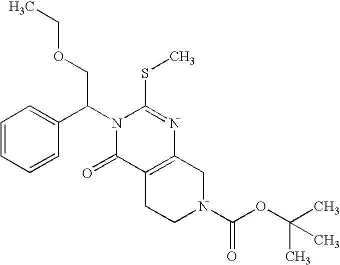 |
| 431 | 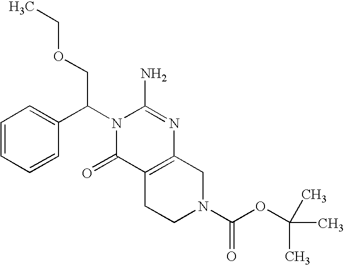 |
| 432 | 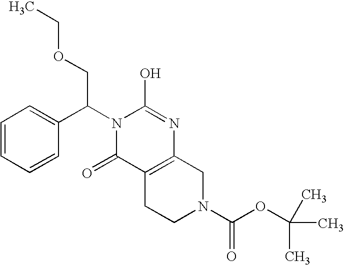 |
| 433 | 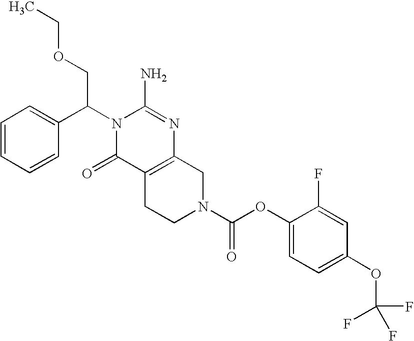 |
| 434 | 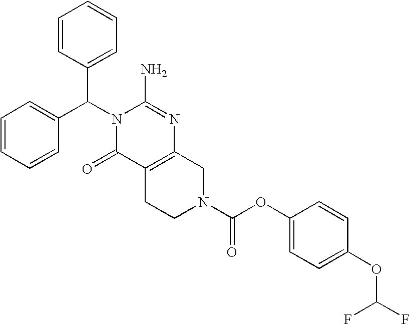 |
| 435 | 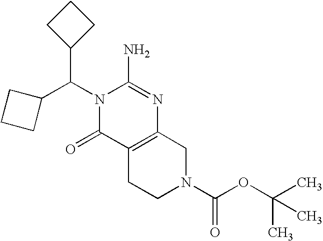 |
| 436 | 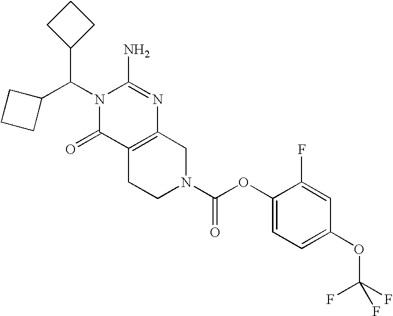 |
| 437 | 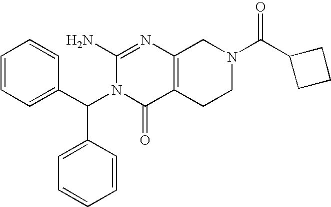 |
| 438 | 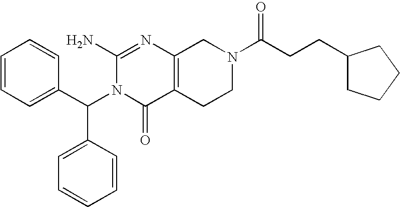 |
| 439 | 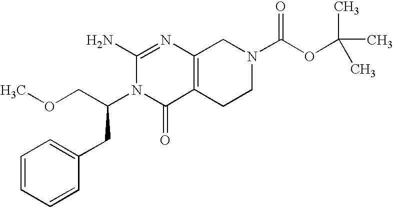 |
| 440 | 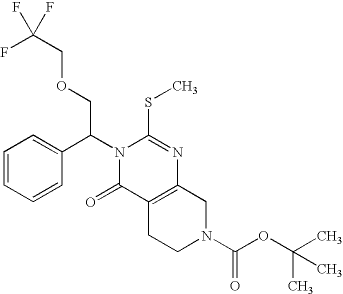 |
| 441 | 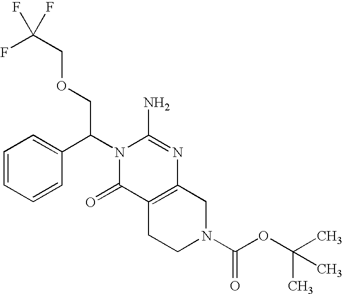 |
| 442 | 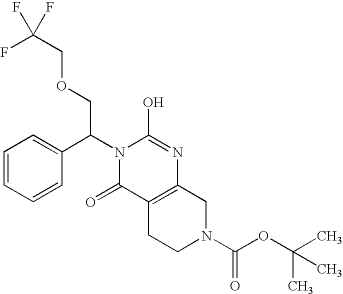 |
| 443 | 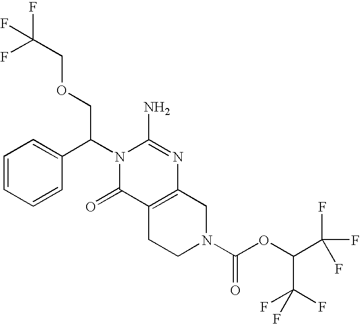 |
| 444 | 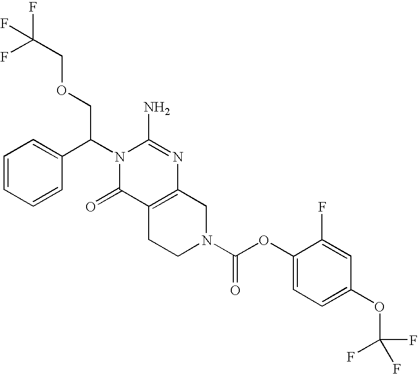 |
| 445 | 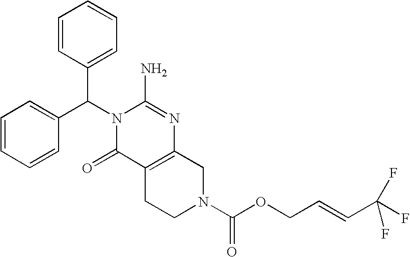 |
| 446 | 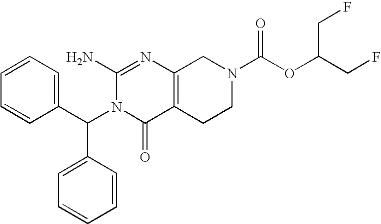 |
| 447 | 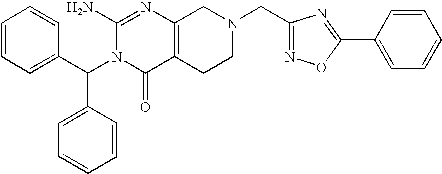 |
| 448 | 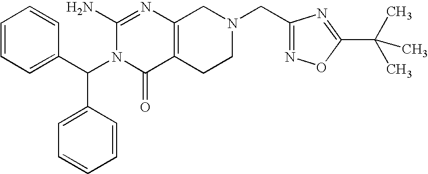 |
| 449 | 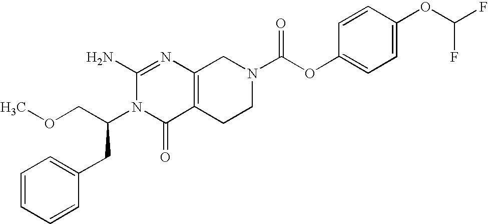 |
| 450 | 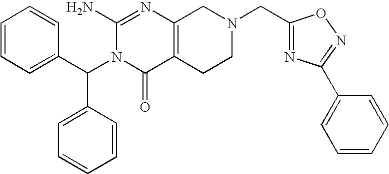 |
| 451 | 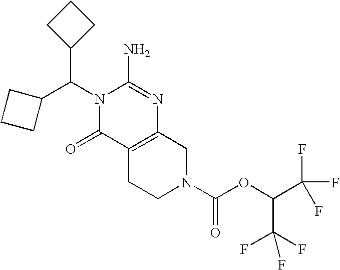 |
| 452 | 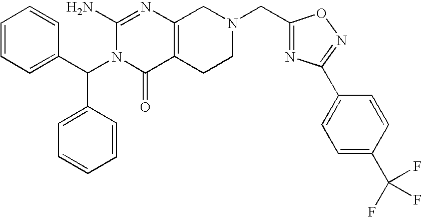 |
| 453 | 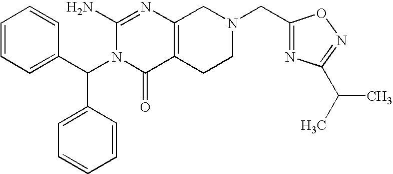 |
| 454 | 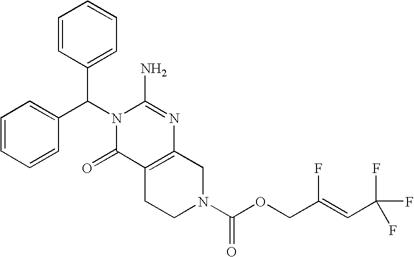 |
| 455 | 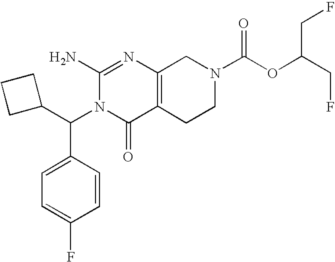 |
| 456 | 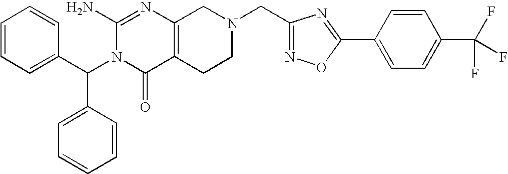 |
| 457 | 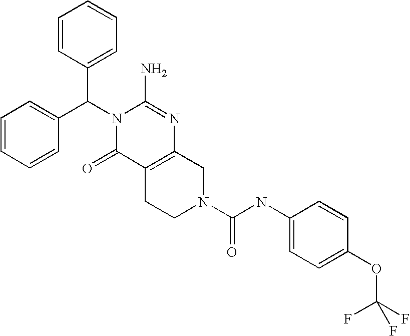 |
| 458 | 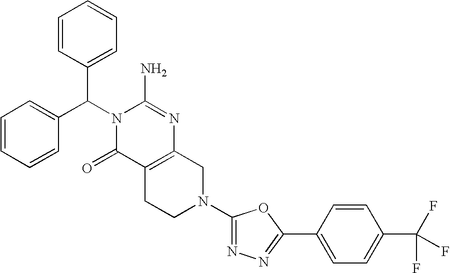 |
| 459 | 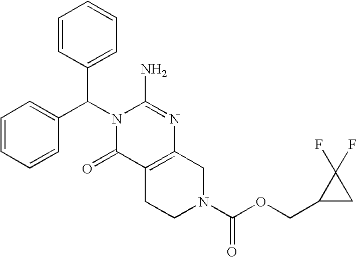 |
| 460 | 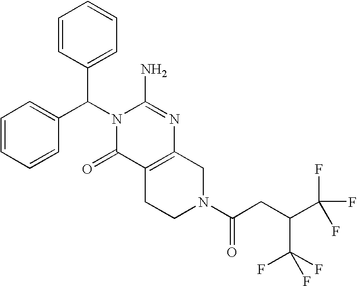 |
| 461 | 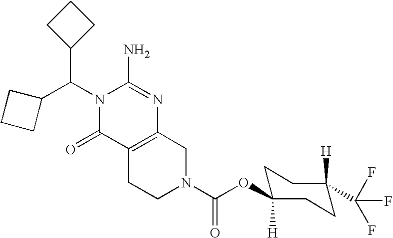 |
| 462 | 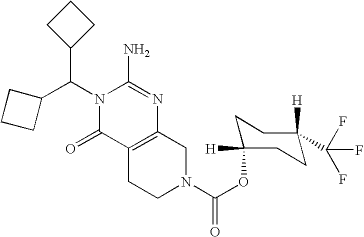 |
| 463 | 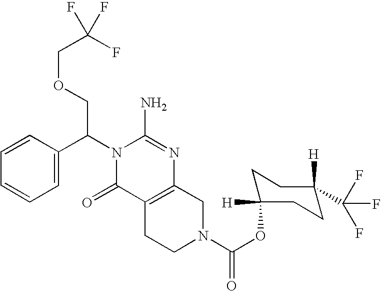 |
| 464 | 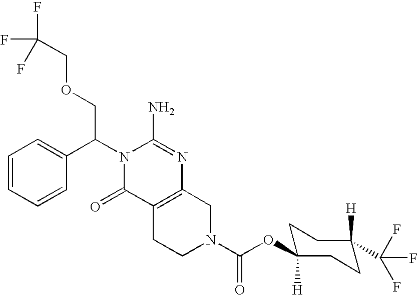 |
| 465 | 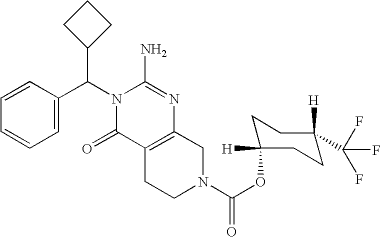 |
| 466 | 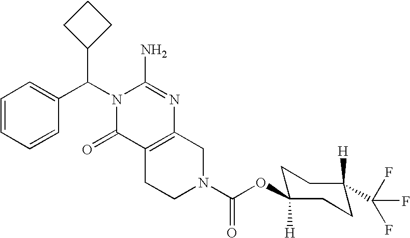 |
| 467 | 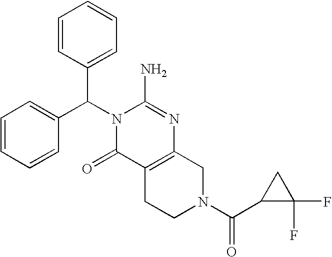 |
| 468 | 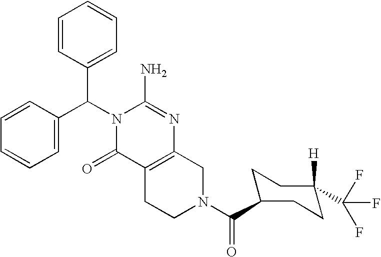 |
| 469 | 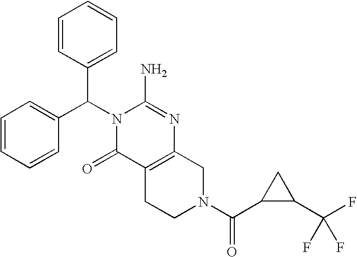 |
| 470 | 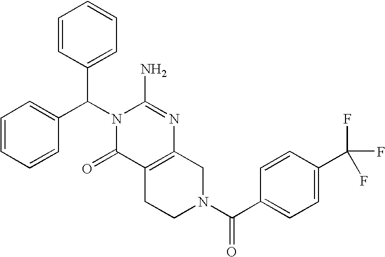 |
| 471 | 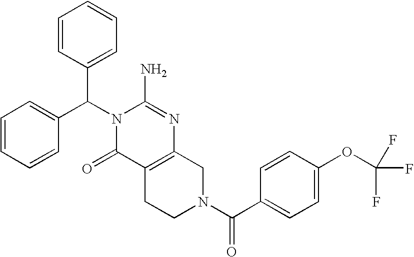 |
| 472 | 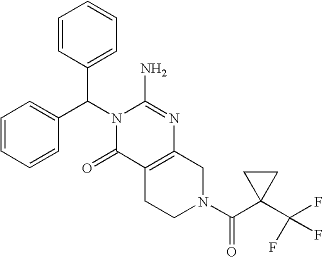 |
| 473 | 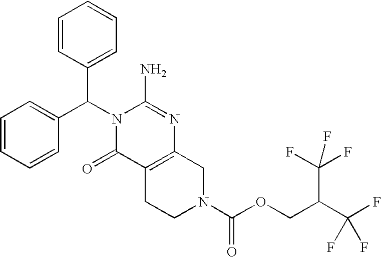 |
| 474 | 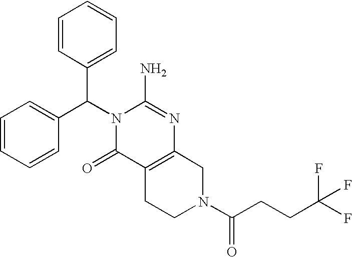 |
| 475 | 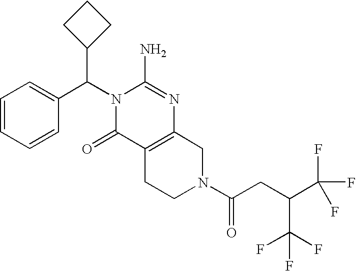 |
| 476 | 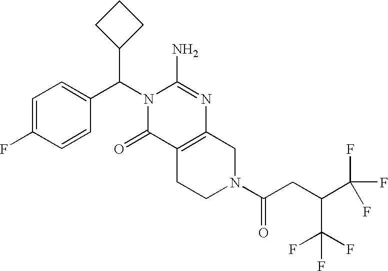 |
| 477 | 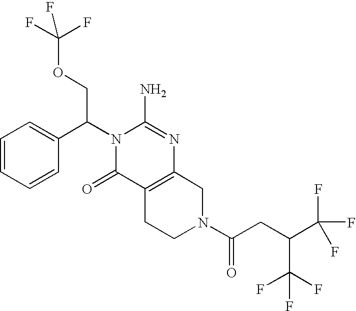 |
| 478 | 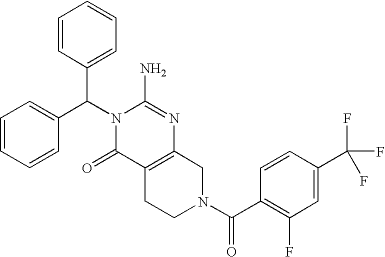 |
| 479 | 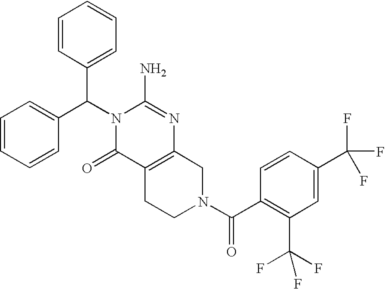 |
| 480 | 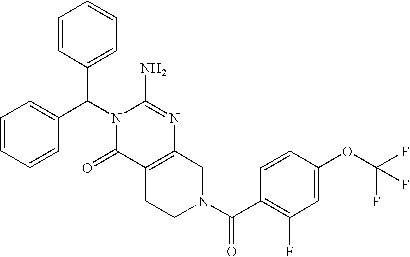 |
| 481 | 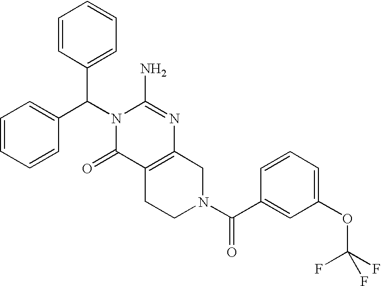 |
| 482 | 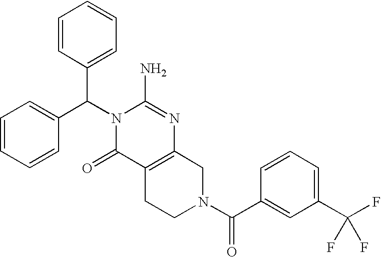 |
| 483 | 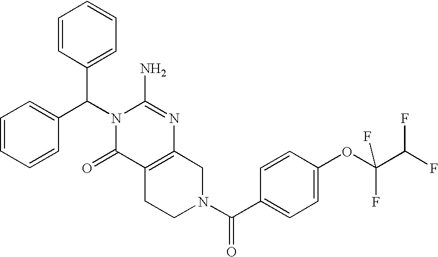 |
| 484 | 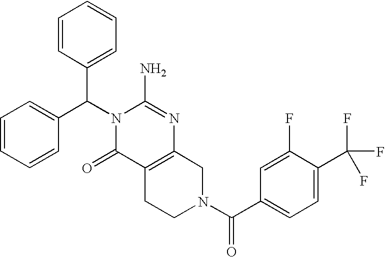 |
| 485 | 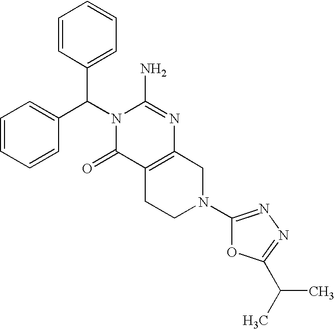 |
| 486 | 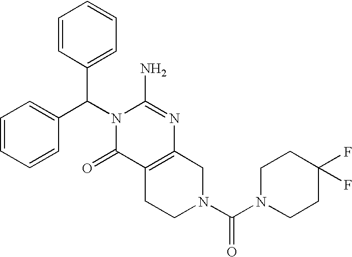 |
| 487 | 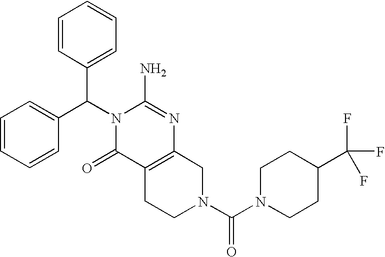 |
| 488 | 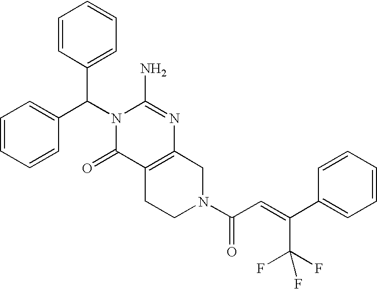 |
| 489 | 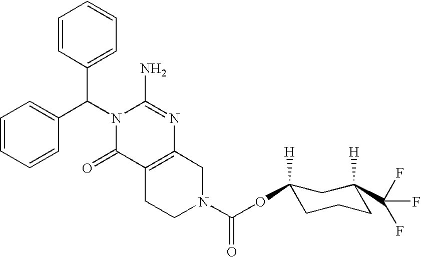 |
| 490 | 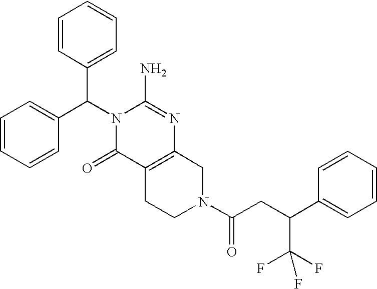 |
| 491 | 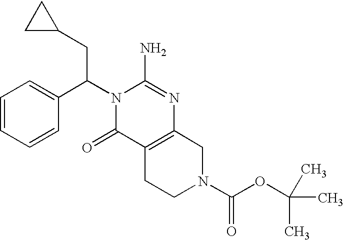 |
| 492 | 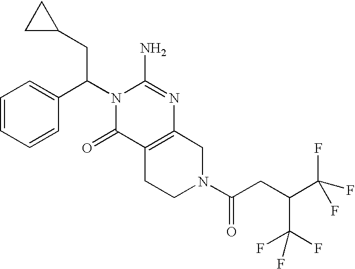 |
| 493 | 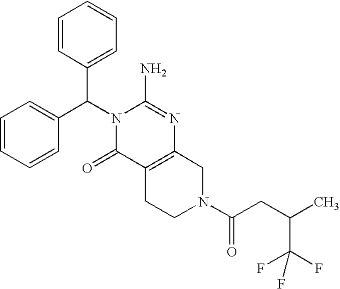 |
| 494 | 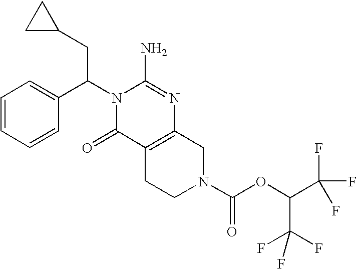 |
| 495 | 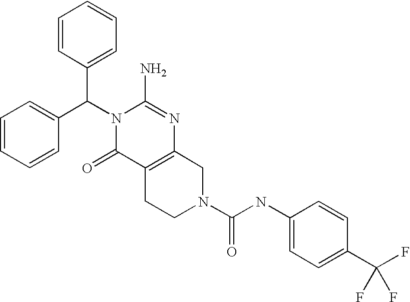 |
| 496 | 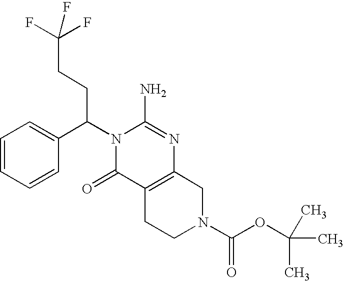 |
| 497 | 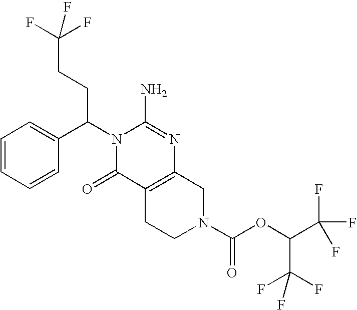 |
| 498 | 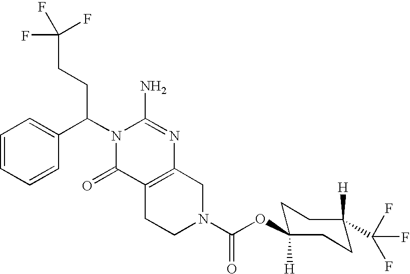 |
| 499 | 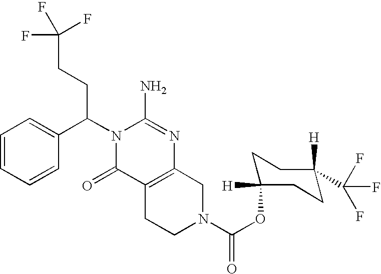 |
| 500 | 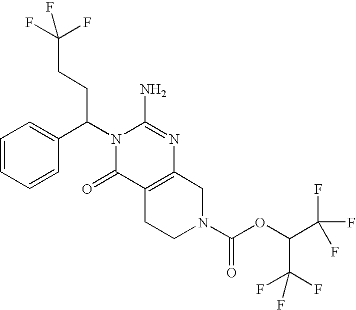 |
| 501 | 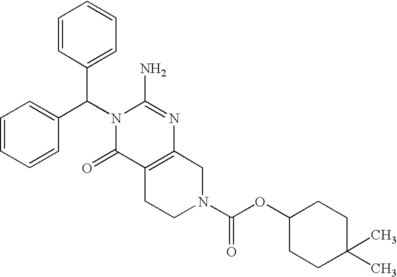 |
| 502 | 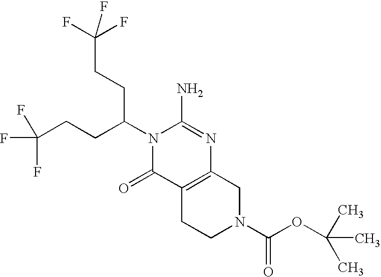 |
| 503 | 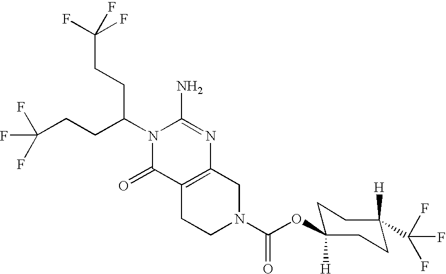 |
| 504 | 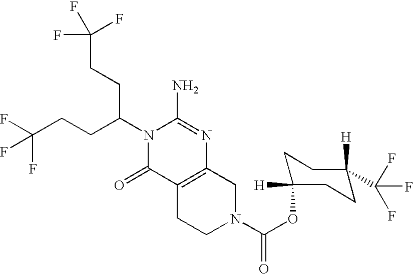 |
| 505 | 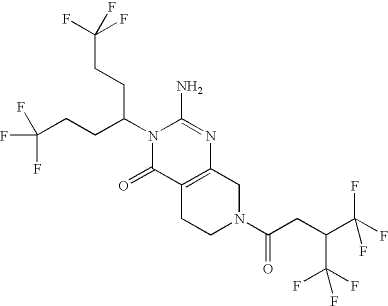 |
| 506 | 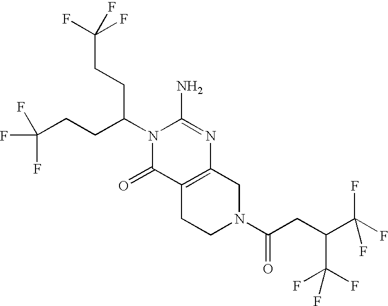 |
| 507 | 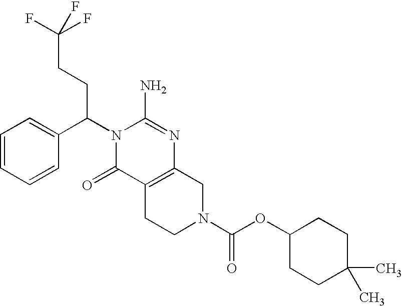 |
| 508 | 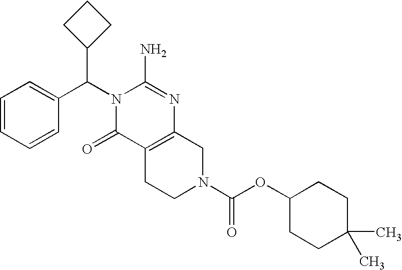 |
| 509 | 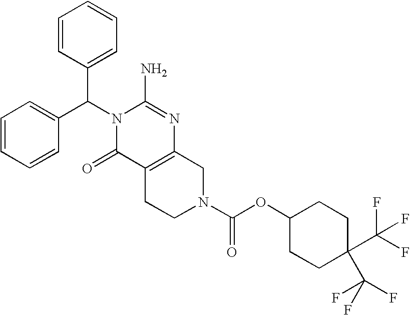 |
| 510 | 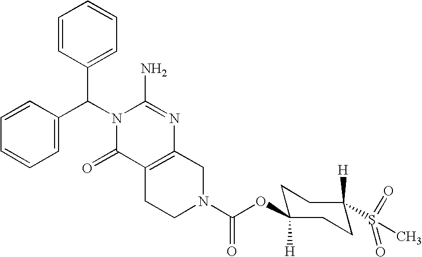 |
| 511 | 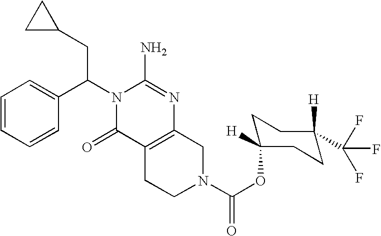 |
| 512 | 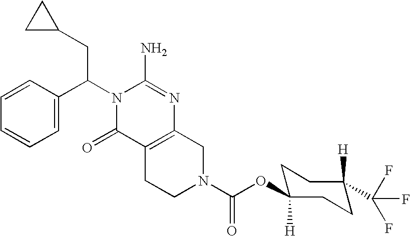 |
| 513 | 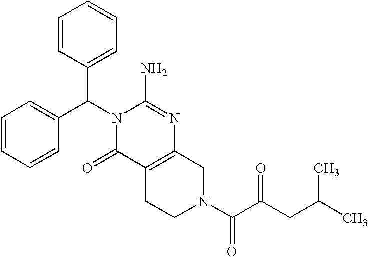 |
| 514 | 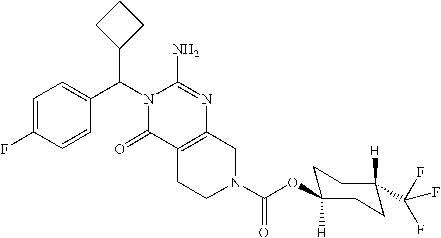 |
| 515 | 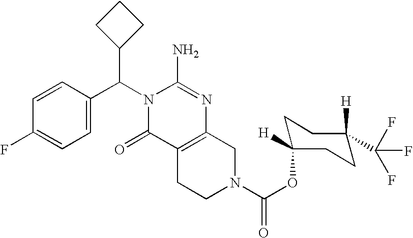 |
| 516 | 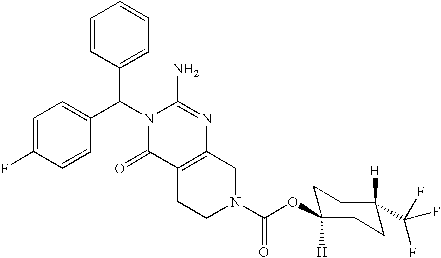 |
| 517 | 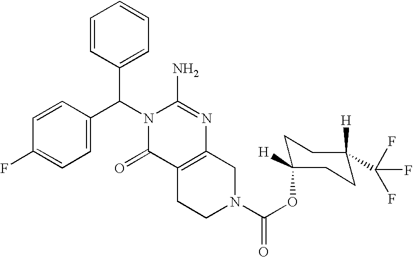 |
| 518 | 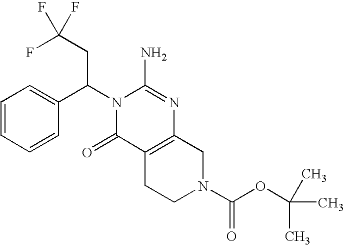 |
| 519 | 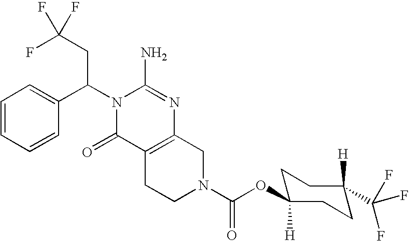 |
| 520 | 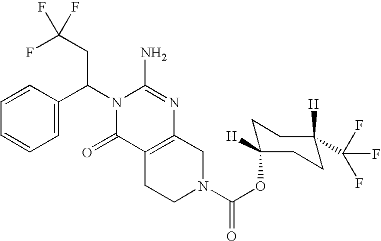 |
| 521 | 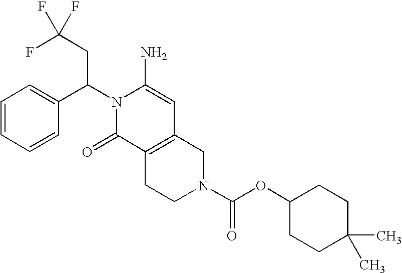 |
| 522 | 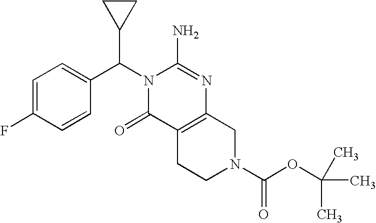 |
| 523 | 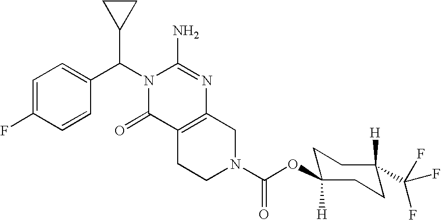 |
| 524 | 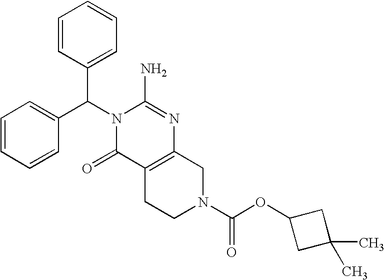 |
| 525 | 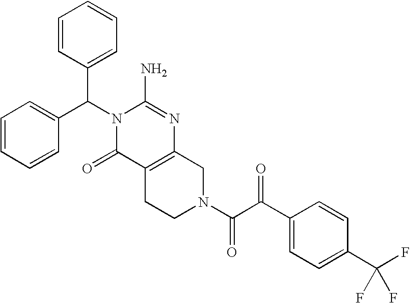 |
| 526 | 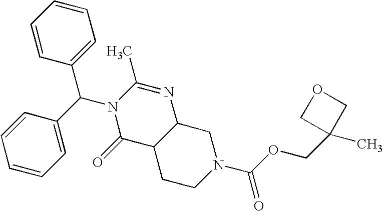 |
| 527 | 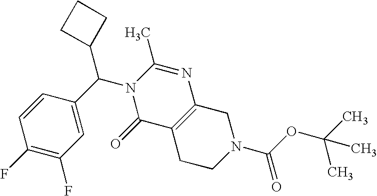 |
| 528 | 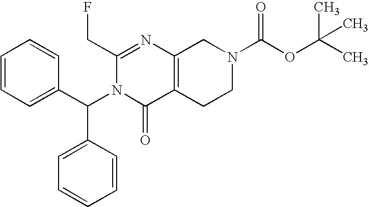 |
| 529 | 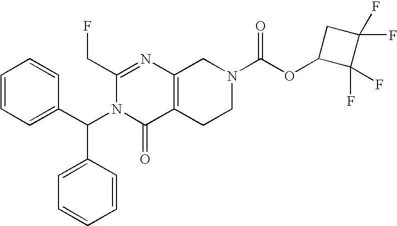 |
| 530 | 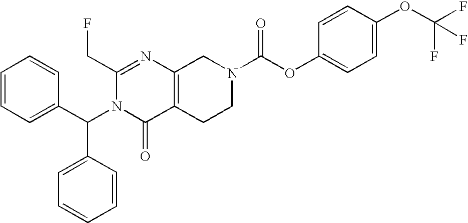 |
| 531 | 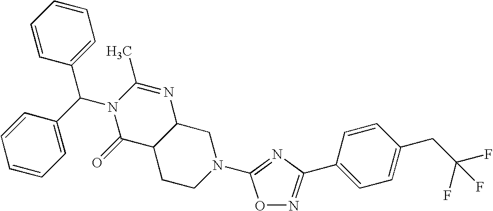 |
| 532 | 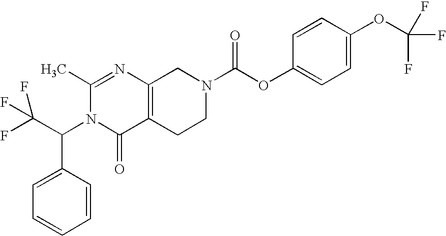 |
| 533 | 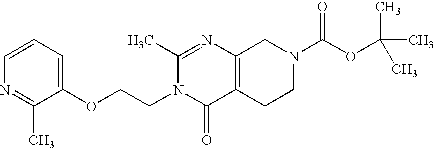 |
| 534 | 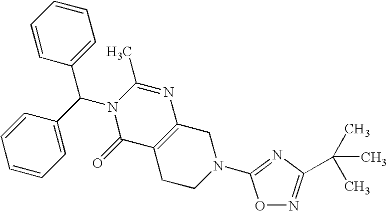 |
| 535 | 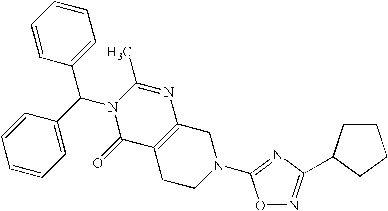 |
| 536 | 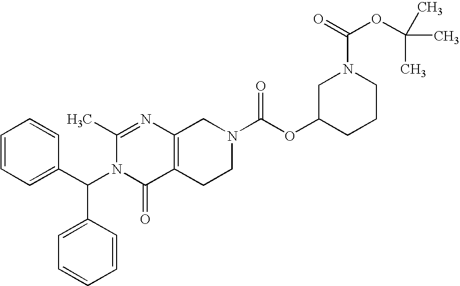 |
| 537 | 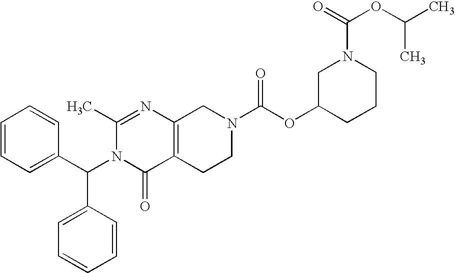 |
| 538 | 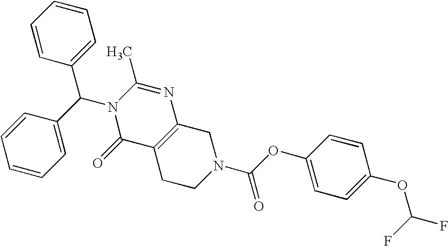 |
| 539 | 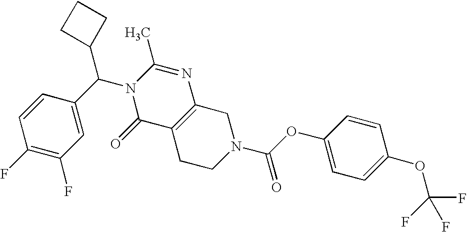 |
| 540 | 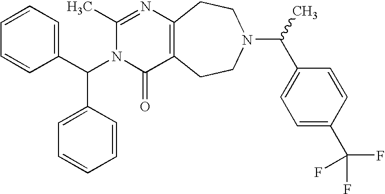 |
| 541 | 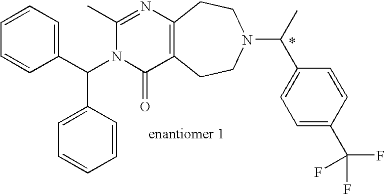 |
| 542 | 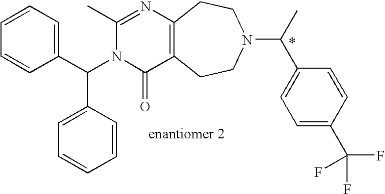 |
| 543 | 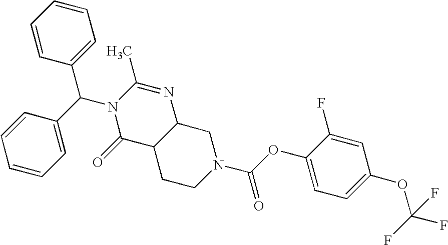 |
| 544 | 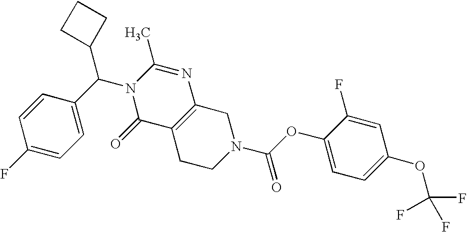 |
| 545 | 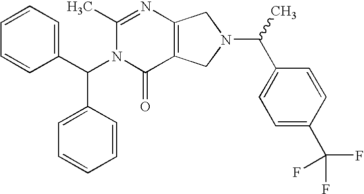 |
| 546 | 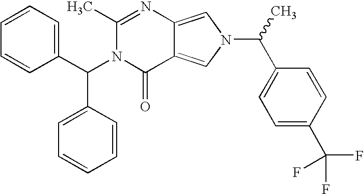 |
| 547 | 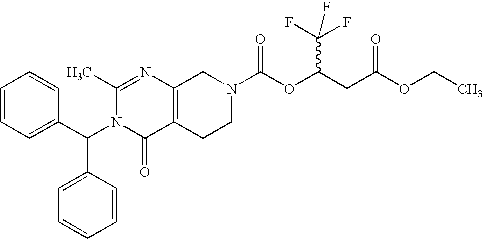 |
| 548 | 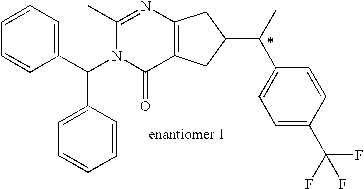 |
| 549 | 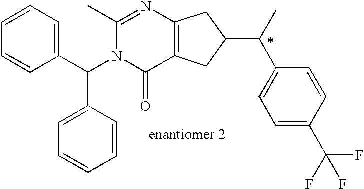 |
| 550 | 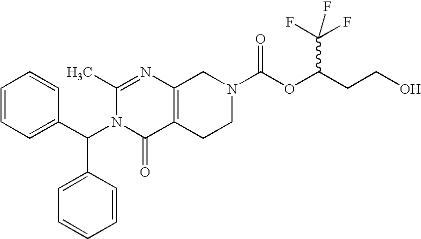 |
| 551 | 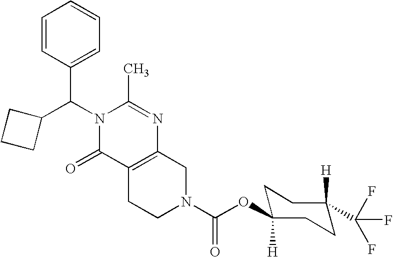 |
| 552 | 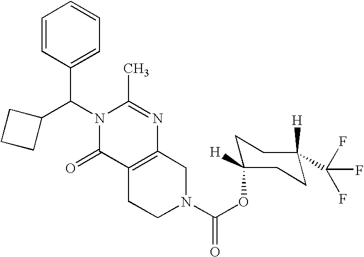 |
| 553 | 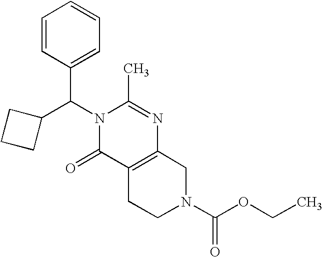 |
| 554 | 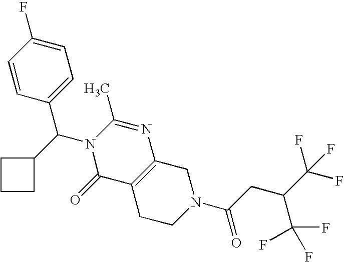 |
| 555 | 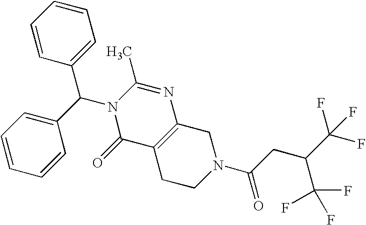 |
| 556 | 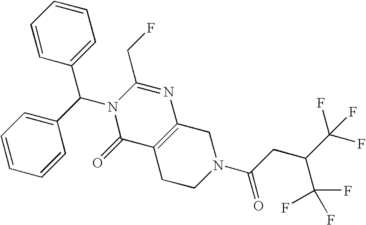 |
| 557 | 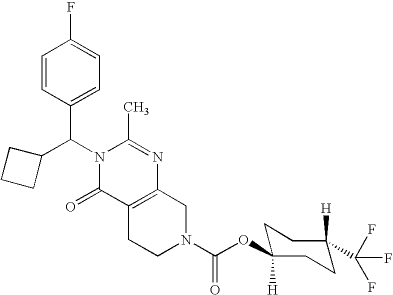 |
| 558 | 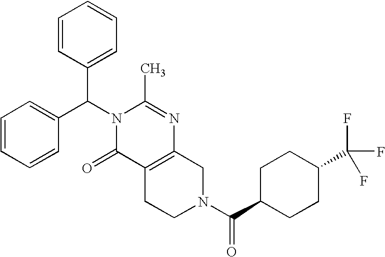 |
| 559 | 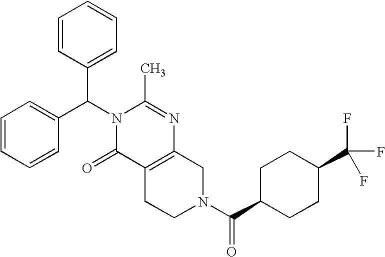 |
| 560 | 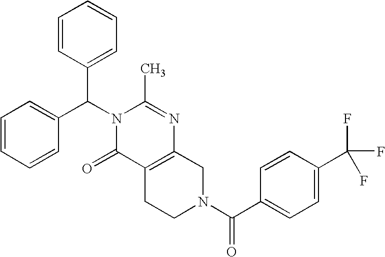 |
| 561 | 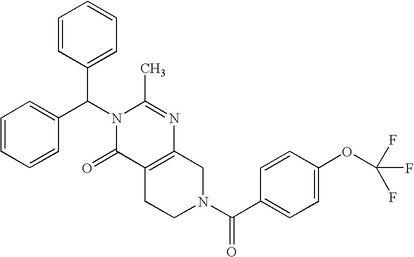 |
| 562 | 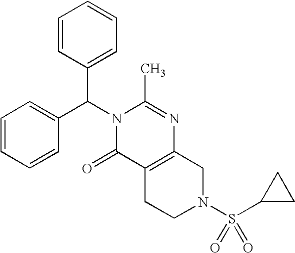 |
| 563 | 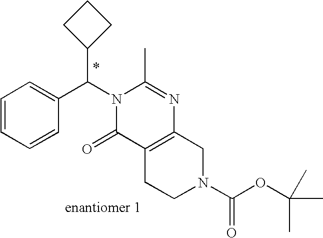 |
| 564 | 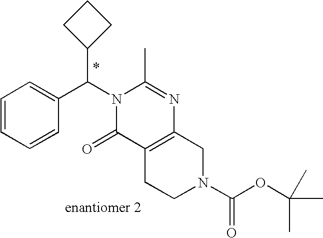 |
| 565 | 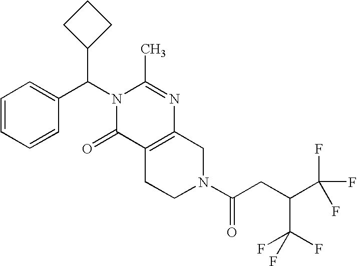 |
| 566 | 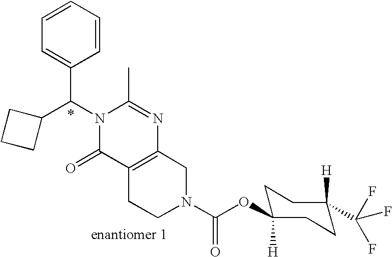 |
| 567 | 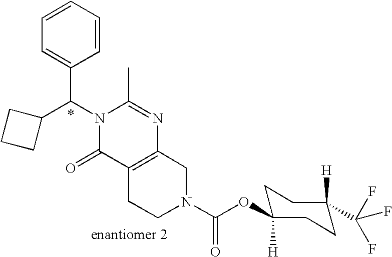 |
| 568 | 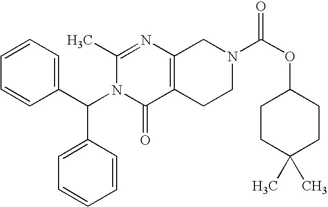 |
| 569 | 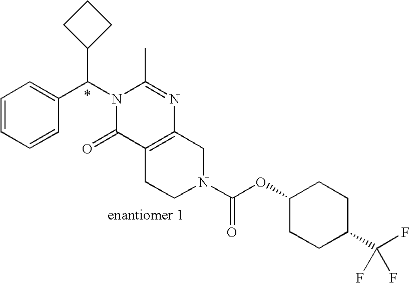 |
| 570 | 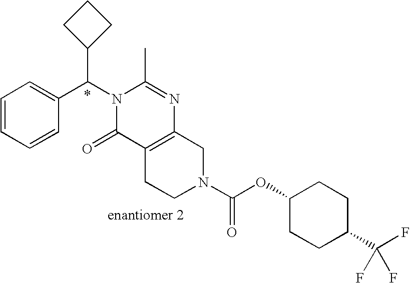 |
| 571 | 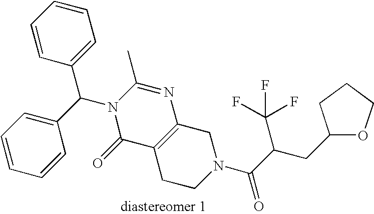 |
| 572 | 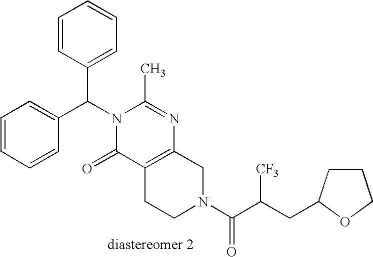 |
| 573 | 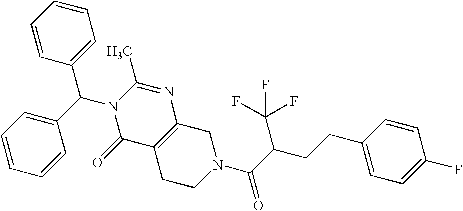 |
| 574 | 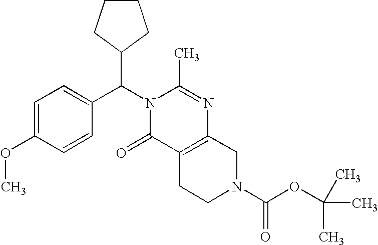 |
| 575 | 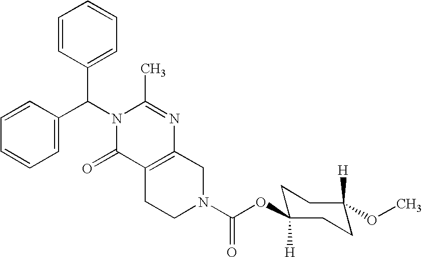 |
| 576 | 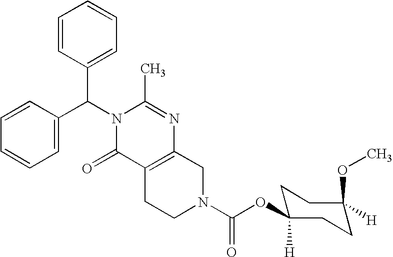 |
| 577 | 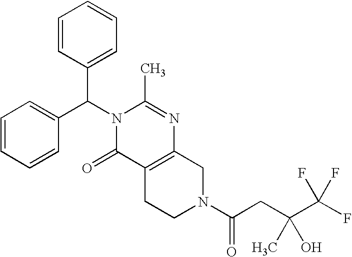 |
| 578 | 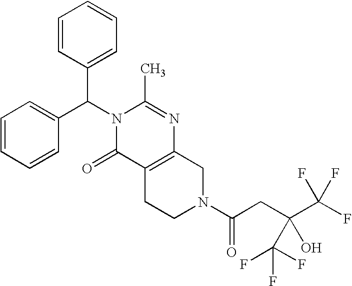 |
| 579 | 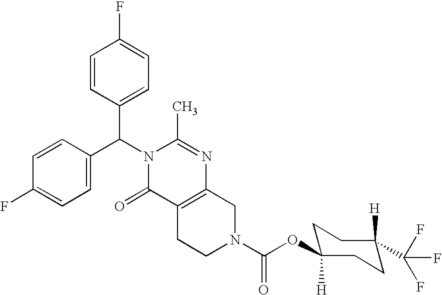 |
| 580 | 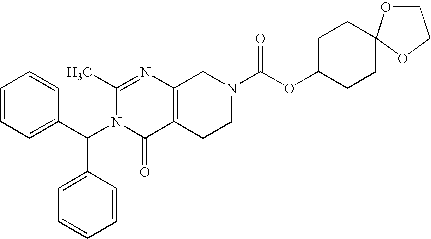 |
| 581 | 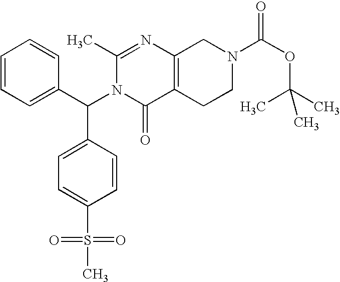 |
| 582 | 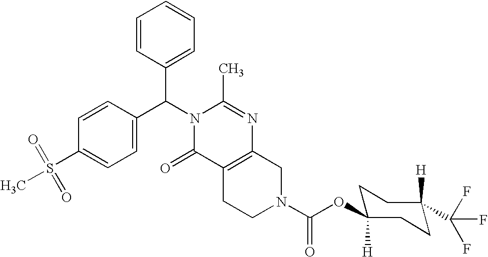 |
| 583 | 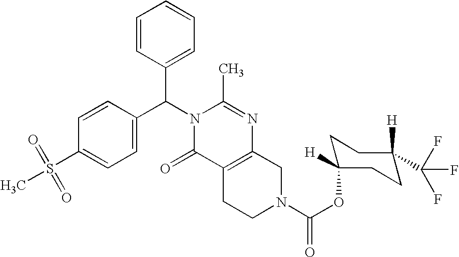 |
| 584 | 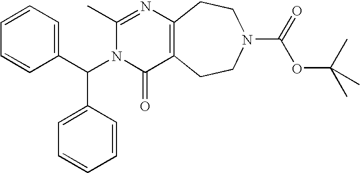 |

and pharmaceutically acceptable salts, solvates, esters and prodrugs thereof.

### Methods For Making the Pyrimidinone Derivatives

Methods useful for making the Pyrimidinone Derivatives are set forth in the Examples below and generalized in Schemes 1-12. Alternative synthetic pathways and analogous structures will be apparent to those skilled in the art or organic synthesis.

Scheme 1 shows a method useful for making compound C, which is a useful intermediate for making the Pyrimidinone Derivatives wherein G is —CH₂— and J is a single bond.

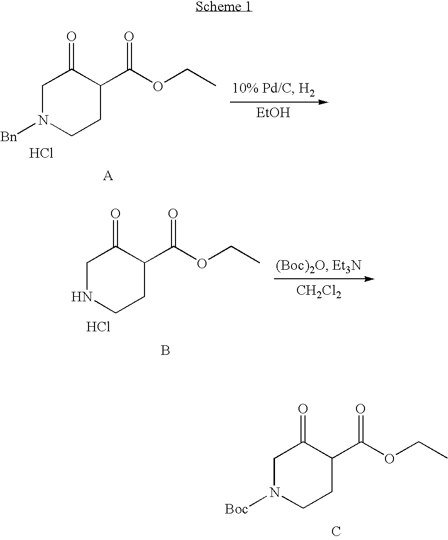

A 4-Oxo-N-benzyl piperidinyl compound of formula A can be deprotected via catalytic hydrogenation using Pd/C to provide the 4-Oxo-piperidinyl compound B. The cyclic amine group of compound B can then be reprotected as its t-butyloxycarbonyl (BOC) derivative to provide intermediate compound C using BOC-anhydride and triethylamine.

Scheme 2 shows a method for making the intermediate piperidine hydrochloride compounds of formula H which are useful intermediates for making the Pyrimidinone Derivatives, wherein J is a single bond and G is —CH₂—.

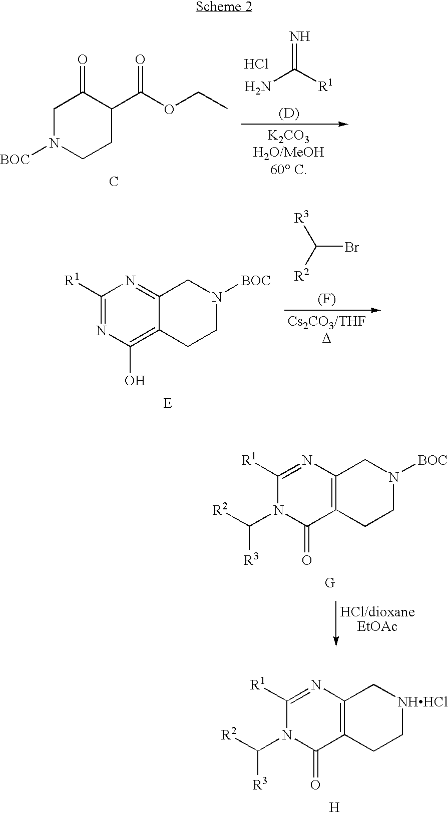

wherein J is a single bond, G is —CH₂—, and R¹, R² and R³ are defined above for the compounds of formula (I).

Compound C can be reacted with an amidine hydrochloride compound of formula D to provide the pyrimidino-piperidine compounds of formula E, which can then be reacted with a compound of formula F in the presence of a carbonate base to provide the substituted pyrimidinone compounds of formula G. The BOC protecting group of a compound of formula G can then be removed using HCl to provide the piperidine hydrochloride compounds of formula H.

Scheme 3 illustrates an alternative method for making the compounds of formula G, which are useful intermediates for making the Pyrimidinone Derivatives, wherein J is a single bond and G is —CH₂.

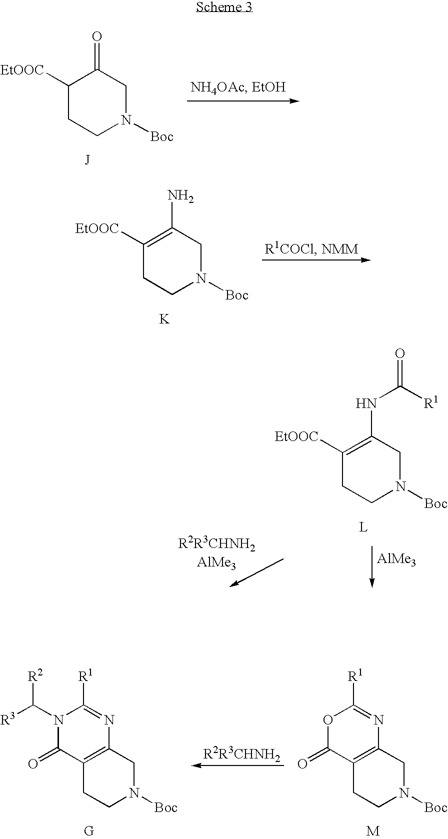

wherein J is a single bond, G is —CH₂—, and R¹, R² and R³ are defined above for the compounds of formula (I).

Ketone compound J can be reacted with ammonium acetate or ammonia in a solvent such as ethanol, at ambient or elevated temperature to provide enamine K. Compound K can then be acylated using an acyl chloride of formula R¹C(O)Cl, typically in the presence of an amine such as N-methylmorpholine (NMM) in an inert solvent such as dichloromethane. The resulting amide compounds of formula L may be treated with trimethylaluminum in inert solvents, such as dichloromethane/heptane to provide the benzoxazinone compounds of formula M, which can then be reacted with an amine of formula R²R³CHNH₂, to provide the intermediate compounds of formula G. Alternatively, a compound of formula L may be reacted with trimethylaluminum and the resulting reaction mixture treated directly with an amine of formula R²R³CHNH₂ to provide the compounds of formula G in a one-pot procedure.

Scheme 4 illustrates a method useful for making the compounds of formula T, which are useful intermediates for making the Pyrimidinone Derivatives, wherein J is a single bond, G is —CH₂—, and R¹ is —NH₂, NH-alkyl, N(alkyl)₂, SH, S-alkyl, or S(O)ₚ-alkyl.

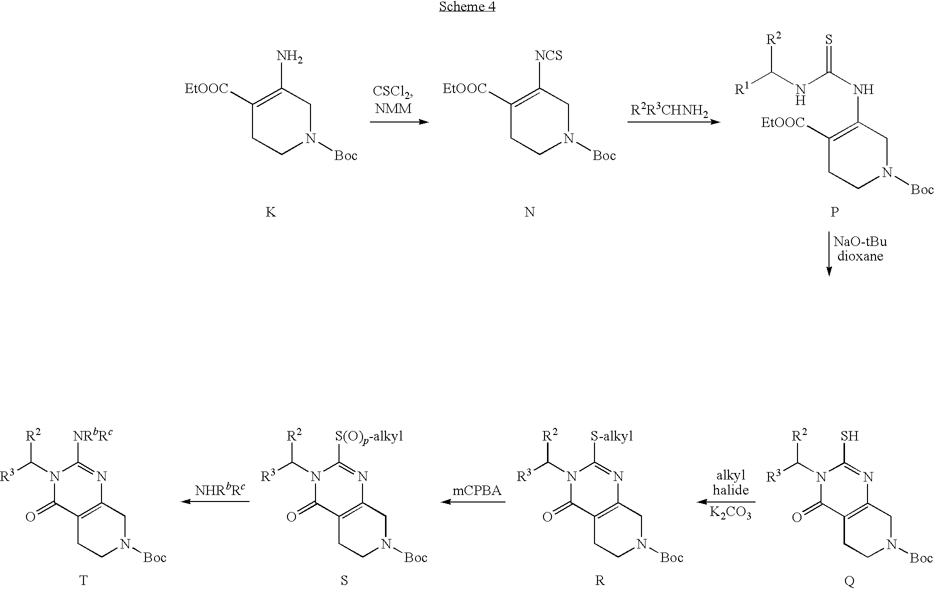

wherein J is a single bond; G is —CH₂—; R¹, R² and R³ are defined above for the compounds of formula (I); and R^{b} and R^{c} are each independently H or alkyl.

Intermediate K can be treated with thiophosgene in the presence of a base such as N-methylmorpholine (NMM) to provide isothiocyanate N. Reaction with an amine of formula R²R³CHNH₂ provides thiourea compounds of formula P, which can then be cyclized using a strong base such as NaO-tBu, to provide the bicyclic intermediates of formula Q. The compounds of formula Q can then be alkylated using, for example, an alkyl halide and a base such as K₂CO₃ to provide the compounds of formula R, which are then oxidized to the corresponding sulfoxide or sulfone compounds of formula S, depending upon choice of oxidizing conditions. Reaction of a sulfone of formula S with ammonia, an alkylamine, or dialkylamine provides amines of formula T.

Scheme 5 illustrates a method useful for making compounds of formula W, which are useful intermediates for making the Pyrimidinone Derivatives, wherein J is a single bond, G is —CH₂— and R¹ is —OR⁹.

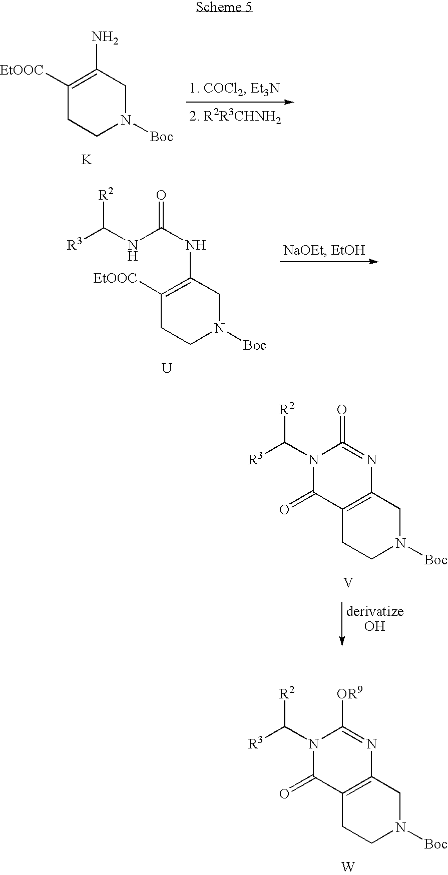

wherein J is a single bond, G is —CH₂— and R², R³ and R⁹ are defined above for the compounds of formula (I).

Intermediate K is treated with phosgene in the presence of a base such as triethylamine, followed by addition of an amine of formula R²R³CHNH₂ to provide the urea compounds of formula U. The compounds of formula U can then be cyclized upon treatment with strong base such as NaOEt to provide the compounds of formula V, which correspond to the Pyrimidinone Derivatives wherein R¹ is —OH. The compounds of formula V may be further derivatized using well-known methods to provide the compounds of formula W, which correspond to the Pyrimidinone Derivatives wherein R¹ is —OR⁹ and R⁹ is other than H.

Scheme 6 illustrates a method useful for making the substituted piperidinone compounds of formula AA, which are useful intermediates for making the Pyrimidinone Derivatives, wherein J is a single bond, G is —CH₂— and R¹¹ is other than H.

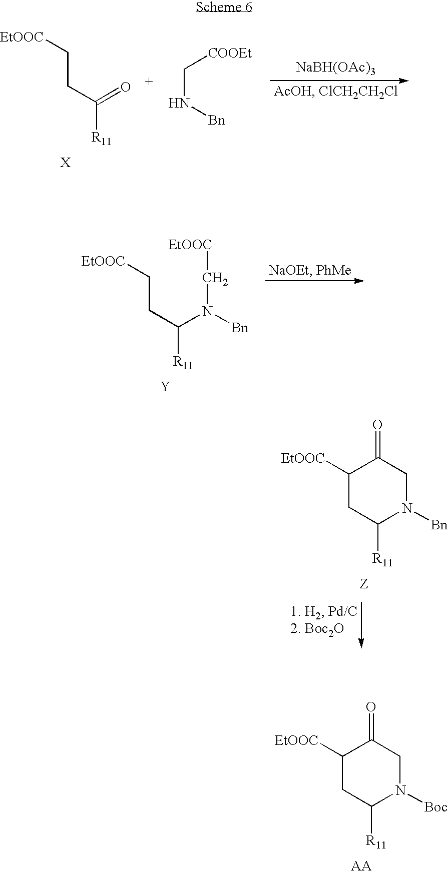

wherein J is a single bond, G is —CH₂— and R¹¹ is defined above for the compounds of formula (I).

A β-ketoester of formula X, readily available using known methods, is reductively aminated with N-benzyl glycine ester using NaBH(OAc)₃ and AcOH to provide the amino diester compounds of formula Y. The compounds of formula Y can then be cyclized by means of a strong base, such as NaOEt, in a non-polar solvent such as toluene, to provide piperidinone compounds of formula Z. Removal of the benzyl protecting group from Z, followed by BOC protection of the resulting amine, provides the piperidinone intermediates of formula AA.

Scheme 7 illustrates a method useful for making substituted piperidinone compounds of formula EE, which are useful intermediates for making the Pyrimidinone Derivatives, wherein J is a single bond, G is —CH₂— and R¹⁰ is other than H.

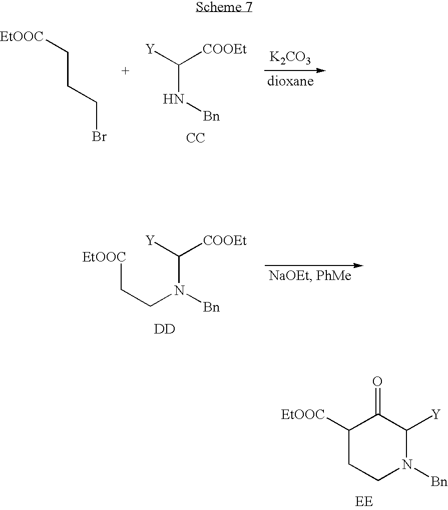

wherein J is a single bond, G is —CH₂— and R¹⁰ is defined above for the compounds of formula (I).

4-Bromobutyric acid ethyl ester is reacted with an α-benzylamino-ester of formula CC to provide the amino-diesters of formula DD. The compounds of formula DD can then be cyclized to compounds of formula EE using a base-mediated condensation.

Scheme 8 shows a method for converting intermediate compounds of formula H to the Pyrimidinone Derivatives of formula GG, wherein J is a single bond, G is —CH₂— and R⁴ is joined via a methylene group.

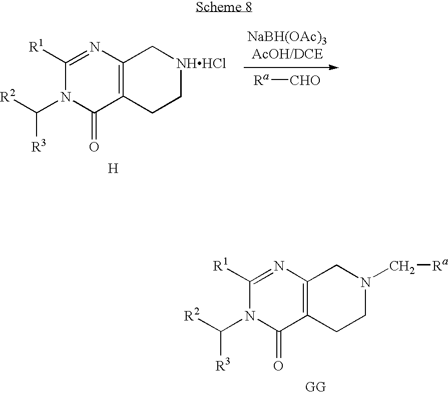

wherein J is a single bond; G is —CH₂—; R¹, R² and R³ are defined above for the compounds of formula (I); and —CH₂R^{a} is representative of all R⁴ substituents, as defined for the compounds of formula (I), that are connected via a methylene group.

The amine hydrochloride compounds of formula H can be reacted with an aldehyde of formula R^{a}—CHO, followed by reduction of the resulting imine using NaBH(OAc)₃ to provide the compounds of formula GG, which correspond to the compounds of formula (I) wherein R⁴ is a substituent that is connected via a methylene group.

Scheme 9 shows a method for converting intermediate compounds of formula H to the Pyrimidinone Derivatives of formula HH, wherein J is a single bond, G is —CH₂— and R⁴ is joined via a SO₂— group.

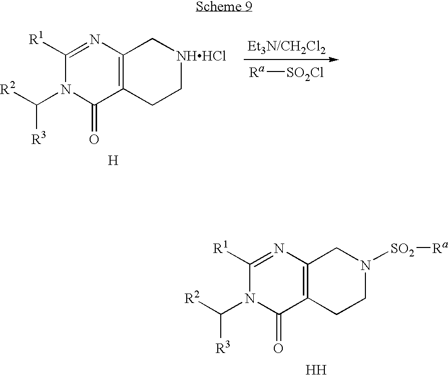

wherein J is a single bond; G is —CH₂, R¹, R² and R³ are defined above for the compounds of formula (I); and —S(O)₂R^{a} is representative of all R⁴ substituents, as defined for the compounds of formula (I), that are connected via a —S(O)₂— group.

The amine hydrochloride compounds of formula H can be reacted with sulfonyl chloride of formula R^{a}—SO₂Cl in the presence of a non-nucleophilic base, such as Et₃N, to provide the compounds of formula HH, which correspond to the compounds of formula (I) wherein R⁴ is a substituent that is connected via a —S(O)₂— group.

Scheme 10 shows a method for converting intermediate compounds of formula H to the Pyrimidinone Derivatives of formula JJ, wherein J is a single bond, G is —CH₂— and R⁴ is joined via a —C(O)NH— group.

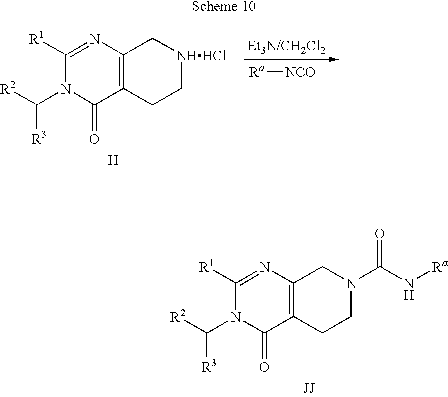

wherein J is a single bond; G is —CH₂—; R¹, R² and R³ are defined above for the compounds of formula (I); and —C(O)NHR^{a} is representative of all R⁴ substituents, as defined for the compounds of formula (I), that are connected via a —C(O)NH— group.

The amine hydrochloride compounds of formula H can be reacted with an isocyanate of formula R^{a}—NCO, in the presence of a non-nucleophilic base, such as Et₃N, to provide the compounds of formula JJ, which correspond to the compounds of formula (I) wherein R⁴ is a substituent that is connected via a —C(O)NH— group.

Scheme 11 shows a method for converting intermediate compounds of formula H to the Pyrimidinone Derivatives of formula KK, wherein J is a single bond, G is —CH₂— and R⁴ is joined via a —C(O)— group.

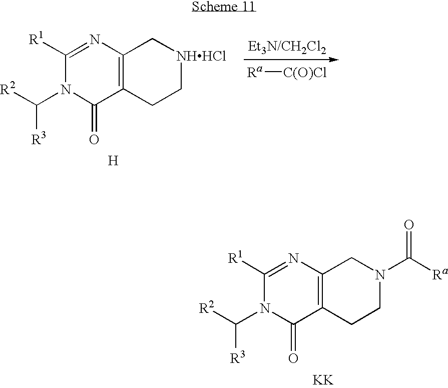

wherein J is a single bond; G is —CH₂—; R¹, R² and R³ are defined above for the compounds of formula (I); and —C(O)R^{a} is representative of all R⁴ substituents, as defined for the compounds of formula (I), that are connected via a —C(O)— group.

The amine hydrochloride compounds of formula H can be reacted with an acid chloride of formula R^{a}—C(O)Cl or an appropriate mixed anhydride, in the presence of a non-nucleophilic base, such as Et₃N, to provide the compounds of formula KK, which correspond to the compounds of formula (I) wherein R⁴ is a substituent that is connected via a —C(O)— group.

Scheme 12 shows a method for converting intermediate compounds of formula H to the Pyrimidinone Derivatives of formula LL, wherein J is a single bond, G is —CH₂— and R⁴ is joined via a —C(O)O— group.

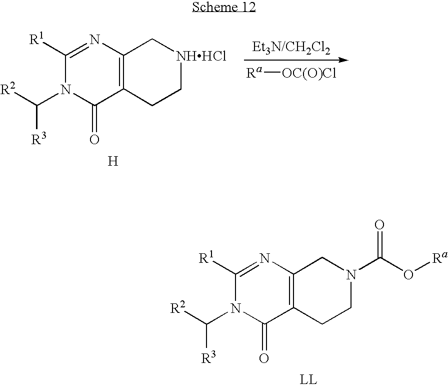

wherein J is a single bond; G is —CH₂—; R¹, R² and R³ are defined above for the compounds of formula (I); and —C(O)O—R^{a} is representative of all R⁴ substituents, as defined for the compounds of formula (I), that are connected via a —C(O)O— group.

The amine hydrochloride compounds of formula H can be reacted with a chloroformate of formula R^{a}—OC(O)Cl in the presence of a non-nucleophilic base, such as Et₃N, to provide the compounds of formula LL, which correspond to the compounds of formula (I) wherein R⁴ is a substituent that is connected via a —C(O)O— group.

As a variant of this method, the compound of formula H may first be reacted with phosgene and then with a compound of formula R^{a}—OH. Alternatively, R^{a}—OH may be reacted first with phosgene and the product of this reaction then reacted with the compound of formula H. Disuccinimidyl carbonate may also be used in place of phosgene.

The starting materials and reagents depicted in Schemes 1-12 are either available from commercial suppliers such as Sigma-Aldrich (St. Louis, Mo.) and Acros Organics Co. (Fair Lawn, N.J.), or can be prepared using methods well-known to those of skill in the art of organic synthesis.

One skilled in the art will recognize that the synthesis of compounds of Formula (I) may require the need for the protection of certain functional groups (i.e., derivatization for the purpose of chemical compatibility with a particular reaction condition). Suitable protecting groups for the various functional groups of the compounds of formula (I) and methods for their installation and removal may be found in Greene et al., Protective Groups in Organic Synthesis, Wiley-Interscience, New York, (1999).

### EXAMPLES

The following examples exemplify illustrative examples of compounds of the present invention and are not to be construed as limiting the scope of the disclosure. Alternative mechanistic pathways and analogous structures within the scope of the invention may be apparent to those skilled in the art.

### General Methods

Solvents, reagents, and intermediates that are commercially available were used as received. Reagents and intermediates that are not commercially available were prepared in the manner described below. ¹H NMR spectra were obtained on a Gemini AS-400 (400 MHz) and are reported as ppm down field from Me₄Si with number of protons, multiplicities, and coupling constants in Hertz indicated parenthetically. Where LC/MS data are presented, analyses was performed using an Applied Biosystems API-100 mass spectrometer and Shimadzu SCL-10A LC column: Altech platinum C18, 3 micron, 33 mm×7 mm ID; gradient flow: 0 min—10% CH₃CN, 5 min—95% CH₃CN, 7 min—95% CH₃CN, 7.5 min—10% CH₃CN, 9 min—stop. The retention time and observed parent ion are given.

### Example 1

### Preparation of Compound 1

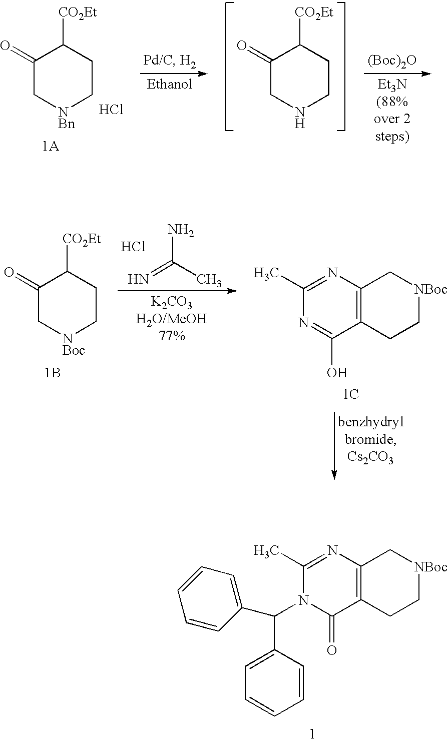

### Step A—Synthesis of Intermediate Compound 1B

A solution of starting material 1A (5.0 g, 16.8 mmol) in ethanol (50 mL) and Pd/C (0.5 g, 10% w/w) was hydrogenated at 1 atm for 15 hours at room temperature. (BOC)₂O (4.0 g, 18.3 mmol) and triethylamine (2.6 mL, 18.6 mmol) were then added to the reaction mixture. The resulting solution was allowed to stir at room temperature for about 3 hours, then filtered through celite. The filtrate was concentrated in vacuo and the resulting residue was redissolved in CH₂Cl₂ and washed with water. The organic phase was collected, dried over Na₂SO₄ and concentrated in vacuo to provide 1B as brown oil (4.0 g, 88%).

### Step B—Synthesis of Intermediate Compound 1C

A mixture of compound 1B (4.0 g, 14.7 mmol) and K₂CO₃ (2.96 g, 21.4 mmol) was diluted with a solution of acetamidine hydrochloride (1.67 g, 17.7 mmol) in water (32 mL) and methanol (8 mL). The resulting reaction was allowed to stir for about 15 hours at 60° C., then cooled to room temperature. The reaction mixture was neutralized using 1N HCl and the organic phase was extracted with CH₂Cl₂ (20 mL). The organic extract was dried over Na₂SO₄ and concentrated in vacuo to provide a crude product which solidified upon trituration hexanes to provide compound 1C as a pale yellow solid (3.0 g, 77%).

### Step C—Synthesis of Intermediate Compound 1

Compound 1C (3.0 g, 11.3 mmol) and benzhydryl bromide (4.7 g, 19.02 mmol) and Cs₂CO₃ (7.6 g, 23.3 mmol) were diluted with THF (180 mL) and the resulting reaction was heated to reflux and allowed to stir at this temperature for about 15 hours. The reaction mixture was cooled to RT, diluted with CH₂Cl₂ (100 mL), and the resulting solution was filtered through Celite. The filtrate was concentrated in vacuo and the resulting residue was purified using flash column chromatography on silica gel (20% acetone-hexanes) to provide compound 1 (3.5 g, 72%).

### Example 2

### Preparation of Compound 6

To a solution of compound 1A (4.00 g, 13.4 mmol) in ethanol (10 mL) was added a suspension of acetamidine hydrochloride (1.43 g, 15.1 mmol) in ethanol (5 mL), followed by a freshly made solution of NaOEt (0.93 g of sodium in 10 mL of ethanol). The reaction was allowed to stir at 100° C. for about 15 hours, then cooled to room temperature and concentrated in vacuo to provide a crude residue which was dissolved in water. The aqueous solution was adjusted to pH 11 using 1N HCl and a solid precipitated out of solution. The solution was then filtered to provide a crude residue which was then reacted with benzyhydryl bromide using the method described in Example 1, Step C to provide compound 6.

### Example 3

### Preparation of Compound 2 (HCl Salt)

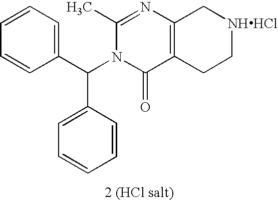

To a solution of compound 1 (3.5 g, 8.1 mmol) in ethyl acetate (14.0 mL) was added 4N HCl in dioxane (7.0 mL). The reaction was allowed to stir at room temperature overnight and the product precipitated out as a white solid. The resulting suspension was then filtered to provide compound 2 as its HCl salt (2.7 g, 91%).

### Example 4

### Preparation of Compound 3

The HCl salt of Compound 2 (0.150 g, 0.41 mmol), p-trifluoromethyl benzaldehyde (0.073 mL, 0.533 mmol) and acetic acid (0.120 mL, 2.0 mmol) were dissolved in 1,2-dichloroethane (3.0 mL) and stirred for 30 minutes. Sodium triacetoxyborohydride (0.3 g, 1.4 mmol) was then added and the reaction mixture stirred overnight. The reaction mixture was diluted with CH₂Cl₂, washed with NaHCO₃ and purified using preparative TLC (3-5% methanol/CH₂Cl₂) to provide compound 3 (0.16 g, 80%).

### Example 5

### Preparation of Compound 14

To a solution of the HCl salt of compound 2 (0.020 g, 0.054 mmol) and diisopropylethylamine (0.028 mL, 0.16 mmol) in CH₂Cl₂ was added isopropylsulfonyl chloride (0.009 mL, 0.08 mmol) and stirred for 3 hours. The reaction mixture was quenched with saturated NH₄Cl and extracted with CH₂Cl₂ and concentrated in vacuo. The reaction was purified using preparative TLC with 3% methanol/CH₂Cl₂ to provide compound 14 (0.021 g, 90%).

### Example 6

### Preparation of Compound 13

To a solution of the HCl salt of compound 2 (0.020 g, 0.054 mmol), tert-butyl isocyanate (0.009 mL, 0.078 mmol) in CH₂Cl₂ was added triethylamine (0.017 mL, 0.12 mmol) and stirred for 4 hours. The reaction mixture was quenched with saturated NH₄Cl and extracted with CH₂Cl₂ and concentrated in vacuo. The reaction was purified using preparative TLC (3% methanol/CH₂Cl₂) to provide compound 13 (0.021 g, 90%).

### Example 7

### Preparation of Compound 15

To a solution of the HCl salt of compound 2 (0.020 g, 0.054 mmol) and triethylamine (0.017 mL, 0.12 mmol) in CH₂Cl₂ was added pivaloyl chloride (0.008 mL, 0.065 mmol) and the resulting reaction was allowed to stir for 4 hours. The reaction mixture was quenched with saturated NH₄Cl and extracted with CH₂Cl₂, then concentrated in vacuo. The resulting residue was purified using preparative TLC (3% methanol/CH₂Cl₂) to provide compound 15 (0.021 g, 95%).

### Example 8

### Preparation of Compound 16

To a solution of the HCl salt of compound 2 (0.020 g, 0.054 mmol) and triethylamine (0.017 mL, 0.12 mmol) in CH₂Cl₂ was added ethyl chloroformate (0.0054 mL, 0.059 mmol) and the resulting reaction was allowed to stir for 4 hours. The reaction mixture was then quenched with saturated NH₄Cl, extracted with CH₂Cl₂, and the organic layer was dried and concentrated in vacuo. The resulting residue was purified using preparative TLC (3% methanol/CH₂Cl₂) to provide compound 16 (0.020 g, 94%).

### Example 9

### Preparation of Compound 157

To a solution of compound 64 (0.020 g, 0.038 mmol) in DMF (1.0 mL) was added NaH (0.008 g, 0.19 mmol, 60%) and the resulting reaction was allowed to stir for 15 min. followed by the addition of MeI (0.005 mL, 0.076 mmol) and allowed to stir for about 15 hours. The reaction mixture was taken up in ethyl acetate (5.0 mL) and washed with saturated NH₄Cl, brine and water and dried over Na₂SO₄ and the organics concentrated in vacuo. The resulting residue was purified using preparative TLC (3% methanol/CH₂Cl₂) to provide compound 157 (0.0165 g, 80%).

### Example 10

### Preparation of Compound 158

To a solution of compound 41 (0.020 g, 0.038 mmol) in DMF/H₂O (0.5 mL/0.012 mL) in a sealable tube was added Pd₂(dba)₃ (0.0018 g, 1.9 μmol, 5 mol %), dppf (0.0026 g, 4.75 μmol, 12.5 mol %), Zn(OAc)₂ (0.0018 g, 0.011 mmol), Zn dust (0.008 g, 0.011 mmol) and Zn(CN)₂ (0.0032 g, 0.027 mmol). The reaction mixture was bubbled with argon and heated in a sealable tube at 100° C. for 4 hours. The reaction mixture was then cooled to room temperature, and diluted with CH₂Cl₂. The organic phase was washed with water, dried and concentrated in vacuo. The resulting residue was purified using flash column chromatography on silica gel (20% acetone/hexanes) to provide compound 158 (0.0144 g, 80%).

### Example 11

### Preparation of Compound 159

To a solution of compound 41 (0.05 g, 0.094 mmol) in DMF (2.0 mL) in a sealable tube was added CuI (0.0054 g, 0.028 mmol), Pd(PPh₃)₄ (0.011 g, 0.0095 mmol), TBAF (0.095 mL of 1.0 M in THF), trimethylsilyl propyne (0.022 mL, 0.14 mmol) and triethylamine (0.044 mL, 0.31 mmol). The reaction mixture was degassed, then heated to 65° C. and allowed to stir at this temperature for about 3 hours. The reaction mixture was then cooled to room temperature and Pd(Cl)₂(PPh₃)₂ (10 mol %) was added and the resulting reaction was allowed to stir for about 15 hours. The reaction mixture was then filtered, concentrated in vacuo, and the resulting residue was purified using flash column chromatography on silica gel (20% acetone/hexanes) to provide compound 159 (0.0276 g, 60%).

### Example 12

### Preparation of Compounds 162 and 167

To a solution of 4-trifluorophenylcyclohexanol (0.225 g, 1.34 mmol) dissolved in CH₃CN (4.0 mL) was added triethylamine (0.56 mL, 4.02 mmol), then disuccinimidyl carbonate (0.307 g, 1.61 mmol). To the resulting mixture was added dropwise a solution of the HCl salt of compound 2 (0.15 g, 0.41 mmol) in CH₂Cl₂ (3.0 mL) and triethylamine (0.1 mL). The resulting reaction was allowed to stir for about 3 hours at room temperature, then was diluted with CH₂Cl₂, washed with water and the organics were concentrated in vacuo. The resulting residue was purified using flash column chromatography (20% acetone/hexanes) to provide the separate isomeric compounds 162 and 167 (0.183 g, combined yield of 85%) with the isomer 167 as the major product.

### Example 13

### Preparation of Compound 168

A solution of 2-chlorobenzoxazole (0.025 g, 0.163 mmol) in dry THF (1.0 mL) was added dropwise to a 0° C., pre-cooled solution of the HCl salt of compound 2 (0.066 g, 0.179 mmol) and triethylamine (0.05 mL) in THF (1.0 mL) under argon. The resulting reaction was allowed to stir at 0° C. for 1 hour, then at room temperature for an additional 2 hours, then filtered. The filtrate was concentrated in vacuo and the resulting residue was purified using column chromatography on silica (20% acetone/hexanes) to provide compound 168 (0.0437 g, 54%).

### Example 14

### Preparation of Compound 177

To a 0° C. solution of 1-(4-methoxyphenyl)-1-cyanocyclopropane (1.0 g, 5.78 mmol) in dichloromethane (10 mL) was added BBr₃ (10 mL, 1.0 M in CH₂Cl₂) dropwise. The resulting reaction was allowed to stir for 2 hours, then water was added and the reaction mixture was extracted with CH₂Cl₂, and the organic phase was dried and concentrated in vacuo. The resulting residue was purified using column chromatography (30% EtOAc/hexanes) to provide a crude intermediate product (0.85 g, 92%), which was then reacted with disuccinimidyl carbonate using the method described in Example 12 to provide compound 177.

### Example 15

### Preparation of Compound 178

To a solution of the HCl salt of compound 2 (0.100 g, 0.272 mmol) in DMF (2.0 mL) in a sealable tube was added triethylamine (0.2 mL, 1.43 mmol), then 2-chloro-6-fluoro-5-trifluoromethyl-1-(2-trimethylsilanyl-ethoxymethyl)-1H-benzimidazole (0.105 g, 0.28 mmol) and the resulting reaction was heated to 100° C. and allowed to stir at this temperature for 2 hours. The reaction mixture was then cooled to room temperature, taken up in ethyl acetate (5.0 mL) and the organic phase was sequentially washed with saturated NH₄Cl, brine and water, then dried over Na₂SO₄ and concentrated in vacuo. The resulting residue was purified using preparative TLC (3% methanol/CH₂Cl₂) to provide compound 178 (0.090 g, 50%).

### Example 16

### Preparation of Compound 179

Compound 178 (0.020 g, 0.03 mmol) was dissolved in 1 mL of 1M TBAF solution and allowed to stir for about 15 hours. The reaction mixture was then purified using preparative TLC (20% acetone/hexanes) to provide compound 179 (0.013 g, 80%).

### Example 17

### Preparation of Compound 195

To a solution of the compound 192 (0.050 g, 0.1 mmol, prepared from by reacting compound 2 with 4-hydroxycyclohexanone according to the method described in Example 12) in CH₂Cl₂ (1.0 mL) was added a solution of oxy-DAST (0.12 g, 0.5 mmol) in CH₂Cl₂ (0.5 mL) and the resulting reaction was allowed to stir for 2 hours at room temperature. Ethanol (0.0012 mL, 0.02 mmol) was then added to the reaction mixture and the resulting reaction was allowed to stir at room temperature until shown to be complete by TLC monitoring. Upon completion, the reaction mixture was poured into saturated NaHCO₃ and after CO₂ evolution ceased, the organics were extracted with CH₂Cl₂, dried over Na₂SO₄, and concentrated in vacuo. The resulting residue was purified using flash column chromatography (20% acetone/hexanes) to provide compound 195 (0.025 g, 50%).

### Example 18

### Preparation of Compounds 193 and 194

To a solution of the compound 192 (0.010 g, 0.02 mmol, prepared from by reacting the HCl salt of compound 2 with 4-hydroxycyclohexanone according to the method described in Example 12) in methanol (1.0 mL) was added sodium borohydride (0.006 g, 0.16 mmol) and the resulting reaction was put under argon atmosphere and allowed to stir at room temperature for 1 hour. The reaction mixture was then diluted with water and extracted with dichloromethane. The combined organics were dried (MgSO₄), filtered and concentrated in vacuo to provide a crude residue which was purified using preparative TLC using 5% methanol/CH₂Cl₂ to provide isomeric compounds 193 and 194 (0.009 g, combined yield 95%).

### Example 18a

### Preparation of Intermediate Compound 18a2

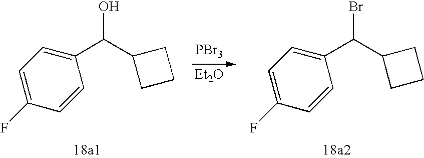

1-Cyclobutyl-1-p-fluorophenyl methanol (18a1, 1.0 g, 5.55 mmol) was dissolved in ether and PBr₃ (0.7 mL, 7.44 mmol) was added dropwise to the solution maintained at 0° C. and the reaction stirred for 1 hour. The reaction mixture was then poured over ice and extracted with ether. The organics were separated and washed with saturated NaHCO₃, brine and dried over K₂CO₃ to provide compound 18a2, which was used without further purification.

One skilled in the art of organic synthesis will recognize how to use this method to prepare all non-commercial bromides used in the N-alkylation procedure described in Example 1.

### Example 19

### Preparation of Compound 199

To a stirred solution of the HCl salt of compound 2 (0.1 g, 0.27 mmol) in CH₂Cl₂ (1.0 mL) was added a solution of NaHCO₃ (0.07 g, 0.82 mmol) in water (0.5 mL). The resulting reaction mixture was vigorously stirred with to 0° C. A solution of cyanogen bromide (0.035 g, 0.324 mmol) in 1.0 mL CH₂Cl₂ was added dropwise to the above reaction and the resulting reaction was allowed to stir for about 15 hours, then Na₂CO₃ was added until a neutral pH was achieved. The resulting suspension was filtered, the collected solid rinsed with CH₂Cl₂, then purified using flash column chromatography (40% acetone/hexanes) to provide compound 199 (0.085 g, 86%).

### Example 20

### Preparation of Compound 200

Step A—Synthesis of N-hydroxy-2-methyl-propionimidoyl Chloride

To a solution of isobutyraldehyde (2.0 mL, 0.022 mol) in methanol (67.0 mL) was slowly added a mixture of NaHCO₃ (3.7 g, 0.044 mol) and hydroxylamine hydrochloride (3.1 g, 0.05 mol) under argon. The mixture was heated to reflux and allowed to stir at this temperature for 45 minutes, then cooled to room temperature and stirred at this temperature for an additional 1 hour. The reaction mixture was diluted with ether and washed with water. Concentration of ether extracts in vacuo provided a crude residue which was diluted with 4N HCl in dioxane (6.5 mL). To the resulting solution was added DMF (30 mL), then oxone (8.0 g) and the reaction mixture was allowed to stir at room temperature for about 15 hours (a slight exotherm was observed). The reaction mixture was then poured into cold water and extracted with ether. The organic layer was washed with 1N HCl, brine, dried over Na₂SO₄ and concentrated in vacuo to provide 1.8 g of N-hydroxy-2-methyl-propionimidoyl chloride which was used directly in the next step.

### Step B—Synthesis of Compound 200

To a solution of compound 199 (0.04 g, 0.11 mmol) and N-hydroxy-2-methyl-propionimidoyl chloride (0.04 g, 0.33 mmol) in ether (4.0 mL) was slowly added triethylamine and the reaction mixture was allowed to stir at room temperature for about 15 hours. The organics were then extracted with CH₂Cl₂, washed with water, dried over Na₂SO₄ and concentrated in vacuo. The resulting residue was purified using flash column chromatography (25% acetone/hexanes) to provide compound 200 (4.8 g, 10%).

### Example 21

### Preparation of Compound 235

Compound 235 was prepared using the method described in Example 20, Step B, using the same p-bromophenyl chloro-oxime but under microwave conditions (100° C., 10 minutes) in 1,2-dimethoxyethane as solvent.

### Example 22

### Preparation of Compound 210

To a solution of compound 199 (0.04 g, 0.11 mmol) in ethanol (4.0 mL) was added hydroxylamine hydrochloride (0.021 g, 0.3 mmol), then K₂CO₃ (0.028 g, 0.2 mmol), and the reaction was stirred at reflux for about 16 hours. The reaction mixture was cooled to room temperature, concentrated in vacuo, and the residue obtained was treated at 0° C. with trimethylacetic anhydride (3.0 mL), then stirred at reflux for 3 hours and cooled to room temperature. The residue was then partitioned between CH₂Cl₂ and saturated aqueous K₂CO₃, and the organics were dried, and concentrated in vacuo. The resulting residue was purified using flash column chromatography (20% acetone/hexanes) to provide compound 210 (0.005 g, 10%).

### Example 23

### Preparation of Compounds 55, 229 and 230

Step A—Synthesis of Phenyl-Pyridin-2-yl-Methanol

To a solution of pyridine-2-carboxaldehyde (1.07 g, 10 mmol) in 50 mL dry THF was added phenylmagnesium bromide (3M in ether, 5 mL, 15 mmol) at 0° C. The reaction was allowed to warm up to room temperature and was allowed to stir for 3 hours. The reaction mixture was diluted with ethyl acetate and quenched with saturated ammonium chloride solution. The organic layer was separated and washed with water and brine. Purification by column chromatography (50% ethyl acetate in hexane) provided phenyl-pyridin-2-yl-methanol in approximately 75% yield.

### Step B—Synthesis of 2-Phenyl-2-Pyridyl Bromomethane

To an ice-cold solution of phenyl-pyridin-2-yl-methanol (500 mg, 2.7 mmol) in 15 mL dry dichloromethane was added thionyl bromide (0.27 mL, 3.5 mmol). The ice-bath was removed and the solution was allowed to stir at room temperature for 4 h after which the solvent was removed under reduced pressure. The resulting oil was taken up in dichloromethane and washed 3 times with saturated sodium bicarbonate solution. The organic phase were washed with brine, dried (magnesium sulfate), filtered and concentrated in vacuo to provide 2-phenyl-2-pyridyl bromomethane in quantitative yield.

### Step C—Synthesis of Compound 55

Compound 55 was synthesized by reacting compound 1C with 2-phenyl-2-pyridyl bromomethane (prepared in Step B), using the procedure described in Example 1.

Step D—Separation of Compound 55 into Compounds 229 and 230

Compound 55 was separated to provide the individual enantiomeric compounds 229 (retention time ˜37 min.) and 230 (retention time ˜44 min.) by using a Chiralpak AD column (10% isopropyl alcohol in hexane at flow rate=75 mL/min.).

### Example 24

### Preparation of Compounds 56 and 57

Compounds 56 and 57 were synthesized by deprotecting compound 55 using the method described in Example 3, then treating the resulting free amine with the corresponding chloroformate using the method described in Example 8.

### Example 25

### Preparation of Compound 58

The BOC group was removed from compound 55 using the method described in Example 3. To a solution of the resulting amine (20 mg, 0.05 mmol) in 2 mL methanol was added 4-trifluoromethyl-benzaldehyde (2 equiv.), sodium cyanoborohydride (2 equiv.) and 3 drops acetic acid. The resulting reaction was allowed to stir at room temperature while being monitored by TLC. After all starting material was consumed, the reaction mixture was quenched with 1N aqueous NaOH solution. The organic layer was separated and the aqueous layer was back extracted twice with dichloromethane. The combined organics were dried and concentrated in vacuo to provide a residue which was purified using flash column chromatography (5% methanol in dichloromethane) to provide compound 58.

### Example 26

### Preparation of Compounds 174 and 175

Step A—Synthesis of Di-(Pyridin-2-yl)-Methanol

To a −78° C. solution of 2-bromopyridine (3.0 g, 19.0 mmol) in 60 mL THF was added n-BuLi (2.5 M in hexane, 7.6 mL, 19.0 mmol). The resulting reaction was allowed to stir at −78° C. for about 15 minutes, then 2-pyridine carboxaldehyde (2.17 mL, 22.8 mmol) was added dropwise at −78° C. The resulting reaction mixture was allowed to stir for 30 minutes at −78° C., then for 2 hours at room temperature after which time the reaction was quenched with saturated aqueous NH₄Cl solution. After diluting the reaction mixture with ethyl acetate, the organic layer was separated and the aqueous layer was back extracted twice with ethyl acetate. The combined organic fractions were washed with brine, dried (magnesium sulfate), filtered, and concentrated in vacuo to provide di-pyridin-2-yl-methanol in 70% yield as a yellow oil.

Step B—Synthesis of Di-(Pyridin-2-yl)-Bromomethane

To a 0° C. solution of di-(pyridin-2-yl)-methanol (0.64 g, 3.44 mmol, prepared in Step A) in 10 mL dichloromethane, was added triethylamine (1.92 mL, 13.76 mmol) followed by methanesulfonyl chloride (0.32 mL, 4.13 mmol). The resulting reaction was allowed to stir at 0° C. for 15 minutes, and was then diluted with ethyl acetate and washed with water. The organic layer was dried (magnesium sulfate), filtered and concentrated in vacuo to provide an intermediate mesylate compound. The intermediate mesylate compound was diluted with 7 mL DMF and to the resulting solution was added LiBr (2.5 g, 28.7 mmol) and the mixture was allowed to stir at room temperature for about 16 hours. The reaction was then quenched with water, and diluted with ethyl acetate. The organic layer was separated and the aqueous layer was back extracted twice with ethyl acetate. The combined organic fractions were washed with brine, dried (magnesium sulfate), filtered and concentrated in vacuo to provide 500 mg of di-pyridin-bromomethane, which was used for the next step without purification.

### Step C—Synthesis of Compound 174

Compound 1C was reacted with di-(pyridin-2-yl)-bromomethane using the procedure described in Example 1 to provide a BOC-protected intermediate, which was then deprotected using the method described in Example 3. The resulting free amine was then reacted with the appropriate chloroformate using the method described in Example 8 to provide Compound 174.

Step D—Synthesis of Compound 175 Compound 175 was synthesized as described in Examples 23 and 25, substituting di-(pyridin-2-yl)bromomethane for 2-(bromomethyl-phenyl)-pyridine in Step C of Example 23.

### Example 27

### Preparation of Compound 183

Compound 183 was synthesized using the method described in Example 1 and substituting propionamidine hydrochloride for acetamidine hydrochloride.

### Example 28

### Preparation of Compound 5

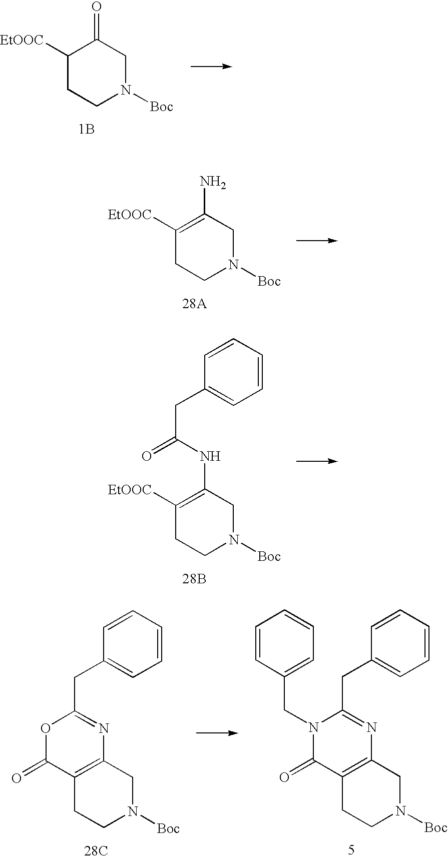

### Step A—Synthesis of Compound 28A

To a solution of compound 1B (14.0 g, 52 mmol) in EtOH (60 mL) was added NH₄OAc (10.0 g, 130 mmol). The resulting reaction was heated to 50° C. and allowed to stir at this temperature for 1 hour, then cooled to room temperature. The reaction mixture was then concentrated in vacuo and partitioned with DCM and water. The organic phase was collected, washed with brine, dried (MgSO₄), and concentrated in vacuo to provide compound 28A as a white solid.

### Step B—Synthesis of Compound 28B

Compound 28A (0.224 g, 0.83 mmol), phenylacetyl chloride (0.13 ml, 0.99 mmol), and pyridine (0.13 ml, 1.7 mmol) were taken up in THF (3 mL). The reaction was heated to 50° C. and allowed to stir at this temperature for 18 hours, then cooled to room temperature. The reaction mixture was then concentrated in vacuo, and purified using preparative layer chromatography to provide compound 28B as an oil.

### Step C—Synthesis of Compound 28C

To a solution of compound 28B (0.103 g, 0.27 mmol) in DCM (1.0 mL) was added Me₃Al (2.0M in toluene, 0.40 ml=0.8 mmol). The resulting reaction was heated to 40° C. and allowed to stir at this temperature for 18 hours, then cooled to room temperature. The reaction mixture was concentrated in vacuo and partitioned with ether and aqueous 1N HCl. The organic phase was collected, dried (MgSO₄), concentrated in vacuo and the residue obtained was purified using preparative layer chromatography to provide compound 28C as a yellow solid.

### Step D—Synthesis of Compound 5

To a solution of compound 28C (0.040 g, 0.12 mmol) in toluene (2.0 mL) was added benzylamine (0.025 g, 0.23 mmol). The resulting reaction was heated to 90° C. and allowed to stir at this temperature for 70 hours, then cooled to room temperature and concentrated in vacuo. The residue obtained was purified using preparative layer chromatography to provide compound 5 as a yellow solid.

### Example 29

### Preparation of Compound 9

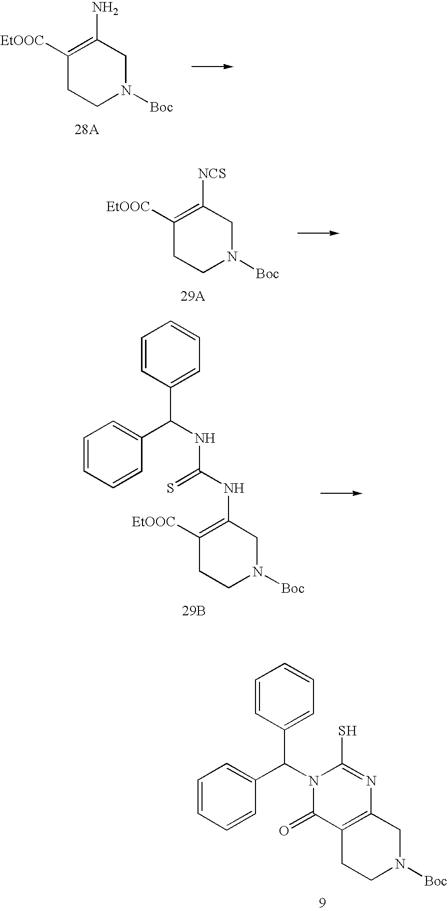

### Step A—Synthesis of Compound 29A

To a 0° C. solution of Compound 28A (4.0 g, 15 mmol) and NMM (4.1 ml, 37 mmol) in DCM (50 mL) was added thiophosgene (1.40 ml, 18 mmol). The resulting reaction was allowed to stir for 1 hour at 0° C. and was then concentrated in vacuo and the resulting residue purified using flash column chromatography on silica (MeOH/CH₂Cl₂) to provide compound 29A as a yellow oil.

### Step B—Synthesis of Compound 29B

A solution of compound 29A (0.85 g, 2.7 mmol), triethylamine (0.38 mL, 2.77 mmol) and benzhydrylamine (0.61 mL, 3.5 mmol) in acetonitrile (20 mL) was heated to 80° C. and allowed to stir at this temperature for 18 hours. The reaction was cooled to room temperature, concentrated in vacuo, and the resulting residue was washed with hexanes to provide compound 29B as a yellow solid.

### Step C—Synthesis of Compound 9

To a solution of compound 29B (entire yield from Step B) in acetonitrile (20 mL) was added NaO-tBu (0.46 g, 4.8 mmol). The resulting reaction was heated to 60° C. and allowed to stir at this temperature for 1 hour, then the reaction mixture was cooled to room temperature and partitioned with EtOAc and 1N HCl. The organic phase was collected, dried (MgSO₄), concentrated in vacuo and the resulting residue was purified using flash column chromatograph on silica (MeOH/CH₂Cl₂) to provide compound 9 as a yellow solid.

### Example 30

### Preparation of Compound 218

Compound 218 was synthesized using the method described in Example 1. The required bromo intermediate was synthesized by reacting the appropriate commercially available alcohol with thionyl bromide according to the method described in Example 23.

### Example 31

### Preparation of Compound 189

Compound 189 was synthesized from compound 5 using the method described in Example 24.

### Example 32

### Preparation of Compound 196

Compound 196 was synthesized from compound 183 using the method described in Example 24.

### Example 33

### Preparation of Compound 197

Compound 197 was synthesized from compound 183 using the method described in Example 25 and substituting 2-fluoro-4-trifluoromethyl benzaldehyde for 4-trifluoromethyl benzaldehyde.

### Example 34

### Preparation of Compound 198

Compound 198 was synthesized using the method described in Example 1, Step C. The required bromide was prepared using the method described in Example 26, Step B, and the alcohol precursor was synthesized using the method described in Example 23, using cyclopentylmagnesium bromide and pyridine-2-carboxaldehyde.

### Example 35

### Preparation of Compound 256

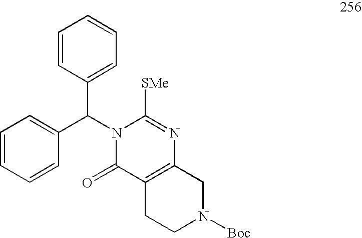

To a solution of compound 9 (0.22 g, 0.49 mmol) and K₂CO₃ (0.068 g, 0.49 mmol) in THF (2.0 mL) was added CH₃I (0.031 mL, 0.50 mmol). The resulting reaction was allowed to stir for 3 hours and was then filtered and the filtrate was concentrated in vacuo. The residue obtained was purified using preparative layer chromatography to provide compound 256 as a white solid.

### Example 36

### Preparation of Compound 257

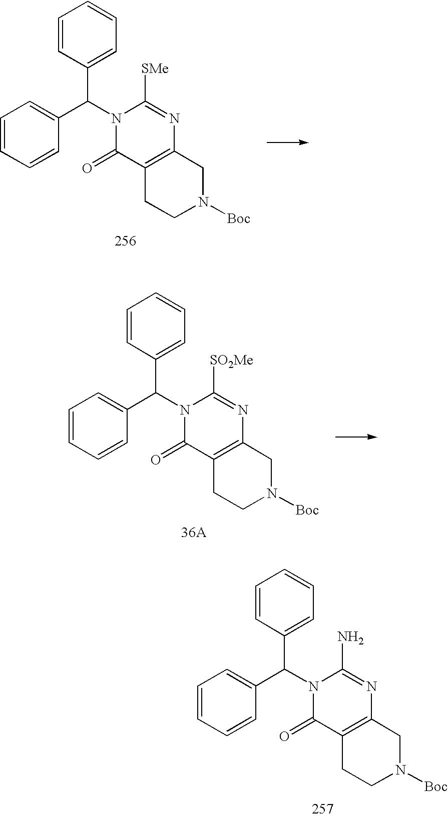

### Step A—Synthesis of Compound 36A

A solution of compound 256, (0.74 g, 1.6 mmol) in DCM (20 mL) was cooled to 0° C. and mCPBA (70%, 0.47 g, 1.9 mmol) was added. The resulting reaction was allowed to stir for 1 hour at 0° C. and K₂CO₃ (1.0 g) was added. The reaction was allowed to stir at room temperature for 30 minutes, then was filtered and concentrated in vacuo to provide compound 36A as a white solid.

### Step B—Synthesis of Compound 257

A solution of compound 256 (entire yield from Step A) and 2.0M NH₃ in isopropanol (4.0 mL) was placed in a sealed tube and the tube was placed in an 80° C. oil bath. The reaction was allowed to stir in the bath for 70 hours and was then cooled to room temperature and the reaction mixture was concentrated in vacuo. The crude residue obtained was purified using preparative layer chromatography to provide compound 257 as a white solid.

### Example 37

### Preparation of Compound 208

To a solution of compound 1 (0.1 g, 0.23 mmol) in 3 mL dichloromethane was added m-chloroperoxybenzoic acid (0.1 g, 0.46 mmol) and the resulting reaction was allowed to stir for 24 hours at room temperature. The reaction was quenched with saturated sodium bicarbonate solution and the organic layer was separated, dried (sodium sulfate), filtered, and concentrated in vacuo. The resulting residue was purified using preparative TLC (3% methanol in dichloromethane) to provide compound 208 (40% yield).

### Example 38

### Preparation of Compound 258

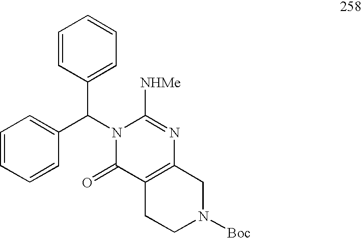

Using the method described in Example 36, and substituting MeNH₂ in THF for NH₃ in isopropanol, compound 258 was prepared.

### Example 39

### Preparation of Compound 211

Compound 211 was synthesized using the method described below in Example 51. The required bromide was prepared by bromination of the corresponding commercially available alcohol using the method described in Example 23, Step B.

### Example 40

### Preparation of Compound 212

Compound 212 was synthesized using the method described above in Example 1. The required bromide was prepared by bromination of the corresponding commercially available alcohol using the method described in Example 23, Step B.

### Example 41

### Preparation of Compound 215

### Step A—Synthesis of 2-Benzylpyrazine

To a solution of 2-methylsulfanyl-pyrazine (1.26 g, 10 mmol) in 15 mL THF was added benzylzinc bromide (0.5M in THF, 40 mL, 20 mmol) followed by Pd(Ph₃P)₄ (1.16 g, 1 mmol). The resulting reaction was heated to 60° C. and allowed to stir at this temperature for 2 hours, after which time the reaction mixture was cooled to room temperature, diluted with ethyl acetate and washed with saturated sodium bicarbonate solution. The organic fraction was dried (sodium sulfate), filtered, and concentrated in vacuo to provide a crude residue which was purified using flash column chromatography (15% ethyl acetate in hexane) to provide 2-benzylpyrazine (0.76 g, 45% yield).

Step B—Synthesis of 1-phenyl-1-(2-pyrazinyl)-bromomethane

To a solution of 2-benzylpyrazine (100 mg, 0.59 mmol) in 5 mL acetonitrile was added 1,3-dibromo-5,5-dimethylhydantoin (183 mg, 0.64 mmol) and the reaction was heated at 65° C. for 3 days. The solvent was evaporated and the crude product was purified using preparative TLC (20% acetone in hexane) to provide 2-(1-bromophenylmethyl)-pyrazine (50 mg, 35% yield) which was used immediately for the next step.

### Step C—Synthesis of Compound 215

Compound 215 was synthesized using the method described in Example 51, using 1-phenyl-1-(2-pyrazinyl)-bromomethane as the bromo intermediate.

### Example 42

### Preparation of Compound 216

Compound 216 was synthesized using the method described in Example 51. The required bromo intermediate was synthesized using the method described in Example 23 using pyrimidine-5-carboxaldehyde and phenylmagnesium bromide.

### Example 43

### Preparation of Compound 259

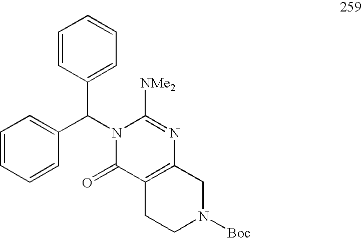

Using the method described in Example 36, and substituting Me₂NH in THF for NH₃ in isopropanol, compound 259 was prepared.

### Example 44

### Preparation of Compounds 219, 223 and 232

Compounds 219, 223 and 232 were synthesized using the method described in Example 1. The required bromo intermediates for making each of these compounds were synthesized by reacting the appropriate commercially available alcohols with thionyl bromide according to the method described in Example 23.

### Example 45

### Preparation of Compound 260

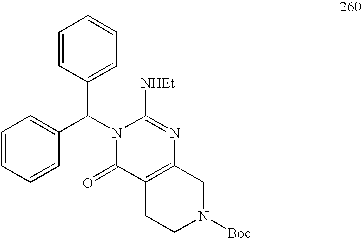

Using the method described in Example 36, and substituting EtNH₂ in THF for NH₃ in isopropanol, compound 260 was prepared.

### Example 46

### Preparation of Compound 224

Step A—Synthesis of Phenyl-Thien-2-yl-Methanol

To a solution of phenyl-thiophen-2-yl-methanone (1.5 g, 7.98 mmol) in 17 mL THF was added sodium borohydride (0.38 g, 10 mmol) followed by 0.5 mL H₂O. The resulting reaction was heated to reflux and allowed to stir at this temperature for 3 hours, then cooled to room temperature, diluted with ethyl acetate and washed with water. The organic fraction was dried (sodium sulfate), filtered, and concentrated in vacuo to provide phenyl-thiophen-2-yl-methanol in quantitative yield, which was used for the next step without further purification.

### Step B—Synthesis of Compound 224

To a 0° C. solution of triphenylphosphine (150 mg, 0.57 mmol) in 3 mL THF was added DIAD (0.1 mL, 0.53 mmol) and the solution was allowed to stir for 30 minutes at 0° C., then was cooled to −78° C. To the resulting cooled yellow suspension was added dropwise a solution of the compound 1C (50 mg, 0.19 mmol) and phenyl-thiophen-2-yl-methanol (36 mg, 0.19 mmol) in 2 mL THF. The reaction was allowed to stir for 4 hours, then was quenched with water and extracted with ethyl acetate. The combined organic fractions were dried (sodium sulfate), filtered, and concentrated in vacuo to provide a crude residue which was purified using preparative TLC (20% acetone in hexane) to provide compound 224 in 35% yield.

### Example 47

### Preparation of Compounds 225, 226 and 231

Compounds 225, 226 and 231 were synthesized using the method described in Example 51. The required bromo intermediates for making each of these compounds were synthesized by reacting the appropriate commercially available alcohols with thionyl bromide according to the method described in Example 23.

### Example 48

### Preparation of Compound 247

A first solution of BF₃.Et₂O (3.23 g, 22.8 mmol) in diethyl ether (6.0 mL) and a second solution of ethyldiazoacetate (3.0 g, 26.3 mmol) in diethyl ether (6.0 mL) were simultaneously and separately added over a 20 minute period to a solution of N-carbethoxy-4-piperidone (3.0 g, 17.3 mmol) in diethyl ether (20.0 mL). The reaction temperature during the addition was maintained at −25 to −30° C. using a dry ice-isopropanol bath. After the addition was complete, the resulting reaction was allowed to stir at −25° C. for 1 hour, then allowed to warm to room temperature. The reaction mixture was washed with 30% K₂CO₃ (100 mL) and extracted with EtOAc (3×100 mL). The combined organics were dried over Na₂SO₄ and concentrated in vacuo to provide a crude orange oil, which was purified using flash column chromatography on silica gel (30% EtOAc/hexanes) to provide an intermediate product (3.7 g, 82% yield). The intermediate product obtained was then treated with the same conditions employed in the preparation of 6 to provide compound 247.

### Example 49

### Preparation of Compound 248

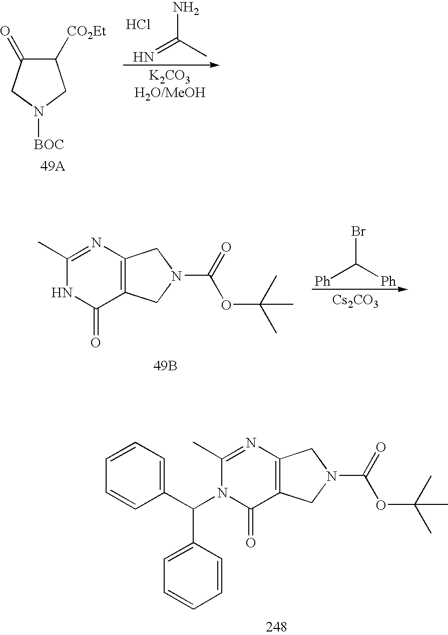

Using the method described in Example 1, Steps B and C, and substituting 1-t-butyl-3-ethyl-4-oxopyrrolidine-1,3-dicarboxylate (49A) for compound 1B, compound 248 was prepared.

### Example 50

### Preparation of Compounds 241, 242 and 243

Compounds 241, 242 and 243 were synthesized respectively from compounds 229, 230 and 223, using the method described in Example 24.

### Example 51

### Preparation of Compounds 211, 215, 216, 225, 226 and 231

Compounds 211, 215, 216, 225, 226 and 231 were prepared using the method set forth below.

Step A—Synthesis of 7-benzyl-2-methyl-5,6,7,8-tetrahydro-3H-pyrido[3,4-d]pyrimidin-4-one

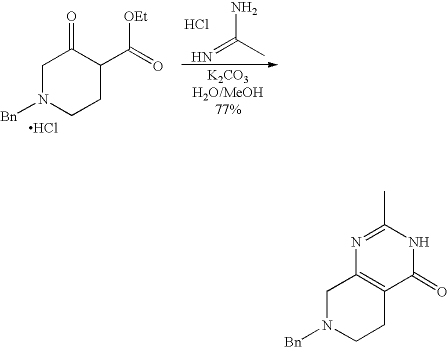

To a solution of 1-benzyl-3-oxo-piperidine-4-carboxylic acid ethyl ester hydrochloride (5.0 g, 16.8 mmol) in 80 mL ethanol was added acetamidine hydrochloride (2.4 g, 25.2 mmol) followed by sodium ethoxide (21% in ethanol, 10.6 mL, 33.6 mmol). The resulting reaction was heated to reflux and allowed to stir at this temperature for 16 hours. The reaction was then cooled to room temperature, diluted with dichloromethane, and the organic phase was washed with water and brine, dried and concentrated in vacuo. the resulting residue was purified using flash column chromatography (5% methanol in dichloromethane) to provide 7-benzyl-2-methyl-5,6,7,8-tetrahydro-3H-pyrido[3,4-d]pyrimidin-4-one in 77% yield.

Step B—Synthesis of (2-methyl-4-oxo-4,5,6,8-tetrahydro-3H-pyrido[3,4-d]pyrimidine-7-carboxylic Acid 4-bromophenyl Ester)

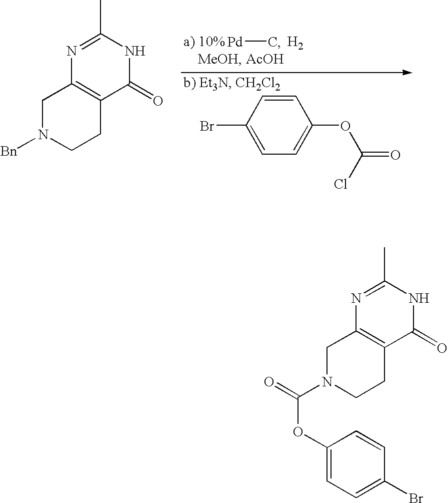

To a solution of 7-benzyl-2-methyl-5,6,7,8-tetrahydro-3H-pyrido[3,4-d]pyrimidin-4-one (2.5 g, 9.8 mmol) in 150 mL methanol was added 0.6 mL acetic acid followed by 10% Pd—C (0.25 g, 10% w/w). The resulting reaction was hydrogenated for 16 hours at 1 atmosphere, then filtered through celite. The filtrate was concentrated in vacuo and the resulting residue (4.9 mmol) was taken up in 50 mL dichloromethane. To this solution was added triethylamine (7.0 mL, 50 mmol) followed by 4-bromophenyl chloroformate (1.0 mL, 7.0 mmol). The resulting reaction was allowed to stir at room temperature for 4 hours, then water was added and the organic layer was separated. The aqueous layer was back extracted twice with dichloromethane and the combined organics were dried (magnesium sulfate), filtered, and concentrated in vacuo. The resulting residue was purified using flash column chromatography to provide (2-methyl-4-oxo-4,5,6,8-tetrahydro-3H-pyrido[3,4-d]pyrimidine-7-carboxylic acid 4-bromophenyl ester) in approximately 62% yield.

### Step C—Preparation of Compounds 211, 215, 216, 225, 226 and 231

(2-methyl-4-oxo-4,5,6,8-tetrahydro-3H-pyrido[3,4-d]pyrimidine-7-carboxylic acid 4-bromo-phenyl ester) was reacted with the appropriate bromo intermediates using the methodology described in Example 1 to provide compounds 211, 215, 216, 225, 226 and 231.

### Example 52

### Preparation of Compounds 331 and 332

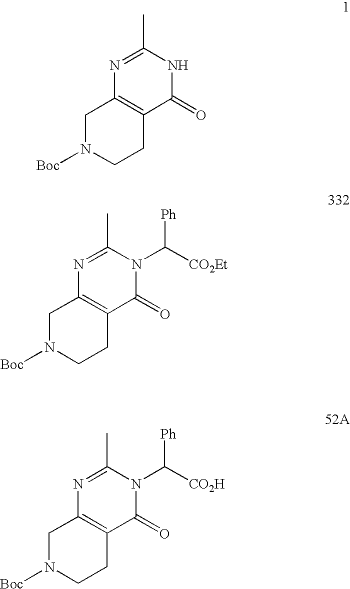

Compound 1 was N-alkylated using ethyl α-bromophenylacetate using the method described in Example 1 to provide compound 332. To a solution of compound 332 (220 mg, 0.52 mmol) in 2 mL ethanol and 2 mL THF was added lithium hydroxide monohydrate (120 mg, 2.85 mmol). After stirring the reaction for 20 hours, 10% aqueous KHSO₄ was added and the reaction was extracted with ethyl acetate. The organic fractions were dried and concentrated to give the crude acid 52A. To a solution of compound 52A in 1 mL DMF was added 12 mg HOBT and 7 mg of cyclobutyl amine followed by 17 mg EDCI. The reaction was stirred for 20 h after which it was quenched with water. Extraction with ethyl acetate followed by concentration and purification (20% acetone in hexanes) resulted in the final compound 331.

### Example 53

### Preparation of Compounds 334, 335 and 336

Compounds 334, 335 and 336 were prepared from compound 332 using the method described in Example 52.

### Example 54

### Preparation of Compounds 337-356 (via Library Synthesis)

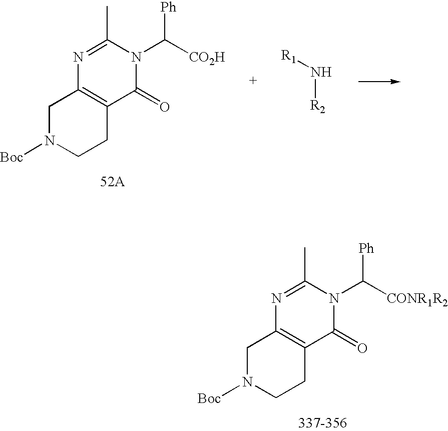

PS-EDC resin (i.e., polystyrene functionalized with EDC-1-(dimethylaminopropyl)-3-ethylcarbodiimide—available from Polymer Laboratories) (0.082 g, 1.42 mmol) was added to 96 wells of a deep well polypropylene microtiter plate followed by a MeCN/THF (3:2) stock solution (1 mL) of the acid 52A (0.021 mmol) and HOBt (i.e., 1-hydroxybenzotriazole hydrate) (0.031 mmol). 1 M stock solutions of each of the individual amines (R₁R₂NH) (0.042 mL, 0.042 mmol) were added to the wells, which were then sealed and shaken at 25° C. for 18 hours. The solutions were filtered through a polypropylene frit into a second microtiter plate containing PS-Isocyanate resin (3 equiv., 0.07 mmol) and PS-Trisamine resin (8 equiv., 0.17 mmol). After the top plate was washed with MeCN (0.5 mL/well), the plate was removed, the bottom microtiter plate was sealed and then shaken at 25° C. for 16 hours. The solutions were filtered through a polypropylene frit into a 96-well collection plate. The wells of the top plate were then washed with MeCN (0.5 mL/well), and the plate removed. The resultant solutions in the collection plate were transferred into vials and the solvent removed in vacuo using a SPEEDVAC. The resulting samples were evaluated by LCMS and those that were >70% pure were submitted for testing.

### Example 55

### Preparation of Compound 357

To a solution of compound 52A (30 mg, 0.075 mmol) in 2 mL DMF was added DIEA (33 μL, 0.19 mmol), acetic hydrazide (14 mg, 0.19 mmol) followed by HATU (72 mg, 0.19 mmol). The reaction was stirred for 4 hours after which it was quenched with saturated ammonium chloride solution. Extraction with ethyl acetate followed by concentration resulted in a dark yellow oil. To a solution of the crude material in 2 mL THF was added PS-BEMP (170 mg, 0.37 mmol) and tosylchloride (18 mg, 0.09 mmol). The reaction was microwaved at 120° C. for 15 minutes after which it was filtered and concentrated. Purification (20% acetone in hexanes) to provide compound 357.

### Example 56

### Preparation of Compound 359

To a solution of compound 332 in 1.5 mL THF and 0.5 mL MeOH was added 4 mg sodium borohydride. The reaction was heated to 65° C. and allowed to stir at this temperature for 16 hours. The reaction mixture was concentrated in vacuo and the residue obtained was purified using flash column chromatography (20% acetone in hexanes) to provide compound 359.

### Example 57

### Preparation of Compound 360

To a solution of compound 52A (20 mg, 0.05 mmol) in 2 mL toluene was added N,N-dimethylformamide di-tert-butyl acetal (0.05 mL, 0.20 mmol). The reaction was heated to 100° C. and allowed to stir at this temperature for 30 min. after which time the reaction mixture was concentrated in vacuo. The resulting residue was purified using flash column chromatography (30% acetone in hexanes) to provide compound 360.

### Example 58

### Preparation of Compound 362

To a solution of compound 361 (20 mg, 0.046 mmol) in 1 mL DMF was added NBS (11 mg, 0.062 mmol). The reaction was stirred at room temperature for 2 hours, then concentrated in vacuo. The resulting residue was purified using flash column chromatography (20% acetone in hexanes) to provide compound 362.

### Example 59

### Preparation of Compound 363

Compound 332 was deprotected using the method described in Example 3 and the deprotected product was converted to compound 363 via coupling with 4-trifluoromethoxyphenol using the method described in Example 12.

### Example 60

### Preparation of Compounds 364 and 365

Compound 364 was synthesized using the method described in Example 1. The corresponding bromide was prepared as described in Tetrahedron 1999, 55, 10155. TBS deprotection of 364 using TBAF/THF provided compound 365.

### Example 61

### Preparation of Compound 367

Compound 367 was prepared from compound 335 using the method described in Example 59.

### Example 62

### Preparation of Compound 368

To a solution of compound 365 in 2 mL dichloromethane was added 2,6-di-tert-butyl pyridine (17 μl, 0.078 mmol), silver triflate (20 mg, 0.078 mmol), and ethyl iodide (6 μL, 0.078 mmol). The reaction was stirred at room temperature for 20 hours after which time the reaction mixture was concentrated in vacuo. The residue obtained was purified using flash column chromatography (20% acetone in hexanes) to provide compound 368.

### Example 63

### Preparation of Compound 370

Compound 370 prepared from compound 368 using the method described in Example 59.

### Example 64

### Preparation of Compound 371

As described in Example 1, the required bromide was prepared from the commercially available alcohol using the method described in Example 23 (step B).

### Example 65

### Preparation of Compounds 374 and 375

Compound 374 was prepared in a method analogous to that described in Example 62. Compound 375 was prepared from compound 374 using the method described in Example 12.

### Example 66

### Preparation of Compound 377

Compound 377 was prepared from compound 371 using the method described in Example 7.

### Example 67

### Preparation of Compound 381

Compound 381 was prepared from compound 371 using the method described in Example 12.

### Example 68

### Preparation of Compound 383

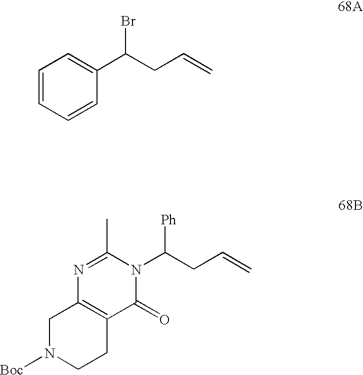

### Step A—Synthesis of Compound 68B

Compound 68A was prepared from commercially available 1-phenyl-3-butene-1-ol using the procedure described in Example 18. N-alkylation of compound 68A using the method described in Example 1 resulted in compound 68B.

### Step B—Synthesis of Compound 383

To a solution 1-methyl-3-nitro-1-nitroso guanidine (52 mg, 0.35 mmol) in 3 mL ether was added dropwise 40% aqueous KOH solution (3 mL) at 0° C. The reaction was stirred for 30 min. after which time the ether layer was added dropwise to an ice-cold solution of 68B (20 mg, 0.05 mmol) and Pd(OAc)₂ (5 mg) in 3 mL ether. The reaction was stirred at room temperature for 20 hours, then concentrated in vacuo to provide a crude residue which was purified using flash column chromatography (20% acetone in hexanes) to provide compound 383.

### Example 69

### Preparation of Compound 389

Compound 389 was prepared from compound 371 using the method described in Example 12.

### Example 70

### Preparation of Compound 390

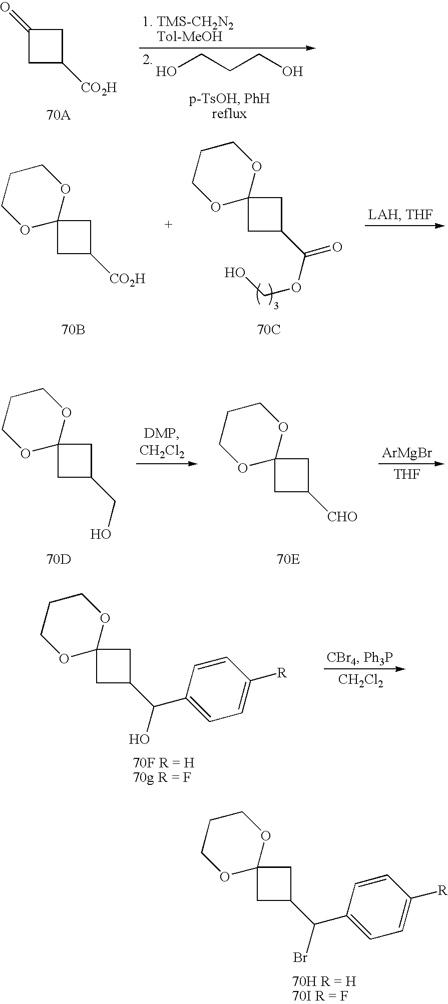

### Step A—Preparation of Compounds 70B and 70C

To a solution of compound 70A (1.0 g, 8.78 mmol) in 65 mL toluene and 16 mL methanol was added TMS-CH₂N₂ (2M in hexanes, 6.6 mL, 13.2 mmol). The reaction was stirred for 1 hour, then concentrated in vacuo and the residue obtained was diluted with 40 mL dry benzene. To the resulting solution was added 1,3-propanediol (1.1 mL, 14.1 mmol) and p-toluenesulfonic acid (0.18 g, 0.94 mmol) and the resulting reaction was heated to reflux and allowed to stir at this temperature for 3 hours, then concentrated in vacuo. The residue obtained was diluted with ethyl acetate, the organic layer was collected, washed with saturated aqueous sodium bicarbonate and water, then dried and concentrated in vacuo to provide a dark yellow oil (70B and 70C) which was used for the next step without purification.

### Step B—Preparation of Compounds 70D

To the crude dark yellow oil (70B and 70C) from above in 80 mL THF was added LAH (1M in TIT, 17.56 mL, 17.56 mmol) and the reaction was stirred for 16 h after which it was quenched carefully with 2.0 mL water. The resulting solution was treated with 2.0 mL 1N NaOH and then with 6.0 mL water. The mixture was stirred at 0° C. for 30 minutes after which it was filtered under vacuum. The residue was washed with hexanes and the resulting filtrate was concentrated. Purification (5% acetone in hexanes) afforded the alcohol 70D (1.1 g).

### Step C—Preparation of Compounds 70E

To a solution of compound 70D (1.1 g, 6.96 mmol) in 80 mL dichloromethane was added sodium bicarbonate (2.34 g, 27.84 mmol) followed by Dess-Martin periodinane (4.45 g, 10.45 mmol). After stirring for 16 hours, the reaction was quenched with satd. NaHCO₃ and satd. Na₂S₂O₃. After stirring for an additional 30 min. the reaction was extracted with dichloromethane. The combined organic fractions were dried and concentrated to give the aldehyde 70E which was used for the next step without purification.

### Step D—Preparation of Compounds 70F

To a solution of crude 70E 3.5 mmol) from above in 40 mL THF was added phenylmagnesium bromide (3M in ether, 2.35 mL, 7.0 mmol). The reaction was stirred for 3 h after which it was quenched with water and extracted with ether. The combined organic fractions were dried, concentrated, and purified (30% acetone in hexanes). Collection of the pure fractions afforded the alcohol 70F (280 mg).

### Step E—Preparation of Compounds 390

To a solution of compound 70F (280 mg, 1.2 mmol) in 20 ml dichloromethane was added triphenyl phosphine (470 mg, 1.8 mmol). The reaction was stirred for 10 min. after which a solution of carbon tetrabromide (600 mg, 1.8 mmol) in 3 mL dichloromethane was added dropwise. The reaction was stirred at room temperature for 1 h after which the solvent was removed. Purification (20% acetone in dichloromethane) resulted in the bromide 70H which was used for N-alkylation described in Example 1 to give 390.

### Example 71

### Preparation of Compound 391

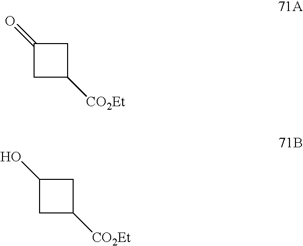

### Step Preparation of Compound 71B

To a solution of the ketone 71A (85 mg, 0.66 mmol) in 2 mL THF and 1.0 mL MeOH at 0° C., was added sodium borohydride (24 mg, 0.66 mmol). The reaction was allowed to stir for 15 minutes, then was quenched with water and extracted with ethyl acetate. The ethyl acetate layer was dried, filtered and concentrated in vacuo to provide compound 71B, which was used in the next step without further purification.

### Step B—Preparation of Compound 391

Compound 71B was converted to compound 391 using the method described in Example 12.

### Example 72

### Preparation of Compound 393

Compound 297 was synthesized from benzaldehyde and p-fluorophenylmagnesium bromide using the method described in Example 23, Steps A-C. N-Boc deprotection of compound 297 (using the method described in Example 3), followed by carbamate formation using the method described in Example 12, provided compound 393.

### Example 73

### Preparation of Compound 396

To a solution of compound 392 (15 mg, 0.03 mmol, prepared from compound 390 using the methods described in Examples 3 and 12) in 3 mL dichloromethane was added DAST (15 mg, 0.09 mmol) and the reaction was stirred for 20 hours. After quenching with water the reaction was extracted with dichloromethane, and the organic phase was dried, filtered and concentrated in vacuo to provide a crude residue which was purified using flash column chromatography (30% acetone in hexanes) to provide compound 396.

### Example 74

### Preparation of Compound 397

To a solution of compound 394 (110 mg, 0.22 mmol, prepared using the method described in Example 69) in 5 mL THF was added lithium borohydride (0.33 mL, 2M in THF, 0.66 mmol). The reaction was stirred for 20 h after which time it was quenched with water and then extracted with ethyl acetate. The organic phase was dried, filtered and concentrated in vacuo to provide a crude residue which was purified using flash column chromatography (5% methanol in dichloromethane) to provide compound 397.

### Example 75

### Preparation of Compounds 399, 400, 401 and 403

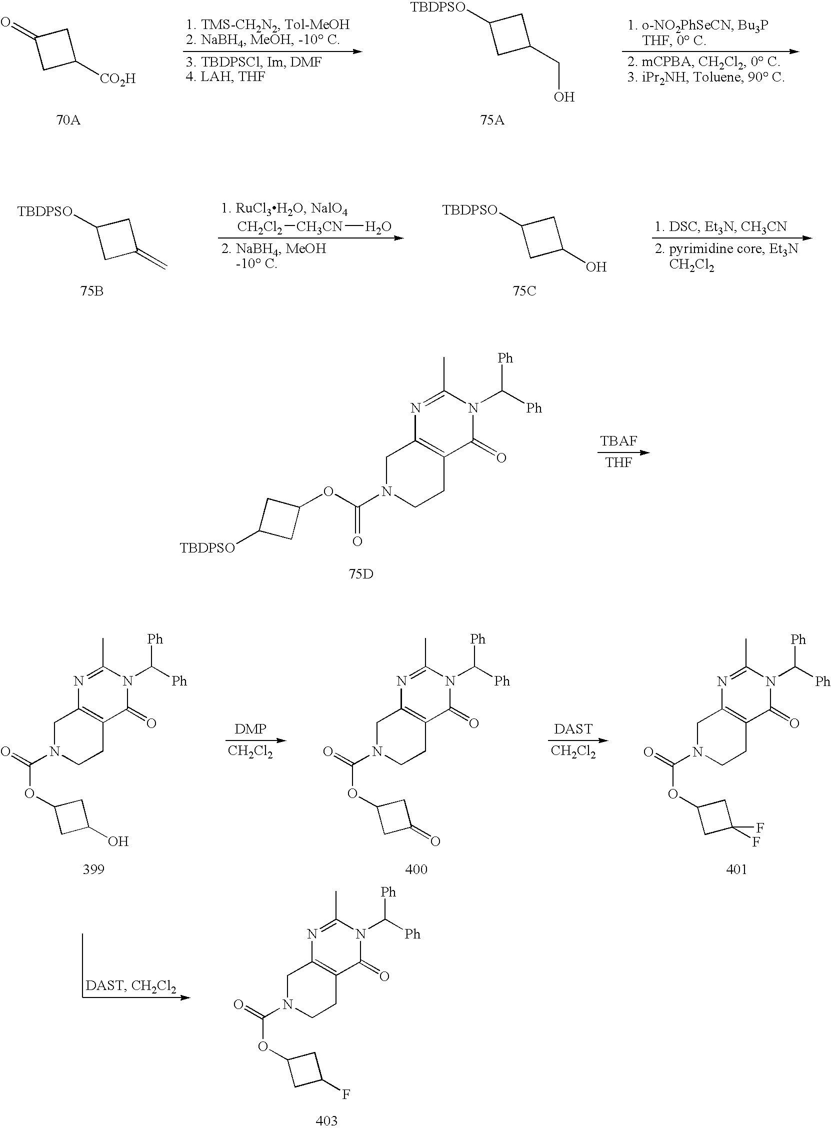

### Step A—Synthesis of Compound 75A

To a solution of compound 70A (1.0 g, 8.78 mmol) in 65 mL toluene and 16 mL methanol was added TMS-CH₂N₂ (2M in hexanes, 6.6 mL, 13.2 mmol). The reaction was stirred for 1 hour, then concentrated in vacuo and the residue obtained was diluted in 60 mL methanol and the resulting solution was cooled to −10° C. and sodium borohydride (350 mg, 9.66 mmol) was added. After stirring for 1 hour, the reaction was quenched with saturated aqueous ammonium chloride solution and extracted with ethyl acetate. The organic phase was dried over MgSO₄, filtered and concentrated to provide a yellow oil which was diluted in 80 mL DMF and to the resulting solution was added TBDPSCl (3.62 g, 13.2 mmol) and imidazole (1.5 g, 22.0 mmol). The reaction was allowed to stir for 16 hours, then quenched with water and extracted with ethyl acetate. The organic phase was dried over MgSO4, filtered and concentrated in vacuo to provide a crude residue which was diluted in 100 mL THF and to the resulting solution was added LAH (1M in THF, 13.26 mL, 13.26 mmol) and the reaction was stirred for 16 hours, then quenched carefully with 2.0 mL water. The resulting solution was treated with 2.0 mL 1N NaOH and then diluted with 6.0 mL water. The mixture was cooled to 0° C. and allowed to stir at this temperature for 30 minutes then was filtered and the filtrate concentrated in vacuo. The residue obtained was washed with hexanes and the resulting filtrate was concentrated in vacuo and the crude product purified using flash column chromatography (10% acetone in hexanes) to provide compound 75A (2.4 g).

### Step B—Synthesis of Compound 75B

To a solution of compound 75A (200 mg, 0.58 mmol) in 8 mL THF was added 2-nitrophenylselenocyanate (395 mg, 1.74 mmol) followed by dropwise addition of tributyl phosphine (0.43 mL, 1.74 mmol). The reaction was stirred for 1 hour, then concentrated in vacuo and diluted with 10 mL dichloromethane. To this solution was added mCPBA (430 mg, 1.74 mmol) at 0° C. After stirring for 1 hour, the reaction was concentrated and diluted with 12 mL toluene. To this solution was added diisopropylamine (0.25 mL, 1.74 mmol) and the reaction was heated at 90° C. for 16 hours. After removing the solvent, the crude material was purified (100% hexanes) to afford the olefin 75B (140 mg).

### Step C—Synthesis of Compound 75C

To a solution of compound 75B (140 mg, 0.44 mmol) in a mixture of 2 mL CH₂Cl₂, 2 mL CH₃CN, and 3 mL water was added sodium periodate (530 mg, 2.46 mmol) followed by ruthenium trichloride monohydrate (3 mg). The reaction was stirred for 20 hours, then concentrated in vacuo and the crude product obtained was reduced to compound 75C using sodium borohydride via the method described in Example 69.

### Synthesis of Compounds 399, 400, 401 and 403

Compound 399 was prepared from compound 75D using the procedure described in Example 60. Compound 400 was prepared from compound 399 using the method described in Example 70. Compound 401 was prepared from compound 400 using the method described in Example 73. Compound 403 was prepared from compound 399 using the method described in Example 73.

### Example 76

### Preparation of Compound 402

To a solution of compound 399 (18 mg, 0.04 mmol) in 1 mL acetonitrile was added Ag₂O (40 mg, 0.17 mmol) followed by MeI (0.015 mL, 0.24 mmol). The reaction was heated to reflux and allowed to stir at this temperature for 20 hours, then the reaction mixture was cooled to room temperature and concentrated in vacuo. The resulting residue was purified using flash column chromatography (20% acetone in hexanes) to give 402 (15 mg).

### Example 77

### Preparation of Compounds 404 and 405

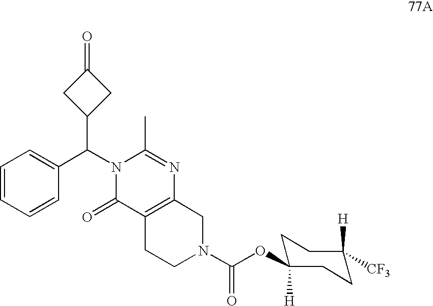

### Step A—Synthesis of Compound 77A

Compound 390 was subjected to the method described in Example 3, then the product of this reaction was reacted according to the method described in Example 12 to provide compound 77A.

### Synthesis of Compounds 404 and 405

Compound 404 was prepared from compound 77A using the method described in Example 73 for synthesis of 396. Reduction of 77A using the method described in Example 71 gave provided compound 405.

### Example 78

### Preparation of Compound 406

Compound 406 was prepared by O-methylation of compound 405 using the method described in Example 76.

### Example 79

### Preparation of Compound 407

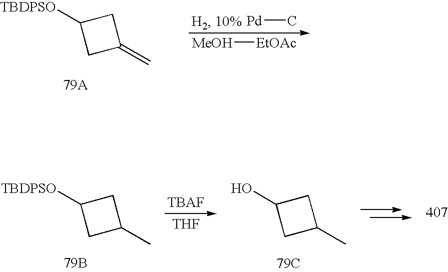

To a solution of compound 79A in 3 mL methanol and 1 mL ethyl acetate was added 10% Pd—C (30 mg). The reaction was hydrogenated at 1 atmospheric pressure for 2 hours after which it was filtered through celite and concentrated in vacuo to provide compound 79B, which was then was converted to compound 79C using the method described in Example 60. Compound 407 was prepared from compound 79C using the procedure described in Example 12.

### Example 80

### Preparation of Compound 447

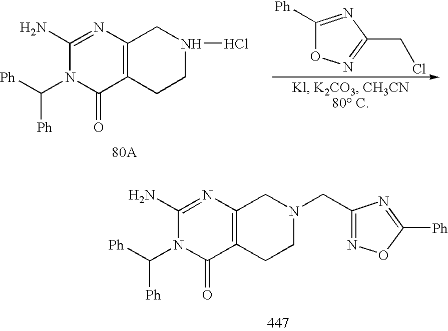

To compound 80A (30 mg, 0.08 mmol) was added 3-(chloromethyl)-5-phenyl-1,2,4-oxadizaole (20 mg, 0.10 mmol), K₂CO₃ (17 mg, 0.12 mmol), KI (14 mg, 0.08 mmol), and CH₃CN (0.3 mL). The solution was heated to 80° C. and allowed to stir at this temperature for 16 hours. The reaction mixture was allowed to cool to room temperature, then concentrated in vacuo and the resulting residue was purified using preparative thin layer chromatography (30% EtOAc/hexanes) to provide compound 447 (14 mg, 36% yield).

### Example 81

### Preparation of Compound 410

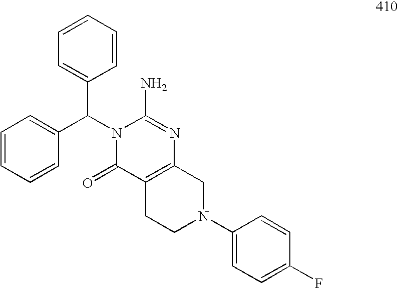

Compound 257 was deprotected using the method described in Example 24. The deprotected product was taken up in toluene (3 mL) in a sealed tube and to the resulting solution was added 4-bromofluorobenzene (0.05 g, 4.5 eq.), Pd₂ dba₃ (0.05 eq.) BINAP (0.10 eq.) and NaO-tBu (1.5 eq.) and the reaction was heated to 110° C. and allowed to stir at this temperature for 18 hours. The reaction mixture was allowed to cool to room temperature, . . . then was concentrated in vacuo and the residue obtained was purified using PLC (20% EtOAc/hexane) to provide compound 410.

### Example 82

### Preparation of Compound 411

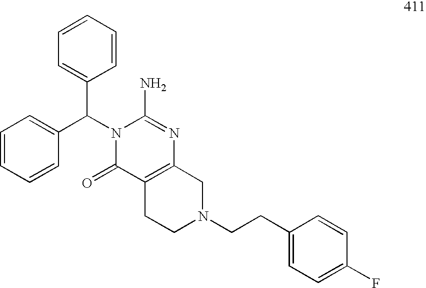

Compound 257 was deprotected using the method described in Example 3. The deprotected product (0.041 g, 0.12 mmol) was then taken up in ethanol (4 mL), and to the resulting solution was added 2-(4-fluorophenyl)ethyl bromide (0.034 g, 0.17 mmol) and K₂CO₃ (0.024 g, 0.17 mmol), and the reaction was heated to 100° C. and stirred at this temperature for 18 hours. Concentration and PLC (3% MeOH/CH₂Cl₂) provided compound 411 as a white film.

### Example 83

### Preparation of Compound 415

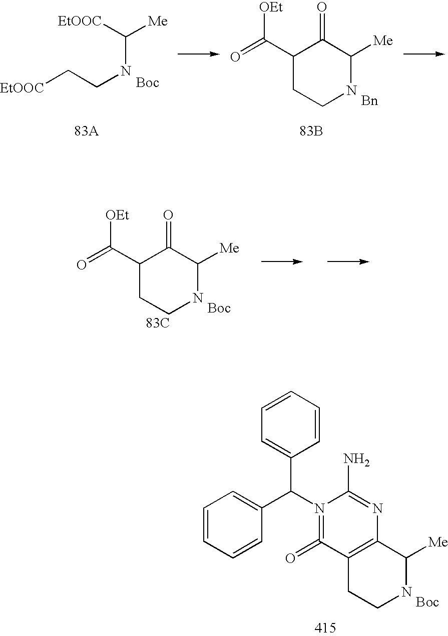

### Step A—Synthesis of Compound 83B

Compound 83A (8.00 g, 24.9 mmol) was combined with Na (0.86 g, 37.4 mga) and EtOH (0.20 mL) in toluene (100 mL) and heated at reflux for 18 h. The reaction was allowed to cool, then acidified with HOAc (10 mL), partitioned with ether and water, washed with 1N NaHCO₃, dried, concentrated and chromatographed on silica (20% EtOAc/hexane) to yield compound 83B as a yellow oil.

### Step B—Synthesis of Compound 83C

Compound 83B (2.00 g, 7.3 mmol) was hydrogenated for 16 h using 10% Pd/C (0.70 g) in EtOH (20 mL) with 1N aq. HCl. (10 mL), and the mixture filtered and concentrated. The residue was treated with Et₃N (2.0 mL) and Boc₂O (2.06 g, 9.4 mmol) in EtOH (30 mL). After 18 h the solution was concentrated, partitioned with ether and water, and washed with 1N HCl. The ether was dried and concentrated to yield crude compound 83C as a yellow oil

Step C—Synthesis of Compound 415 Compound 83C was converted to 415, a white solid, according to the procedures of Examples 28, 29, 35, and 36.

### Example 84

### Preparation of Compound 429

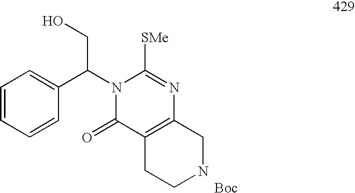

(±)-Phenylglycinol was converted to Compound 429 using the methods described in Examples 29 and 35.

### Example 85

### Preparation of Compound 430

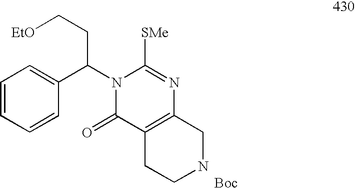

Compound 429 (0.35 g, 0.84 mmol) was combined with EtI (0.27 mL, 3.35 mmol), 2,6-di(t-butyl)pyridine (0.64 g, 3.35 mmol) and AgOTf (0.86 g, 3.35 mmol) in CH₂Cl₂ (20 mL). The mixture was stirred 64 h, filtered, concentrated, and purified by PLC (20% EtOAc/hexane) to give compound 430 as a white solid.

### Example 86

### Preparation of Compound 4411

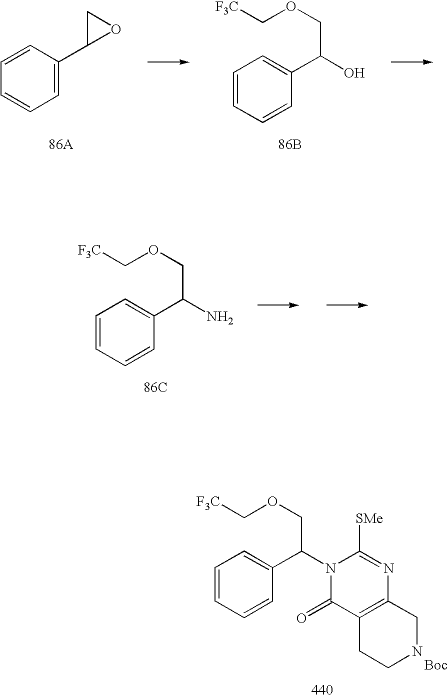

### Step A—Synthesis of Compound 86B

Compound 86A (3.74 g, 32 mmol) in DMF (40 mL) was treated with trifluoroethanol (15 mL, 20.6 g, 206 mmol) and NaO-tBu (0.60 g, 6.3 mmol). The reaction was heated in a sealed tube 18 h at 100° C., partitioned with ether and water, dried and concentrated to yield compound 86B as a yellow oil.

### Step B—Synthesis of Compound 86C

To a solution of compound 86B (2.54 g, 11.5 mmol) in CH₂Cl₂ (20 mL) at 0° C. were added MsCl (1.58 g, 13.8 mmol) and Et₃N (1.40 g, 13.8 mmol), and the resulting reaction was allowed to stir at 0° C. for 3 h. The reaction was concentrated, treated with ether, filtered and concentrated in vacuo. The resulting oily residue was taken up in THF (10 mL), diluted with conc. aq. NH₃ (20 mL) and the resulting solution placed in a sealed tube and heated at 70° C. for 18 h. The reaction mixture was allowed to cool, partitioned with ether and water, and extracted with 1N HCl. The extract was basified with NaOH, extracted with ether, and the ether phase dried (MgSO₄), filtered and concentrated in vacuo to provide compound 86C as a yellow oil.

### Step C—Synthesis of Compound 440

Compound 86C was converted to compound 440 using the procedures of Examples 29 and 35.

### Example 87

### Preparation of Compound 443

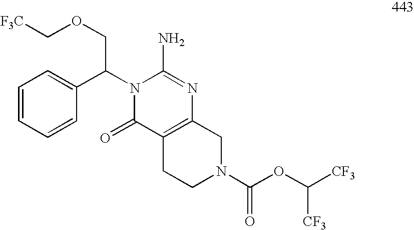

### Step A—Synthesis of Compound 87A

Compound 440 was converted to 87A using the procedure of Example 36.

### Step B—Synthesis of Compound 443

Compound 87A was deprotected using the method described in Example 3. The resulting HCl salt (0.030 g, 0.086 mmol) in CH₂Cl₂ (12 mL) was treated with 20% COCl₂ in toluene (0.055 mL) and Et₃N (0.036 mL). After 2 hours, the mixture was concentrated, treated with ether, filtered, concentrated, and taken up in THF (2 mL). To the solution were added hexafluoro-2-propanol (0.043 g, 0.26 mmol) and NaO-tBu (0.025 g, 0.26 mmol). After 2 hours, the mixture was concentrated and purified by PLC (3% MeOH/CH₂Cl₂) to provide compound 443 as a yellow solid.

### Example 88

### Preparation of Compound 457

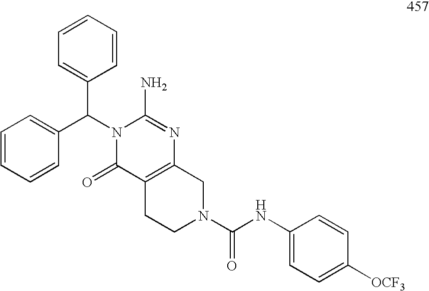

Compound 257 was deprotected using the method described in Example 3. The deprotected product (0.020 g, 0.054 mmol) in MeCN (1.5 mL) was treated with 4-(trifluoromethoxy)phenyl isocyanate (0.013 g, 0.064 mol) and Et₃N (0.022 mL). The mixture was heated at 80° C. for 18 h. Concentration and purification by PLC (3% MeOH/CH₂Cl₂) yielded the title compound as a white solid.

### Example 89

### Preparation of Compound 458

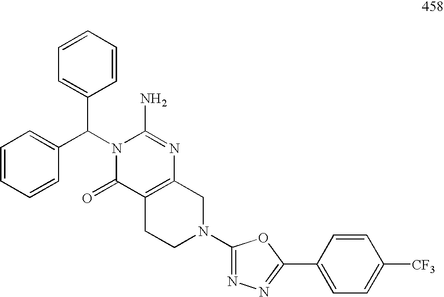

Compound 257 was deprotected using the method described in Example 3. The deprotected Boc-compound (0.050 g, 0.19 mmol) in CH₂Cl₂ (2 mL) was treated with 20% COCl₂ in toluene (0.086 mL) for 1 h. 4-(Trifluoromethyl)benzhydrazide (0.033 g, 0.16 mmol) and Et₃N (0.050 mL) were added. After 4 days the reaction was concentrated and purified by PLC (4% MeOH/CH₂Cl₂) to obtain a white solid. This was treated with POCl₃ (0.046 mL) and pyridine (0.025 mL) in ClCH₂CH₂Cl (2 mL) at 80° C. for 3 h. Concentration and PLC yielded the title compound as a white solid.

### Example 90

### Preparation of Compound 486

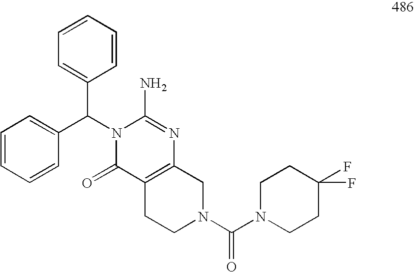

Similarly to Example 89, the deprotected Boc-compound (0.020 g, 0.054 mmol) was treated with COCl₂ for 1 h, concentrated, and taken up in THF (2 ml). 4,4-Difluoropiperidine hydrochloride (0.017 g) and Et₃N (0.040 mL) were added. The reaction was heated at 60° C. for 3 h. Concentration and PLC yielded the title compound as a yellow solid.

Utilizing 4-(trifluoromethyl)piperidine hydrochloride led to compound 487, a white solid. Utilizing 4-(trifluoromethyl)aniline (heating period 18 h) led to compound 495, a white solid.

### Example 91

### Preparation of Compound 496

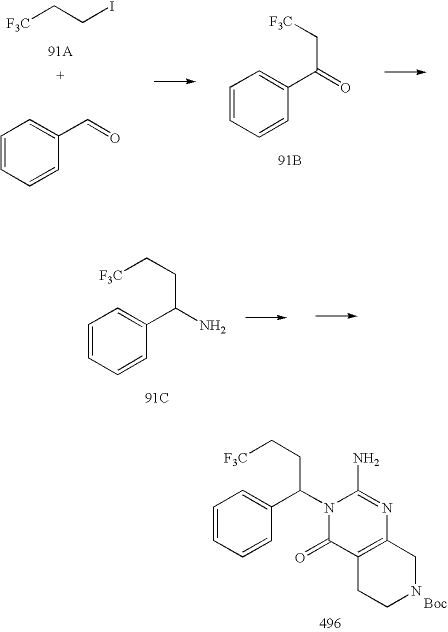

### Step A—Synthesis of Compound 91B

Compound 91A (5.0 g, 22 mol) was added to Mg turnings (0.70 g, 29 mga) and catalytic iodine in ether (30 mL). After 1 h, the reaction was cooled to 0° C. and treated with benzaldehyde (2.08 g, 20 mmol). After 1 h, satd. NH₄Cl was added (100 mL). The ether was concentrated to leave a yellow oil, taken up in CH₂Cl₂ (40 mL), and treated with PCC (12.0 g, 56 mmol) for 4 h. Hexane (30 ml) was added, the solid filtered and concentrated to give crude compound 91B as a yellow solid.

### Step B—Synthesis of Compound 91C

Compound 91B (2.80 g, 13.8 mmol) was heated in a mixture of formic acid (20 mL) and formamide (50 mL) at 150° C. for 4 h. The crude product was isolated by ether extraction and heated at reflux with conc. HCl (20 mL) for 1 h. The mixture was concentrated, partitioned with ether and water, the aqueous basified with NaOH, extracted with ether, dried (MgSO₄), and concentrated to yield compound 91C as a colorless oil.

Step C—Synthesis of Compound 496 Compound 91C was converted to 496 using the procedures of Examples 29, 35 and 36.

### Example 92

### Preparation of Compound 502

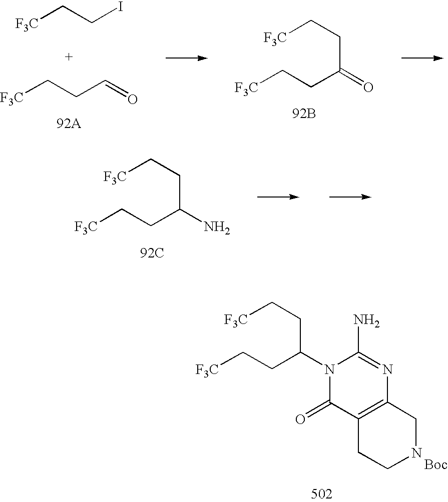

### Step A—Synthesis of Compound 92B

Similarly to Example 90, Step A, convert compound 92A to yield crude 92B as a yellow oil.

### Step B—Synthesis of Compound 92C

Similarly to Example 90, Step B, convert compound 92B to yield compound 92C as a yellow oil.

### Step C—Synthesis of Compound 502

Compound 92C was converted to compound 502 using the procedures of Examples 29, and 36.

### Example 93

### Preparation of Compound 513

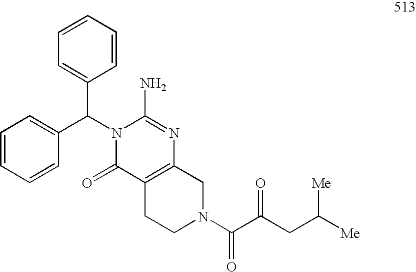

Compound 257 was deprotected using the method described in Example 3. The HCl salt (0.030 g, 0.082 mmol) in DMF (1 mL) was treated with 2-oxo-4-methylpentanoic acid (0.016 g, 0.12 mmol), EDCI (0.024 g, 0.12 mmol), HOBt hydrate (0.17 g) and NMM (0.040 mL). After 20 hours, the reaction was concentrated and purified by PLC to yield the title compound as a white solid.

### Example 94

### Preparation of Compound 518

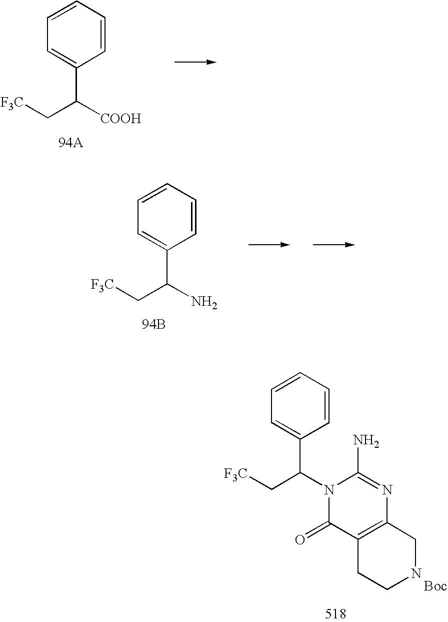

### Step A—Synthesis of Compound 94B

A solution of compound 94A (3.70 g 17 mmol) and Et₃N (2.84 mL) in acetone (40 mL) at 0° C. was treated with EtOCOCl (1.82 mL). After 2 h, NaN₃ (1.88 g, 29 mmol) in water (20 mL) was added dropwise. After 3 h, the reaction was partitioned between ether and water. The ether phase was dried, filtered and concentrated in vacuo to provide an oil, which was diluted with toluene (20 mL) and heated at 80° C. for 18 h. Conc. HCl (20 mL) was added and the mixture stirred 18 h. The reaction mixture was then partitioned between ether and water. Basification of the aqueous with NaOH, extraction with ether, drying and concentration gave compound 94B as a yellow oil.

### Step B—Synthesis of Compound 518

Compound 94B was converted to compound 518 using the procedures of Examples 29, and 36.

### Example 95

### Preparation of Compound 528

Compound 528 was prepared by the method used for the preparation of compound 1 in Example 1 and replacing acetamidine with fluoroacetamidine.

### Example 96

### Preparation of Compound 529

Compound 529 was prepared from the amine obtained by deprotection of 528 (using the method in Example 3) and by reacting the deprotected material with 2,2,3,3, tetrafluorocyclobutanol using the method described in Example 12.

### Example 97

### Preparation of Compound 530

Compound 530 was prepared from the amine obtained by deprotection of compound 528 (using the deprotection method described in Example 3), by treating the amine with 4-trifluoromethyl phenol via the method described in Example 12.

### Example 98

### Preparation of Compound 531

To a solution of compound 199 (0.06 g, 0.168 mmol) and N-hydroxy-4-(trifluoromethoxy)benzimidamide (0.048 g, 0.22 mmol) in EtOAc (1.0 mL) was added dropwise a solution of ZnCl₂ (1N in ether, 0.22 mL). A precipitate was formed on addition. The reaction was allowed to stir for 15 hours and the supernatant was decanted, filtered and the resulting residue was rinsed twice with ether. The precipitate collected was dried under vacuum and taken up in conc. HCl (0.5 mL) and ethanol (1.0 mL). The resulting reaction was heated to reflux and allowed to stir at this temperature for 1 hour. The reaction mixture was then cooled and solid Na₂CO₃ was added to basify the solution. The resulting solution was extracted with dichloromethane, the organic phase was dried, filtered and concentrated in vacuo. The residue obtained was purified via flash column chromatography (40% acetone/hexanes) and the product obtained was reacted with Cs₂CO₃ and benzhydryl bromide using the method described in Example 1, step C to provide compound 531 (12.0 mg, 13% yield) as the major product.

### Example 99

### Preparation of Compounds 534 and 535

Compounds 534 and 535 were prepared analogously to compound 531 by using the method described in Example 96 and using the corresponding commercially available aldoximes.

### Example 100

### Preparation of Compounds 584, 540, 541 and 542

### Step A—Synthesis of Compound 584

Compound 584 was prepared from tert-butyl 4-oxoazepane-1-carboxylate using the method described in Example 48.

Step B—Synthesis of Compound 540 Compound 584 was deprotected using the method describe in Example 3 and the resulting amine (0.15 g, 0.58 mmol), 2,2,6,6-tetramethylpiperidine (0.097 g, 0.69 mmol) and 1-(4-(trifluoromethyl)phenyl)ethyl methanesulfonate (0.311 g, 1.16 mmol) were taken up in acetonitrile and the resulting reaction was heated to reflux and allowed to stir at this temperature for 3 hours. The reaction mixture was then cooled to room temperature and after standard work-up was purified by column chromatography (4% MeOH/CH₂Cl₂) to provide compound 540 (0.025 g, 8.3% yield).

### Step C—Synthesis of Compounds 541 and 542

The racemic compound 540 was resolved on a ChiralPak AD column using 5% isopropanol/heptanes to provide the enantiomeric compounds 541 and 542.

### Example 101

### Preparation of Compounds 408, 465, 466, 475 and 508

Compound 109 (prepared from 1-amino-1-phenylcyclobutylmethane using the methods described in Examples 29 and 35) was converted to compound 408 using the methods described in Examples 36 and 103, Step C.

Compound 109 was also converted to compounds 465 and 466 and 508 using the methods described in Examples 36 and 12. By the same procedures compound 508 was produced.

Compound 109 was also converted to compound 475 using the methods described in Examples 36 and 93.

### Example 102

### Preparation of Compound 409

Compound 257 was converted to compound 409 using the method described in Example 87, Step B.

### Example 103

### Preparation of Compound 412

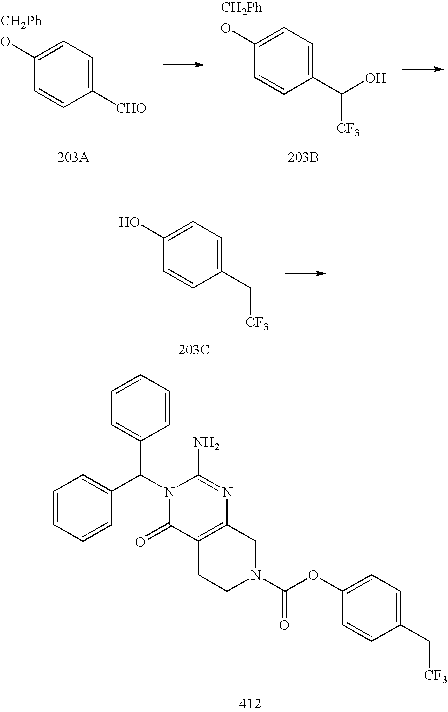

### Step A—Synthesis of Compound 203B

To a solution of compound 203A (3.0 g, 14.1 mmol) and TMS-CF₃ (3.24 mL) in THF (10 mL), was added 1.0M TBAF solution in THF (14.2 mL, 14.2 mmol). The reaction was stirred 18 hours, then hexane was added, and the organic phase was washed 3 times with 2N HCl. The organic phase was dried (MgSO₄) and concentrated in vacuo to provide compound 203B as a yellow solid.

### Step B—Synthesis of Compound 203C

To a solution of compound 203B (3.2 g, 11.3 mmol) in toluene (60 mL) was added SOCl₂ (1.65 mL) and pyridine (0.10 mL). The reaction was heated to 70° C. and allowed to stir at this temperature for 3 hours, then was cooled to room temperature and concentrated in vacuo. The residue obtained was then taken up in ether, washed with 1N HCl, dried (MgSO₄) and concentrated in vacuo to provide a yellow solid residue which was dissolved in 1:1 MeOH/EtOAc (30 mL), then 10% Pd/C (0.3 g) was added, and the reaction was hydrogenated at 45° C. for 6 hours. The reaction mixture was filtered and the filtrate concentrated in vacuo to provide compound 203C as a yellow solid.

### Step C—Synthesis of Compound 412

Compound 257 was deprotected using the method described in Example 3 and the product obtained (0.030 g, 0.082 mmol) was diluted with CH₂Cl₂ (2 mL) and to the resulting solution was added 20% COCl₂ in toluene (0.086 mL) and Et₃N (0.025 mL). The reaction was stirred for 1 hour, then compound 103C (0.029 g, 0.16 mmol) and Et₃N (0.050 mL) were added. The resulting reaction was stirred for 18 hours, then concentrated in vacuo and purified using PLC (3% MeOH/CH₂Cl₂) to provide compound 412 as a white solid.

### Example 104

### Preparation of Compound 413

In similar fashion to Example 101, employ compounds 109 and 103C to produce compound 413, a white solid.

### Example 105

### Preparation of Compound 414, 516, and 517

Using the method described in Example 101 and employing 1-(4-fluorophenyl)-1-aminophenylmethane and compound 103C compound 414 was prepared as, a white solid. Utilizing 1-(4-fluorophenyl)-1-aminophenylmethane and the method described in Example 12, compounds 516 and 517 were prepared.

### Example 106

### Preparation of Compound 416

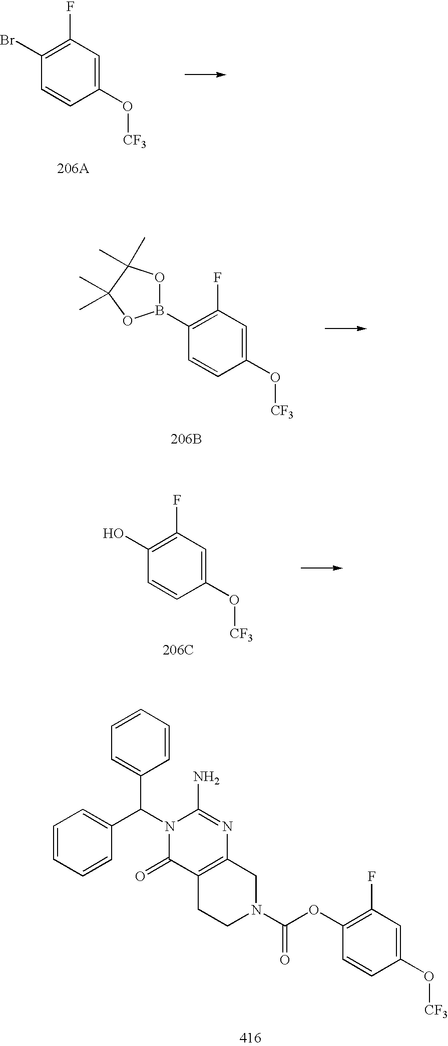

### Step A—Synthesis of Compound 206B

Compound 206A (2.14 g, 8.3 mmol) was combined with PdCl₂(dppf) (0.20 g, 0.25 mmol), bis(pinicolato)diboron (2.52 g, 9.9 mmol), and KOAc (2.43 g, 24 mmol) in DMSO (10 mL). The mixture was put under N₂ atmosphere, heated to 100° C. and allowed to stir at this temperature for 4 hours, then partitioned with water and 1:1 EtOAc/hexane. The organic phase was dried (MgSO₄) and concentrated in vacuo, and the resulting residue was purified using flash column chromatography on silica (10% EtOAc/hexane) to provide compound 206B as a white solid.

### Step B—Synthesis of Compound 206C

Compound 206B (0.79 g, 2.6 mmol) was taken up in EtOH (10 mL) and to the resulting solution was added 50% H₂O₂ (0.15 mL). The reaction was allowed to stir for 0.5 hours and was then concentrated in vacuo. The residue obtains was purified PLC gave compound 206C as a yellow oil.

### Step C—Synthesis of Compound 416

In similar fashion to Example 203, Step C, employ compound 57 and compound 106C to produce compound 416 as a white solid.

### Example 107

### Preparation of Compounds 417 and 418

Employing compound 257 and 4-(trifluoromethyl)benzyl alcohol in the procedure described in Example 203, Step C, provided compound 417 as a yellow solid. Similarly, using the same procedure with 4-(trifluoromethoxy)benzyl alcohol, provided compound 418 as a white solid.

### Example 108

### Preparation of Compounds 419, 420, and 422

Compound 257 was deprotected using the method described in Example 3 and to the product was added with 20% COCl₂ in toluene (0.051 mL) and Et₃N (0.034 mL). The resulting reaction was stirred for 30 minutes, concentrated in vacuo and treated with 1:1 EtOAc/ether, filtered, and concentrated again. The residue obtained was taken up in THF (1.5 mL) and to the resulting solution was added (trifluoromethyl)benzyl alcohol (0.043 g, 0.24 mmol) and NaO-tBu (0.024 g, 0.24 mmol). The reaction was stirred for 30 minutes, concentrated in vacuo and the residue obtained was purified using PLC to obtain compound 419 as a yellow solid.

Using the above method and substituting 4-(trifluoromethoxy)benzyl alcohol for compound 257, compound 420 was provided as a yellow solid. Similarly, 1-Phenylethanol was substituted for compound 257 to provide compound 422 as a yellow solid.

### Example 109

### Preparation of Compound 421

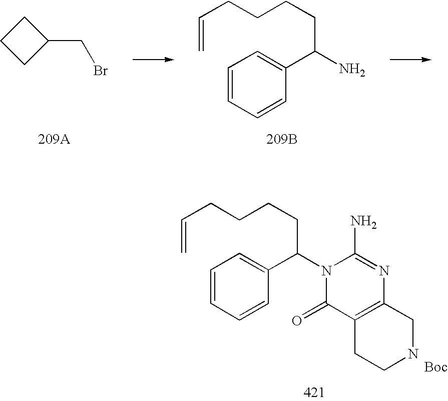

### Step A—Synthesis of Compound 209B

To compound 209A (4.00 g, 27 mmol) in THF (40 mL) was added Mg turnings (0.64 g, 27 mg) and the solution was heated to 70° C. and allowed to stir at this temperature for 2 hours. Benzonitrile (2.28 mL) and CuCl (0.046 g) were then added and the reaction was stirred for an additional 2 hours. LiAlH₄ (1.0M in THF, 27 mL) was then added and the reaction was stirred for an additional 5 hours. The reaction mixture was allowed to cool to room temperature and allowed to stir for 18 hours, then water was added dropwise to the reaction mixture (5 mL), followed by the addition of 1N NaOH (50 mL). The resulting mixture was extracted with EtOAc, and the extract washed with 1N HCl (3×25 mL). The aqueous was basified with NaOH to pH 10 and extracted with ether. The ethereal layer was dried (MgSO₄) and concentrated in vacuo to provide compound 209B as a yellow oil.

### Step B—Synthesis of Compound 421

Compound 421, a white solid, was prepared from compound 209B using the methods described in Examples 29, 35 and 36.

### Example 110

### Preparation of Compound 423

Compound 423, a white solid, was prepared from compound 421 employing the method of Example 103, Step C.

### Example 111

### Preparation of Compounds 424 and 425

Compound 424 was prepared from 1,2-diphenylpropane using the methods described in Examples 29, 35, and 365. This was then converted to compound 425 using the method described in Example 103, Step C.

### Example 115

### Preparation of Compounds 426 and 428

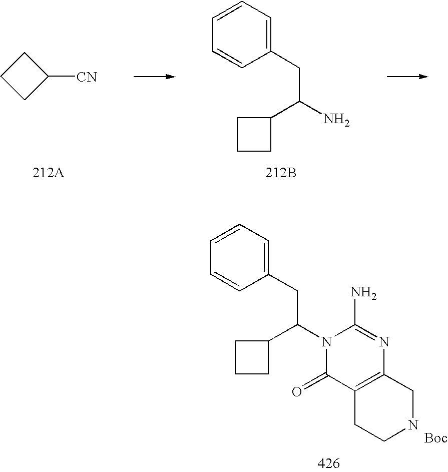

### Step A—Synthesis of Compound 212B

Using the method described in Example 209, Step A, and using compound 212A (2.50 g, 31 mmol) and benzylmagnesium bromide (1.0 M, 37 mL), compound 212B was obtained as a brown oil.

### Step B—Synthesis of Compound 426

Compound 426, a yellow solid, was prepared from compound 212B using the methods described in Examples 29, 35 and 36. This was converted to compound 428, a white solid, employing the method of Example 103, Step C.

### Example 113

### Preparation of Compounds 427, 434, 445, 446, 454, 459, 489, 501, 509, 510, and 524

Compound 427, a yellow solid, was prepared from cyclobutanol employing the method of Example 103, Step C Similarly prepared were: compound 434 from 4-(difluoromethoxy)phenol, compound 445 from 4,4,4-trifluorobut-2-ene-1-ol, compound 446 from 1,3-difluoropropan-2-ol, compound 454 from 2,4,4,4-tetrafluorobut-2-ene-1-ol, compound 459 from (2,2-difluorocyclopropyl)methanol, compound 489 from cis-(3-trifluoromethyl)cyclohexanol, compound 501 from 4,4-dimethylcyclohexanol, compound 509 from 4,4-bis(trifluoromethyl)cyclohexanol, compound 510 from 4-trans-(methylsulfonyl)cyclohexanol, and compound 524 from 3,3-dimethylcyclobutanol.

### Example 114

### Preparation of Compounds 431, 432, 433, 441, 442, 444, 463, and 464

Compounds 431 and 432 were prepared from Compound 430 using the method described in Example 36. Compound 433, a white solid, was prepared from Compound 431 using the method described in Example 103, Step C.

In a similar fashion, compound 440 was converted to compounds 441 and 442; compound 441 was converted to compound 444.

Compound 441 was also converted to compounds 463 and 464 using the method described in Example 12.

### Example 115

### Preparation of Compounds 519, 520, and 521

Compound 518 was converted to compounds 519 and 520 using the method described in Example 12. Compound 521 was produced using the method described in Example 103,

### Step C.

### Example 116

### Preparation of Compounds 435, 436, 439, 449, 451, 461, and 462

Compound 435 was prepared from 1,1-di(cyclobutyl)aminomethane using the methods described in Examples 29 and 35. This was converted to compound 436 using the method described in Example 103, Step C. Compound 435 was converted to compound 451 using the method described in Example 87, Step B, and compound 435 was converted to compounds 461 and 462 using the method described in Example 12.

Similarly, compound 439 was prepared starting with (S)-2-amino-1-methoxy-3-phenylpropane and subsequently converted to compound 449.

### Example 117

### Preparation of Compounds 497, 498, 499, and 500

Compound 496 was converted to compounds 498 and 499 using the method described in Example 12; compound 496 to compound 500 using the method described in Example 87,

Step B; and compound 496 was converted to compound 497 using the method described in Example 93.

### Example 118

### Preparation of Compounds 503, 504, 505, 506, and 507

Compound 502 to compounds 503 and 504 using the method described in Example 12, and to compound 507 using the method described in Example 103, Step C. Compound 502 was converted to compound 506 using the method described in Example 87, Step B; and compound 502 was converted to compound 505 using the method described in Example 93.

### Example 119

### Preparation of Compounds 455, 476, 514, and 515

Using the method described in Example 201, and employing 1-amino-1-(4-fluorophenyl)cyclobutylmethane as a starting material, compound 455 was prepared.

Employing the amine hydrochloride used to prepare compound 455 and the reagents and methodology of Example 12, compounds 514 and 515 were prepared. Using this same amine and 3-trifluoromethyl-4,4,4-trifluorobutanoic acid with the method of Example 93, compound 476 was prepared.

### Example 120

### Preparation of Compounds 444, 463, 464, and 477

Compound 441 was converted to compounds 463 and 464 using the method described in Example 12. Compound 441 was also converted to compound 444 Compound 441 was converted Example 103, Step C. Compound 441 was converted to compound 477 using the method described in Example 93.

### Example 121

### Preparation of Compound 491

### 1-Amino-1-phenyl-2-cyclopropylethane (prepared as described in WO2004/033440) was converted to compound 491, using the methods described above in Examples 29, 35, and 36

### Example 122

### Preparation of Compound 492, 494, 511, and 512

Compound 491 was converted to compounds 511 and 512 using the method described in Example 12; compound 491 was converted to compound 494 using the method described in Example 87, Step B; and compound 491 was converted to compound 492 using the method described in Example 93.

### Example 123

### Preparation of Compounds 522 and 523

1-Amino-1-(4-fluorophenyl)cyclopropylmethane (prepared from (4-fluorophenyl)cyclopropylketone using the method described in Example 120) was converted to compound 522 using the methods described in Examples 29, 35, and 36. Compound 522 was converted to compound 523 using the method described in Example 12.

### Example 124

### Preparation of Compound 485

Compound 257 was deprotected using the method described in Example 3 and the product obtained (0.100 g, 0.27 mmol) was taken up in THF (1 mL) and to the resulting solution was added 20% COCl₂ in toluene (0.0.17 mL) and Et₃N (0.23 mL) and the reaction was allowed to stir for 30 minutes. Boc-hydrazide (0.071 g, 0.53 mmol) was then added and the reaction was allowed to stir for an additional 20 hours, then concentrated in vacuo. The resulting residue was purified using PLC to obtain a white solid, which was diluted with 4M HCl in dioxane (2.0 mL). The resulting solution was allowed to stir for 2 hours, then was concentrated in vacuo to provide a yellow solid residue.

The yellow solid residue (0.025 g, 0.056 mmol) was taken up in CH₂Cl₂ (1 mL) and to the resulting solution was added isobutyryl chloride (0.010 mL) and Et₃N. The reaction was allowed to stir for 1 hour, then was concentrated in vacuo to provide a residue which was diluted with ClCH₂CH₂Cl (3 mL). To the resulting solution was added POCl₃ (0.050 mL) and pyridine (0.025 mL), and the resulting reaction was heated at 70° C. and allowed to stir at this temperature for 18 hours. The reaction mixture was allowed to cool to room temperature, then was concentrated in vacuo to provide a crude residue which was purified using PLC to provide compound 485.

### Example 125

### Preparation of Compounds 448, 450, 452, 453 and 456

Using the method described in Example 80, compound 80A was reacted with the appropriate halide reactant (in place of 3-(chloromethyl)-5-phenyl-1,2,4-oxadizaole) to provide compounds 448, 450, 452, 453 and 456.

### Example 126

### Preparation of Amides from Compound 257

Using the method described in Example 93, compounds were prepared using the indicated carboxylic acids in place of 2-oxo-4-methylpentanoic acid:
cyclobutanecarboxylic acid to produce compound 437;
3-cyclopentylpropanoic acid to produce compound 438;
3-trifluoromethyl-4,4,4-trifluorobutanoic acid to produce compound 460;
2,2-difluorocyclopropanecarboxylic acid to produce compound 467;
trans-4-(trifluoromethyl)cyclohexanecarboxylic acid to produce compound 468;
2-(trifluoromethyl)cyclopropanecarboxylic acid to produce compound 469;
4-(trifluoromethyl)benzoic acid to produce compound 470;
4-(trifluoromethoxy)benzoic acid to produce compound 471;
1-(trifluoromethyl)cyclopropanecarboxylic acid to produce compound 472;
2-(1,1,1,3,3,3-hexafluoro-2-propyloxy)acetic acid to produce compound 473;
4,4,4-trifluorobutanoic acid to produce compound 474;
2-fluoro-4-(trifluoromethyl)benzoic acid to produce compound 478;
3,5-bis(trifluoromethyl)benzoic acid to produce compound 479;
2-fluoro-4-(trifluoromethoxy)benzoic acid to produce compound 480;
3-(trifluoromethoxy)benzoic acid to produce compound 481;
3-(trifluoromethyl)benzoic acid to produce compound 482;
4-(1,1,2,2-tetrafluoroethoxy)benzoic acid to produce compound 483;
3-fluoro-4-(trifluoromethyl)benzoic acid to produce compound 484;
3-phenyl-4,4,4-trifluorobut-2-enoic acid to produce compound 488;
3-phenyl-4,4,4-trifluorobutanoic acid to produce compound 490;
3-methyl-4,4,4-trifluorobutanoic acid to produce compound 493; and
2-oxo-4′-(trifluoromethyl)phenylacetic acid to produce compound 525.

### Example 127

### Procedure for Making a Library of Amides at R⁴

The HCl salt of compound 2 (0.64 g, 1.93 mmol) was neutralized with a tertiary amine resin (diethyl aminomethyl polystyrene), then a stock solution was prepared by dissolving the resin mixture in MeCN:THF (3:2, 100 mL). PS-EDC resin (0.038 g, 0.058 mmol) was then added to 96-wells of a deep well polypropylene microtiter plate followed by the stock solution (1 mL) of compound 2 (0.0193 mmol) and HOBT (0.029 mmol). 1 M stock solutions of each of the individual carboxylic acid coupling partners (0.023 mL, 0.023 mmol) used to prepare various amides from compound 2 were added to the wells, which were then sealed and shaken at 25° C. for 20 hours. The solutions obtained were filtered through a polypropylene frit into a second microtiter plate containing PS-isocyanate resin (3 equivalents, 0.058 mmol) and PS-trisamine resin (6 equivalents, 0.116 mmol). After the top plate was washed with MeCN (0.5 mL/well), the top plate was removed and the bottom microtiter plate was sealed and then shaken at 25° C. for 16 hours. The resulting solutions were then filtered through a polypropylene frit into a 96-well collection plate. The wells of the top plate were washed with MeCN (0.5 mL/well), and the top plate removed. The resultant solutions in the collection plate were transferred into vials and concentrated in vacuo using a SpeedVac. The resulting amides were analyzed using LCMS and those >70% pure were submitted for further analysis.

### Example 128

### Procedure for Making a Library of Carbamates at R⁴

A stock solution was prepared by dissolving compound 2 (0.280 g, 0.76 mmol) in 1,2-dichloroethane (35.0 mL). The compound 2 stock solution (1.0 mL, 0.022 mmol) was then added to 32-wells of a deep well polypropylene microtiter plate, followed by the addition of PS-DIEA (3.51 mmol/g, 0.042 g) to each well. 1M stock solutions of each of the individual chloroformate coupling partners (1.0 M in DCE, 2.0 equiv, 0.05 mL) used to make the carbamates with compound 2 were added to the wells, which were then sealed and shaken at 25° C. for 20 hours. The solutions were filtered through a polypropylene frit into a second microtiter plate containing PS-isocyanate resin (3 equiv., 0.066 mmol) and PS-trisamine resin (6 equiv., 0.132 mmol). After the top plate was washed with MeCN (0.5 mL/well), the top plate was removed, and the bottom microtiter plate was sealed and shaken at 25° C. for 16 hours. The solutions were filtered through a polypropylene frit into a 32-well collection plate. The wells of the top plate were then washed with MeCN (0.5 mL/well), and the top plate removed. The resulting solutions in the collection plate were transferred into vials and concentrated in vacuo using a SpeedVac. The resulting crude carbamates were analyzed using LCMS and those >70% pure were submitted for further analysis.

### Example 129

### LCMS Data for Selected Compounds

LCMS data for selected Pyrimidinone Derivatives is provided below in Table 1, wherein the compound numbers correspond to the compound numbering set forth in the above specification.

TABLE 1
LCMS Data For Selected Pyrimidinone Derivatives

| | LCMS |
|---|---|
| Compound | [M + 1] |
| 1 | 432 |
| 2 | 332 |
| 3 | 490 |
| 4 | 360 |
| 6 | 422 |
| 7 | 500 |
| 8 | 423 |
| 10 | 467 |
| 11 | 475 |
| 12 | 418 |
| 13 | 431 |
| 14 | 438 |
| 15 | 416 |
| 16 | 404 |
| 17 | 390 |
| 18 | 418 |
| 19 | 422 |
| 20 | 428 |
| 21 | 432 |
| 22 | 432 |
| 23 | 434 |
| 24 | 444 |
| 25 | 446 |
| 26 | 452 |
| 27 | 460 |
| 28 | 466 |
| 29 | 466 |
| 30 | 470 |
| 31 | 482 |
| 32 | 486 |
| 33 | 488 |
| 34 | 497 |
| 35 | 500 |
| 36 | 506 |
| 37 | 510 |
| 38 | 511 |
| 39 | 514 |
| 40 | 520 |
| 41 | 530 |
| 42 | 534 |
| 43 | 554 |
| 44 | 554 |
| 45 | 490 |
| 46 | 450 |
| 47 | 520 |
| 48 | 540 |
| 49 | 504 |
| 50 | 490 |
| 51 | 480 |
| 52 | 447 |
| 53 | 544 |
| 54 | 424 |
| 55 | 433 |
| 56 | 507 |
| 57 | 531 |
| 58 | 491 |
| 59 | 508 |
| 60 | 478 |
| 61 | 414 |
| 62 | 442 |
| 63 | 436 |
| 64 | 529 |
| 65 | 400 |
| 66 | 414 |
| 67 | 420 |
| 68 | 426 |
| 69 | 426 |
| 70 | 428 |
| 71 | 430 |
| 72 | 430 |
| 73 | 430 |
| 74 | 437 |
| 75 | 439 |
| 76 | 442 |
| 77 | 442 |
| 78 | 450 |
| 79 | 455 |
| 80 | 456 |
| 81 | 456 |
| 82 | 461 |
| 83 | 464 |
| 84 | 464 |
| 85 | 466 |
| 86 | 466 |
| 87 | 466 |
| 88 | 466 |
| 89 | 468 |
| 90 | 470 |
| 91 | 470 |
| 92 | 476 |
| 93 | 476 |
| 94 | 476 |
| 95 | 478 |
| 96 | 480 |
| 97 | 480 |
| 98 | 482 |
| 99 | 482 |
| 100 | 482 |
| 101 | 492 |
| 102 | 494 |
| 103 | 504 |
| 104 | 504 |
| 105 | 504 |
| 106 | 517 |
| 107 | 517 |
| 108 | 518 |
| 110 | 543 |
| 111 | 503 |
| 112 | 571 |
| 113 | 571 |
| 114 | 418 |
| 115 | 404 |
| 116 | 416 |
| 117 | 428 |
| 118 | 430 |
| 119 | 430 |
| 120 | 430 |
| 121 | 444 |
| 122 | 444 |
| 123 | 446 |
| 124 | 446 |
| 125 | 456 |
| 126 | 456 |
| 127 | 468 |
| 128 | 470 |
| 129 | 480 |
| 130 | 484 |
| 131 | 484 |
| 132 | 484 |
| 133 | 464 |
| 134 | 466 |
| 135 | 480 |
| 136 | 484 |
| 137 | 484 |
| 138 | 493 |
| 139 | 506 |
| 140 | 518 |
| 141 | 534 |
| 142 | 492 |
| 143 | 492 |
| 144 | 524 |
| 145 | 518 |
| 146 | 476 |
| 147 | 550 |
| 148 | 528 |
| 149 | 466 |
| 150 | 512 |
| 151 | 529 |
| 152 | 502 |
| 153 | 519 |
| 155 | 480 |
| 156 | 459 |
| 157 | 543 |
| 158 | 477 |
| 159 | 490 |
| 160 | 548 |
| 161 | 500 |
| 162 | 526 |
| 163 | 520 |
| 164 | 508 |
| 165 | 476 |
| 166 | 502 |
| 167 | 526 |
| 168 | 449 |
| 169 | 473 |
| 170 | 472 |
| 171 | 566 |
| 172 | 522 |
| 173 | 424 |
| 174 | 532 |
| 175 | 492 |
| 176 | 522 |
| 177 | 517 |
| 178 | 664 |
| 179 | 534 |
| 180 | 460 |
| 181 | 470 |
| 182 | 472 |
| 183 | 446 |
| 184 | 470 |
| 185 | 462 |
| 189 | 530 |
| 190 | 430 |
| 191 | 472 |
| 192 | 472 |
| 193 | 474 |
| 194 | 474 |
| 195 | 494 |
| 196 | 544 |
| 197 | 522 |
| 198 | 425 |
| 199 | 357 |
| 200 | 442 |
| 201 | 460 |
| 202 | 502 |
| 203 | 502 |
| 204 | 534 |
| 207 | 510 |
| 208 | 448 |
| 210 | 456 |
| 277 | 598 |
| 212 | 500 |
| 213 | 410 |
| 214 | 508 |
| 215 | 532 |
| 216 | 532 |
| 218 | 466 |
| 219 | 500 |
| 220 | 450 |
| 221 | 396 |
| 222 | 494 |
| 223 | 468 |
| 224 | 438 |
| 225 | 566 |
| 226 | 564 |
| 227 | 382 |
| 228 | 480 |
| 229 | 433 |
| 230 | 433 |
| 231 | 566 |
| 232 | 468 |
| 233 | 418 |
| 234 | 432 |
| 235 | 554 |
| 236 | 550 |
| 237 | 428 |
| 238 | 526 |
| 239 | 466 |
| 240 | 498 |
| 241 | 503 |
| 242 | 503 |
| 243 | 538 |
| 247 | 417 |
| 248 | 417 |
| 249 | 485 |
| 250 | 493 |
| 251 | 317 |
| 252 | 492 |
| 253 | 516 |
| 256 | 463 |
| 257 | 432 |
| 258 | 446 |
| 259 | 460 |
| 260 | 460 |
| 261 | 487 |
| 262 | 531 |
| 263 | 508 |
| 264 | 520 |
| 265 | 447 |
| 267 | 502 |
| 268 | 405 |
| 269 | 374 |
| 270 | 445 |
| 271 | 500 |
| 272 | 520 |
| 273 | 503 |
| 274 | 503 |
| 275 | 499 |
| 276 | 468 |
| 211 | 469 |
| 278 | 556 |
| 279 | 567 |
| 280 | 538 |
| 281 | 479 |
| 282 | 526 |
| 283 | 531 |
| 284 | 565 |
| 285 | 554 |
| 286 | 597 |
| 287 | 535 |
| 288 | 455 |
| 289 | 525 |
| 290 | 555 |
| 291 | 590 |
| 292 | 538 |
| 293 | 574 |
| 294 | 477 |
| 295 | 535 |
| 296 | 515 |
| 297 | 449 |
| 298 | 540 |
| 299 | 547 |
| 300 | 487 |
| 301 | 536 |
| 302 | 520 |
| 303 | 501 |
| 304 | 537 |
| 305 | 553 |
| 306 | 523 |
| 307 | 551 |
| 308 | 534 |
| 309 | 498 |
| 310 | 531 |
| 311 | 552 |
| 312 | 446 |
| 313 | 552 |
| 314 | 556 |
| 315 | 552 |
| 316 | 526 |
| 317 | 526 |
| 318 | 438 |
| 319 | 481 |
| 320 | 477 |
| 321 | 450 |
| 322 | 556 |
| 323 | 554 |
| 324 | 446 |
| 325 | 574 |
| 326 | 519 |
| 327 | 552 |
| 328 | 550 |
| 329 | 570 |
| 330 | 520 |
| 331 | 453 |
| 332 | 428 |
| 333 | 432 |
| 334 | 482 |
| 335 | 442 |
| 336 | 454 |
| 337 | 439 |
| 338 | 453 |
| 339 | 457 |
| 340 | 469 |
| 341 | 481 |
| 342 | 495 |
| 343 | 507 |
| 344 | 413 |
| 345 | 427 |
| 346 | 453 |
| 347 | 467 |
| 348 | 469 |
| 349 | 471 |
| 350 | 493 |
| 351 | 495 |
| 352 | 503 |
| 353 | 515 |
| 354 | 544 |
| 355 | 427 |
| 356 | 455 |
| 357 | 438 |
| 358 | 434 |
| 359 | 414 |
| 360 | 456 |
| 361 | 438 |
| 362 | 516 |
| 363 | 532 |
| 364 | 500 |
| 365 | 386 |
| 366 | 498 |
| 367 | 546 |
| 368 | 414 |
| 369 | 526 |
| 370 | 518 |
| 371 | 412 |
| 372 | 518 |
| 373 | 404 |
| 374 | 432 |
| 375 | 526 |
| 376 | 526 |
| 377 | 504 |
| 378 | 506 |
| 379 | 508 |
| 380 | 508 |
| 381 | 506 |
| 382 | 506 |
| 383 | 410 |
| 384 | 525 |
| 385 | 527 |
| 386 | 448 |
| 387 | 443 |
| 388 | 471 |
| 389 | 466 |
| 390 | 482 |
| 391 | 482 |
| 392 | 478 |
| 393 | 544 |
| 394 | 502 |
| 395 | 480 |
| 396 | 500 |
| 397 | 460 |
| 398 | 544 |
| 399 | 446 |
| 400 | 444 |
| 401 | 466 |
| 402 | 460 |
| 403 | 448 |
| 404 | 540 |
| 405 | 520 |
| 406 | 534 |
| 407 | 444 |
| 408 | 533 |
| 409 | 503 |
| 410 | 426 |
| 411 | 454 |
| 412 | 535 |
| 413 | 513 |
| 414 | 553 |
| 415 | 447 |
| 416 | 569 |
| 417 | 491 |
| 418 | 507 |
| 419 | 535 |
| 420 | 551 |
| 421 | 425 |
| 422 | 481 |
| 423 | 547 |
| 424 | 461 |
| 425 | 583 |
| 426 | 425 |
| 427 | 431 |
| 428 | 547 |
| 429 | 418 |
| 430 | 446 |
| 431 | 415 |
| 432 | 416 |
| 433 | 537 |
| 434 | 519 |
| 435 | 389 |
| 436 | 511 |
| 437 | 415 |
| 438 | 457 |
| 439 | 415 |
| 440 | 500 |
| 441 | 469 |
| 442 | 470 |
| 443 | 563 |
| 444 | 591 |
| 445 | 485 |
| 446 | 455 |
| 447 | 491 |
| 448 | 471 |
| 449 | 510 |
| 450 | 491 |
| 451 | 483 |
| 452 | 559 |
| 453 | 457 |
| 454 | 503 |
| 455 | 451 |
| 456 | 559 |
| 457 | 536 |
| 458 | 545 |
| 459 | 467 |
| 460 | 525 |
| 461 | 483 |
| 462 | 483 |
| 463 | 563 |
| 464 | 563 |
| 465 | 505 |
| 466 | 505 |
| 467 | 437 |
| 468 | 511 |
| 469 | 469 |
| 470 | 505 |
| 471 | 521 |
| 472 | 469 |
| 473 | 541 |
| 474 | 457 |
| 475 | 503 |
| 476 | 521 |
| 477 | 547 |
| 478 | 523 |
| 479 | 573 |
| 480 | 539 |
| 481 | 521 |
| 482 | 505 |
| 483 | 553 |
| 484 | 523 |
| 485 | 443 |
| 486 | 480 |
| 487 | 512 |
| 488 | 531 |
| 489 | 527 |
| 490 | 533 |
| 491 | 411 |
| 492 | 503 |
| 493 | 471 |
| 494 | 505 |
| 495 | 520 |
| 496 | 453 |
| 497 | 545 |
| 498 | 547 |
| 499 | 547 |
| 500 | 547 |
| 501 | 487 |
| 502 | 473 |
| 503 | 567 |
| 504 | 567 |
| 505 | 565 |
| 506 | 567 |
| 507 | 507 |
| 508 | 465 |
| 509 | 595 |
| 510 | 537 |
| 511 | 505 |
| 512 | 505 |
| 513 | 445 |
| 514 | 523 |
| 515 | 523 |
| 516 | 545 |
| 517 | 545 |
| 518 | 439 |
| 519 | 533 |
| 520 | 533 |
| 521 | 493 |
| 522 | 415 |
| 523 | 509 |
| 524 | 459 |
| 525 | 533 |
| 526 | 460 |
| 527 | 446 |
| 528 | 450 |
| 529 | 520 |
| 530 | 554 |
| 531 | 560 |
| 532 | 528 |
| 533 | 401 |
| 534 | 456 |
| 535 | 468 |
| 536 | 559 |
| 537 | 545 |
| 538 | 518 |
| 539 | 550 |
| 540 | 518 |
| 541 | 518 |
| 542 | 518 |
| 543 | 554 |
| 544 | 550 |
| 545 | 490 |
| 546 | 488 |
| 547 | 544 |
| 548 | 490 |
| 549 | 490 |
| 550 | 502 |
| 551 | 504 |
| 552 | 504 |
| 553 | 382 |
| 554 | 520 |
| 555 | 524 |
| 556 | 542 |
| 557 | 522 |
| 558 | 510 |
| 559 | 510 |
| 560 | 504 |
| 561 | 520 |
| 562 | 436 |
| 563 | 410 |
| 564 | 410 |
| 565 | 502 |
| 566 | 504 |
| 567 | 504 |
| 568 | 485 |
| 569 | 504 |
| 570 | 504 |
| 571 | 526 |
| 572 | 526 |
| 573 | 564 |
| 574 | 454 |
| 575 | 488 |
| 576 | 488 |
| 577 | 486 |
| 578 | 540 |
| 579 | 562 |
| 580 | 516 |
| 581 | 510 |
| 582 | 604 |
| 583 | 604 |
| 584 | 446 |

### Example 130 cAMP Assay

The ability of the Pyrimidinone Derivatives to activate GPR119 and stimulate increases in cAMP levels was determined using the LANCE™ cAMP kit (Perkin Elmer). HEK293 cells expressing human GPR119 were maintained in culture flasks at 37° C./5% CO₂ in DMEM containing 10% fetal bovine serum, 100 U/ml Pen/Strep, and 0.5 mg/ml geneticin. The media was changed to Optimem and cells were incubated overnight at 37° C./5% CO₂. The Optimem was then aspirated and the cells were removed from the flasks using room temperature Hank's balanced saline solution (HBSS). The cells were pelleted using centrifugation (1300 rpm, 7 minutes, room temperature), then resuspended in stimulation buffer (HBSS, 0.1% BSA, 5 mM HEPES, 15 μM RO-20) at 2.5×10⁶ cells/mL. Alexa Fluor 647-anti cAMP antibody (1:100) was then added to the cell suspension and incubated for 30 minutes. A representative compound of formula (I) (6 μl at 2× concentration) in stimulation buffer containing 2% DMSO were then added to white 384 well Matrix plates. Cell suspension mix (6 μl) was added to each well and incubated with the compound of formula (I) for 30 minutes. A cAMP standard curve was also created in each assay according to the kit protocol. Standard concentrations of cAMP in stimulation buffer (6 μl) were added to white 384 well plates. Subsequently, 6 μl of 1:100 anti-cAMP antibody was added to each well. Following the 30 minute incubation period, 12 μl of detection mix (included in kit) was added to all wells and incubated for 2-3 hours at room temperature. Fluorescence was detected on the plates using an Envision instrument. The level of cAMP in each well is determined by extrapolation from the cAMP standard curve.

Using this assay, EC₅₀ values for various illustrative Pyrimidinone Derivatives of the present invention were calculated and range from about 50 nM to about 14000 nM.

### Example 131

### Effect of the Pyrimidinone Derivatives in Oral Glucose Tolerance Test

Male C57B1/6NCrl mice (6-8 week old) were fasted overnight and randomly dosed with either vehicle (20% hydroxypropyl-β-cyclodextrin) or a representative compound of the invention (at 3, 10 or 30 mg/kg) via oral gavage (n=8 mice/group). Glucose was administered to the animals 30 minutes post-dosing (3 g/kg p.o.). Blood glucose was measured prior to administration of test compound and glucose, and at 20 minutes after glucose administration using a hand-held glucometer (Ascensia Elite, Bayer).

Using this protocol, the effects of various illustrative Pyrimidinone Derivatives of the present invention were measured and indicate that the Pyrimidinone Derivatives are effective in lowering blood glucose levels after glucose challenge.

### Example 132

### Effects of the Pyrimidinone Derivatives in an Animal Model of Diabetes

Four week old male C57B1/6NCrl mice were used to generate a nongenetic model of type 2 diabetes mellitus as previously described (Metabolism 47(6): 663-668, 1998). Briefly, mice were made insulin resistant by high fat feeding (60% of kcal as fat) and hyperglycemia was induced with a low dose of streptozotocin (100 mg/kg i.p.). Eight weeks after streptozotocin administration, mice were placed into one of 4 groups (n=13/gp) receiving the following treatments: vehicle (20% hydroxypropyl-β-cyclodextrin p.o.), a compound of the invention (30 mg/kg p.o.), glipizide (20 mg/kg p.o.) or exendin-4 (10 ug/kg i.p.). Mice were dosed once daily for 13 consecutive days, and blood glucose was measured daily using a hand held glucometer (Ascensia Elite, Bayer).

Using this protocol, it was demonstrated that an illustrative Pyrimidinone Derivative of the present invention produced a sustained decrease in blood glucose. Accordingly, the compounds of the invention are useful for treating diabetes.

### Uses of the Pyrimidinone Derivatives

The Pyrimidinone Derivatives are useful in human and veterinary medicine for treating or preventing a Condition in a patient. In accordance with the invention, the Pyrimidinone Derivatives can be administered to a patient in need of treatment or prevention of a Condition.

### Treatment of Obesity and Obesity-Related Disorders

The Pyrimidinone Derivatives can be useful for treating obesity or an obesity-related disorder in a patient. Accordingly, in one embodiment, the invention provides methods for treating obesity or an obesity-related disorder in a patient, wherein the method comprises administering to the patient an effective amount of one or more Pyrimidinone Derivatives, or a pharmaceutically acceptable salt, solvate, ester or prodrug thereof.

### Treatment of Diabetes

The Pyrimidinone Derivatives can be useful for treating diabetes in a patient. Accordingly, in one embodiment, the present invention provides a method for treating diabetes in a patient, comprising administering to the patient an effective amount of one or more Pyrimidinone Derivatives.

Examples of diabetes treatable or preventable using the Pyrimidinone Derivatives include, but are not limited to, type I diabetes (insulin-dependent diabetes mellitus), type II diabetes (non-insulin dependent diabetes mellitus), idiopathic type I diabetes (Type Ib), latent autoimmumne diabetes in adults, early-onset type 2 diabetes (EOD), youth-onset atypical diabetes (YOAD), maturity onset diabetes of the young (MODY), malnutrition-related diabetes, gestational diabetes, autoimmune diabetes, insulinopathies, diabetes due to pancreatic disease, diabetes associated with other endocrine diseases (such as Cushing's Syndrome, acromegaly, pheochromocytoma, glucagonoma, primary aldosteronism or somatostatinoma), type A insulin resistance syndrome, type B insulin resistance syndrome, lipatrophic diabetes and diabetes induced by β-cell toxins.

### Treatment of a Diabetic Complication

The Pyrimidinone Derivatives can be useful for treating a diabetic complication in a patient. Accordingly, in one embodiment, the present invention provides a method for treating a diabetic complication in a patient, comprising administering to the patient an effective amount of one or more Pyrimidinone Derivatives.

Examples of diabetic complications treatable or preventable using the Pyrimidinone Derivatives include, but are not limited to, diabetic cataract, glaucoma, retinopathy, aneuropathy (such as diabetic neuropathy, polyneuropathy, mononeuropathy, autonomic neuropathy, microaluminuria and progressive diabetic neuropathyl), nephropathy, gangrene of the feet, immune-complex vasculitis, systemic lupsus erythematosus (SLE), atherosclerotic coronary arterial disease, peripheral arterial disease, nonketotic hyperglycemic-hyperosmolar coma, foot ulcers, joint problems, a skin or mucous membrane complication (such as an infection, a shin spot, a candidal infection or necrobiosis lipoidica diabeticorumobesity), hyperlipidemia, cataract, hypertension, syndrome of insulin resistance, coronary artery disease, a fungal infection, a bacterial infection, and cardiomyopathy.

### Treatment of a Metabolic Disorder

The Pyrimidinone Derivatives can be useful for treating a metabolic disorder in a patient. Accordingly, in one embodiment, the invention provides methods for treating a metabolic disorder in a patient, wherein the method comprises administering to the patient an effective amount of one or more Pyrimidinone Derivatives, or a pharmaceutically acceptable salt, solvate, ester or prodrug thereof.

Examples of metabolic disorders treatable include, but are not limited to, metabolic syndrome (also known as “Syndrome X”), impaired glucose tolerance, impaired fasting glucose, hypercholesterolemia, hyperlipidemia, hypertriglyceridemia, low HDL levels, hypertension, phenylketonuria, post-prandial lipidemia, a glycogen-storage disease, Gaucher's Disease, Tay-Sachs Disease, Niemann-Pick Disease, ketosis and acidosis.

In one embodiment, the metabolic disorder is hypercholesterolemia.

In another embodiment, the metabolic disorder is hyperlipidemia.

In another embodiment, the metabolic disorder is hypertriglyceridemia.

In still another embodiment, the metabolic disorder is metabolic syndrome.

In a further embodiment, the metabolic disorder is low HDL levels.

### Treatment of a Cardiovascular Disease

The Pyrimidinone Derivatives can be useful for treating a cardiovascular disease in a patient. Accordingly, in one embodiment, the invention provides methods for treating a cardiovascular disease in a patient, wherein the method comprises administering to the patient an effective amount of one or more Pyrimidinone Derivatives, or a pharmaceutically acceptable salt, solvate, ester or prodrug thereof.

Examples of cardiovascular diseases treatable or preventable using the present methods include, but are not limited to, atherosclerosis, congestive heart failure, circulatory shock, coronary artery disease, left ventricular hypertrophy, angina pectoris, cardiomyopathy, myocardial infarction and a cardiac arrhythmia.

In one embodiment, the cardiovascular disease is atherosclerosis.

In another embodiment, the cardiovascular disease is congestive heart failure.

### Combination Therapy

In one embodiment, the present invention provides methods for treating a Condition in a patient, the method comprising administering to the patient one or more Pyrimidinone Derivatives, or a pharmaceutically acceptable salt, solvate, ester or prodrug thereof and at least one additional therapeutic agent that is not a Pyrimidinone Derivative, wherein the amounts administered are together effective to treat or prevent a Condition.

Non-limiting examples of additional therapeutic agents useful in the present methods for treating or preventing a Condition include, anti-obesity agents, antidiabetic agents, any agent useful for treating metabolic syndrome, any agent useful for treating a cardiovascular disease, cholesterol biosynthesis inhibitors, cholesterol absorption inhibitors, bile acid sequestrants, probucol derivatives, IBAT inhibitors, nicotinic acid receptor (NAR) agonists, ACAT inhibitors, cholesteryl ester transfer protein (CETP) inhibitors, low-density lipoprotein (LDL) activators, fish oil, water-soluble fibers, plant sterols, plant stanols, fatty acid esters of plant stanols, or any combination of two or more of these additional therapeutic agents.

Non-limiting examples of anti-obesity agents useful in the present methods for treating a Condition include CB1 antagonists or inverse agonists such as rimonabant, neuropeptide Y antagonists, MCR4 agonists, MCH receptor antagonists, histamine H₃ receptor antagonists or inverse agonists, metabolic rate enhancers, nutrient absorption inhibitors, leptin, appetite suppressants and lipase inhibitors.

Non-limiting examples of appetite suppressant agents useful in the present methods for treating or preventing a Condition include cannabinoid receptor 1 (CB₁) antagonists or inverse agonists (e.g., rimonabant); Neuropeptide Y (NPY1, NPY2, NPY4 and NPY5) antagonists; metabotropic glutamate subtype 5 receptor (mGluR5) antagonists (e.g., 2-methyl-6-(phenylethynyl)-pyridine and 3[(2-methyl-1,4-thiazol-4-yl)ethynyl]pyridine); melanin-concentrating hormone receptor (MCH1R and MCH2R) antagonists; melanocortin receptor agonists (e.g., Melanotan-II and Mc4r agonists); serotonin uptake inhibitors (e.g., dexfenfluramine and fluoxetine); serotonin (5HT) transport inhibitors (e.g., paroxetine, fluoxetine, fenfluramine, fluvoxamine, sertaline and imipramine); norepinephrine (NE) transporter inhibitors (e.g., desipramine, talsupram and nomifensine); ghrelin antagonists; leptin or derivatives thereof; opioid antagonists (e.g., nalmefene, 3-methoxynaltrexone, naloxone and nalterxone); orexin antagonists; bombesin receptor subtype 3 (BRS3) agonists; Cholecystokinin-A (CCK-A) agonists; ciliary neurotrophic factor (CNTF) or derivatives thereof (e.g., butabindide and axokine); monoamine reuptake inhibitors (e.g., sibutramine); glucagon-like peptide 1 (GLP-1) agonists; topiramate; and phytopharm compound 57.

Non-limiting examples of metabolic rate enhancers useful in the present methods for treating or preventing a Condition include acetyl-CoA carboxylase-2 (ACC2) inhibitors; beta adrenergic receptor 3 (P3) agonists; diacylglycerol acyltransferase inhibitors (DGAT1 and DGAT2); fatty acid synthase (FAS) inhibitors (e.g., Cerulenin); phosphodiesterase (PDE) inhibitors (e.g., theophylline, pentoxifylline, zaprinast, sildenafil, aminone, milrinone, cilostamide, rolipram and cilomilast); thyroid hormone agonists; uncoupling protein activators (UCP-1, 2 or 3) (e.g., phytanic acid, 4-[(E)-2-(5,6,7,8-tetramethyl-2-naphthalenyl)-1-propenyl]benzoic acid and retinoic acid); acyl-estrogens (e.g., oleoyl-estrone); glucocorticoid antagonists; 11-beta hydroxy steroid dehydrogenase type 1 (11β HSD-1) inhibitors; melanocortin-3 receptor (Mc3r) agonists; and stearoyl-CoA desaturase-1 (SCD-1) compounds.

Non-limiting examples of nutrient absorption inhibitors useful in the present methods for treating or preventing a Condition include lipase inhibitors (e.g., orlistat, lipstatin, tetrahydrolipstatin, teasaponin and diethylumbelliferyl phosphate); fatty acid transporter inhibitors; dicarboxylate transporter inhibitors; glucose transporter inhibitors; and phosphate transporter inhibitors.

Non-limiting examples of cholesterol biosynthesis inhibitors useful in the present methods for treating or preventing a Condition include HMG-CoA reductase inhibitors, squalene synthase inhibitors, squalene epoxidase inhibitors, and mixtures thereof.

Non-limiting examples of cholesterol absorption inhibitors useful in the present methods for treating or preventing a Condition include ezetimibe. In one embodiment, the cholesterol absorption inhibitor is ezetimibe.

HMG-CoA reductase inhibitors useful in the present methods for treating or preventing a Condition include, but are not limited to, statins such as lovastatin, pravastatin, fluvastatin, simvastatin, atorvastatin, cerivastatin, CI-981, resuvastatin, rivastatin, pitavastatin, rosuvastatin or L-659,699 ((E,E)-11-[3′R-(hydroxy-methyl)-4′-oxo-2′R-oxetanyl]-3,5,7R-trimethyl-2,4-undecadienoic acid).

Squalene synthesis inhibitors useful in the present methods for treating or preventing a Condition include, but are not limited to, squalene synthetase inhibitors; squalestatin 1; and squalene epoxidase inhibitors, such as NB-598 ((E)-N-ethyl-N-(6,6-dimethyl-2-hepten-4-ynyl)-3-[(3,3′-bithiophen-5-yl)methoxy]benzene-methanamine hydrochloride).

Bile acid sequestrants useful in the present methods for treating or preventing a Condition include, but are not limited to, cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colestipol (a copolymer of diethylenetriamine and 1-chloro-2,3-epoxypropane, such as COLESTID® tablets which are available from Pharmacia), colesevelam hydrochloride (such as WelChol® Tablets (poly(allylamine hydrochloride) cross-linked with epichlorohydrin and alkylated with 1-bromodecane and (6-bromohexyl)-trimethylammonium bromide) which are available from Sankyo), water soluble derivatives such as 3,3-ioene, N-(cycloalkyl) alkylamines and poliglusam, insoluble quaternized polystyrenes, saponins and mixtures thereof. Suitable inorganic cholesterol sequestrants include bismuth salicylate plus montmorillonite clay, aluminum hydroxide and calcium carbonate antacids.

Probucol derivatives useful in the present methods for treating or preventing a Condition include, but are not limited to, AGI-1067 and others disclosed in U.S. Pat. Nos. 6,121,319 and 6,147,250.

IBAT inhibitors useful in the present methods for treating or preventing a Condition include, but are not limited to, benzothiepines such as therapeutic compounds comprising a 2,3,4,5-tetrahydro-1-benzothiepine 1,1-dioxide structure such as are disclosed in International Publication No. WO 00/38727.

Nicotinic acid receptor agonists useful in the present methods for treating or preventing a Condition include, but are not limited to, those having a pyridine-3-carboxylate structure or a pyrazine-2-carboxylate structure, including acid forms, salts, esters, zwitterions and tautomers, where available. Other examples of nicotinic acid receptor agonists useful in the present methods include nicotinic acid, niceritrol, nicofuranose and acipimox. An example of a suitable nicotinic acid product is NIASPAN® (niacin extended-release tablets) which are available from Kos Pharmaceuticals, Inc. (Cranbury, N.J.). Further nicotinic acid receptor agonists useful in the present methods for treating or preventing a Condition include, but are not limited to, the compounds disclosed in U.S. Patent Publication Nos. 2006/0264489 and 2007/0066630, and U.S. patent application Ser. No. 11/771,538, each of which is incorporated herein by reference.

ACAT inhibitors useful in the present methods for treating or preventing a Condition include, but are not limited to, avasimibe, HL-004, lecimibide and CL-277082 (N-(2,4-difluorophenyl)-N-[[4-(2,2-dimethylpropyl)phenyl]-methyl]-N-heptylurea). See P. Chang et al., “Current, New and Future Treatments in Dyslipidaemia and Atherosclerosis”, Drugs 2000 July; 60(1); 55-93, which is incorporated by reference herein.

CETP inhibitors useful in the present methods for treating or preventing a Condition include, but are not limited to, those disclosed in International Publication No. WO 00/38721 and U.S. Pat. No. 6,147,090, which are incorporated herein by reference.

LDL-receptor activators useful in the present methods for treating or preventing a Condition include, but are not limited to, include HOE-402, an imidazolidinyl-pyrimidine derivative that directly stimulates LDL receptor activity. See M. Huettinger et al., “Hypolipidemic activity of HOE-402 is Mediated by Stimulation of the LDL Receptor Pathway”, Arterioscler. Thromb. 1993; 13:1005-12.

Natural water-soluble fibers useful in the present methods for treating or preventing a Condition include, but are not limited to, psyllium, guar, oat and pectin.

Fatty acid esters of plant stanols useful in the present methods for treating or preventing a Condition include, but are not limited to, the sitostanol ester used in BENECOL® margarine.

Non-limiting examples of antidiabetic agents useful in the present methods for treating a Condition include insulin sensitizers, β-glucosidase inhibitors, DPP-IV inhibitors, insulin secretagogues, hepatic glucose output lowering compounds, antihypertensive agents, sodium glucose uptake transporter 2 (SGLT-2) inhibitors, insulin and insulin-containing compositions, and anti-obesity agents as set forth above.

In one embodiment, the antidiabetic agent is an insulin secretagogue. In one embodiment, the insulin secretagogue is a sulfonylurea.

Non-limiting examples of sulfonylureas useful in the present methods include glipizide, tolbutamide, glyburide, glimepiride, chlorpropamide, acetohexamide, gliamilide, gliclazide, gliquidone, glibenclamide and tolazamide.

In another embodiment, the insulin secretagogue is a meglitinide.

Non-limiting examples of meglitinides useful in the present methods for treating a Condition include repaglinide, mitiglinide, and nateglinide.

In still another embodiment, the insulin secretagogue is GLP-1 or a GLP-1 mimetic.

Non-limiting examples of GLP-1 mimetics useful in the present methods include Byetta-Exanatide, Liraglutinide, CJC-1131 (ConjuChem, Exanatide-LAR (Amylin), BIM-51077 (Ipsen/LaRoche), ZP-10 (Zealand Pharmaceuticals), and compounds disclosed in International Publication No. WO 00/07617.

Other non-limiting examples of insulin secretagogues useful in the present methods include exendin, GIP and secretin.

In another embodiment, the antidiabetic agent is an insulin sensitizer.

Non-limiting examples of insulin sensitizers useful in the present methods include

PPAR activators or agonists, such as troglitazone, rosiglitazone, pioglitazone and englitazone; biguanidines such as metformin and phenformin; PTP-1B inhibitors; and glucokinase activators.

In another embodiment, the antidiabetic agent is a 13-Glucosidase inhibitor.

Non-limiting examples of β-Glucosidase inhibitors useful the present methods include miglitol, acarbose, and voglibose.

In another embodiment, the antidiabetic agent is an hepatic glucose output lowering agent.

Non-limiting examples of hepatic glucose output lowering agents useful in the present methods include Glucophage and Glucophage XR.

In yet another embodiment, the antidiabetic agent is insulin, including all formulations of insulin, such as long acting and short acting forms of insulin.

Non-limiting examples of orally administrable insulin and insulin containing compositions include AL-401 from Autoimmune, and the compositions disclosed in U.S. Pat. Nos. 4,579,730; 4,849,405; 4,963,526; 5,642,868; 5,763,396; 5,824,638; 5,843,866; 6,153,632; 6,191,105; and International Publication No. WO 85/05029, each of which is incorporated herein by reference.

In another embodiment, the antidiabetic agent is a DPP-IV inhibitor.

Non-limiting examples of DPP-IV inhibitors useful in the present methods include sitagliptin, saxagliptin (Januvia™, Merck), denagliptin, vildagliptin (Galvus™, Novartis), alogliptin, alogliptin benzoate, ABT-279 and ABT-341 (Abbott), ALS-2-0426 (Alantos), ARI-2243 (Arisaph), BI-A and BI-B (Boehringer Ingelheim), SYR-322 (Takeda), MP-513 (Mitsubishi), DP-893 (Pfizer), RO-0730699 (Roche) or a combination of sitagliptin/metformin HCl (Janumet™, Merck).

In a further embodiment, the antidiabetic agent is a SGLT-2 inhibitor.

Non-limiting examples of SGLT-2 inhibitors useful in the present methods include dapagliflozin and sergliflozin, AVE2268 (Sanofi-Aventis) and T-1095 (Tanabe Seiyaku).

Non-limiting examples of antihypertensive agents useful in the present methods for treating a Condition include β-blockers and calcium channel blockers (for example diltiazem, verapamil, nifedipine, amlopidine, and mybefradil), ACE inhibitors (for example captopril, lisinopril, enalapril, spirapril, ceranopril, zefenopril, fosinopril, cilazopril, and quinapril), AT-1 receptor antagonists (for example losartan, irbesartan, and valsartan), renin inhibitors and endothelin receptor antagonists (for example sitaxsentan).

In one embodiment, the antidiabetic agent is an agent that slows or blocks the breakdown of starches and certain sugars.

Non-limiting examples of antidiabetic agents that slow or block the breakdown of starches and certain sugars and are suitable for use in the compositions and methods of the present invention include alpha-glucosidase inhibitors and certain peptides for increasing insulin production. Alpha-glucosidase inhibitors help the body to lower blood sugar by delaying the digestion of ingested carbohydrates, thereby resulting in a smaller rise in blood glucose concentration following meals. Non-limiting examples of suitable alpha-glucosidase inhibitors include acarbose; miglitol; camiglibose; certain polyamines as disclosed in WO 01/47528 (incorporated herein by reference); voglibose. Non-limiting examples of suitable peptides for increasing insulin production including amlintide (CAS Reg. No. 122384-88-7 from Amylin; pramlintide, exendin, certain compounds having Glucagon-like peptide-1 (GLP-1) agonistic activity as disclosed in International Publication No. WO 00/07617.

Other specific additional therapeutic agents useful in the present methods for treating or preventing a Condition include, but are not limited to, rimonabant, 2-methyl-6-(phenylethynyl)-pyridine, 3[(2-methyl-1,4-thiazol-4-yl)ethynyl]pyridine, Melanotan-II, dexfenfluramine, fluoxetine, paroxetine, fenfluramine, fluvoxamine, sertaline, imipramine, desipramine, talsupram, nomifensine, leptin, nalmefene, 3-methoxynaltrexone, naloxone, nalterxone, butabindide, axokine, sibutramine, topiramate, phytopharm compound 57, Cerulenin, theophylline, pentoxifylline, zaprinast, sildenafil, aminone, milrinone, cilostamide, rolipram, cilomilast, phytanic acid, 4-[(E)-2-(5,6,7,8-tetramethyl-2-naphthalenyl)-1-propenyl]benzoic acid, retinoic acid, oleoyl-estrone, orlistat, lipstatin, tetrahydrolipstatin, teasaponin and diethylumbelliferyl phosphate.

In one embodiment, the present combination therapies for treating or preventing diabetes comprise administering a compound of formula (I), an antidiabetic agent and/or an antiobesity agent.

In another embodiment, the present combination therapies for treating or preventing diabetes comprise administering a compound of formula (I) and an antidiabetic agent.

In another embodiment, the present combination therapies for treating or preventing diabetes comprise administering a compound of formula (I) and an anti-obesity agent.

In one embodiment, the present combination therapies for treating or preventing obesity comprise administering a compound of formula (I), an antidiabetic agent and/or an antiobesity agent.

In another embodiment, the present combination therapies for treating or preventing obesity comprise administering a compound of formula (I) and an antidiabetic agent.

In another embodiment, the present combination therapies for treating or preventing obesity comprise administering a compound of formula (I) and an anti-obesity agent.

In one embodiment, the present combination therapies for treating or preventing metabolic syndrome comprise administering a compound of formula (I) and one or more additional therapeutic agents selected from: anti-obesity agents, antidiabetic agents, any agent useful for treating metabolic syndrome, any agent useful for treating a cardiovascular disease, cholesterol biosynthesis inhibitors, sterol absorption inhibitors, bile acid sequestrants, probucol derivatives, IBAT inhibitors, nicotinic acid receptor (NAR) agonists, ACAT inhibitors, cholesteryl ester transfer protein (CETP) inhibitors, low-density lipoprotein (LDL) activators, fish oil, water-soluble fibers, plant sterols, plant stanols and fatty acid esters of plant stanols.

In one embodiment, the additional therapeutic agent is a cholesterol biosynthesis inhibitor. In another embodiment, the cholesterol biosynthesis inhibitor is a squalene synthetase inhibitor. In another embodiment, the cholesterol biosynthesis inhibitor is a squalene epoxidase inhibitor. In still another embodiment, the cholesterol biosynthesis inhibitor is an HMG-CoA reductase inhibitor. In another embodiment, the HMG-CoA reductase inhibitor is a statin. In yet another embodiment, the statin is lovastatin, pravastatin, simvastatin or atorvastatin.

In one embodiment, the additional therapeutic agent is a cholesterol absorption inhibitor. In another embodiment, the cholesterol absorption inhibitor is ezetimibe.

In one embodiment, the additional therapeutic agent comprises a cholesterol absorption inhibitor and a cholesterol biosynthesis inhibitor. In another embodiment, the additional therapeutic agent comprises a cholesterol absorption inhibitor and a statin. In another embodiment, the additional therapeutic agent comprises ezetimibe and a statin. In another embodiment, the additional therapeutic agent comprises ezetimibe and simvastatin.

In one embodiment, the present combination therapies for treating or preventing metabolic syndrome comprise administering a compound of formula (I), an antidiabetic agent and/or an antiobesity agent.

In another embodiment, the present combination therapies for treating or preventing metabolic syndrome comprise administering a compound of formula (I) and an antidiabetic agent.

In another embodiment, the present combination therapies for treating or preventing metabolic syndrome comprise administering a compound of formula (I) and an anti-obesity agent.

In one embodiment, the present combination therapies for treating or preventing a cardiovascular disease comprise administering one or more compounds of formula (I), and an additional agent useful for treating or preventing a cardiovascular disease.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts).

In one embodiment, the one or more Pyrimidinone Derivatives are administered during a time when the additional therapeutic agent(s) exert their prophylactic or therapeutic effect, or vice versa.

In another embodiment, the one or more Pyrimidinone Derivatives and the additional therapeutic agent(s) are administered in doses commonly employed when such agents are used as monotherapy for treating a Condition.

In another embodiment, the one or more Pyrimidinone Derivatives and the additional therapeutic agent(s) are administered in doses lower than the doses commonly employed when such agents are used as monotherapy for treating a Condition.

In still another embodiment, the one or more Pyrimidinone Derivatives and the additional therapeutic agent(s) act synergistically and are administered in doses lower than the doses commonly employed when such agents are used as monotherapy for treating a Condition.

In one embodiment, the one or more Pyrimidinone Derivatives and the additional therapeutic agent(s) are present in the same composition. In one embodiment, this composition is suitable for oral administration. In another embodiment, this composition is suitable for intravenous administration.

The one or more Pyrimidinone Derivatives and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

In one embodiment, the administration of one or more Pyrimidinone Derivatives and the additional therapeutic agent(s) may inhibit the resistance of a Condition to these agents.

In one embodiment, when the patient is treated for diabetes or a diabetic complication, the additional therapeutic agent is an antidiabetic agent which is not a Pyrimidinone Derivative. In another embodiment, the additional therapeutic agent is an agent useful for reducing any potential side effect of a Pyrimidinone Derivative. Such potential side effects include, but are not limited to, nausea, vomiting, headache, fever, lethargy, muscle aches, diarrhea, general pain, and pain at an injection site.

In one embodiment, the additional therapeutic agent is used at its known therapeutically effective dose. In another embodiment, the additional therapeutic agent is used at its normally prescribed dosage. In another embodiment, the additional therapeutic agent is used at less than its normally prescribed dosage or its known therapeutically effective dose.

The doses and dosage regimen of the other agents used in the combination therapies of the present invention for the treatment or prevention of a Condition can be determined by the attending clinician, taking into consideration the approved doses and dosage regimen in the package insert; the age, sex and general health of the patient; and the type and severity of the viral infection or related disease or disorder. When administered in combination, the Pyrimidinone Derivative(s) and the other agent(s) for treating diseases or conditions listed above can be administered simultaneously or sequentially. This particularly useful when the components of the combination are given on different dosing schedules, e.g., one component is administered once daily and another every six hours, or when the preferred pharmaceutical compositions are different, e.g. one is a tablet and one is a capsule. A kit comprising the separate dosage forms is therefore advantageous.

Generally, a total daily dosage of the one or more Pyrimidinone Derivatives and the additional therapeutic agent(s) can when administered as combination therapy, range from about 0.1 to about 2000 mg per day, although variations will necessarily occur depending on the target of the therapy, the patient and the route of administration. In one embodiment, the dosage is from about 0.2 to about 100 mg/day, administered in a single dose or in 2-4 divided doses. In another embodiment, the dosage is from about 1 to about 500 mg/day, administered in a single dose or in 2-4 divided doses. In another embodiment, the dosage is from about 1 to about 200 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In a further embodiment, the dosage is from about 1 to about 20 mg/day, administered in a single dose or in 2-4 divided doses.

### Compositions and Administration

In one embodiment, the invention provides compositions comprising an effective amount of one or more compounds of formula (I) or a pharmaceutically acceptable salt, solvate, ester or prodrug thereof, and a pharmaceutically acceptable carrier.

For preparing pharmaceutical compositions from the compounds of formula (I), inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

In one embodiment, the Pyrimidinone Derivative is administered orally.

In one embodiment, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation is from about 0.1 to about 2000 mg. Variations will necessarily occur depending on the target of the therapy, the patient and the route of administration. In one embodiment, the unit dose dosage is from about 0.2 to about 1000 mg. In another embodiment, the unit dose dosage is from about 1 to about 500 mg. In another embodiment, the unit dose dosage is from about 1 to about 100 mg/day. In still another embodiment, the unit dose dosage is from about 1 to about 50 mg. In yet another embodiment, the unit dose dosage is from about 1 to about 10 mg.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 300 mg/day, preferably 1 mg/day to 75 mg/day, in two to four divided doses.

When the invention comprises a combination of one or more Pyrimidinone Derivatives and an additional therapeutic agent, the two active components may be co-administered simultaneously or sequentially, or a single pharmaceutical composition comprising one or more Pyrimidinone Derivatives and an additional therapeutic agent in a pharmaceutically acceptable carrier can be administered. The components of the combination can be administered individually or together in any conventional dosage form such as capsule, tablet, powder, cachet, suspension, solution, suppository, nasal spray, etc. The dosage of the additional therapeutic agent can be determined from published material, and may range from about 1 to about 1000 mg per dose. In one embodiment, when used in combination, the dosage levels of the individual components are lower than the recommended individual dosages because of the advantageous effect of the combination.

In one embodiment, the components of a combination therapy regime are to be administered simultaneously, they can be administered in a single composition with a pharmaceutically acceptable carrier.

In another embodiment, when the components of a combination therapy regime are to be administered separately or sequentially, they can be administered in separate compositions, each containing a pharmaceutically acceptable carrier.

The components of the combination therapy can be administered individually or together in any conventional dosage form such as capsule, tablet, powder, cachet, suspension, solution, suppository, nasal spray, etc.

### Kits

In one aspect, the present invention provides a kit comprising an effective amount of one or more Compounds of Formula (I), or a pharmaceutically acceptable salt or solvate of the compound and a pharmaceutically acceptable carrier, vehicle or diluent.

In another aspect the present invention provides a kit comprising an amount of one or more Pyrimidinone Derivatives, or a pharmaceutically acceptable salt or solvate of the compound and an amount of at least one additional therapeutic agent listed above, wherein the combined amounts are effective for treating or preventing diabetes, a diabetic complication impaired glucose tolerance or impaired fasting glucose in a patient.

When the components of a combination therapy regime are to be administered in more than one composition, they can be provided in a kit comprising in a single package, one or more containers, each comprising one or more Pyrimidinone Derivatives in a pharmaceutically acceptable carrier, and a separate container comprising an additional therapeutic agent in a pharmaceutically acceptable carrier, with the active components of each composition being present in amounts such that the combination is therapeutically effective.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

1. A compound having the formula: 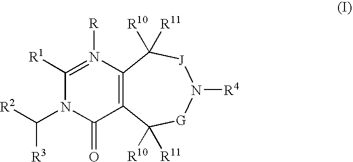
or a pharmaceutically acceptable salt, solvate, ester or prodrug thereof,
wherein
J is a single bond, —C(R¹⁰)(R¹¹)— or —C(R¹⁰)(R¹¹)—C(e)(R¹¹)—;
G is a single bond, —C(R¹⁰)(R¹¹)— or —C(R¹⁰)(R¹¹)—C(e)(R¹¹)—, such that: (i) if J is —C(R¹⁰)(R¹¹)—, then G is —C(R¹⁰)(R¹¹)— or —C(R¹⁰)(R¹¹)—C(R¹⁰)(R¹¹)—; and (ii) if J is —C(R¹⁰)(R¹¹)—C(R¹⁰)(R¹¹)—, then G is a single bond;
R is absent or R is oxygen, such that when R is oxygen, this is understood to represent the N-oxide form of the nitrogen atom to which R is attached;
R¹ is H, alkyl, haloalkyl, —N(R⁹)₂, —SR⁹, —S(O)_{q}N(R⁶)₂, —S(O)ₚR⁷, —OR⁹, -(alkylene)ₙ-aryl, -(alkylene)-cycloalkyl, -(alkylene)ₙ-cycloalkenyl, -(alkylene)ₙ-heterocycloalkyl, -(alkylene)-heteroaryl, -(alkylene)-heterocycloalkenyl, —C(O)-aryl, —C(O)-alkyl, -alkylene-O-aryl, -alkylene-O-alkyl or —C(O)NH₂, wherein an aryl, cycloalkyl, cycloalkenyl, heterocycloalkyl, heterocycloalkenyl or heteroaryl group can be optionally substituted with up to 3 substituents, which can be the same or different, and are selected from alkyl, haloalkyl, hydroxyalkyl, aryl, halo, —OH, —O-haloalkyl, -alkylene-O-alkyl, —S(O)ₚR⁷, —CN, —N(R⁶)₂, —C(O)R⁵, —C(O)OR⁵, —C(O)N(R⁶)₂, —NHC(O)R⁵, —NHS(O)_{q}R⁷ and —S(O)_{q}N(R⁶)₂;
R² is alkyl, -alkenyl, -alkynyl, -(alkylene)-aryl, -(alkylene)-cycloalkyl, -(alkylene)ₙ-cycloalkenyl, -(alkylene)-heterocycloalkyl, -(alkylene)ₙ-heteroaryl, -(alkylene)ₙ-heterocycloalkenyl, -(alkylene)ₙ-OC(O)N(R⁶)₂, hydroxyalkyl, haloalkyl, -alkylene-alkenyl, —C(O)-aryl, —C(O)-alkyl, —C(O)-heterocycloalkyl, —C(O)-heteroaryl, -alkylene-O-aryl, -alkylene-O-alkyl, -alkylene-O-haloalkyl, —C(O)OR⁵, or —C(O)N(R⁶)₂, wherein an aryl, cycloalkyl, cycloalkenyl, heterocycloalkyl, heterocycloalkenyl or heteroaryl group can be optionally substituted with up to 3 substituents, which can be the same or different, and are selected from alkyl, haloalkyl, hydroxyalkyl, aryl, halo, —OH, —O-haloalkyl, —O-alkyl, -alkylene-O-alkyl, —Si(alkyl)₃, —S(O)ₚR⁷, —CN, —N(R⁶)₂, —C(O)R⁵, —C(O)OR⁵, —C(O)N(R⁶)₂, —NHC(O)R⁵, —NHS(O)_{q}R⁷ and —S(O)_{q}N(R⁶)₂, and wherein a cycloalkyl group may form a spirocycle with a heterocycloalkyl group or with another cycloalkyl group, or R² and R³ and the carbon atom to which they are both attached, combine to form an aryl, cycloalkyl, cycloalkenyl, heterocycloalkyl, heterocycloalkenyl or heteroaryl group, wherein any of these groups is unsubstituted or substituted with up to 3 substituents, which can be the same or different, and which are selected from alkyl, haloalkyl, hydroxyalkyl, halo, —OH, —O-haloalkyl, —O-alkyl, —O-aryl, -alkylene-O-alkyl, —CN, —N(R⁶)₂, —C(O)R⁵, —C(O)OR⁵, —C(O)N(R⁶)₂, —NHC(O)R⁵, —NHS(O)_{q}R⁷, —S(O)ₚR⁷ and —S(O)_{q}N(R⁶)₂;
R³ is alkyl, -(alkylene)-aryl, -(alkylene)ₙ-cycloalkyl, -(alkylene)ₙ-cycloalkenyl, -(alkylene)ₙ-heterocycloalkyl, -(alkylene)ₙ-heteroaryl, -(alkylene)ₙ-heterocycloalkenyl, —C(O)-aryl, —C(O)-alkyl, -alkylene-O-aryl, -alkylene-O-alkyl, —C(O)OR⁵, or —C(O)N(R⁶)₂, wherein an aryl, cycloalkyl, cycloalkenyl, heterocycloalkyl, heterocycloalkenyl or heteroaryl group can be optionally substituted with up to 3 substituents, which can be the same or different, and are selected from alkyl, haloalkyl, hydroxyalkyl, aryl, halo, —OH, —O-haloalkyl, —O-alkyl, -alkylene-O-alkyl, —S(O)ₚR⁷, —CN, —N(R⁶)₂, —C(O)R⁵, —C(O)OR⁵, —C(O)N(R⁶)₂, —NHC(O)R⁵, —NHS(O)_{q}R⁷ and —S(O)_{q}N(R⁶)₂, or R² and R³ and the carbon atom to which they are both attached, combine to form an aryl, cycloalkyl, cycloalkenyl, heterocycloalkyl, heterocycloalkenyl or heteroaryl group, wherein any of these groups is unsubstituted or substituted with up to 3 substituents, which can be the same or different, and which are selected from alkyl, haloalkyl, hydroxyalkyl, halo, —OH, —O-haloalkyl, —O-alkyl, —O-aryl, -alkylene-O-alkyl, —CN, —N(R⁶)₂, —C(O)R⁵, —C(O)OR⁵, —C(O)N(R⁶)₂, —NHC(O)R⁵, —NHS(O)_{q}R⁷, —S(O)ₚR⁷ and —S(O)_{q}N(R⁶)₂;
R⁴ is H, alkyl, alkenyl, —C(O)R⁵, —S(O)_{q}R⁷, -alkylene-O-alkyl, -alkylene-O-aryl, -alkylene-S-alkyl, -alkylene-S-aryl, -alkylene-NH-alkyl, -alkylene-NH-aryl, -alkylene-NC(O)O-alkyl, —C(O)OR⁵, —C(O)N(R⁶)₂, —C(O)NH—OR⁹, -alkylene-O-haloalkyl, -(alkylene)ₙ-aryl, -(alkylene)ₙ-cycloalkyl, -(alkylene)ₙ-cycloalkenyl, -(alkylene)ₙ-heterocycloalkyl, -(alkylene)ₙ-heterocycloalkenyl, -(alkylene)ₙ-heteroaryl, -(alkenylene)ₙ-aryl, -(alkenylene)ₙ-cycloalkyl, -(alkenylene)ₙ-cycloalkenyl, -(alkenylene)ₙ-heterocycloalkyl, -(alkenylene)ₙ-heterocycloalkenyl or -(alkenylene)ₙ-heteroaryl, wherein any alkylene or alkenylene group can be optionally substituted with one or more substituents independently selected from alkyl, haloalkyl, hydroxyalkyl, —O-alkyl, aryl, cycloalkyl, cycloalkenyl, heterocycloalkyl, heterocycloalkenyl or heteroaryl, and wherein any aryl, cycloalkyl, cycloalkenyl, heterocycloalkyl, heterocycloalkenyl or heteroaryl group can be optionally substituted with up to 3 substituents, which can be the same or different, and are selected from: alkyl, aryl, heterocycloalkyl, heteroaryl, -alkylene-O-alkylene-Si(alkyl)₃, —NH₂, —NH-alkyl, —N(alkyl)₂, -hydroxyalkyl, —S(O)ₚR⁷, —O-alkyl, —O-aryl, —C(O)O-alkyl, —C(O)O-haloalkyl, halo, —NO₂, —CN, heteroaryl, haloalkyl, —O-haloalkyl, and -(alkynylene)ₙ-aryl;
R⁵ is alkyl, alkenyl, alkynyl, haloalkyl, haloalkenyl, -alkylene-O-aryl, -alkylene-S-aryl, -alkylene-N(R⁸)C(O)O-alkyl, -(alkylene)-aryl, -(alkylene)-cycloalkyl, -(alkylene)-cycloalkenyl, -(alkylene)ₙ-heterocycloalkyl, -(alkylene)ₙ-heterocycloalkenyl or -(alkylene)-heteroaryl, wherein a cycloalkyl group may form a spirocycle with a heterocycloalkyl group or with another cycloalkyl group, and wherein an aryl, alkenyl, alkynyl, cycloalkyl, cycloalkenyl, heterocycloalkyl, heterocycloalkenyl or heteroaryl group can be unsubstituted or substituted with up to 4 substituents, which can be the same or different, and are selected from alkyl, haloalkyl, hydroxyalkyl, halo, —OH, —O-haloalkyl, —O-alkyl, —O-aryl, —S-haloalkyl, -alkylene-O-alkyl, —CN, —N(R⁹)₂, —C(O)H, —C(O)R⁹, —C(O)OR⁹, —C(O)N(R⁹)₂, —NHC(O)R⁹, —NHS(O)_{q}R⁹, —S(O)ₚR⁹ and —S(O)_{q}N(R⁹)₂;
each occurrence of R⁶ is independently H, alkyl, -(alkylene)-aryl, -(alkylene)ₙ-cycloalkyl, -(alkylene)-cycloalkenyl, -(alkylene)ₙ-heterocycloalkyl, -(alkylene)ₙ-heterocycloalkenyl or -(alkylene)ₙ-heteroaryl, wherein any of the above groups, excluding H, can be unsubstituted or substituted with from 1 to 3 substituents, which can be the same or different, and which are selected from alkyl, haloalkyl, hydroxyalkyl, halo, —OH, —O-haloalkyl, —O-alkyl, —O-aryl, -alkylene-O-alkyl, —CN, —N(R⁹)₂, —C(O)H, —C(O)R⁹, —C(O)OR⁹, —C(O)N(R⁹)—NHC(O)R⁹, —NHS(O)_{q}R⁹, —S(O)ₚR⁹ and —S(O)_{q}N(R⁹)₂;
each occurrence of R⁷ is independently alkyl, aryl, heterocycloalkyl, heteroaryl or cycloalkyl, wherein any of the above groups, can be unsubstituted or substituted with from 1 to 3 substituents, which can be the same or different, and which are selected from alkyl, haloalkyl, hydroxyalkyl, halo, —OH, —O-haloalkyl, —O-alkyl, —O-aryl, -alkylene-O-alkyl, —CN, —N(R⁹)₂, —C(O)H, —C(O)R⁹, —C(O)OR⁹, —C(O)N(R⁹)₂, —NHC(O)R⁹, —NHS(O)_{q}R⁹, —S(O)ₚ, R⁹ and —S(O)_{q}N(R⁹)₂;
each occurrence of R⁸ is independently H or alkyl;
each occurrence of R⁹ is independently H, alkyl, -(alkylene)-aryl, heterocycloalkyl, heteroaryl or cycloalkyl;
each occurrence of R¹⁰ is independently H, alkyl, -(alkylene)ₙ-aryl, heterocycloalkyl, heteroaryl or cycloalkyl;
each occurrence of R¹¹ is independently H, alkyl, -(alkylene)-aryl, heterocycloalkyl, heteroaryl or cycloalkyl;
each occurrence of n is independently 0 or 1;
each occurrence of p is independently 0, 1 or 2; and
each occurrence of q is independently 1 or 2.

2. The compound of claim 1, wherein J is a single bond and G is —C(R¹⁰)(R¹¹)—.

3. The compound of claim 2, wherein G is —CH₂—.

4. The compound of claim 1, wherein R¹ is alkyl.

5. The compound of claim 4, wherein R¹ is methyl.

6. The compound of claim 1, wherein R¹ is fluoromethyl.

7. The compound of claim 1, wherein R¹ is —N(R⁹)₂.

8. The compound of claim 7, wherein R¹ is —NH₂.

9. The compound of claim 2, wherein R² and R³ are each independently aryl, heteroaryl or cycloalkyl.

10. The compound of claim 9, wherein R² is phenyl, pyridyl fluorophenyl, 3-fluorophenyl, cyclobutyl, benzyl or 3,4-difluorophenyl.

11. The compound of claim 9, wherein R³ is phenyl, pyridyl, 4-fluorophenyl, 3-fluorophenyl, cyclopropylmethyl, ethoxymethyl, trifluoroethoxymethyl, n-butyl, cyclopropyl, cyclobutyl, cyclopentyl or cyclohexyl.

12. The compound of claim 11, wherein R² and R³ are each phenyl.

13. The compound of claim 11, wherein R² and R³ are each 4-fluorophenyl.

14. The compound of claim 11, wherein R² is phenyl and R³ is 4-fluorophenyl.

15. The compound of claim 9, wherein R⁴ is —C(O)R⁵ or —C(O)OR⁵.

16. The compound of claim 2 wherein R² is phenyl or pyridyl; R³ is phenyl, pyridyl, cyclopropyl, cyclobutyl, cyclopentyl or cyclohexyl; and R⁴ is C(O)R⁵ or —C(O)OR⁵.

17. The compound of claim 16, wherein R² is phenyl or pyridyl.

18. The compound of claim 17, wherein R³ is phenyl.

19. The compound of claim 16, wherein R¹ is methyl.

20. The compound of claim 18, wherein R¹ is methyl.

21. The compound of claim 16, wherein R⁴ is —C(O)OR⁵.

22. The compound of claim 21, wherein R⁵ is alkyl, haloalkyl, aryl, -alkylene-aryl, heteroaryl, cycloalkyl or —CH₂-cycloalkyl.

23. The compound of claim 22, wherein R⁵ is —CH₂CH(CF₃)₂.

24. The compound of claim 9, wherein R⁴ is heteroaryl.

25. The compound of claim 24, wherein R⁴ is 1,2,4-oxadiazolyl.

26. The compound of claim 22, wherein R⁵ is haloalkyl.

27. The compound of claim 16, wherein R¹ is —NH₂.

28. The compound of claim 18, wherein R¹ is —NH₂.

29. The compound of claim 27, wherein R⁴ is —C(O)OR³.

30. The compound of claim 29, wherein R⁵ is alkyl, haloalkyl, aryl, -alkylene-aryl, heteroaryl, cycloalkyl or —CH₂-cycloalkyl.

31. The compound of claim 22, wherein R¹ is methyl or —NH₂, R² is phenyl, R³ is phenyl, or cycloalkyl, R⁴ is —C(O)OR⁵ and R⁵ is -tert-butyl, —CH₂CCl₃, —C(CH₃)₂CCl₃, —CH₂CF₂CF₃, —CH(CF₃)₂, —CH₂CH(CF₃)₂,

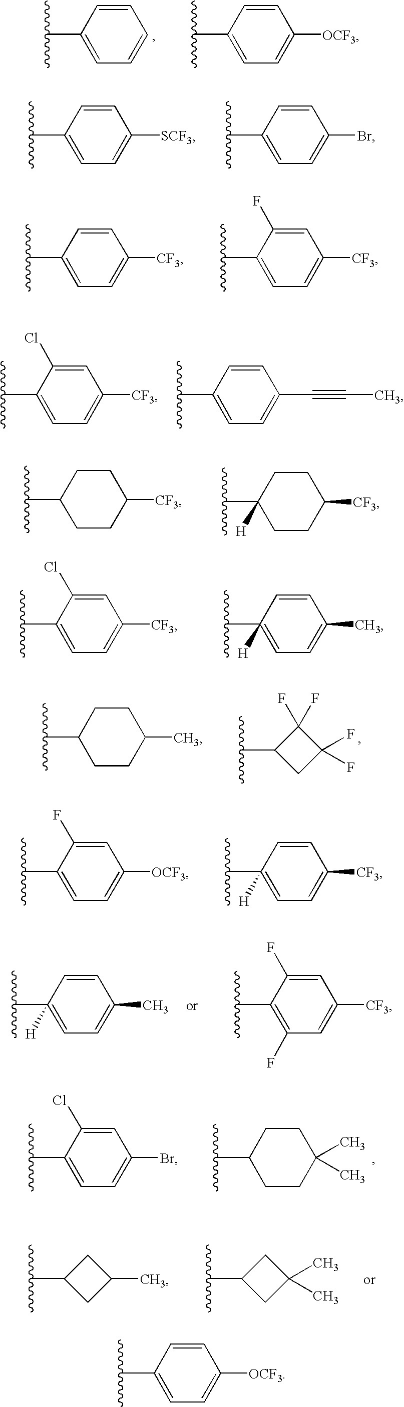

32. The compound of claim 21, wherein R² and R³ are each phenyl.

33. The compound of claim 22, wherein R² and R³ are each 4-fluorophenyl.

34. The compound of claim 22, wherein R² is phenyl and R³ is 4-fluorophenyl.

35. A compound having the structure: 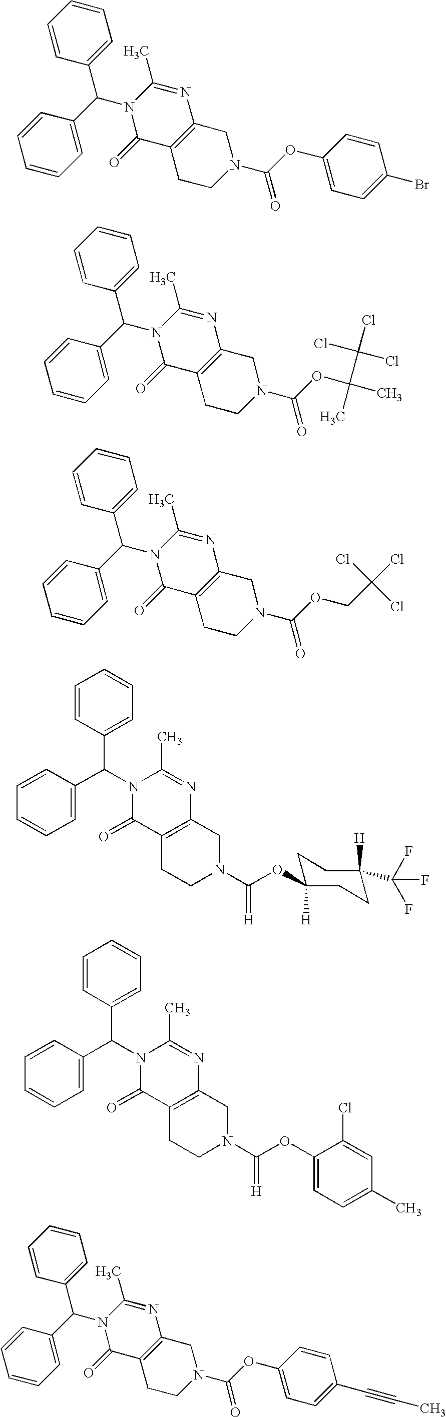 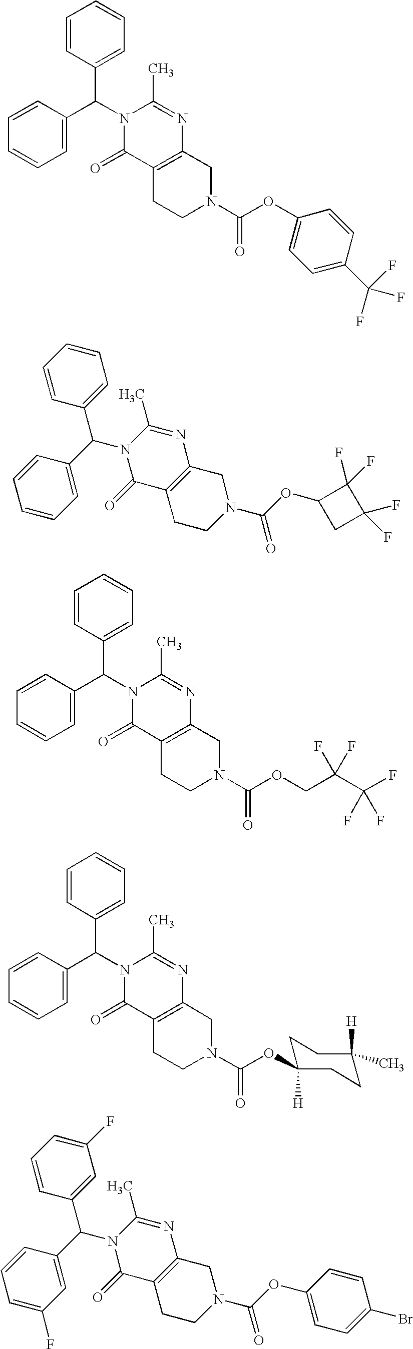 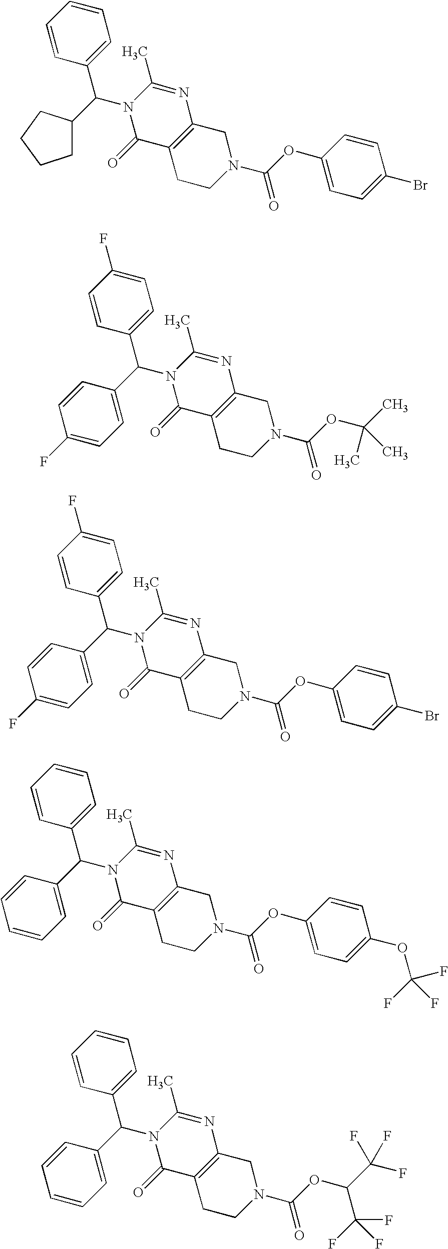 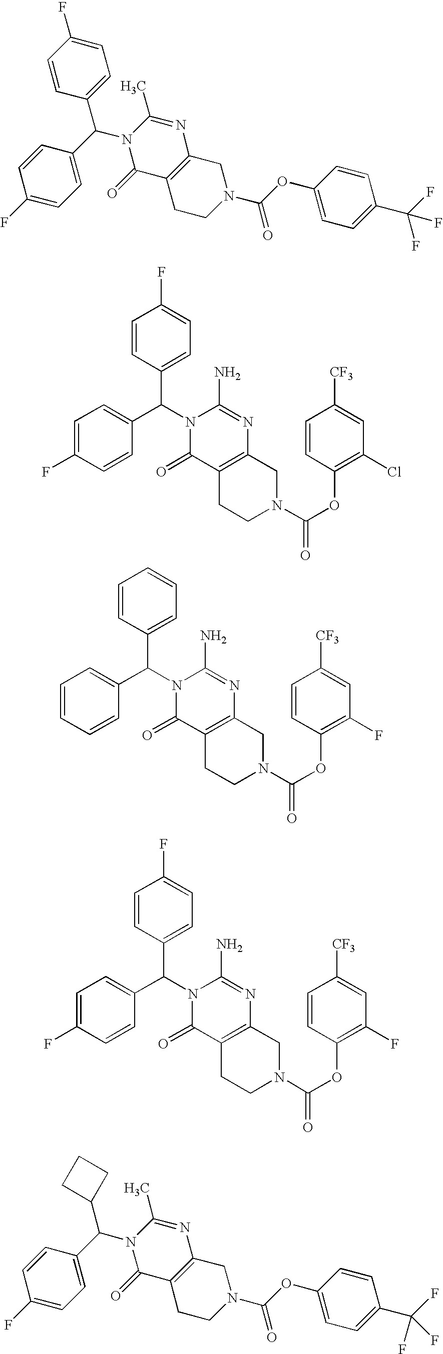 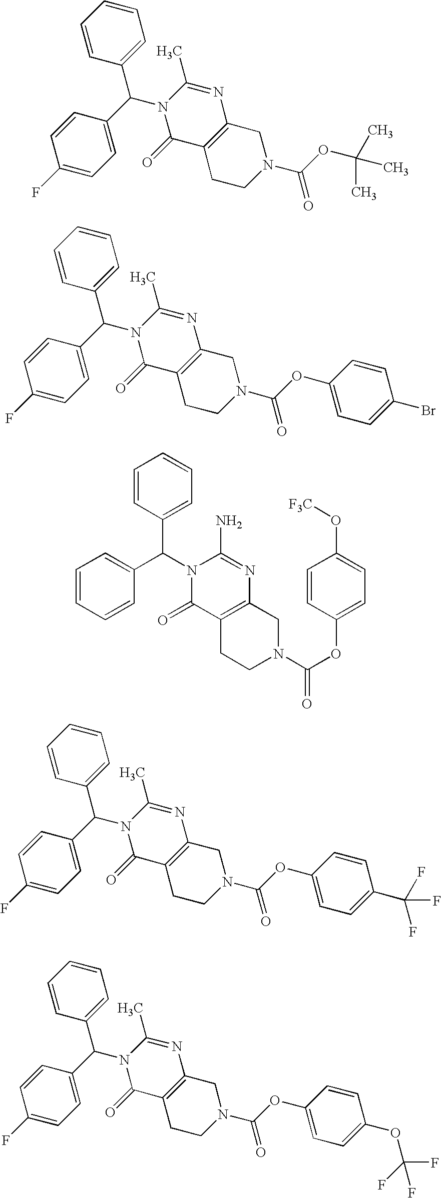 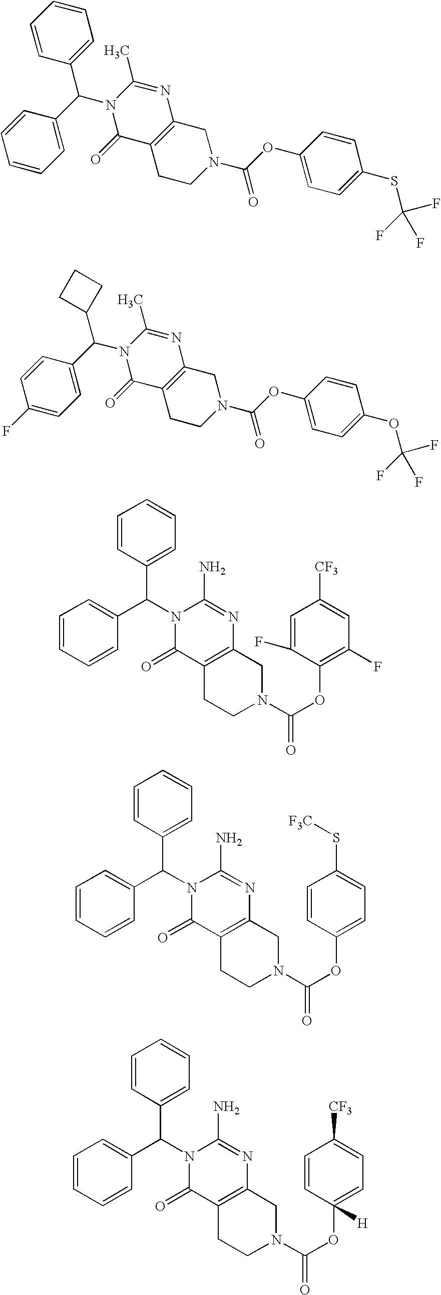 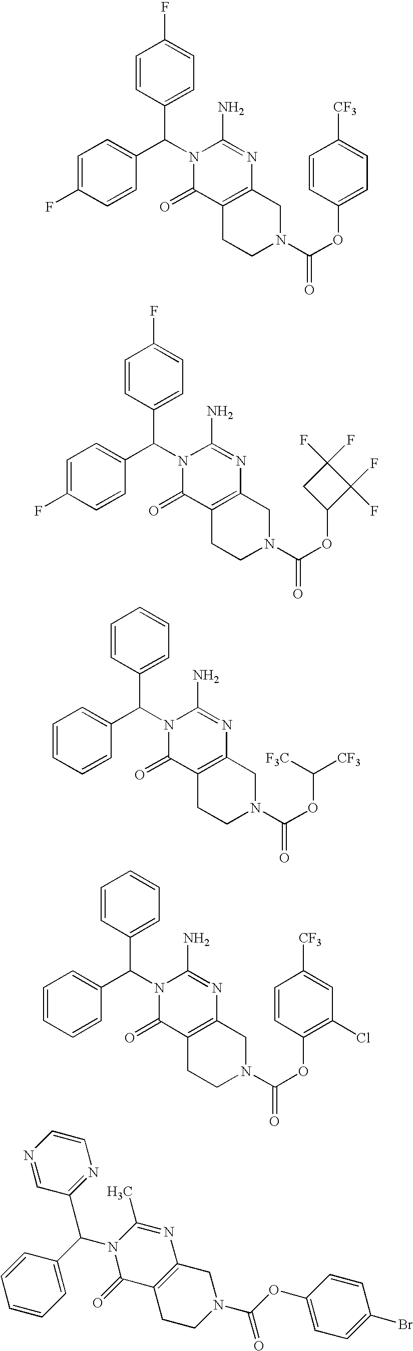 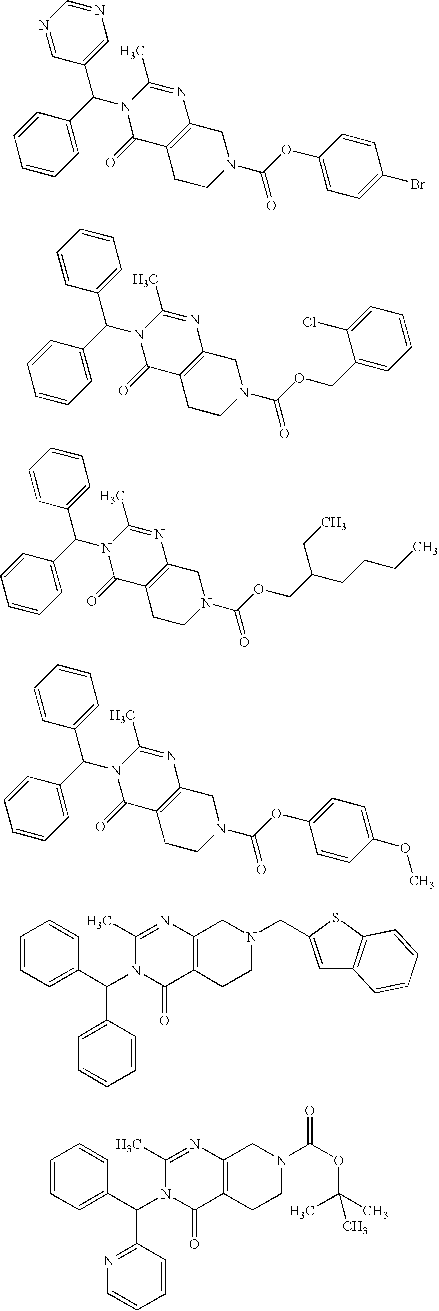 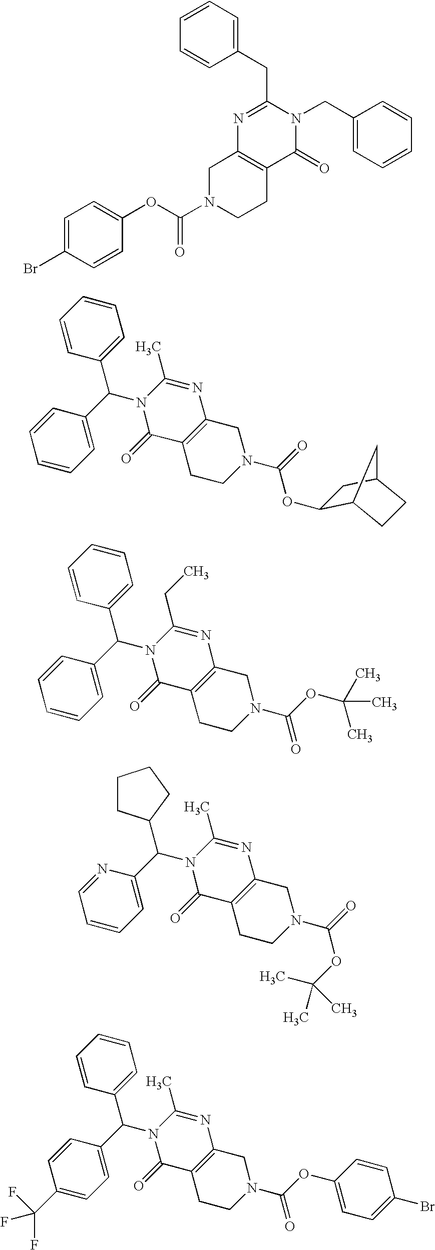 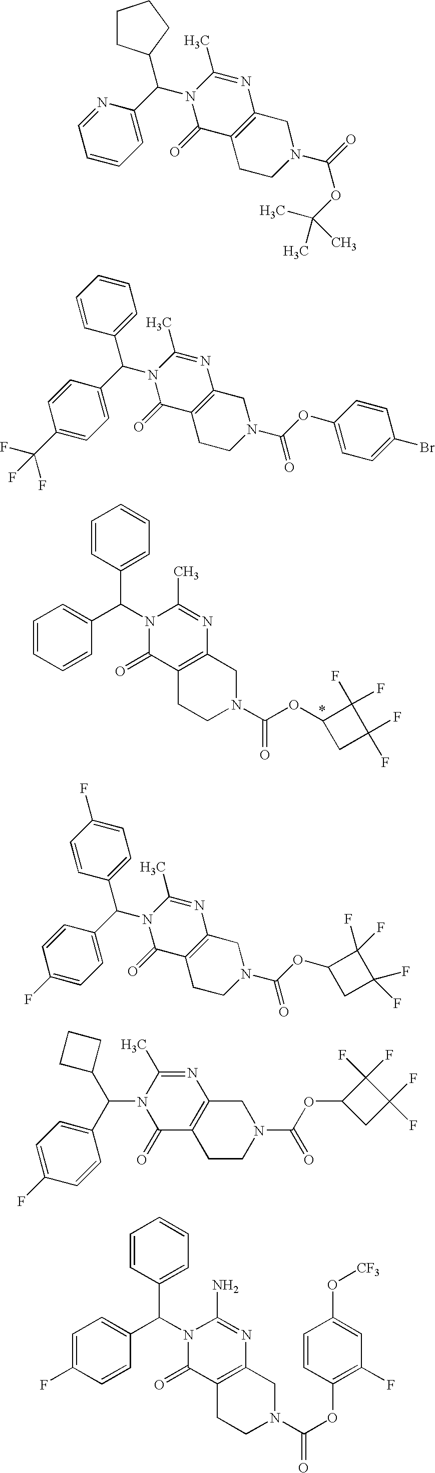 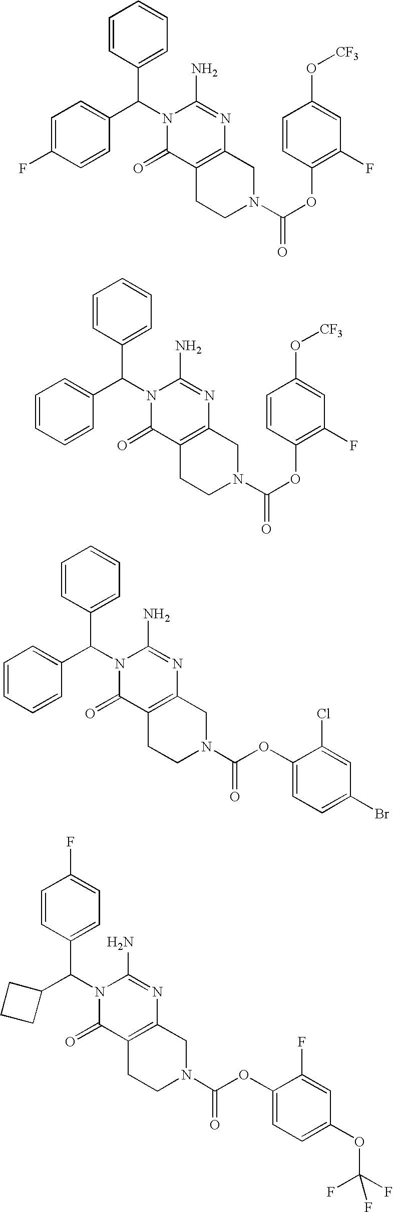 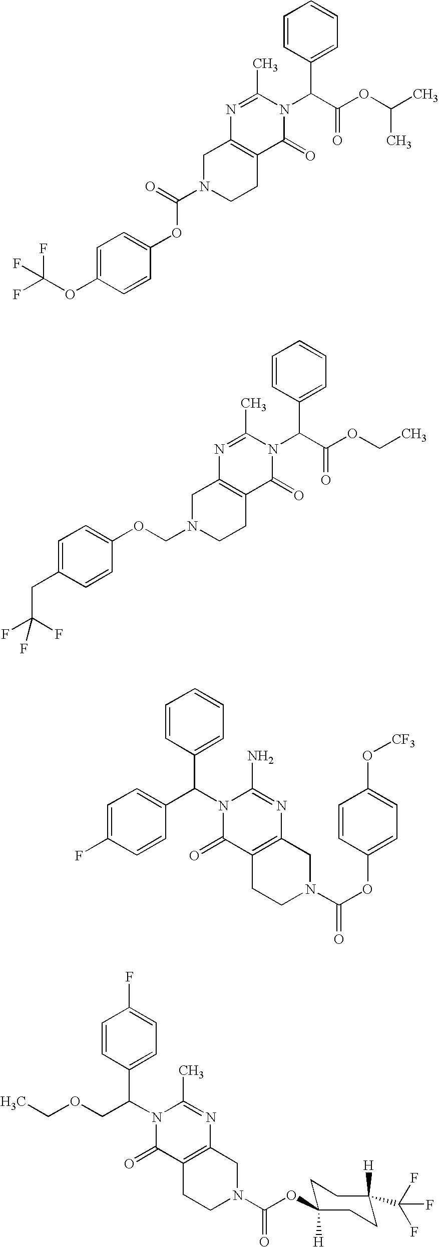 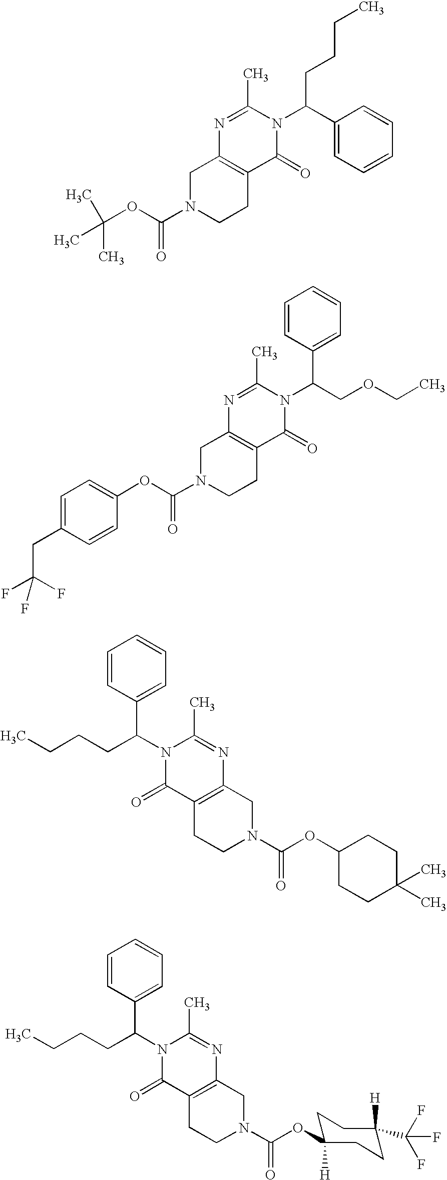 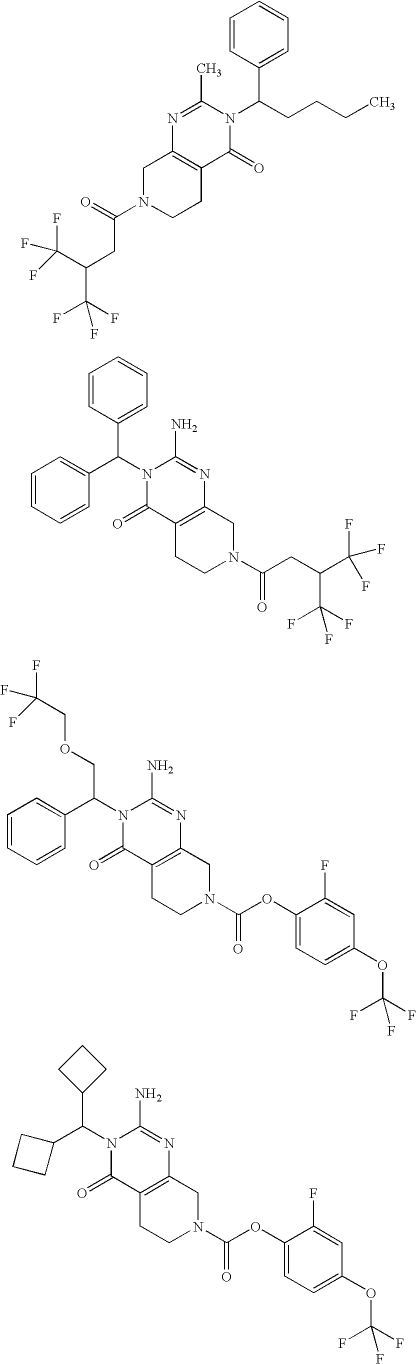 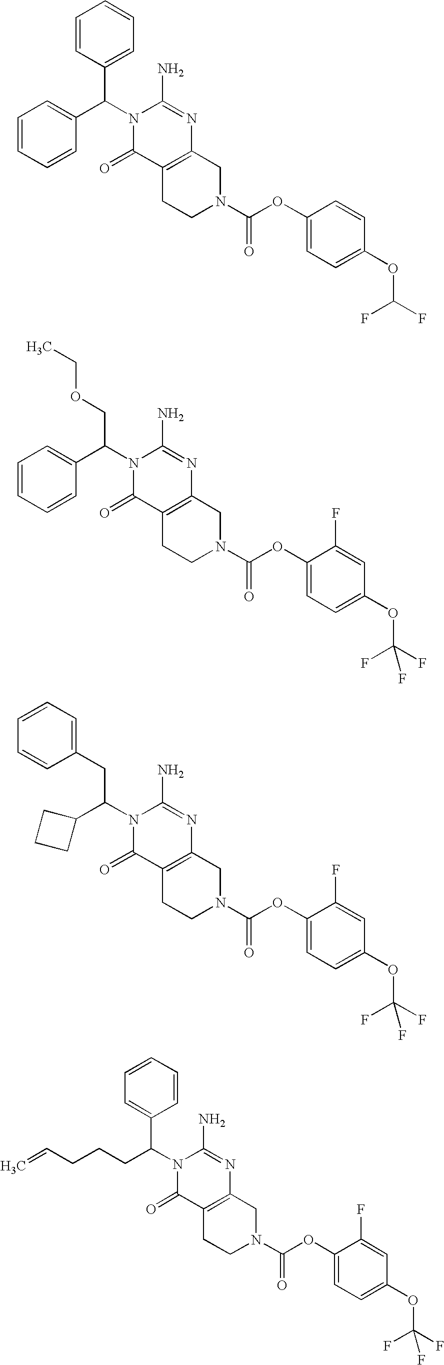 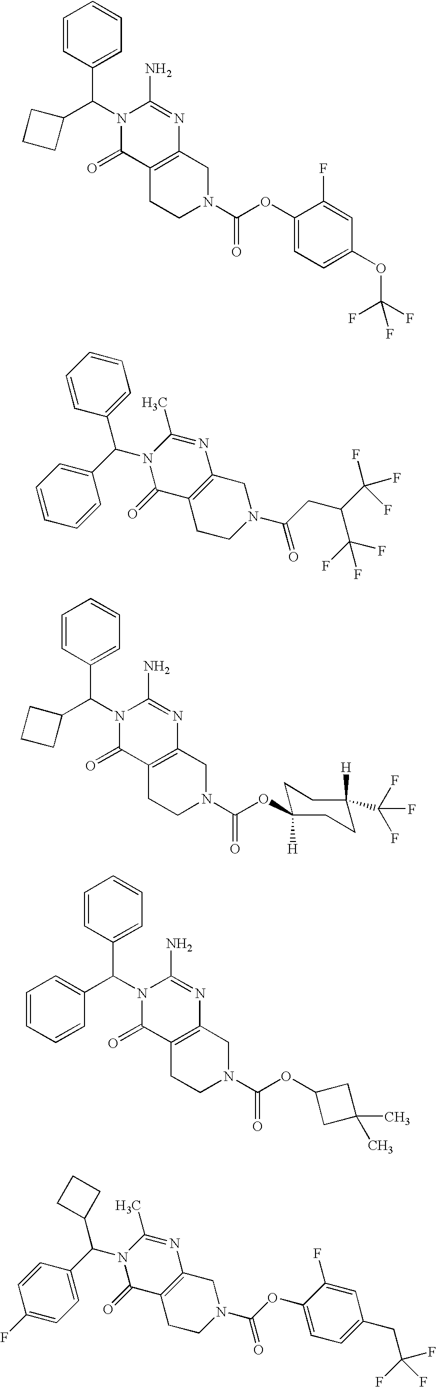 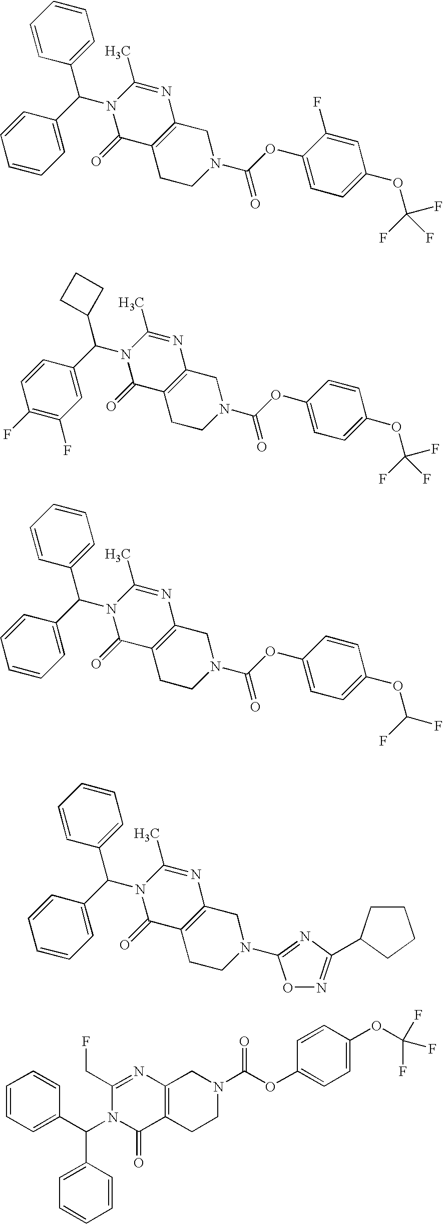 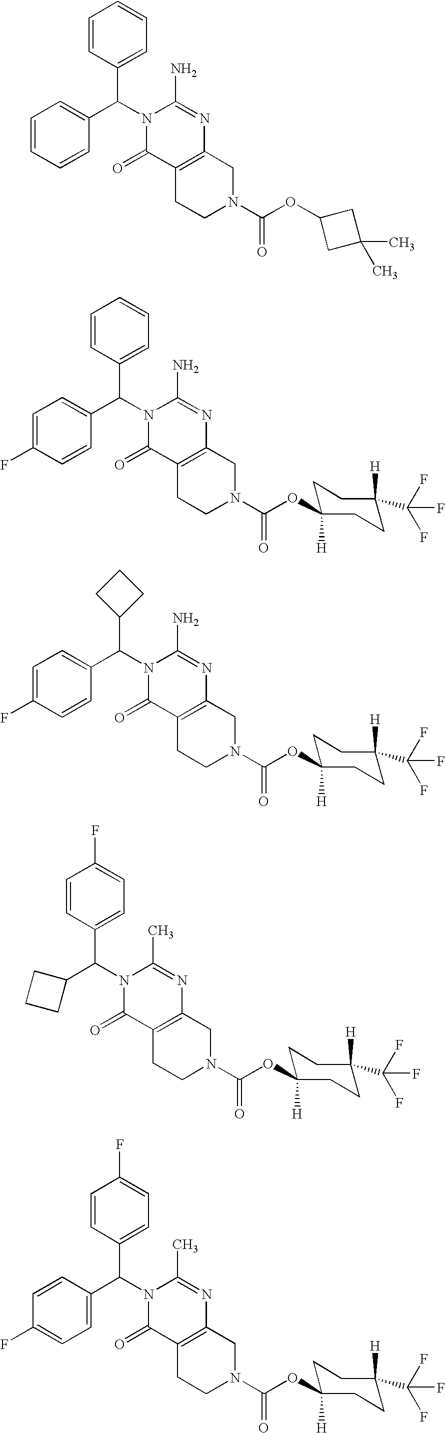 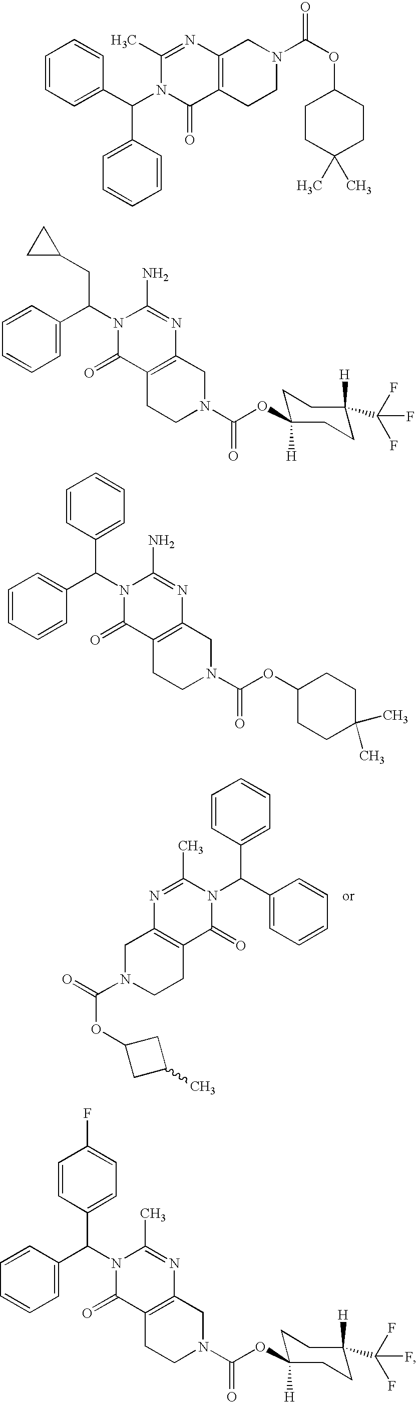
or a pharmaceutically acceptable salt, solvate, ester or prodrug thereof.

36. A pharmaceutical composition comprising one or more compounds of claim 1 or a pharmaceutically acceptable salt, solvate, ester or prodrug thereof, and at least one pharmaceutically acceptable carrier.

37. (canceled)

38. The composition of claim 36, further comprising at least one antidiabetic agent and/or at least one antiobesity agent that is different from the compounds of claim 1.

39-40. (canceled)

41. A method for treating diabetes, obesity or metabolic syndrome in a patient, the method comprising administering to the patient an effective amount of one or more compounds of claim 1 or a pharmaceutically acceptable salt, solvate, ester or prodrug thereof.

42-46. (canceled)

47. The method of claim 41, further comprising administering to the patient at least one antidiabetic agent and/or at least one antiobesity agent that is different from the compounds of claim 1, and wherein the amounts administered are together effective to treat diabetes, obesity or metabolic syndrome.

48-50. (canceled)

51. The method of claim 47, wherein the at least one antidiabetic agent is selected from an insulin sensitizer, a α-glucosidase inhibitor, a DPP-IV inhibitor, an insulin secretagogue, an hepatic glucose output lowering compound, an antihypertensive agent, a sodium glucose uptake transporter 2 (SGLT-2) inhibitor, an insulin-containing composition, and an antiobesity agent.

52-64. (canceled)

65. The method of claim 47, wherein the at least one antiobesity agent is selected from a neuropeptide Y antagonist, an MCR4 agonist, an MCH receptor antagonist, a protein hormone, an AMP kinase activator, a CB1 antagonist, a GLP-1 agonist and a lipase inhibitor.

66-67. (canceled)

68. The method of claim 41, wherein the treating is for diabetes.

69. The composition of claim 38, wherein the at least one antiobesity agent is selected from a neuropeptide Y antagonist, an MCR4 agonist, an MCH receptor antagonist, a protein hormone, an AMP kinase activator, a CB1 antagonist, a GLP-1 agonist and a lipase inhibitor.

70-71. (canceled)

72. The composition of claim 38, wherein the at least one antidiabetic agent is selected from an insulin sensitizer, a α-glucosidase inhibitor, a DPP-IV inhibitor, an insulin secretagogue, an hepatic glucose output lowering compound, an antihypertensive agent, a sodium glucose uptake transporter 2 (SGLT-2) inhibitor, insulin, an insulin-containing composition, and an antiobesity agent.

73-87. (canceled)
